SEQUENCE LISTING (461 sequences)

SEQ ID NO: 1-9 - skipped (9 entries)

SEQ ID NO: 10 - DNA, 834 bp, Homo sapiens
  source 1..834: mol_type = genomic DNA
gtagttgaaaagtggtcttgaaaaatactaaaatgaaggccactctatcagaatatcaaagtgtttctccttaatcacaaagagaaaacgagttaacctaaaaagattgtgaacacagtcattatgaaaataatgctctgaggtatcgaaaaagtatttgagattaattatcacatgaagggataacaagctaatttaaaaaactttttgaatacagtcataaactctccctaagactgtttaatttcttaaacatcttactttaaaaatgaatgcagtttagaagttgatatgctgtttgcacaaactagcagttgataagctaagattggaaatgaaattcagatagttaaaaaaagccttttcagtttcggtcagcctcgccttattttagaaacgcaaattgtccaggtgttgttttgctcagtagagcactttcagatctgggcctgggcaaaaccacctcttcacaaccagaagtgataaatttaccaattgtgtttttttgcttcctaaaatagactctcgcggtgacctgcttcctgccacctgctgtgggtgccggagacccccatgcagccatcttgactctaattcatcatctgcttccagcttcgctcaattaattaaaaaaataaacttgatttatgatggtcaaaacgcagtcccgcatcggggccgacagcactgtgctagtatttcttagctgagcttgctttggcctcaattccagacacatatcactcatgggtgttaatcaaatgataagaatttcaaatacttggacagttaaaaaaattaatatacttgaaaatctctcacatttttaagtca

SEQ ID NO: 11 - DNA, 1002 bp, Mus musculus
  source 1..1002: mol_type = genomic DNA
agtctagataattgcattcatttaaaaaaaaagtctttctcctaaaatgaatactcagaaagtggtcttgaaaaagatttgtgaagccgttttgaccagaatgtcaaagtcttaatagtaaggcaaaacaaacaactaaaaaagatcatgaacaaagtcactgtaaatgcttcgggtattggaaaagaattgaatggagaccaataatcagagggaagaataatagagtaattttaagaagttttctaaatatattagaaattaaagacactaaagtccttcaatttcttacataacctaattttgaaaatgaattctaaatacattttagaagtcgataaacttaagtttggggaaactagaactactcaagctaaaattaaaaggttgaactcaataagttaaaagaggacctctccagtttcggctgaatcctcaacttattttagaaatgcaaattacccaggtggtgttttgctcagcctggactttcggtttggtggggctggacagagtgtttcaaaaccacttcttcaaaccacagctacaagtttacctagtggttttattttcccttccccaaatagccttgccacatgacctgcttcctgccagctgctgcaggtgttctggttctgatcggccatcttgactccaactcaacattgctcaattcatttaaaaatatttgaaacttaatttattattgttaaaagtcagttctgaataggttatgagagagcctcactcccattcctcggttaaactttaagtaatatcagttctacacaaacaagacctcaaactgattgacaagaattttggacatttaaaaaaatgagtacttgaaaaccctctcacattttaaagtcacagtatttaactatttttcctaggaaccaacttaagagtaaaagcaacatcttctaatattccatacacatacttctgtgttcctttgaaagctggacttttgcaggctccaccagacctctctagaca

SEQ ID NO: 12 - DNA, 170 bp, Homo sapiens
  source 1..170: mol_type = genomic DNA
tgtaaccatatgcagcaattttcagccataccaggagaggtcaactgtaatcttggccacctgcctaagaggaagtggctagcttcacttctgaccctcagcaactgccaggtggcctcttggaaatccccctctgggggattccacccgttgggtgggagagcagtagt

SEQ ID NO: 13 - DNA, 295 bp, Mus musculus
  source 1..295: mol_type = genomic DNA
ttactacctctgtcacccaagagttggcatctcaacagaggggactttccgagaggccatctggcagttgcttaagatcagaagtgaagtctgccagttcctcccaggcaggtggcccagattacagttgacctgttctggtgtggctaaaaattgtcccatgtggttacaaaccattagaccagggtctgatgaattgctcagaatatttctggacacccaaatacagaccctggcttaaggccctgtccatacagtaggtttagcttggctacaccaaaggaagccatacaga

SEQ ID NO: 14 - DNA, 722 bp, Homo sapiens
  source 1..722: mol_type = genomic DNA
ctacatggacgtctggggcaaagggaccacggtcaccgtctcctcaggtaagaatggccactctagggcctttgttttctgctactgcctgtggggtttcctgagcattgcaggttggtcctcggggcatgttccgaggggacctgggcggactggccaggaggggatgggcactggggtgccttgaggatctgggagcctctgtggattttccgatgcctttggaaaatgggactcaggttgggtgcgtctgatggagtaactgagcctgggggcttggggagccacatttggacgagatgcctgaacaaaccaggggtcttagtgatggctgaggaatgtgtctcaggagcggtgtctgtaggactgcaagatcgctgcacagcagcgaatcgtgaaatattttctttagaattatgaggtgcgctgtgtgtcaacctgcatcttaaattctttattggctggaaagagaactgtcggagtgggtgaatccagccaggagggacgcgtagccccggtcttgatgagagcagggttgggggcaggggtagcccagaaacggtggctgccgtcctgacaggggcttagggaggctccaggacctcagtgccttgaagctggtttccatgagaaaaggattgtttatcttaggaggcatgcttactgttaaaagacaggatatgtttgaagtggcttctgagaaaaatggttaagaaaattat

SEQ ID NO: 15 - DNA, 1308 bp, Homo sapiens
  source 1..1308: mol_type = genomic DNA
ctcactttaggataagttttaggtaaaatgtgcatcattatcctgaattatttcagttaagcatgttagttggtggcataagagaaaactcaatcagatagtgctgaagacaggactgtggagacaccttagaaggacagattctgttccgaatcaccgatgcggcgtcagcaggactggcctagcggaggctctgggagggtggctgccaggcccggcctgggctttgggtctccccggactacccagagctgggatgcgtggcttctgctgccgggccgactggctgctcaggccccagcccttgttaatggacttggaggaatgattccatgccaaagctttgcaaggctcgcagtgaccaggcgcccgacatggtaagagacaggcagccgccgctgctgcatttgcttctcttaaaactttgtatttgacgtcttatttccactagaaggggaactggtcttaattgcttgatgaagagcaggagactcatttatgtgagtcttttgagtgaccattgtctgggtcactcccatttaactttccctaaagcccatttgaaggagaggtcgcacgagctgctccacaacctctgaatggggatggcatgggtaatgatgcttgagaacataccaagccccactggcatcgcccttgtctaagtcattgactgtaggtcatcatcgcacccttgaaagtagcccatgccttccaaagcgatttatggtaaatggcagaattttaagtggcaaattcagataaaatgcatttcttggttgtttccaatgatgactgttatctagagggaatttaaaggcaggggtttactgcagactcagaagggaggggatgctccgggaaggtggaggctctgagcatctcaataccctcctcttggtgcagaagatatgctgccacttctagagcaaggggacctgctcatttttatcacagcacaggctcctaaattcttggtctcattctcaagatgttttaatgactttaaagcagcaaagaaatattccacccaggtagtggagggtggtaatgattggtaatgctttggaaccaaaacccaggtggcgctggggcaggactgcagggaactggggtatcaagtagagggagacaaaagatggaagccagcctggctgtgcaggaacccggcaatgagatggctttagctgagacaagcaggtctggtgggctgaccatttctggccatgacaactccatccagctttcagaaatggactcagatgggcaaaactgacctaagctgacctagactaaacaaggctgaac

SEQ ID NO: 16 - DNA, 582 bp, Mus musculus
  source 1..582: mol_type = genomic DNA
ggactactggggtcaaggaacctcagtcaccgtctcctcaggtaagaatggcctctccaggtctttatttttaacctttgttatggagttttctgagcattgcagactaatcttggatatttgtccctgagggagccggctgagagaagttgggaaataaactgtctagggatctcagagcctttaggacagattatctccacatctttgaaaaactaagaatctgtgtgatggtgttggtggagtccctggatgatgggatagggactttggaggctcatttgaagaagatgctaaaacaatcctatggctggagggatagttggggctgtagttggagattttcagtttttagaataaaagtattagttgtggaatatacttcaggaccacctctgtgacagcatttatacagtatccgatgcatagggacaaagagtggagtggggcactttctttagatttgtgaggaatgttccgcactagattgtttaaaacttcatttgttggaaggagagctgtcttagtgattgagtcaagggagaaaggcatctagcctcggtctcaaaagggtagttgctg

SEQ ID NO: 17 - DNA, 1528 bp, Mus musculus
  source 1..1528: mol_type = genomic DNA
ttatttcagttgaacatgctggttggtggttgagaggacactcagtcagtcagtgacgtgaagggcttctaagccagtccacatgctctgtgtgaactccctctggccctgcttattgttgaatgggccaaaggtctgagaccaggctgctgctgggtaggcctggactttgggtctcccacccagacctgggaatgtatggttgtggcttctgccacccatccacctggctgctcatggaccagccagcctcggtggctttgaaggaacaattccacacaaagactctggacctctccgaaaccaggcaccgcaaatggtaagccagaggcagccacagctgtggctgctgctcttaaagcttgtaaactgtttctgcttaagagggactgagtcttcagtcattgctttagggggagaaagagacatttgtgtgtcttttgagtaccgttgtctgggtcactcacatttaactttccttgaaaaactagtaaaagaaaaatgttgcctgttaaccaataatcatagagctcatggtactttgaggaaatcttagaaagcgtgtatacaattgtctggaattatttcagttaagtgtattagttgaggtactgatgctgtctctacttcagttatacatgtgggtttgaattttgaatctattctggctcttcttaagcagaaaatttagataaaatggatacctcagtggtttttaatggtgggtttaatatagaaggaatttaaattggaagctaatttagaatcagtaaggagggacccaggctaagaaggcaatcctgggattctggaagaaaagatgtttttagtttttatagaaaacactactacattcttgatctacaactcaatgtggtttaatgaatttgaagttgccagtaaatgtacttcctggttgttaaagaatggtatcaaaggacagtgcttagatccgaggtgagtgtgagaggacaggggctggggtatggatacgcagaaggaaggccacagctgtacagaattgagaaagaatagagacctgcagttgaggccagcaggtcggctggactaactctccagccacagtaatgacccagacagagaaagccagactcataaagcttgctgagcaaaattaagggaacaaggttgagagccctagtaagcgaggctctaaaaagcacagctgagctgagatgggtgggcttctctgagtgcttctaaaatgcgctaaactgaggtgattactctgaggtaagcaaagctgggcttgagccaaaatgaagtagactgtaatgaactggaatgagctgggccgctaagctaaactaggctggcttaaccgagatgagccaaactggaatgaacttcattaatctaggttgaatagagctaaactctactgcctacactggactgttctgagctgagatgagctggggtgagctcagctatgctacgctgtgttggggtgagctgatctgaaatgagatactctggagtagctgagatggggtgagatggggtg

SEQ ID NO: 18 - DNA, 1130 bp, Homo sapiens
  source 1..1130: mol_type = genomic DNA
tagtcctatcaagttgttgcagaccaaaataatctccttaaatatcacttttgagatcagctggggtaaacgacagcaacacaatgacaaatcattaaactattttagagattatgaaattaaaatactcagattaaaattttcctatcacagaattaaggtactggaaaatatgtttaagtttttattaatcacattgctataggtttagatattttgtacaactgaaataaaatcacacactggcagctacatttttgaaagttaaaaacatggtcacgaatatatcttattttaaaatcagttaatataccttaatggtatttaatgccaaattcaaagtgaattgatcaagccctcagtggccaggtcatgggtgtgatttttactctgaaagaattacatatttctttctttttggttgagcttttgttatttaaatacatttgatgagaggatattgaaataattaaatagcactgaaaaaaaaaaaagctttaaattatttacaatcccctaatggaaattttcactaatgagatatcataatgaatgtgaattttatttctgaaatctctaataaatcagtcttctccctggttttcccagctcagcgcccattacgtttctgttctctttcccttagtggcattatttgtatcactgtgcatcaggaaagctggctacggcagcatcaatcgggcagacacagggtggccacggccactagcggcaaggcggctgccccaagagcgcggtggcatggccaccaaagccactcaatcgagaaagaccgcggctctgtctacagctcgcggtgccacggccttcttggcagaataaaaatgtagacaagtaataacagaggataatgaaagaacatactctttaaaatatttcctatttttttcacagacccacggtcattaaaaaatgcaattatttactttttttcatttaaacacatttctttgagattgagcttttgggaataaccacctttccaccattacaataagagataatttcacgtttagtctaatgtacaaattggatttttaaaaaatgagctctatctgtgaagcccttattcctatagaatgtgtctttttgagtttattacttattac

SEQ ID NO: 19 - DNA, 792 bp, Mus musculus
  source 1..792: mol_type = genomic DNA
ctgtcttgagttttgcaataaaagtgggatagcagagttgagtgagccgtaggctgagttctctcttttgtctcctaagtttttatgactacaaaaatcagtagtatgtcctgaaataatcattaagctgtttgaaagtatgactgcttgccatgtagataccatggcttgctgaataatcagaagaggtgtgactcttattctaaaatttgtcacaaaatgtcaaaatgagagactctgtaggaacgagtccttgacagacagctcaaggggtttttttcctttgtctcatttctacatgaaagtaaatttgaaatgatcttttttattataagagtagaaatacagttgggtttgaactatatgttttaatggccacggttttgtaagacatttggtcctttgttttcccagttattactcgattgtaattttatatcgccagcaatggactgaaacggtccgcaacctcttctttacaactgggtgacctcgcggctgtgccagccatttggcgttcaccctgccgctaagggccatgtgaacccccgcggtagcatcccttgctccgcgtggaccactttcctgaggcacagtgataggaacagagccactaatctgaagagaacagagatgtgacagactacactaatgtgagaaaaacaaggaaagggtgacttattggagatttcagaaataaaatgcatttattattatattcccttattttaattttctattagggaattagaaagggcataaactgctttatccagtgttatattaaaagct

SEQ ID NO: 20 - DNA, 23 bp, Homo sapiens
  source 1..23: mol_type = genomic DNA
ggtcctcggggcatgttccgagg

SEQ ID NO: 21 - DNA, 23 bp, Homo sapiens
  source 1..23: mol_type = genomic DNA
gggcatgttccgaggggacctgg

SEQ ID NO: 22 - DNA, 23 bp, Homo sapiens
  source 1..23: mol_type = genomic DNA
gcattgcaggttggtcctcgggg

SEQ ID NO: 23 - DNA, 23 bp, Homo sapiens
  source 1..23: mol_type = genomic DNA
tcctcggggcatgttccgagggg

SEQ ID NO: 24 - DNA, 23 bp, Homo sapiens
  source 1..23: mol_type = genomic DNA
ggcatgttccgaggggacctggg

SEQ ID NO: 25 - DNA, 23 bp, Homo sapiens
  source 1..23: mol_type = genomic DNA
gtctcaggagcggtgtctgtagg

SEQ ID NO: 26 - DNA, 23 bp, Homo sapiens
  source 1..23: mol_type = genomic DNA
agcattgcaggttggtcctcggg

SEQ ID NO: 27 - DNA, 23 bp, Homo sapiens
  source 1..23: mol_type = genomic DNA
cctgggcggactggccaggaggg

SEQ ID NO: 28 - DNA, 23 bp, Homo sapiens
  source 1..23: mol_type = genomic DNA
actggggtgccttgaggatctgg

SEQ ID NO: 29 - DNA, 23 bp, Homo sapiens
  source 1..23: mol_type = genomic DNA
ccccagtgcccatcccctcctgg

SEQ ID NO: 30 - DNA, 23 bp, Homo sapiens
  source 1..23: mol_type = genomic DNA
ctaagacccctggtttgttcagg

SEQ ID NO: 31 - DNA, 23 bp, Homo sapiens
  source 1..23: mol_type = genomic DNA
tgtggattttccgatgcctttgg

SEQ ID NO: 32 - DNA, 23 bp, Homo sapiens
  source 1..23: mol_type = genomic DNA
aggaccaacctgcaatgctcagg

SEQ ID NO: 33 - DNA, 23 bp, Homo sapiens
  source 1..23: mol_type = genomic DNA
ctcaggttgggtgcgtctgatgg

SEQ ID NO: 34 - DNA, 23 bp, Homo sapiens
  source 1..23: mol_type = genomic DNA
ccctcctggccagtccgcccagg

SEQ ID NO: 35 - DNA, 23 bp, Homo sapiens
  source 1..23: mol_type = genomic DNA
ggccaggaggggatgggcactgg

SEQ ID NO: 36 - DNA, 23 bp, Homo sapiens
  source 1..23: mol_type = genomic DNA
gagatgcctgaacaaaccagggg

SEQ ID NO: 37 - DNA, 23 bp, Homo sapiens
  source 1..23: mol_type = genomic DNA
aggggtcttagtgatggctgagg

SEQ ID NO: 38 - DNA, 23 bp, Homo sapiens
  source 1..23: mol_type = genomic DNA
atgggcactggggtgccttgagg

SEQ ID NO: 39 - DNA, 23 bp, Homo sapiens
  source 1..23: mol_type = genomic DNA
ttccgatgcctttggaaaatggg

SEQ ID NO: 40 - DNA, 23 bp, Homo sapiens
  source 1..23: mol_type = genomic DNA
ctgacgccgcatcggtgattcgg

SEQ ID NO: 41 - DNA, 23 bp, Homo sapiens
  source 1..23: mol_type = genomic DNA
ttagacaagggcgatgccagtgg

SEQ ID NO: 42 - DNA, 23 bp, Homo sapiens
  source 1..23: mol_type = genomic DNA
cgtgcgacctctccttcaaatgg

SEQ ID NO: 43 - DNA, 23 bp, Homo sapiens
  source 1..23: mol_type = genomic DNA
agcatatcttctgcaccaagagg

SEQ ID NO: 44 - DNA, 23 bp, Homo sapiens
  source 1..23: mol_type = genomic DNA
atattccacccaggtagtggagg

SEQ ID NO: 45 - DNA, 23 bp, Homo sapiens
  source 1..23: mol_type = genomic DNA
gtgcgacctctccttcaaatggg

SEQ ID NO: 46 - DNA, 23 bp, Homo sapiens
  source 1..23: mol_type = genomic DNA
aggtccccttgctctagaagtgg

SEQ ID NO: 47 - DNA, 23 bp, Homo sapiens
  source 1..23: mol_type = genomic DNA
ctctagataacagtcatcattgg

SEQ ID NO: 48 - DNA, 23 bp, Homo sapiens
  source 1..23: mol_type = genomic DNA
ttgtctaagtcattgactgtagg

SEQ ID NO: 49 - DNA, 23 bp, Homo sapiens
  source 1..23: mol_type = genomic DNA
ccaaagcgatttatggtaaatgg

SEQ ID NO: 50 - DNA, 23 bp, Homo sapiens
  source 1..23: mol_type = genomic DNA
tcttttgagtgaccattgtctgg

SEQ ID NO: 51 - DNA, 23 bp, Homo sapiens
  source 1..23: mol_type = genomic DNA
ccatttaccataaatcgctttgg

SEQ ID NO: 52 - DNA, 23 bp, Homo sapiens
  source 1..23: mol_type = genomic DNA
agggcgatgccagtggggcttgg

SEQ ID NO: 53 - DNA, 23 bp, Homo sapiens
  source 1..23: mol_type = genomic DNA
agctaaagccatctcattgccgg

SEQ ID NO: 54 - DNA, 23 bp, Homo sapiens
  source 1..23: mol_type = genomic DNA
ccacaacctctgaatggggatgg

SEQ ID NO: 55 - DNA, 23 bp, Homo sapiens
  source 1..23: mol_type = genomic DNA
ttaattgcttgatgaagagcagg

SEQ ID NO: 56 - DNA, 23 bp, Homo sapiens
  source 1..23: mol_type = genomic DNA
tagacaagggcgatgccagtggg

SEQ ID NO: 57 - DNA, 23 bp, Homo sapiens
  source 1..23: mol_type = genomic DNA
aagctgacctagactaaacaagg

SEQ ID NO: 58 - DNA, 23 bp, Homo sapiens
  source 1..23: mol_type = genomic DNA
gcaggaacccggcaatgagatgg

SEQ ID NO: 59 - DNA, 23 bp, Homo sapiens
  source 1..23: mol_type = genomic DNA
tctgttccgaatcaccgatgcgg

SEQ ID NO: 60 - DNA, 23 bp, Mus musculus
  source 1..23: mol_type = genomic DNA
caactacccttttgagaccgagg

SEQ ID NO: 61 - DNA, 23 bp, Mus musculus
  source 1..23: mol_type = genomic DNA
ttatacagtatccgatgcatagg

SEQ ID NO: 62 - DNA, 23 bp, Mus musculus
  source 1..23: mol_type = genomic DNA
tatacagtatccgatgcataggg

SEQ ID NO: 63 - DNA, 23 bp, Mus musculus
  source 1..23: mol_type = genomic DNA
catctagcctcggtctcaaaagg

SEQ ID NO: 64 - DNA, 23 bp, Mus musculus
  source 1..23: mol_type = genomic DNA
cactctttgtccctatgcatcgg

SEQ ID NO: 65 - DNA, 23 bp, Mus musculus
  source 1..23: mol_type = genomic DNA
atctagcctcggtctcaaaaggg

SEQ ID NO: 66 - DNA, 23 bp, Mus musculus
  source 1..23: mol_type = genomic DNA
aagttttaaacaatctagtgcgg

SEQ ID NO: 67 - DNA, 23 bp, Mus musculus
  source 1..23: mol_type = genomic DNA
aagatgctaaaacaatcctatgg

SEQ ID NO: 68 - DNA, 23 bp, Mus musculus
  source 1..23: mol_type = genomic DNA
tgctaaaacaatcctatggctgg

SEQ ID NO: 69 - DNA, 23 bp, Mus musculus
  source 1..23: mol_type = genomic DNA
aagtccctatcccatcatccagg

SEQ ID NO: 70 - DNA, 23 bp, Mus musculus
  source 1..23: mol_type = genomic DNA
gggagaaaggcatctagcctcgg

SEQ ID NO: 71 - DNA, 23 bp, Mus musculus
  source 1..23: mol_type = genomic DNA
tgagcattgcagactaatcttgg

SEQ ID NO: 72 - DNA, 23 bp, Mus musculus
  source 1..23: mol_type = genomic DNA
ttagttgtggaatatacttcagg

SEQ ID NO: 73 - DNA, 23 bp, Mus musculus
  source 1..23: mol_type = genomic DNA
tggtggagtccctggatgatggg

SEQ ID NO: 74 - DNA, 23 bp, Mus musculus
  source 1..23: mol_type = genomic DNA
gtggagataatctgtcctaaagg

SEQ ID NO: 75 - DNA, 23 bp, Mus musculus
  source 1..23: mol_type = genomic DNA
agtccctatcccatcatccaggg

SEQ ID NO: 76 - DNA, 23 bp, Mus musculus
  source 1..23: mol_type = genomic DNA
atcttggatatttgtccctgagg

SEQ ID NO: 77 - DNA, 23 bp, Mus musculus
  source 1..23: mol_type = genomic DNA
gggatagttggggctgtagttgg

SEQ ID NO: 78 - DNA, 23 bp, Mus musculus
  source 1..23: mol_type = genomic DNA
caggtaagaatggcctctccagg

SEQ ID NO: 79 - DNA, 23 bp, Mus musculus
  source 1..23: mol_type = genomic DNA
tctctcagccggctccctcaggg

SEQ ID NO: 80 - DNA, 23 bp, Mus musculus
  source 1..23: mol_type = genomic DNA
ccgaaaccaggcaccgcaaatgg

SEQ ID NO: 81 - DNA, 23 bp, Mus musculus
  source 1..23: mol_type = genomic DNA
caccgcaaatggtaagccagagg

SEQ ID NO: 82 - DNA, 23 bp, Mus musculus
  source 1..23: mol_type = genomic DNA
ggcttaccatttgcggtgcctgg

SEQ ID NO: 83 - DNA, 23 bp, Mus musculus
  source 1..23: mol_type = genomic DNA
tgcggtgcctggtttcggagagg

SEQ ID NO: 84 - DNA, 23 bp, Mus musculus
  source 1..23: mol_type = genomic DNA
cagctatgctacgctgtgttggg

SEQ ID NO: 85 - DNA, 23 bp, Mus musculus
  source 1..23: mol_type = genomic DNA
aaggacagtgcttagatccgagg

SEQ ID NO: 86 - DNA, 23 bp, Mus musculus
  source 1..23: mol_type = genomic DNA
tcagtcagtcagtgacgtgaagg

SEQ ID NO: 87 - DNA, 23 bp, Mus musculus
  source 1..23: mol_type = genomic DNA
catgctggttggtggttgagagg

SEQ ID NO: 88 - DNA, 23 bp, Mus musculus
  source 1..23: mol_type = genomic DNA
tcttttgagtaccgttgtctggg

SEQ ID NO: 89 - DNA, 23 bp, Mus musculus
  source 1..23: mol_type = genomic DNA
tggcccattcaacaataagcagg

SEQ ID NO: 90 - DNA, 23 bp, Mus musculus
  source 1..23: mol_type = genomic DNA
ctgggccgctaagctaaactagg

SEQ ID NO: 91 - DNA, 23 bp, Mus musculus
  source 1..23: mol_type = genomic DNA
gccagcctagtttagcttagcgg

SEQ ID NO: 92 - DNA, 23 bp, Mus musculus
  source 1..23: mol_type = genomic DNA
tgaagtagactgtaatgaactgg

SEQ ID NO: 93 - DNA, 23 bp, Mus musculus
  source 1..23: mol_type = genomic DNA
gacctgggaatgtatggttgtgg

SEQ ID NO: 94 - DNA, 23 bp, Mus musculus
  source 1..23: mol_type = genomic DNA
ggtatggatacgcagaaggaagg

SEQ ID NO: 95 - DNA, 23 bp, Mus musculus
  source 1..23: mol_type = genomic DNA
gttgagagccctagtaagcgagg

SEQ ID NO: 96 - DNA, 23 bp, Mus musculus
  source 1..23: mol_type = genomic DNA
gccgctaagctaaactaggctgg

SEQ ID NO: 97 - DNA, 23 bp, Mus musculus
  source 1..23: mol_type = genomic DNA
tcagctatgctacgctgtgttgg

SEQ ID NO: 98 - DNA, 23 bp, Mus musculus
  source 1..23: mol_type = genomic DNA
ttttagagcctcgcttactaggg

SEQ ID NO: 99 - DNA, 23 bp, Mus musculus
  source 1..23: mol_type = genomic DNA
ctctatgattattggttaacagg

SEQ ID NO: 100 - DNA, 23 bp, Homo sapiens
  source 1..23: mol_type = genomic DNA
gccatgccaccgcgctcttgggg

SEQ ID NO: 101 - DNA, 23 bp, Homo sapiens
  source 1..23: mol_type = genomic DNA
cactcaatcgagaaagaccgcgg

SEQ ID NO: 102 - DNA, 23 bp, Homo sapiens
  source 1..23: mol_type = genomic DNA
ggccacggccactagcggcaagg

SEQ ID NO: 103 - DNA, 23 bp, Homo sapiens
  source 1..23: mol_type = genomic DNA
gcatcaatcgggcagacacaggg

SEQ ID NO: 104 - DNA, 23 bp, Homo sapiens
  source 1..23: mol_type = genomic DNA
gcgagctgtagacagagccgcgg

SEQ ID NO: 105 - DNA, 23 bp, Homo sapiens
  source 1..23: mol_type = genomic DNA
cgcggtctttctcgattgagtgg

SEQ ID NO: 106 - DNA, 23 bp, Homo sapiens
  source 1..23: mol_type = genomic DNA
cgccttgccgctagtggccgtgg

SEQ ID NO: 107 - DNA, 23 bp, Homo sapiens
  source 1..23: mol_type = genomic DNA
gccccaagagcgcggtggcatgg

SEQ ID NO: 108 - DNA, 23 bp, Homo sapiens
  source 1..23: mol_type = genomic DNA
agcatcaatcgggcagacacagg

SEQ ID NO: 109 - DNA, 23 bp, Homo sapiens
  source 1..23: mol_type = genomic DNA
aaacgtaatgggcgctgagctgg

SEQ ID NO: 110 - DNA, 23 bp, Homo sapiens
  source 1..23: mol_type = genomic DNA
tctcgattgagtggctttggtgg

SEQ ID NO: 111 - DNA, 23 bp, Homo sapiens
  source 1..23: mol_type = genomic DNA
gtctacagctcgcggtgccacgg

SEQ ID NO: 112 - DNA, 23 bp, Homo sapiens
  source 1..23: mol_type = genomic DNA
cacggccactagcggcaaggcgg

SEQ ID NO: 113 - DNA, 23 bp, Homo sapiens
  source 1..23: mol_type = genomic DNA
ttggtctgcaacaacttgatagg

SEQ ID NO: 114 - DNA, 23 bp, Homo sapiens
  source 1..23: mol_type = genomic DNA
aacgtaatgggcgctgagctggg

SEQ ID NO: 115 - DNA, 23 bp, Homo sapiens
  source 1..23: mol_type = genomic DNA
ggccatgccaccgcgctcttggg

SEQ ID NO: 116 - DNA, 23 bp, Homo sapiens
  source 1..23: mol_type = genomic DNA
aattatttacaatcccctaatgg

SEQ ID NO: 117 - DNA, 23 bp, Homo sapiens
  source 1..23: mol_type = genomic DNA
aatcagttaatataccttaatgg

SEQ ID NO: 118 - DNA, 23 bp, Homo sapiens
  source 1..23: mol_type = genomic DNA
atatcacttttgagatcagctgg

SEQ ID NO: 119 - DNA, 23 bp, Homo sapiens
  source 1..23: mol_type = genomic DNA
gacacattctataggaataaggg

SEQ ID NO: 120 - DNA, 23 bp, Mus musculus
  source 1..23: mol_type = genomic DNA
tgctaccgcgggggttcacatgg

SEQ ID NO: 121 - DNA, 23 bp, Mus musculus
  source 1..23: mol_type = genomic DNA
agcaagggatgctaccgcggggg

SEQ ID NO: 122 - DNA, 23 bp, Mus musculus
  source 1..23: mol_type = genomic DNA
aagtggtccacgcggagcaaggg

SEQ ID NO: 123 - DNA, 23 bp, Mus musculus
  source 1..23: mol_type = genomic DNA
taattttatatcgccagcaatgg

SEQ ID NO: 124 - DNA, 23 bp, Mus musculus
  source 1..23: mol_type = genomic DNA
attacaatcgagtaataactggg

SEQ ID NO: 125 - DNA, 23 bp, Mus musculus
  source 1..23: mol_type = genomic DNA
ggagcaagggatgctaccgcggg

SEQ ID NO: 126 - DNA, 23 bp, Mus musculus
  source 1..23: mol_type = genomic DNA
cagggtgaacgccaaatggctgg

SEQ ID NO: 127 - DNA, 23 bp, Mus musculus
  source 1..23: mol_type = genomic DNA
gagcaagggatgctaccgcgggg

SEQ ID NO: 128 - DNA, 23 bp, Mus musculus
  source 1..23: mol_type = genomic DNA
aaatggctggcacagccgcgagg

SEQ ID NO: 129 - DNA, 23 bp, Mus musculus
  source 1..23: mol_type = genomic DNA
cggagcaagggatgctaccgcgg

SEQ ID NO: 130 - DNA, 23 bp, Mus musculus
  source 1..23: mol_type = genomic DNA
tcgccagcaatggactgaaacgg

SEQ ID NO: 131 - DNA, 23 bp, Mus musculus
  source 1..23: mol_type = genomic DNA
tcacatggcccttagcggcaggg

SEQ ID NO: 132 - DNA, 23 bp, Mus musculus
  source 1..23: mol_type = genomic DNA
aattacaatcgagtaataactgg

SEQ ID NO: 133 - DNA, 23 bp, Mus musculus
  source 1..23: mol_type = genomic DNA
ttcacatggcccttagcggcagg

SEQ ID NO: 134 - DNA, 23 bp, Mus musculus
  source 1..23: mol_type = genomic DNA
aaagtggtccacgcggagcaagg

SEQ ID NO: 135 - DNA, 23 bp, Mus musculus
  source 1..23: mol_type = genomic DNA
tctgattattcagcaagccatgg

SEQ ID NO: 136 - DNA, 23 bp, Mus musculus
  source 1..23: mol_type = genomic DNA
agcaagccatggtatctacatgg

SEQ ID NO: 137 - DNA, 23 bp, Mus musculus
  source 1..23: mol_type = genomic DNA
gtagcatcccttgctccgcgtgg

SEQ ID NO: 138 - DNA, 23 bp, Mus musculus
  source 1..23: mol_type = genomic DNA
ggcgttcaccctgccgctaaggg

SEQ ID NO: 139 - DNA, 23 bp, Mus musculus
  source 1..23: mol_type = genomic DNA
cctcaggaaagtggtccacgcgg

SEQ ID NO: 140 - RNA, 20 nt, Synthetic construct
  source 1..20: mol_type = other RNA
ggtcctcggggcatgttccg

SEQ ID NO: 141 - RNA, 20 nt, Synthetic construct
  source 1..20: mol_type = other RNA
gggcatgttccgaggggacc

SEQ ID NO: 142 - RNA, 20 nt, Synthetic construct
  source 1..20: mol_type = other RNA
gcattgcaggttggtcctcg

SEQ ID NO: 143 - RNA, 20 nt, Synthetic construct
  source 1..20: mol_type = other RNA
tcctcggggcatgttccgag

SEQ ID NO: 144 - RNA, 20 nt, Synthetic construct
  source 1..20: mol_type = other RNA
ggcatgttccgaggggacct

SEQ ID NO: 145 - RNA, 20 nt, Synthetic construct
  source 1..20: mol_type = other RNA
gtctcaggagcggtgtctgt

SEQ ID NO: 146 - RNA, 20 nt, Synthetic construct
  source 1..20: mol_type = other RNA
agcattgcaggttggtcctc

SEQ ID NO: 147 - RNA, 20 nt, Synthetic construct
  source 1..20: mol_type = other RNA
cctgggcggactggccagga

SEQ ID NO: 148 - RNA, 20 nt, Synthetic construct
  source 1..20: mol_type = other RNA
actggggtgccttgaggatc

SEQ ID NO: 149 - RNA, 20 nt, Synthetic construct
  source 1..20: mol_type = other RNA
ccccagtgcccatcccctcc

SEQ ID NO: 150 - RNA, 20 nt, Synthetic construct
  source 1..20: mol_type = other RNA
ctaagacccctggtttgttc

SEQ ID NO: 151 - RNA, 20 nt, Synthetic construct
  source 1..20: mol_type = other RNA
tgtggattttccgatgcctt

SEQ ID NO: 152 - RNA, 20 nt, Synthetic construct
  source 1..20: mol_type = other RNA
aggaccaacctgcaatgctc

SEQ ID NO: 153 - RNA, 20 nt, Synthetic construct
  source 1..20: mol_type = other RNA
ctcaggttgggtgcgtctga

SEQ ID NO: 154 - RNA, 20 nt, Synthetic construct
  source 1..20: mol_type = other RNA
ccctcctggccagtccgccc

SEQ ID NO: 155 - RNA, 20 nt, Synthetic construct
  source 1..20: mol_type = other RNA
ggccaggaggggatgggcac

SEQ ID NO: 156 - RNA, 20 nt, Synthetic construct
  source 1..20: mol_type = other RNA
gagatgcctgaacaaaccag

SEQ ID NO: 157 - RNA, 20 nt, Synthetic construct
  source 1..20: mol_type = other RNA
aggggtcttagtgatggctg

SEQ ID NO: 158 - RNA, 20 nt, Synthetic construct
  source 1..20: mol_type = other RNA
atgggcactggggtgccttg

SEQ ID NO: 159 - RNA, 20 nt, Synthetic construct
  source 1..20: mol_type = other RNA
ttccgatgcctttggaaaat

SEQ ID NO: 160 - RNA, 20 nt, Synthetic construct
  source 1..20: mol_type = other RNA
ctgacgccgcatcggtgatt

SEQ ID NO: 161 - RNA, 20 nt, Synthetic construct
  source 1..20: mol_type = other RNA
ttagacaagggcgatgccag

SEQ ID NO: 162 - RNA, 20 nt, Synthetic construct
  source 1..20: mol_type = other RNA
cgtgcgacctctccttcaaa

SEQ ID NO: 163 - RNA, 20 nt, Synthetic construct
  source 1..20: mol_type = other RNA
agcatatcttctgcaccaag

SEQ ID NO: 164 - RNA, 20 nt, Synthetic construct
  source 1..20: mol_type = other RNA
atattccacccaggtagtgg

SEQ ID NO: 165 - RNA, 20 nt, Synthetic construct
  source 1..20: mol_type = other RNA
gtgcgacctctccttcaaat

SEQ ID NO: 166 - RNA, 20 nt, Synthetic construct
  source 1..20: mol_type = other RNA
aggtccccttgctctagaag

SEQ ID NO: 167 - RNA, 20 nt, Synthetic construct
  source 1..20: mol_type = other RNA
ctctagataacagtcatcat

SEQ ID NO: 168 - RNA, 20 nt, Synthetic construct
  source 1..20: mol_type = other RNA
ttgtctaagtcattgactgt

SEQ ID NO: 169 - RNA, 20 nt, Synthetic construct
  source 1..20: mol_type = other RNA
ccaaagcgatttatggtaaa

SEQ ID NO: 170 - RNA, 20 nt, Synthetic construct
  source 1..20: mol_type = other RNA
tcttttgagtgaccattgtc

SEQ ID NO: 171 - RNA, 20 nt, Synthetic construct
  source 1..20: mol_type = other RNA
ccatttaccataaatcgctt

SEQ ID NO: 172 - RNA, 20 nt, Synthetic construct
  source 1..20: mol_type = other RNA
agggcgatgccagtggggct

SEQ ID NO: 173 - RNA, 20 nt, Synthetic construct
  source 1..20: mol_type = other RNA
agctaaagccatctcattgc

SEQ ID NO: 174 - RNA, 20 nt, Synthetic construct
  source 1..20: mol_type = other RNA
ccacaacctctgaatgggga

SEQ ID NO: 175 - RNA, 20 nt, Synthetic construct
  source 1..20: mol_type = other RNA
ttaattgcttgatgaagagc

SEQ ID NO: 176 - RNA, 20 nt, Synthetic construct
  source 1..20: mol_type = other RNA
tagacaagggcgatgccagt

SEQ ID NO: 177 - RNA, 20 nt, Synthetic construct
  source 1..20: mol_type = other RNA
aagctgacctagactaaaca

SEQ ID NO: 178 - RNA, 20 nt, Synthetic construct
  source 1..20: mol_type = other RNA
gcaggaacccggcaatgaga

SEQ ID NO: 179 - RNA, 20 nt, Synthetic construct
  source 1..20: mol_type = other RNA
tctgttccgaatcaccgatg

SEQ ID NO: 180 - RNA, 20 nt, Synthetic construct
  source 1..20: mol_type = other RNA
caactacccttttgagaccg

SEQ ID NO: 181 - RNA, 20 nt, Synthetic construct
  source 1..20: mol_type = other RNA
ttatacagtatccgatgcat

SEQ ID NO: 182 - RNA, 20 nt, Synthetic construct
  source 1..20: mol_type = other RNA
tatacagtatccgatgcata

SEQ ID NO: 183 - RNA, 20 nt, Synthetic construct
  source 1..20: mol_type = other RNA
catctagcctcggtctcaaa

SEQ ID NO: 184 - RNA, 20 nt, Synthetic construct
  source 1..20: mol_type = other RNA
cactctttgtccctatgcat

SEQ ID NO: 185 - RNA, 20 nt, Synthetic construct
  source 1..20: mol_type = other RNA
atctagcctcggtctcaaaa

SEQ ID NO: 186 - RNA, 20 nt, Synthetic construct
  source 1..20: mol_type = other RNA
aagttttaaacaatctagtg

SEQ ID NO: 187 - RNA, 20 nt, Synthetic construct
  source 1..20: mol_type = other RNA
aagatgctaaaacaatccta

SEQ ID NO: 188 - RNA, 20 nt, Synthetic construct
  source 1..20: mol_type = other RNA
tgctaaaacaatcctatggc

SEQ ID NO: 189 - RNA, 20 nt, Synthetic construct
  source 1..20: mol_type = other RNA
aagtccctatcccatcatcc

SEQ ID NO: 190 - RNA, 20 nt, Synthetic construct
  source 1..20: mol_type = other RNA
gggagaaaggcatctagcct

SEQ ID NO: 191 - RNA, 20 nt, Synthetic construct
  source 1..20: mol_type = other RNA
tgagcattgcagactaatct

SEQ ID NO: 192 - RNA, 20 nt, Synthetic construct
  source 1..20: mol_type = other RNA
ttagttgtggaatatacttc

SEQ ID NO: 193 - RNA, 20 nt, Synthetic construct
  source 1..20: mol_type = other RNA
tggtggagtccctggatgat

SEQ ID NO: 194 - RNA, 20 nt, Synthetic construct
  source 1..20: mol_type = other RNA
gtggagataatctgtcctaa

SEQ ID NO: 195 - RNA, 20 nt, Synthetic construct
  source 1..20: mol_type = other RNA
agtccctatcccatcatcca

SEQ ID NO: 196 - RNA, 20 nt, Synthetic construct
  source 1..20: mol_type = other RNA
atcttggatatttgtccctg

SEQ ID NO: 197 - RNA, 20 nt, Synthetic construct
  source 1..20: mol_type = other RNA
gggatagttggggctgtagt

SEQ ID NO: 198 - RNA, 20 nt, Synthetic construct
  source 1..20: mol_type = other RNA
caggtaagaatggcctctcc

SEQ ID NO: 199 - RNA, 20 nt, Synthetic construct
  source 1..20: mol_type = other RNA
tctctcagccggctccctca

SEQ ID NO: 200 - RNA, 20 nt, Synthetic construct
  source 1..20: mol_type = other RNA
ccgaaaccaggcaccgcaaa

SEQ ID NO: 201 - RNA, 20 nt, Synthetic construct
  source 1..20: mol_type = other RNA
caccgcaaatggtaagccag

SEQ ID NO: 202 - RNA, 20 nt, Synthetic construct
  source 1..20: mol_type = other RNA
ggcttaccatttgcggtgcc

SEQ ID NO: 203 - RNA, 20 nt, Synthetic construct
  source 1..20: mol_type = other RNA
tgcggtgcctggtttcggag

SEQ ID NO: 204 - RNA, 20 nt, Synthetic construct
  source 1..20: mol_type = other RNA
cagctatgctacgctgtgtt

SEQ ID NO: 205 - RNA, 20 nt, Synthetic construct
  source 1..20: mol_type = other RNA
aaggacagtgcttagatccg

SEQ ID NO: 206 - RNA, 20 nt, Synthetic construct
  source 1..20: mol_type = other RNA
tcagtcagtcagtgacgtga

SEQ ID NO: 207 - RNA, 20 nt, Synthetic construct
  source 1..20: mol_type = other RNA
catgctggttggtggttgag

SEQ ID NO: 208 - RNA, 20 nt, Synthetic construct
  source 1..20: mol_type = other RNA
tcttttgagtaccgttgtct

SEQ ID NO: 209 - RNA, 20 nt, Synthetic construct
  source 1..20: mol_type = other RNA
tggcccattcaacaataagc

SEQ ID NO: 210 - RNA, 20 nt, Synthetic construct
  source 1..20: mol_type = other RNA
ctgggccgctaagctaaact

SEQ ID NO: 211 - RNA, 20 nt, Synthetic construct
  source 1..20: mol_type = other RNA
gccagcctagtttagcttag

SEQ ID NO: 212 - RNA, 20 nt, Synthetic construct
  source 1..20: mol_type = other RNA
tgaagtagactgtaatgaac

SEQ ID NO: 213 - RNA, 20 nt, Synthetic construct
  source 1..20: mol_type = other RNA
gacctgggaatgtatggttg

SEQ ID NO: 214 - RNA, 20 nt, Synthetic construct
  source 1..20: mol_type = other RNA
ggtatggatacgcagaagga

SEQ ID NO: 215 - RNA, 20 nt, Synthetic construct
  source 1..20: mol_type = other RNA
gttgagagccctagtaagcg

SEQ ID NO: 216 - RNA, 20 nt, Synthetic construct
  source 1..20: mol_type = other RNA
gccgctaagctaaactaggc

SEQ ID NO: 217 - RNA, 20 nt, Synthetic construct
  source 1..20: mol_type = other RNA
tcagctatgctacgctgtgt

SEQ ID NO: 218 - RNA, 20 nt, Synthetic construct
  source 1..20: mol_type = other RNA
ttttagagcctcgcttacta

SEQ ID NO: 219 - RNA, 20 nt, Synthetic construct
  source 1..20: mol_type = other RNA
ctctatgattattggttaac

SEQ ID NO: 220 - RNA, 20 nt, Synthetic construct
  source 1..20: mol_type = other RNA
gccatgccaccgcgctcttg

SEQ ID NO: 221 - RNA, 20 nt, Synthetic construct
  source 1..20: mol_type = other RNA
cactcaatcgagaaagaccg

SEQ ID NO: 222 - RNA, 20 nt, Synthetic construct
  source 1..20: mol_type = other RNA
ggccacggccactagcggca

SEQ ID NO: 223 - RNA, 20 nt, Synthetic construct
  source 1..20: mol_type = other RNA
gcatcaatcgggcagacaca

SEQ ID NO: 224 - RNA, 20 nt, Synthetic construct
  source 1..20: mol_type = other RNA
gcgagctgtagacagagccg

SEQ ID NO: 225 - RNA, 20 nt, Synthetic construct
  source 1..20: mol_type = other RNA
cgcggtctttctcgattgag

SEQ ID NO: 226 - RNA, 20 nt, Synthetic construct
  source 1..20: mol_type = other RNA
cgccttgccgctagtggccg

SEQ ID NO: 227 - RNA, 20 nt, Synthetic construct
  source 1..20: mol_type = other RNA
gccccaagagcgcggtggca

SEQ ID NO: 228 - RNA, 20 nt, Synthetic construct
  source 1..20: mol_type = other RNA
agcatcaatcgggcagacac

SEQ ID NO: 229 - RNA, 20 nt, Synthetic construct
  source 1..20: mol_type = other RNA
aaacgtaatgggcgctgagc

SEQ ID NO: 230 - RNA, 20 nt, Synthetic construct
  source 1..20: mol_type = other RNA
tctcgattgagtggctttgg

SEQ ID NO: 231 - RNA, 20 nt, Synthetic construct
  source 1..20: mol_type = other RNA
gtctacagctcgcggtgcca

SEQ ID NO: 232 - RNA, 20 nt, Synthetic construct
  source 1..20: mol_type = other RNA
cacggccactagcggcaagg

SEQ ID NO: 233 - RNA, 20 nt, Synthetic construct
  source 1..20: mol_type = other RNA
ttggtctgcaacaacttgat

SEQ ID NO: 234 - RNA, 20 nt, Synthetic construct
  source 1..20: mol_type = other RNA
aacgtaatgggcgctgagct

SEQ ID NO: 235 - RNA, 20 nt, Synthetic construct
  source 1..20: mol_type = other RNA
ggccatgccaccgcgctctt

SEQ ID NO: 236 - RNA, 20 nt, Synthetic construct
  source 1..20: mol_type = other RNA
aattatttacaatcccctaa

SEQ ID NO: 237 - RNA, 20 nt, Synthetic construct
  source 1..20: mol_type = other RNA
aatcagttaatataccttaa

SEQ ID NO: 238 - RNA, 20 nt, Synthetic construct
  source 1..20: mol_type = other RNA
atatcacttttgagatcagc

SEQ ID NO: 239 - RNA, 20 nt, Synthetic construct
  source 1..20: mol_type = other RNA
gacacattctataggaataa

SEQ ID NO: 240 - RNA, 20 nt, Synthetic construct
  source 1..20: mol_type = other RNA
tgctaccgcgggggttcaca

SEQ ID NO: 241 - RNA, 20 nt, Synthetic construct
  source 1..20: mol_type = other RNA
agcaagggatgctaccgcgg

SEQ ID NO: 242 - RNA, 20 nt, Synthetic construct
  source 1..20: mol_type = other RNA
aagtggtccacgcggagcaa

SEQ ID NO: 243 - RNA, 20 nt, Synthetic construct
  source 1..20: mol_type = other RNA
taattttatatcgccagcaa

SEQ ID NO: 244 - RNA, 20 nt, Synthetic construct
  source 1..20: mol_type = other RNA
attacaatcgagtaataact

SEQ ID NO: 245 - RNA, 20 nt, Synthetic construct
  source 1..20: mol_type = other RNA
ggagcaagggatgctaccgc

SEQ ID NO: 246 - RNA, 20 nt, Synthetic construct
  source 1..20: mol_type = other RNA
cagggtgaacgccaaatggc

SEQ ID NO: 247 - RNA, 20 nt, Synthetic construct
  source 1..20: mol_type = other RNA
gagcaagggatgctaccgcg

SEQ ID NO: 248 - RNA, 20 nt, Synthetic construct
  source 1..20: mol_type = other RNA
aaatggctggcacagccgcg

SEQ ID NO: 249 - RNA, 20 nt, Synthetic construct
  source 1..20: mol_type = other RNA
cggagcaagggatgctaccg

SEQ ID NO: 250 - RNA, 20 nt, Synthetic construct
  source 1..20: mol_type = other RNA
tcgccagcaatggactgaaa

SEQ ID NO: 251 - RNA, 20 nt, Synthetic construct
  source 1..20: mol_type = other RNA
tcacatggcccttagcggca

SEQ ID NO: 252 - RNA, 20 nt, Synthetic construct
  source 1..20: mol_type = other RNA
aattacaatcgagtaataac

SEQ ID NO: 253 - RNA, 20 nt, Synthetic construct
  source 1..20: mol_type = other RNA
ttcacatggcccttagcggc

SEQ ID NO: 254 - RNA, 20 nt, Synthetic construct
  source 1..20: mol_type = other RNA
aaagtggtccacgcggagca

SEQ ID NO: 255 - RNA, 20 nt, Synthetic construct
  source 1..20: mol_type = other RNA
tctgattattcagcaagcca

SEQ ID NO: 256 - RNA, 20 nt, Synthetic construct
  source 1..20: mol_type = other RNA
agcaagccatggtatctaca

SEQ ID NO: 257 - RNA, 20 nt, Synthetic construct
  source 1..20: mol_type = other RNA
gtagcatcccttgctccgcg

SEQ ID NO: 258 - RNA, 20 nt, Synthetic construct
  source 1..20: mol_type = other RNA
ggcgttcaccctgccgctaa

SEQ ID NO: 259 - RNA, 20 nt, Synthetic construct
  source 1..20: mol_type = other RNA
cctcaggaaagtggtccacg

SEQ ID NO: 260 - DNA, 36 bp, Synthetic construct
  source 1..36: mol_type = other DNA
catcggatactgtataaatgctgtcacagaggtggt

SEQ ID NO: 261 - DNA, 36 bp, Synthetic construct
  source 1..36: mol_type = other DNA
catagggacaaagagtggagtggggcactttcttta

SEQ ID NO: 262 - DNA, 36 bp, Synthetic construct
  source 1..36: mol_type = other DNA
gacaccgctcctgagacacattcctcagccatcact

SEQ ID NO: 263 - DNA, 36 bp, Synthetic construct
  source 1..36: mol_type = other DNA
tgtaggactgcaagatcgctgcacagcagcgaatcg

SEQ ID NO: 264 - DNA, 37 bp, Synthetic construct
  source 1..37: mol_type = other DNA
gggaccaacctgcaatgctcaggaaaccccacaggca

SEQ ID NO: 265 - DNA, 37 bp, Synthetic construct
  source 1..37: mol_type = other DNA
ttcggggcatgttccgaggggacctgggcggactggc

SEQ ID NO: 266 - DNA, 36 bp, Synthetic construct
  source 1..36: mol_type = other DNA
tattactcgattgtaattttatatcgccagcaatgg

SEQ ID NO: 267 - DNA, 36 bp, Synthetic construct
  source 1..36: mol_type = other DNA
actgggaaaacaaaggaccaaatgtcttacaaaacc

SEQ ID NO: 268 - DNA, 36 bp, Synthetic construct
  source 1..36: mol_type = other DNA
ctccgcgtggaccactttcctgaggcacagtgatag

SEQ ID NO: 269 - DNA, 36 bp, Synthetic construct
  source 1..36: mol_type = other DNA
caagggatgctaccgcgggggttcacatggccctta

SEQ ID NO: 270 - DNA, 36 bp, Synthetic construct
  source 1..36: mol_type = other DNA
cggtagcatcccttgctccgcgtggaccactttcct

SEQ ID NO: 271 - DNA, 36 bp, Synthetic construct
  source 1..36: mol_type = other DNA
cgggggttcacatggcccttagcggcagggtgaacg

SEQ ID NO: 272 - DNA, 36 bp, Synthetic construct
  source 1..36: mol_type = other DNA
aatcgagaaagaccgcggctctgtctacagctcgcg

SEQ ID NO: 273 - DNA, 36 bp, Synthetic construct
  source 1..36: mol_type = other DNA
gagtggctttggtggccatgccaccgcgctcttggg

SEQ ID NO: 274 - DNA, 36 bp, Synthetic construct
  source 1..36: mol_type = other DNA
ctctgtctacagctcgcggtgccacggccttcttgg

SEQ ID NO: 275 - DNA, 36 bp, Synthetic construct
  source 1..36: mol_type = other DNA
ccgcggtctttctcgattgagtggctttggtggcca

SEQ ID NO: 276 - DNA, 36 bp, Synthetic construct
  source 1..36: mol_type = other DNA
aggccgtggcaccgcgagctgtagacagagccgcgg

SEQ ID NO: 277 - DNA, 36 bp, Synthetic construct
  source 1..36: mol_type = other DNA
gtggcatggccaccaaagccactcaatcgagaaaga

SEQ ID NO: 278 - DNA, 127 bp, Synthetic construct
  source 1..127: mol_type = other DNA
cttcgagacatgtacagaccatttagatgtagtatcaaagcctaatatctcaatcttaaaatagaatcctaacctgagacactcacttgtccatcggatactgtataaatgctgtcacagaggtggt

SEQ ID NO: 279 - DNA, 86 bp, Synthetic construct
  source 1..86: mol_type = other DNA
cttctcccattctaaatgcatgttggggggattctgggccttcaggaccacatagggacaaagagtggagtggggcactttcttta

SEQ ID NO: 280 - DNA, 127 bp, Synthetic construct
  source 1..127: mol_type = other DNA
gtgcacagcgctcttcccgctgcagaacaaaccccaaccccaggatgcactcctcactgtgaacccacattttattggcctaaagattacggacaccgctcctgagacacattcctcagccatcact

SEQ ID NO: 281 - DNA, 86 bp, Synthetic construct
  source 1..86: mol_type = other DNA
gtctggggatagcggggagccaggtgtactgggccaggcaagggctttggtgtaggactgcaagatcgctgcacagcagcgaatcg

SEQ ID NO: 282 - DNA, 128 bp, Synthetic construct
  source 1..128: mol_type = other DNA
gtgcacagcgctcttcccgctgcagaacaaaccccaaccccaggatgcactcctcactgtgaacccacattttattggcctaaagattacggggaccaacctgcaatgctcaggaaaccccacaggca

SEQ ID NO: 283 - DNA, 87 bp, Synthetic construct
  source 1..87: mol_type = other DNA
gtctggggatagcggggagccaggtgtactgggccaggcaagggctttggttcggggcatgttccgaggggacctgggcggactggc

SEQ ID NO: 284 - protein, 536 aa, Homo sapiens
MEFSSPSREECPKPLSRVSIMAGSLTGLLLLQAVSWASGARPCIPKSFGYSSVVCVCNATYCDSFDPPTFPALGTFSRYESTRSGRRMELSMGPIQANHTGTGLLLTLQPEQKFQKVKGFGGAMTDAAALNILALSPPAQNLLLKSYFSEEGIGYNIIRVPMASCDFSIRTYTYADTPDDFQLHNFSLPEEDTKLKIPLIHRALQLAQRPVSLLASPWTSPTWLKTNGAVNGKGSLKGQPGDIYHQTWARYFVKFLDAYAEHKLQFWAVTAENEPSAGLLSGYPFQCLGFTPEHQRDFIARDLGPTLANSTHHNVRLLMLDDQRLLLPHWAKVVLTDPEAAKYVHGIAVHWYLDFLAPAKATLGETHRLFPNTMLFASEACVGSKFWEQSVRLGSWDRGMQYSHSIITNLLYHVVGWTDWNLALNPEGGPNWVRNFVDSPIIVDITKDTFYKQPMFYHLGHFSKFIPEGSQRVGLVASQKNDLDAVALMHPDGSAVVVVLNRSSKDVPLTIKDPAVGFLETISPGYSIHTYLWRRQ

SEQ ID NO: 285 - protein, 629 aa, Homo sapiens
MPRYGASLRQSCPRSGREQGQDGTAGAPGLLWMGLVLALALALALALSDSRVLWAPAEAHPLSPQGHPARLHRIVPRLRDVFGWGNLTCPICKGLFTAINLGLKKEPNVARVGSVAIKLCNLLKIAPPAVCQSIVHLFEDDMVEVWRRSVLSPSEACGLLLGSTCGHWDIFSSWNISLPTVPKPPPKPPSPPAPGAPVSRILFLTDLHWDHDYLEGTDPDCADPLCCRRGSGLPPASRPGAGYWGEYSKCDLPLRTLESLLSGLGPAGPFDMVYWTGDIPAHDVWHQTRQDQLRALTTVTALVRKFLGPVPVYPAVGNHESIPVNSFPPPFIEGNHSSRWLYEAMAKAWEPWLPAEALRTLRIGGFYALSPYPGLRLISLNMNFCSRENFWLLINSTDPAGQLQWLVGELQAAEDRGDKVHIIGHIPPGHCLKSWSWNYYRIVARYENTLAAQFFGHTHVDEFEVFYDEETLSRPLAVAFLAPSATTYIGLNPGYRVYQIDGNYSRSSHVVLDHETYILNLTQANIPGAIPHWQLLYRARETYGLPNTLPTAWHNLVYRMRGDMQLFQTFWFLYHKGHPPSEPCGTPCRLATLCAQLSARADSPALCRHLMPDGSLPEAQSLWPRPLFC

SEQ ID NO: 286 - protein, 952 aa, Homo sapiens
MGVRHPPCSHRLLAVCALVSLATAALLGHILLHDFLLVPRELSGSSPVLEETHPAHQQGASRPGPRDAQAHPGRPRAVPTQCDVPPNSRFDCAPDKAITQEQCEARGCCYIPAKQGLQGAQMGQPWCFFPPSYPSYKLENLSSSEMGYTATLTRTTPTFFPKDILTLRLDVMMETENRLHFTIKDPANRRYEVPLETPHVHSRAPSPLYSVEFSEEPFGVIVRRQLDGRVLLNTTVAPLFFADQFLQLSTSLPSQYITGLAEHLSPLMLSTSWTRITLWNRDLAPTPGANLYGSHPFYLALEDGGSAHGVFLLNSNAMDVVLQPSPALSWRSTGGILDVYIFLGPEPKSVVQQYLDVVGYPFMPPYWGLGFHLCRWGYSSTAITRQVVENMTRAHFPLDVQWNDLDYMDSRRDFTFNKDGFRDFPAMVQELHQGGRRYMMIVDPAISSSGPAGSYRPYDEGLRRGVFITNETGQPLIGKVWPGSTAFPDFTNPTALAWWEDMVAEFHDQVPFDGMWIDMNEPSNFIRGSEDGCPNNELENPPYVPGVVGGTLQAATICASSHQFLSTHYNLHNLYGLTEAIASHRALVKARGTRPFVISRSTFAGHGRYAGHWTGDVWSSWEQLASSVPEILQFNLLGVPLVGADVCGFLGNTSEELCVRWTQLGAFYPFMRNHNSLLSLPQEPYSFSEPAQQAMRKALTLRYALLPHLYTLFHQAHVAGETVARPLFLEFPKDSSTWTVDHQLLWGEALLITPVLQAGKAEVTGYFPLGTWYDLQTVPVEALGSLPPPPAAPREPAIHSEGQWVTLPAPLDTINVHLRAGYIIPLQGPGLTTTESRQQPMALAVALTKGGEARGELFWDDGESLEVLERGAYTQVIFLARNNTIVNELVRVTSEGAGLQLQKVTVLGVATAPQQVLSNGVPVSNFTYSPDTKVLDICVSLLMGEQFLVSWC

SEQ ID NO: 287 - protein, 429 aa, Homo sapiens
MQLRNPELHLGCALALRFLALVSWDIPGARALDNGLARTPTMGWLHWERFMCNLDCQEEPDSCISEKLFMEMAELMVSEGWKDAGYEYLCIDDCWMAPQRDSEGRLQADPQRFPHGIRQLANYVHSKGLKLGIYADVGNKTCAGFPGSFGYYDIDAQTFADWGVDLLKFDGCYCDSLENLADGYKHMSLALNRTGRSIVYSCEWPLYMWPFQKPNYTEIRQYCNHWRNFADIDDSWKSIKSILDWTSFNQERIVDVAGPGGWNDPDMLVIGNFGLSWNQQVTQMALWAIMAAPLFMSNDLRHISPQAKALLQDKDVIAINQDPLGKQGYQLRQGDNFEVWERPLSGLAWAVAMINRQEIGGPRSYTIAVASLGKGVACNPACFITQLLPVKRKLGFYEWTSRLRSHINPTGTVLLQLENTMQMSLKDLL

SEQ ID NO: 288 - protein, 2351 aa, Homo sapiens [sequence omitted]

SEQ ID NO: 289 - protein, 456 aa, Homo sapiens
MIMAESPGLITICLLGYLLSAECTVFLDHENANKILNRPKRYNSGKLEEFVQGNLERECMEEKCSFEEAREVFENTERTTEFWKQYVDGDQCESNPCLNGGSCKDDINSYECWCPFGFEGKNCELDVTCNIKNGTCEQFCKNSADNKVVCSCTEGYRLAENQKSCEPAVPFPCGRVSVSQTSKLTRAEAVFPDVDYVNSTEAETILDNITQSTQSFNDFTRVVGGEDAKPGQFPWQVVLNGKVDAFCGGSIVNEKWIVTAAHCVETGVKITVVAGEHNIEETEHTEQKRNVIRIIPHHNYNAAINKYNHDIALLELDEPLVLNSYVTPICIADKEYTNIFLKFGSGYVSGWGRVFHKGRSALVLQYLRVPLVDRATCLRSTKFTIYNNMFCAGLHEGARDSCQGDSGGPHVTEVEGTSFLTGIISWGEECAMKGKYGIYTKVSRYVNWIKEKTKLT

SEQ ID NO: 290 - protein, 625 aa, Homo sapiens
MIFLYQVVHFILFTSVSGECVTQLLKDTCFEGGDITTVFTPSAKYCQVVCTYHPRCLLFTFTAESPSEDPTRWFTCVLKDSVTETLPRVNRTAAISGYSFKQCSHQISACNKDIYVDLDMKGINYNSSVAKSAQECQERCTDDVHCHFFTYATRQFPSLEHRNICLLKHTQTGTPTRITKLDKVVSGFSLKSCALSNLACIRDIFPNTVFADSNIDSVMAPDAFVCGRICTHHPGCLFFTFFSQEWPKESQRNLCLLKTSESGLPSTRIKKSKALSGFSLQSCRHSIPVFCHSSFYHDTDFLGEELDIVAAKSHEACQKLCTNAVRCQFFTYTPAQASCNEGKGKCYLKLSSNGSPTKILHGRGGISGYTLRLCKMDNECTTKIKPRIVGGTASVRGEWPWQVTLHTTSPTQRHLCGGSIIGNQWILTAAHCFYGVESPKILRVYSGILNQSEIKEDTSFFGVQEIIIHDQYKMAESGYDIALLKLETTVNYTDSQRPICLPSKGDRNVIYTDCWVTGWGYRKLRDKIQNTLQKAKIPLVTNEECQKRYRGHKITHKMICAGYREGGKDACKGDSGGPLSCKHNEVWHLVGITSWGEGCAQRERPGVYTNVVEYVDWILEKTQAV

SEQ ID NO: 291 - protein, 110 aa, Homo sapiens
MALWMRLLPLLALLALWGPDPAAAFVNQHLCGSHLVEALYLVCGERGFFYTPKTRREAEDLQVGQVELGGGPGAGSLQPLALEGSLQKRGIVEQCCTSICSLYQLENYCN

SEQ ID NO: 292 - protein, 418 aa, Homo sapiens [sequence omitted]

SEQ ID NO: 293 - DNA, 6991 bp, Synthetic construct
  source 1..6991: mol_type = other DNA
cagctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctaccctaagtttttatgactacaaaaatcagtagtatgtcctgaaataatcattaagctgtttgaaagtatgactgcttgccatgtagataccatggcttgctgaataatcagaagaggtgtgactcttattctaaaatttgtcacaaaatgtcaaaatgagagactctgtaggaacgagtccttgacagacagctcaaggggtttttttcctttgtctcatttctacatgaaagtaaatttgaaatgatcttttttattataagagtagaaatacagttgggtttgaactatatgttttaatggccacggttttgtaagacatttggtcctttgttttcccagttattactcgattgtaattttatatcgccagcaatggactgaaacggtccgcaacctcttctttacaactgggtgacctcgcggctgtgccagccatttggcgttcaccctgccgctaagggccatgtgaacccccggttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtcagatcctcactctcttccgcatcgctgtctgcgagggccagctgttgggctcgcggttgaggacaaactcttcgcggtctttccagtactcttggatcggaaacccgtcggcctccgaacggtactccgccaccgagggacctgagcgagtccgcatcgaccggatcggaaaacctctcgagaaaggcgtctaaccagtcacagtcgcaaggtaggctgagcaccgtggcgggcggcagcgggtggcggtcggggttgtttctggcggaggtgctgctgatgatgtaattaaagtaggcggtcttgagacggcggatggtcgaggtgaggtgtggcaggcttgagatccagctgttggggtgagtactccctctcaaaagcgggcattacttctgcgctaagattgtcagtttccaaaaacgaggaggatttgatattcacctggcccgatctggccatacacttgagtgacaatgacatccactttgcctttctctccacaggtgtccactcccaggtccaagtttaaacttgccgccaccatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctctctgcaggacggcgagttcatctacaaggtgaagctgcgcggcaccaacttcccctccgacggccccgtaatgcagaagaagactatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtgagcggccgcttaattaaaagcttgtcgacggatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggtttttttccggtcggtagcatcccttgctccgcgtggaccactttcctgaggcacagtgataggaacagagccactaatctgaagagaacagagatgtgacagactacactaatgtgagaaaaacaaggaaagggtgacttattggagatttcagaaataaaatgcatttattattatattcccttattttaattttctattagggaattagaaagggcataaactgctttatccagtgttatattaaaagcttaatgtatataatcttttagaggtaaaatctacagccagcaaaagtcatggtaaatattctttgactgaactctcactaaactcctctaaattatatgtcatattaactggttaaattaatataaatttgtgacatgaccttaactggttaggtaggatatttttcttcatgcaaaaatatgactaataataatttagcacaaaaatatttcccaatactttaattctgtgatagaaaaatgtttaactcagctactatgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgccagctggcgtaatagcgaagaggcccgcaccgatcgcccttcccaacagttgcgcagcctgaatggcgaatggcgattccgttgcaatggctggcggtaatattgttctggatattaccagcaaggccgatagtttgagttcttctactcaggcaagtgatgttattactaatcaaagaagtattgcgacaacggttaatttgcgtgatggacagactcttttactcggtggcctcactgattataaaaacacttctcaggattctggcgtaccgttcctgtctaaaatccctttaatcggcctcctgtttagctcccgctctgattctaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttaaatatttgcttatacaatcttcctgtttttggggcttttctgattatcaaccggggtacatatgattgacatgctagttttacgattaccgttcatcgattctcttgtttgctccagactctcaggcaatgacctgatagcctttgtagagacctctcaaaaatagctaccctctccggcatgaatttatcagctagaacggttgaatatcatattgatggtgatttgactgtctccggcctttctcacccgtttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgttggaatcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatg

SEQ ID NO: 294 - DNA, 7011 bp, Synthetic construct
  source 1..7011: mol_type = other DNA
cagctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctaccctaagtttttatgactacaaaaatcagtagtatgtcctgaaataatcattaagctgtttgaaagtatgactgcttgccatgtagataccatggcttgctgaataatcagaagaggtgtgactcttattctaaaatttgtcacaaaatgtcaaaatgagagactctgtaggaacgagtccttgacagacagctcaaggggtttttttcctttgtctcatttctacatgaaagtaaatttgaaatgatcttttttattataagagtagaaatacagttgggtttgaactatatgttttaatggccacggttttgtaagacatttggtcctttgttttcccagttattactcgattgtaattttatatcgccagcaatggactgaaacggtccgcaacctcttctttacaactgggtgacctcgcggctgtgccagccatttggcgttcaccctgccgctaagggccatgtgaacccccggttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtcagatcctcactctcttccgcatcgctgtctgcgagggccagctgttgggctcgcggttgaggacaaactcttcgcggtctttccagtactcttggatcggaaacccgtcggcctccgaacggtactccgccaccgagggacctgagcgagtccgcatcgaccggatcggaaaacctctcgagaaaggcgtctaaccagtcacagtcgcaaggtaggctgagcaccgtggcgggcggcagcgggtggcggtcggggttgtttctggcggaggtgctgctgatgatgtaattaaagtaggcggtcttgagacggcggatggtcgaggtgaggtgtggcaggcttgagatccagctgttggggtgagtactccctctcaaaagcgggcattacttctgcgctaagattgtcagtttccaaaaacgaggaggatttgatattcacctggcccgatctggccatacacttgagtgacaatgacatccactttgcctttctctccacaggtgtccactcccaggtccaagtttaaactttaatacgactcactataggggccgccaccatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctctctgcaggacggcgagttcatctacaaggtgaagctgcgcggcaccaacttcccctccgacggccccgtaatgcagaagaagactatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtgagcggccgcttaattaaaagcttgtcgacggatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggtttttttccggtcggtagcatcccttgctccgcgtggaccactttcctgaggcacagtgataggaacagagccactaatctgaagagaacagagatgtgacagactacactaatgtgagaaaaacaaggaaagggtgacttattggagatttcagaaataaaatgcatttattattatattcccttattttaattttctattagggaattagaaagggcataaactgctttatccagtgttatattaaaagcttaatgtatataatcttttagaggtaaaatctacagccagcaaaagtcatggtaaatattctttgactgaactctcactaaactcctctaaattatatgtcatattaactggttaaattaatataaatttgtgacatgaccttaactggttaggtaggatatttttcttcatgcaaaaatatgactaataataatttagcacaaaaatatttcccaatactttaattctgtgatagaaaaatgtttaactcagctactatgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgccagctggcgtaatagcgaagaggcccgcaccgatcgcccttcccaacagttgcgcagcctgaatggcgaatggcgattccgttgcaatggctggcggtaatattgttctggatattaccagcaaggccgatagtttgagttcttctactcaggcaagtgatgttattactaatcaaagaagtattgcgacaacggttaatttgcgtgatggacagactcttttactcggtggcctcactgattataaaaacacttctcaggattctggcgtaccgttcctgtctaaaatccctttaatcggcctcctgtttagctcccgctctgattctaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttaaatatttgcttatacaatcttcctgtttttggggcttttctgattatcaaccggggtacatatgattgacatgctagttttacgattaccgttcatcgattctcttgtttgctccagactctcaggcaatgacctgatagcctttgtagagacctctcaaaaatagctaccctctccggcatgaatttatcagctagaacggttgaatatcatattgatggtgatttgactgtctccggcctttctcacccgtttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgttggaatcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatg

SEQ ID NO: 295 - DNA, 6202 bp, Synthetic construct
  source 1..6202: mol_type = other DNA
cagctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctaccctaagtttttatgactacaaaaatcagtagtatgtcctgaaataatcattaagctgtttgaaagtatgactgcttgccatgtagataccatggcttgctgaataatcagaagaggtgtgactcttattctaaaatttgtcacaaaatgtcaaaatgagagactctgtaggaacgagtccttgacagacagctcaaggggtttttttcctttgtctcatttctacatgaaagtaaatttgaaatgatcttttttattataagagtagaaatacagttgggtttgaactatatgttttaatggccacggttttgtaagacatttggtcctttgttttcccagttattactcgattgtaattttatatcgccagcaatggactgaaacggtccgcaacctcttctttacaactgggtgacctcgcggctgtgccagccatttggcgttcaccctgccgctaagggccatgtgaacccccggaacagagaaacaggagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctctatataagcagagctcgtttagtgaaccgtcagatcgccgccaccatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctctctgcaggacggcgagttcatctacaaggtgaagctgcgcggcaccaacttcccctccgacggccccgtaatgcagaagaagactatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtgagcggccgcttaattaaaagcttgtcgacggatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggtttttttccggtcggtagcatcccttgctccgcgtggaccactttcctgaggcacagtgataggaacagagccactaatctgaagagaacagagatgtgacagactacactaatgtgagaaaaacaaggaaagggtgacttattggagatttcagaaataaaatgcatttattattatattcccttattttaattttctattagggaattagaaagggcataaactgctttatccagtgttatattaaaagcttaatgtatataatcttttagaggtaaaatctacagccagcaaaagtcatggtaaatattctttgactgaactctcactaaactcctctaaattatatgtcatattaactggttaaattaatataaatttgtgacatgaccttaactggttaggtaggatatttttcttcatgcaaaaatatgactaataataatttagcacaaaaatatttcccaatactttaattctgtgatagaaaaatgtttaactcagctactatgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgccagctggcgtaatagcgaagaggcccgcaccgatcgcccttcccaacagttgcgcagcctgaatggcgaatggcgattccgttgcaatggctggcggtaatattgttctggatattaccagcaaggccgatagtttgagttcttctactcaggcaagtgatgttattactaatcaaagaagtattgcgacaacggttaatttgcgtgatggacagactcttttactcggtggcctcactgattataaaaacacttctcaggattctggcgtaccgttcctgtctaaaatccctttaatcggcctcctgtttagctcccgctctgattctaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttaaatatttgcttatacaatcttcctgtttttggggcttttctgattatcaaccggggtacatatgattgacatgctagttttacgattaccgttcatcgattctcttgtttgctccagactctcaggcaatgacctgatagcctttgtagagacctctcaaaaatagctaccctctccggcatgaatttatcagctagaacggttgaatatcatattgatggtgatttgactgtctccggcctttctcacccgtttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgttggaatcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatg

SEQ ID NO: 296 - DNA, 6207 bp, Synthetic construct
  source 1..6207: mol_type = other DNA
cagctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctaccctaagtttttatgactacaaaaatcagtagtatgtcctgaaataatcattaagctgtttgaaagtatgactgcttgccatgtagataccatggcttgctgaataatcagaagaggtgtgactcttattctaaaatttgtcacaaaatgtcaaaatgagagactctgtaggaacgagtccttgacagacagctcaaggggtttttttcctttgtctcatttctacatgaaagtaaatttgaaatgatcttttttattataagagtagaaatacagttgggtttgaactatatgttttaatggccacggttttgtaagacatttggtcctttgttttcccagttattactcgattgtaattttatatcgccagcaatggactgaaacggtccgcaacctcttctttacaactgggtgacctcgcggctgtgccagccatttggcgttcaccctgccgctaagggccatgtgaacccccggaacagagaaacaggagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctctatataagcagagctcgtttagtgaaccgtcagatcaccgggccgccaccatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctctctgcaggacggcgagttcatctacaaggtgaagctgcgcggcaccaacttcccctccgacggccccgtaatgcagaagaagactatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtgagcggccgcttaattaaaagcttgtcgacggatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggtttttttccggtcggtagcatcccttgctccgcgtggaccactttcctgaggcacagtgataggaacagagccactaatctgaagagaacagagatgtgacagactacactaatgtgagaaaaacaaggaaagggtgacttattggagatttcagaaataaaatgcatttattattatattcccttattttaattttctattagggaattagaaagggcataaactgctttatccagtgttatattaaaagcttaatgtatataatcttttagaggtaaaatctacagccagcaaaagtcatggtaaatattctttgactgaactctcactaaactcctctaaattatatgtcatattaactggttaaattaatataaatttgtgacatgaccttaactggttaggtaggatatttttcttcatgcaaaaatatgactaataataatttagcacaaaaatatttcccaatactttaattctgtgatagaaaaatgtttaactcagctactatgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgccagctggcgtaatagcgaagaggcccgcaccgatcgcccttcccaacagttgcgcagcctgaatggcgaatggcgattccgttgcaatggctggcggtaatattgttctggatattaccagcaaggccgatagtttgagttcttctactcaggcaagtgatgttattactaatcaaagaagtattgcgacaacggttaatttgcgtgatggacagactcttttactcggtggcctcactgattataaaaacacttctcaggattctggcgtaccgttcctgtctaaaatccctttaatcggcctcctgtttagctcccgctctgattctaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttaaatatttgcttatacaatcttcctgtttttggggcttttctgattatcaaccggggtacatatgattgacatgctagttttacgattaccgttcatcgattctcttgtttgctccagactctcaggcaatgacctgatagcctttgtagagacctctcaaaaatagctaccctctccggcatgaatttatcagctagaacggttgaatatcatattgatggtgatttgactgtctccggcctttctcacccgtttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgttggaatcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatg

SEQ ID NO: 297 - DNA, 6048 bp, Synthetic construct
  source 1..6048: mol_type = other DNA
cagctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctaccctaagtttttatgactacaaaaatcagtagtatgtcctgaaataatcattaagctgtttgaaagtatgactgcttgccatgtagataccatggcttgctgaataatcagaagaggtgtgactcttattctaaaatttgtcacaaaatgtcaaaatgagagactctgtaggaacgagtccttgacagacagctcaaggggtttttttcctttgtctcatttctacatgaaagtaaatttgaaatgatcttttttattataagagtagaaatacagttgggtttgaactatatgttttaatggccacggttttgtaagacatttggtcctttgttttcccagttattactcgattgtaattttatatcgccagcaatggactgaaacggtccgcaacctcttctttacaactgggtgacctcgcggctgtgccagccatttggcgttcaccctgccgctaagggccatgtgaacccccgaaaaatgtatgacccttggcatggataatatatgtatttagaagggtcctttaagacacatgtgatgctctagtaagttttctgctgacaccaactccctgtgggtgaaacaggagcaggtgccctttgaccaacagtacacactctgctgagctgatttgcatatggagtcattatacaacatcccaaccgggccgccaccatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctctctgcaggacggcgagttcatctacaaggtgaagctgcgcggcaccaacttcccctccgacggccccgtaatgcagaagaagactatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtgagcggccgcttaattaaaagcttgtcgacggatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggtttttttccggtcggtagcatcccttgctccgcgtggaccactttcctgaggcacagtgataggaacagagccactaatctgaagagaacagagatgtgacagactacactaatgtgagaaaaacaaggaaagggtgacttattggagatttcagaaataaaatgcatttattattatattcccttattttaattttctattagggaattagaaagggcataaactgctttatccagtgttatattaaaagcttaatgtatataatcttttagaggtaaaatctacagccagcaaaagtcatggtaaatattctttgactgaactctcactaaactcctctaaattatatgtcatattaactggttaaattaatataaatttgtgacatgaccttaactggttaggtaggatatttttcttcatgcaaaaatatgactaataataatttagcacaaaaatatttcccaatactttaattctgtgatagaaaaatgtttaactcagctactatgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgccagctggcgtaatagcgaagaggcccgcaccgatcgcccttcccaacagttgcgcagcctgaatggcgaatggcgattccgttgcaatggctggcggtaatattgttctggatattaccagcaaggccgatagtttgagttcttctactcaggcaagtgatgttattactaatcaaagaagtattgcgacaacggttaatttgcgtgatggacagactcttttactcggtggcctcactgattataaaaacacttctcaggattctggcgtaccgttcctgtctaaaatccctttaatcggcctcctgtttagctcccgctctgattctaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttaaatatttgcttatacaatcttcctgtttttggggcttttctgattatcaaccggggtacatatgattgacatgctagttttacgattaccgttcatcgattctcttgtttgctccagactctcaggcaatgacctgatagcctttgtagagacctctcaaaaatagctaccctctccggcatgaatttatcagctagaacggttgaatatcatattgatggtgatttgactgtctccggcctttctcacccgtttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgttggaatcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatg

SEQ ID NO: 298 - DNA, 7822 bp, Synthetic construct
  source 1..7822: mol_type = other DNA
cagctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctaccctaagtttttatgactacaaaaatcagtagtatgtcctgaaataatcattaagctgtttgaaagtatgactgcttgccatgtagataccatggcttgctgaataatcagaagaggtgtgactcttattctaaaatttgtcacaaaatgtcaaaatgagagactctgtaggaacgagtccttgacagacagctcaaggggtttttttcctttgtctcatttctacatgaaagtaaatttgaaatgatcttttttattataagagtagaaatacagttgggtttgaactatatgttttaatggccacggttttgtaagacatttggtcctttgttttcccagttattactcgattgtaattttatatcgccagcaatggactgaaacggtccgcaacctcttctttacaactgggtgacctcgcggctgtgccagccatttggcgttcaccctgccgctaagggccatgtgaacccccggaacagagaaacaggagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctctatataagcagagctcgtttagtgaaccgtcagatcgccgccaccatgctcgagggagtgcaggtggagactatctccccaggagacgggcgcaccttccccaagcgcggccagacctgcgtggtgcactacaccgggatgcttgaagatggaaagaaagttgattcctcccgggacagaaacaagccctttaagtttatgctaggcaagcaggaggtgatccgaggctgggaagaaggggttgcccagatgagtgtgggtcagagagccaaactgactatatctccagattatgcctatggtgccactgggcacccaggcatcatcccaccacatgccactctcgtcttcgatgtggagcttctaaaactggaatctggcggtggatccggagtcgacggatttggtgatgtcggtgctcttgagagtttgaggggaaatgcagatttggcttacatcctgagcatggagccctgtggccactgcctcattatcaacaatgtgaacttctgccgtgagtccgggctccgcacccgcactggctccaacatcgactgtgagaagttgcggcgtcgcttctcctcgctgcatttcatggtggaggtgaagggcgacctgactgccaagaaaatggtgctggctttgctggagctggcgcggcaggaccacggtgctctggactgctgcgtggtggtcattctctctcacggctgtcaggccagccacctgcagttcccaggggctgtctacggcacagatggatgccctgtgtcggtcgagaagattgtgaacatcttcaatgggaccagctgccccagcctgggagggaagcccaagctctttttcatccaggcctgtggtggggagcagaaagaccatgggtttgaggtggcctccacttcccctgaagacgagtcccctggcagtaaccccgagccagatgccaccccgttccaggaaggtttgaggaccttcgaccagctggacgccatatctagtttgcccacacccagtgacatctttgtgtcctactctactttcccaggttttgtttcctggagggaccccaagagtggctcctggtacgttgagaccctggacgacatctttgagcagtgggctcactctgaagacctgcagtccctcctgcttagggtcgctaatgctgtttcggtgaaagggatttataaacagatgcctggttgctttaatttcctccggaaaaaacttttctttaaaacatcagtcgactatccgtacgacgtaccagactacgcactcgactaagaattcatcgagcgggatcaattccgccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgataataccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaatttttttccggtcggtagcatcccttgctccgcgtggaccactttcctgaggcacagtgataggaacagagccactaatctgaagagaacagagatgtgacagactacactaatgtgagaaaaacaaggaaagggtgacttattggagatttcagaaataaaatgcatttattattatattcccttattttaattttctattagggaattagaaagggcataaactgctttatccagtgttatattaaaagcttaatgtatataatcttttagaggtaaaatctacagccagcaaaagtcatggtaaatattctttgactgaactctcactaaactcctctaaattatatgtcatattaactggttaaattaatataaatttgtgacatgaccttaactggttaggtaggatatttttcttcatgcaaaaatatgactaataataatttagcacaaaaatatttcccaatactttaattctgtgatagaaaaatgtttaactcagctactatgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgccagctggcgtaatagcgaagaggcccgcaccgatcgcccttcccaacagttgcgcagcctgaatggcgaatggcgattccgttgcaatggctggcggtaatattgttctggatattaccagcaaggccgatagtttgagttcttctactcaggcaagtgatgttattactaatcaaagaagtattgcgacaacggttaatttgcgtgatggacagactcttttactcggtggcctcactgattataaaaacacttctcaggattctggcgtaccgttcctgtctaaaatccctttaatcggcctcctgtttagctcccgctctgattctaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttaaatatttgcttatacaatcttcctgtttttggggcttttctgattatcaaccggggtacatatgattgacatgctagttttacgattaccgttcatcgattctcttgtttgctccagactctcaggcaatgacctgatagcctttgtagagacctctcaaaaatagctaccctctccggcatgaatttatcagctagaacggttgaatatcatattgatggtgatttgactgtctccggcctttctcacccgtttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgttggaatcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatg

SEQ ID NO: 299 - DNA, 7827 bp, Synthetic construct
  source 1..7827: mol_type = other DNA
cagctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctaccctaagtttttatgactacaaaaatcagtagtatgtcctgaaataatcattaagctgtttgaaagtatgactgcttgccatgtagataccatggcttgctgaataatcagaagaggtgtgactcttattctaaaatttgtcacaaaatgtcaaaatgagagactctgtaggaacgagtccttgacagacagctcaaggggtttttttcctttgtctcatttctacatgaaagtaaatttgaaatgatcttttttattataagagtagaaatacagttgggtttgaactatatgttttaatggccacggttttgtaagacatttggtcctttgttttcccagttattactcgattgtaattttatatcgccagcaatggactgaaacggtccgcaacctcttctttacaactgggtgacctcgcggctgtgccagccatttggcgttcaccctgccgctaagggccatgtgaacccccggaacagagaaacaggagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctctatataagcagagctcgtttagtgaaccgtcagatcaccgggccgccaccatgctcgagggagtgcaggtggagactatctccccaggagacgggcgcaccttccccaagcgcggccagacctgcgtggtgcactacaccgggatgcttgaagatggaaagaaagttgattcctcccgggacagaaacaagccctttaagtttatgctaggcaagcaggaggtgatccgaggctgggaagaaggggttgcccagatgagtgtgggtcagagagccaaactgactatatctccagattatgcctatggtgccactgggcacccaggcatcatcccaccacatgccactctcgtcttcgatgtggagcttctaaaactggaatctggcggtggatccggagtcgacggatttggtgatgtcggtgctcttgagagtttgaggggaaatgcagatttggcttacatcctgagcatggagccctgtggccactgcctcattatcaacaatgtgaacttctgccgtgagtccgggctccgcacccgcactggctccaacatcgactgtgagaagttgcggcgtcgcttctcctcgctgcatttcatggtggaggtgaagggcgacctgactgccaagaaaatggtgctggctttgctggagctggcgcggcaggaccacggtgctctggactgctgcgtggtggtcattctctctcacggctgtcaggccagccacctgcagttcccaggggctgtctacggcacagatggatgccctgtgtcggtcgagaagattgtgaacatcttcaatgggaccagctgccccagcctgggagggaagcccaagctctttttcatccaggcctgtggtggggagcagaaagaccatgggtttgaggtggcctccacttcccctgaagacgagtcccctggcagtaaccccgagccagatgccaccccgttccaggaaggtttgaggaccttcgaccagctggacgccatatctagtttgcccacacccagtgacatctttgtgtcctactctactttcccaggttttgtttcctggagggaccccaagagtggctcctggtacgttgagaccctggacgacatctttgagcagtgggctcactctgaagacctgcagtccctcctgcttagggtcgctaatgctgtttcggtgaaagggatttataaacagatgcctggttgctttaatttcctccggaaaaaacttttctttaaaacatcagtcgactatccgtacgacgtaccagactacgcactcgactaagaattcatcgagcgggatcaattccgccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgataataccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaatttttttccggtcggtagcatcccttgctccgcgtggaccactttcctgaggcacagtgataggaacagagccactaatctgaagagaacagagatgtgacagactacactaatgtgagaaaaacaaggaaagggtgacttattggagatttcagaaataaaatgcatttattattatattcccttattttaattttctattagggaattagaaagggcataaactgctttatccagtgttatattaaaagcttaatgtatataatcttttagaggtaaaatctacagccagcaaaagtcatggtaaatattctttgactgaactctcactaaactcctctaaattatatgtcatattaactggttaaattaatataaatttgtgacatgaccttaactggttaggtaggatatttttcttcatgcaaaaatatgactaataataatttagcacaaaaatatttcccaatactttaattctgtgatagaaaaatgtttaactcagctactatgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgccagctggcgtaatagcgaagaggcccgcaccgatcgcccttcccaacagttgcgcagcctgaatggcgaatggcgattccgttgcaatggctggcggtaatattgttctggatattaccagcaaggccgatagtttgagttcttctactcaggcaagtgatgttattactaatcaaagaagtattgcgacaacggttaatttgcgtgatggacagactcttttactcggtggcctcactgattataaaaacacttctcaggattctggcgtaccgttcctgtctaaaatccctttaatcggcctcctgtttagctcccgctctgattctaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttaaatatttgcttatacaatcttcctgtttttggggcttttctgattatcaaccggggtacatatgattgacatgctagttttacgattaccgttcatcgattctcttgtttgctccagactctcaggcaatgacctgatagcctttgtagagacctctcaaaaatagctaccctctccggcatgaatttatcagctagaacggttgaatatcatattgatggtgatttgactgtctccggcctttctcacccgtttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgttggaatcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatg

SEQ ID NO: 300 - DNA, 7663 bp, Synthetic construct
  source 1..7663: mol_type = other DNA
cagctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctaccctaagtttttatgactacaaaaatcagtagtatgtcctgaaataatcattaagctgtttgaaagtatgactgcttgccatgtagataccatggcttgctgaataatcagaagaggtgtgactcttattctaaaatttgtcacaaaatgtcaaaatgagagactctgtaggaacgagtccttgacagacagctcaaggggtttttttcctttgtctcatttctacatgaaagtaaatttgaaatgatcttttttattataagagtagaaatacagttgggtttgaactatatgttttaatggccacggttttgtaagacatttggtcctttgttttcccagttattactcgattgtaattttatatcgccagcaatggactgaaacggtccgcaacctcttctttacaactgggtgacctcgcggctgtgccagccatttggcgttcaccctgccgctaagggccatgtgaacccccgaaaaatgtatgacccttggcatggataatatatgtatttagaagggtcctttaagacacatgtgatgctctagtaagttttctgctgacaccaactccctgtgggtgaaacaggagcaggtgccctttgaccaacagtacacactctgctgagctgatttgcatatggagtcattatacaacatcccagccgccaccatgctcgagggagtgcaggtggagactatctccccaggagacgggcgcaccttccccaagcgcggccagacctgcgtggtgcactacaccgggatgcttgaagatggaaagaaagttgattcctcccgggacagaaacaagccctttaagtttatgctaggcaagcaggaggtgatccgaggctgggaagaaggggttgcccagatgagtgtgggtcagagagccaaactgactatatctccagattatgcctatggtgccactgggcacccaggcatcatcccaccacatgccactctcgtcttcgatgtggagcttctaaaactggaatctggcggtggatccggagtcgacggatttggtgatgtcggtgctcttgagagtttgaggggaaatgcagatttggcttacatcctgagcatggagccctgtggccactgcctcattatcaacaatgtgaacttctgccgtgagtccgggctccgcacccgcactggctccaacatcgactgtgagaagttgcggcgtcgcttctcctcgctgcatttcatggtggaggtgaagggcgacctgactgccaagaaaatggtgctggctttgctggagctggcgcggcaggaccacggtgctctggactgctgcgtggtggtcattctctctcacggctgtcaggccagccacctgcagttcccaggggctgtctacggcacagatggatgccctgtgtcggtcgagaagattgtgaacatcttcaatgggaccagctgccccagcctgggagggaagcccaagctctttttcatccaggcctgtggtggggagcagaaagaccatgggtttgaggtggcctccacttcccctgaagacgagtcccctggcagtaaccccgagccagatgccaccccgttccaggaaggtttgaggaccttcgaccagctggacgccatatctagtttgcccacacccagtgacatctttgtgtcctactctactttcccaggttttgtttcctggagggaccccaagagtggctcctggtacgttgagaccctggacgacatctttgagcagtgggctcactctgaagacctgcagtccctcctgcttagggtcgctaatgctgtttcggtgaaagggatttataaacagatgcctggttgctttaatttcctccggaaaaaacttttctttaaaacatcagtcgactatccgtacgacgtaccagactacgcactcgactaagaattcatcgagcgggatcaattccgccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgataataccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaatttttttccggtcggtagcatcccttgctccgcgtggaccactttcctgaggcacagtgataggaacagagccactaatctgaagagaacagagatgtgacagactacactaatgtgagaaaaacaaggaaagggtgacttattggagatttcagaaataaaatgcatttattattatattcccttattttaattttctattagggaattagaaagggcataaactgctttatccagtgttatattaaaagcttaatgtatataatcttttagaggtaaaatctacagccagcaaaagtcatggtaaatattctttgactgaactctcactaaactcctctaaattatatgtcatattaactggttaaattaatataaatttgtgacatgaccttaactggttaggtaggatatttttcttcatgcaaaaatatgactaataataatttagcacaaaaatatttcccaatactttaattctgtgatagaaaaatgtttaactcagctactatgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgccagctggcgtaatagcgaagaggcccgcaccgatcgcccttcccaacagttgcgcagcctgaatggcgaatggcgattccgttgcaatggctggcggtaatattgttctggatattaccagcaaggccgatagtttgagttcttctactcaggcaagtgatgttattactaatcaaagaagtattgcgacaacggttaatttgcgtgatggacagactcttttactcggtggcctcactgattataaaaacacttctcaggattctggcgtaccgttcctgtctaaaatccctttaatcggcctcctgtttagctcccgctctgattctaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttaaatatttgcttatacaatcttcctgtttttggggcttttctgattatcaaccggggtacatatgattgacatgctagttttacgattaccgttcatcgattctcttgtttgctccagactctcaggcaatgacctgatagcctttgtagagacctctcaaaaatagctaccctctccggcatgaatttatcagctagaacggttgaatatcatattgatggtgatttgactgtctccggcctttctcacccgtttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgttggaatcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatg

SEQ ID NO: 301 - DNA, 7378 bp, Synthetic construct
  source 1..7378: mol_type = other DNA
cagctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctaccctaagtttttatgactacaaaaatcagtagtatgtcctgaaataatcattaagctgtttgaaagtatgactgcttgccatgtagataccatggcttgctgaataatcagaagaggtgtgactcttattctaaaatttgtcacaaaatgtcaaaatgagagactctgtaggaacgagtccttgacagacagctcaaggggtttttttcctttgtctcatttctacatgaaagtaaatttgaaatgatcttttttattataagagtagaaatacagttgggtttgaactatatgttttaatggccacggttttgtaagacatttggtcctttgttttcccagttattactcgattgtaattttatatcgccagcaatggactgaaacggtccgcaacctcttctttacaactgggtgacctcgcggctgtgccagccatttggcgttcaccctgccgctaagggccatgtgaacccccggttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtcagatcctcactctcttccgcatcgctgtctgcgagggccagctgttgggctcgcggttgaggacaaactcttcgcggtctttccagtactcttggatcggaaacccgtcggcctccgaacggtactccgccaccgagggacctgagcgagtccgcatcgaccggatcggaaaacctctcgagaaaggcgtctaaccagtcacagtcgcaaggtaggctgagcaccgtggcgggcggcagcgggtggcggtcggggttgtttctggcggaggtgctgctgatgatgtaattaaagtaggcggtcttgagacggcggatggtcgaggtgaggtgtggcaggcttgagatccagctgttggggtgagtactccctctcaaaagcgggcattacttctgcgctaagattgtcagtttccaaaaacgaggaggatttgatattcacctggcccgatctggccatacacttgagtgacaatgacatccactttgcctttctctccacaggtgtccactcccaggtccaagtttaaactttaatacgactcactataggggccgccaccaagcttggtaccatgcagctgaggaacccagaactacatctgggctgcgcgcttgcgcttcgcttcctggccctcgtttcctgggacatccctggggctagagcactggacaatggattggcaaggacgcctaccatgggctggctgcactgggagcgcttcatgtgcaaccttgactgccaggaagagccagattcctgcatcagtgagaagctcttcatggagatggcagagctcatggtctcagaaggctggaaggatgcaggttatgagtacctctgcattgatgactgttggatggctccccaaagagattcagaaggcagacttcaggcagaccctcagcgctttcctcatgggattcgccagctagctaattatgttcacagcaaaggactgaagctagggatttatgcagatgttggaaataaaacctgcgcaggcttccctgggagttttggatactacgacattgatgcccagacctttgctgactggggagtagatctgctaaaatttgatggttgttactgtgacagtttggaaaatttggcagatggttataagcacatgtccttggccctgaataggactggcagaagcattgtgtactcctgtgagtggcctctttatatgtggccctttcaaaagcccaattatacagaaatccgacagtactgcaatcactggcgaaattttgctgacattgatgattcctggaaaagtataaagagtatcttggactggacatcttttaaccaggagagaattgttgatgttgctggaccagggggttggaatgacccagatatgttagtgattggcaactttggcctcagctggaatcagcaagtaactcagatggccctctgggctatcatggctgctcctttattcatgtctaatgacctccgacacatcagccctcaagccaaagctctccttcaggataaggacgtaattgccatcaatcaggaccccttgggcaagcaagggtaccagcttagacagggagacaactttgaagtgtgggaacgacctctctcaggcttagcctgggctgtagctatgataaaccggcaggagattggtggacctcgctcttataccatcgcagttgcttccctgggtaaaggagtggcctgtaatcctgcctgcttcatcacacagctcctccctgtgaaaaggaagctagggttctatgaatggacttcaaggttaagaagtcacataaatcccacaggcactgttttgcttcagctagaaaatacaatgcagatgtcattaaaagacttactttaatttttttcggtagcatcccttgctccgcgtggaccactttcctgaggcacagtgataggaacagagccactaatctgaagagaacagagatgtgacagactacactaatgtgagaaaaacaaggaaagggtgacttattggagatttcagaaataaaatgcatttattattatattcccttattttaattttctattagggaattagaaagggcataaactgctttatccagtgttatattaaaagcttaatgtatataatcttttagaggtaaaatctacagccagcaaaagtcatggtaaatattctttgactgaactctcactaaactcctctaaattatatgtcatattaactggttaaattaatataaatttgtgacatgaccttaactggttaggtaggatatttttcttcatgcaaaaatatgactaataataatttagcacaaaaatatttcccaatactttaattctgtgatagaaaaatgtttaactcagctactatgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgccagctggcgtaatagcgaagaggcccgcaccgatcgcccttcccaacagttgcgcagcctgaatggcgaatggcgattccgttgcaatggctggcggtaatattgttctggatattaccagcaaggccgatagtttgagttcttctactcaggcaagtgatgttattactaatcaaagaagtattgcgacaacggttaatttgcgtgatggacagactcttttactcggtggcctcactgattataaaaacacttctcaggattctggcgtaccgttcctgtctaaaatccctttaatcggcctcctgtttagctcccgctctgattctaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttaaatatttgcttatacaatcttcctgtttttggggcttttctgattatcaaccggggtacatatgattgacatgctagttttacgattaccgttcatcgattctcttgtttgctccagactctcaggcaatgacctgatagcctttgtagagacctctcaaaaatagctaccctctccggcatgaatttatcagctagaacggttgaatatcatattgatggtgatttgactgtctccggcctttctcacccgtttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgttggaatcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatg

SEQ ID NO: 302 - DNA, 6537 bp, Synthetic construct
  source 1..6537: mol_type = other DNA
cagctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctaccctaagtttttatgactacaaaaatcagtagtatgtcctgaaataatcattaagctgtttgaaagtatgactgcttgccatgtagataccatggcttgctgaataatcagaagaggtgtgactcttattctaaaatttgtcacaaaatgtcaaaatgagagactctgtaggaacgagtccttgacagacagctcaaggggtttttttcctttgtctcatttctacatgaaagtaaatttgaaatgatcttttttattataagagtagaaatacagttgggtttgaactatatgttttaatggccacggttttgtaagacatttggtcctttgttttcccagttattactcgattgtaattttatatcgccagcaatggactgaaacggtccgcaacctcttctttacaactgggtgacctcgcggctgtgccagccatttggcgttcaccctgccgctaagggccatgtgaacccccggaacagagaaacaggagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctctatataagcagagctcgtttagtgaaccgtcagatcaccgggccgccaccatgaagcttttgagcagagatacgcggctggtctgtgagcttgcgctttgtcccctggctttagttttctggagcattcttggggtcagagcattggacaatggcttggcgcggactcctactatgggctggctgcattgggaacgtttcatgtgcaaccttgactgccaagaagagcctgatgcctgcataagtgagcaactgttcatgcagatggcagagctcatggtctctgatggctggcgggatgcaggttatgactatctctgcatagatgactgttggatggctcccgagagggattcaaagggcaggcttcaggcagatccccaacgctttcctagtgggatcaaacacctcgcaaattacgtccacagcaaaggattgaagctagggatttatgcagatgttgggaataaaacctgtgcaggtttccccgggagttttggatcctatgacattgatgcgcagacatttgctgactggggcgtagatctgctaaaatttgatggttgtcactgtgacagtgtagtatccttggagaatggttataagtacatggccttggccttgaacaggacaggccgaagcattgtatactcctgtgagtggccactttatttgagaccctttcataagcccaattatacagatatccaatattactgcaatcattggagaaattttgatgatgtttatgattcctgggaaagcataaagaatatcttgtcttggacagtggtttaccagaaggagattgttgaagtcgctggaccaggcagctggaatgacccagacatgttagtgatcggcaactttggcctcagttgggaccagcaggtgacacagatggccctctgggctatcatggccgctcccctactcatgtccaacgatctccgacaaatcagctctcaagccaaagctctgcttcagaataaggatgtaattgccatcaaccaagaccccttgggcaagcagggctactgtttcagaaaggaaaaccacattgaggtttgggaacggccactctccaacctagcctgggctgtggctgtgagaaacctgcaggagattggtggaccttgtccttataccatccagatttcttccctgggtagaggactagcctgcaatcctggctgcatcattactcagcttctccccgagaaagtacacctaggcttctatgaatggactttgaccttaaaaactcgagtaaacccctcaggcactgttttgtttcggttagaaagataatttttttccggtcggtagcatcccttgctccgcgtggaccactttcctgaggcacagtgataggaacagagccactaatctgaagagaacagagatgtgacagactacactaatgtgagaaaaacaaggaaagggtgacttattggagatttcagaaataaaatgcatttattattatattcccttattttaattttctattagggaattagaaagggcataaactgctttatccagtgttatattaaaagcttaatgtatataatcttttagaggtaaaatctacagccagcaaaagtcatggtaaatattctttgactgaactctcactaaactcctctaaattatatgtcatattaactggttaaattaatataaatttgtgacatgaccttaactggttaggtaggatatttttcttcatgcaaaaatatgactaataataatttagcacaaaaatatttcccaatactttaattctgtgatagaaaaatgtttaactcagctactatgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgccagctggcgtaatagcgaagaggcccgcaccgatcgcccttcccaacagttgcgcagcctgaatggcgaatggcgattccgttgcaatggctggcggtaatattgttctggatattaccagcaaggccgatagtttgagttcttctactcaggcaagtgatgttattactaatcaaagaagtattgcgacaacggttaatttgcgtgatggacagactcttttactcggtggcctcactgattataaaaacacttctcaggattctggcgtaccgttcctgtctaaaatccctttaatcggcctcctgtttagctcccgctctgattctaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttaaatatttgcttatacaatcttcctgtttttggggcttttctgattatcaaccggggtacatatgattgacatgctagttttacgattaccgttcatcgattctcttgtttgctccagactctcaggcaatgacctgatagcctttgtagagacctctcaaaaatagctaccctctccggcatgaatttatcagctagaacggttgaatatcatattgatggtgatttgactgtctccggcctttctcacccgtttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgttggaatcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatg

SEQ ID NO: 303 - DNA, 6499 bp, Synthetic construct
  source 1..6499: mol_type = other DNA
cagctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctaccctaagtttttatgactacaaaaatcagtagtatgtcctgaaataatcattaagctgtttgaaagtatgactgcttgccatgtagataccatggcttgctgaataatcagaagaggtgtgactcttattctaaaatttgtcacaaaatgtcaaaatgagagactctgtaggaacgagtccttgacagacagctcaaggggtttttttcctttgtctcatttctacatgaaagtaaatttgaaatgatcttttttattataagagtagaaatacagttgggtttgaactatatgttttaatggccacggttttgtaagacatttggtcctttgttttcccagttattactcgattgtaattttatatcgccagcaatggactgaaacggtccgcaacctcttctttacaactgggtgacctcgcggctgtgccagccatttggcgttcaccctgccgctaagggccatgtgaacccccggaacagagaaacaggagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctctatataagcagagctcgtttagtgaaccgtcagatcgccgccaccatgaggaggatgcagctgctgctgctgatcgccctgagcctggccctggtgaccaacagcttggacaatggcttggcgcggactcctactatgggctggctgcattgggaacgtttcatgtgcaaccttgactgccaagaagagcctgatgcctgcataagtgagcaactgttcatgcagatggcagagctcatggtctctgatggctggcgggatgcaggttatgactatctctgcatagatgactgttggatggctcccgagagggattcaaagggcaggcttcaggcagatccccaacgctttcctagtgggatcaaacacctcgcaaattacgtccacagcaaaggattgaagctagggatttatgcagatgttgggaataaaacctgtgcaggtttccccgggagttttggatcctatgacattgatgcgcagacatttgctgactggggcgtagatctgctaaaatttgatggttgtcactgtgacagtgtagtatccttggagaatggttataagtacatggccttggccttgaacaggacaggccgaagcattgtatactcctgtgagtggccactttatttgagaccctttcataagcccaattatacagatatccaatattactgcaatcattggagaaattttgatgatgtttatgattcctgggaaagcataaagaatatcttgtcttggacagtggtttaccagaaggagattgttgaagtcgctggaccaggcagctggaatgacccagacatgttagtgatcggcaactttggcctcagttgggaccagcaggtgacacagatggccctctgggctatcatggccgctcccctactcatgtccaacgatctccgacaaatcagctctcaagccaaagctctgcttcagaataaggatgtaattgccatcaaccaagaccccttgggcaagcagggctactgtttcagaaaggaaaaccacattgaggtttgggaacggccactctccaacctagcctgggctgtggctgtgagaaacctgcaggagattggtggaccttgtccttataccatccagatttcttccctgggtagaggactagcctgcaatcctggctgcatcattactcagcttctccccgagaaagtacacctaggcttctatgaatggactttgaccttaaaaactcgagtaaacccctcaggcactgttttgtttcggttagaaagataatttttttccggtcggtagcatcccttgctccgcgtggaccactttcctgaggcacagtgataggaacagagccactaatctgaagagaacagagatgtgacagactacactaatgtgagaaaaacaaggaaagggtgacttattggagatttcagaaataaaatgcatttattattatattcccttattttaattttctattagggaattagaaagggcataaactgctttatccagtgttatattaaaagcttaatgtatataatcttttagaggtaaaatctacagccagcaaaagtcatggtaaatattctttgactgaactctcactaaactcctctaaattatatgtcatattaactggttaaattaatataaatttgtgacatgaccttaactggttaggtaggatatttttcttcatgcaaaaatatgactaataataatttagcacaaaaatatttcccaatactttaattctgtgatagaaaaatgtttaactcagctactatgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgccagctggcgtaatagcgaagaggcccgcaccgatcgcccttcccaacagttgcgcagcctgaatggcgaatggcgattccgttgcaatggctggcggtaatattgttctggatattaccagcaaggccgatagtttgagttcttctactcaggcaagtgatgttattactaatcaaagaagtattgcgacaacggttaatttgcgtgatggacagactcttttactcggtggcctcactgattataaaaacacttctcaggattctggcgtaccgttcctgtctaaaatccctttaatcggcctcctgtttagctcccgctctgattctaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttaaatatttgcttatacaatcttcctgtttttggggcttttctgattatcaaccggggtacatatgattgacatgctagttttacgattaccgttcatcgattctcttgtttgctccagactctcaggcaatgacctgatagcctttgtagagacctctcaaaaatagctaccctctccggcatgaatttatcagctagaacggttgaatatcatattgatggtgatttgactgtctccggcctttctcacccgtttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgttggaatcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatg

SEQ ID NO: 304 - DNA, 6504 bp, Synthetic construct
  source 1..6504: mol_type = other DNA
cagctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctaccctaagtttttatgactacaaaaatcagtagtatgtcctgaaataatcattaagctgtttgaaagtatgactgcttgccatgtagataccatggcttgctgaataatcagaagaggtgtgactcttattctaaaatttgtcacaaaatgtcaaaatgagagactctgtaggaacgagtccttgacagacagctcaaggggtttttttcctttgtctcatttctacatgaaagtaaatttgaaatgatcttttttattataagagtagaaatacagttgggtttgaactatatgttttaatggccacggttttgtaagacatttggtcctttgttttcccagttattactcgattgtaattttatatcgccagcaatggactgaaacggtccgcaacctcttctttacaactgggtgacctcgcggctgtgccagccatttggcgttcaccctgccgctaagggccatgtgaacccccggaacagagaaacaggagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctctatataagcagagctcgtttagtgaaccgtcagatcaccgggccgccaccatgaggaggatgcagctgctgctgctgatcgccctgagcctggccctggtgaccaacagcttggacaatggcttggcgcggactcctactatgggctggctgcattgggaacgtttcatgtgcaaccttgactgccaagaagagcctgatgcctgcataagtgagcaactgttcatgcagatggcagagctcatggtctctgatggctggcgggatgcaggttatgactatctctgcatagatgactgttggatggctcccgagagggattcaaagggcaggcttcaggcagatccccaacgctttcctagtgggatcaaacacctcgcaaattacgtccacagcaaaggattgaagctagggatttatgcagatgttgggaataaaacctgtgcaggtttccccgggagttttggatcctatgacattgatgcgcagacatttgctgactggggcgtagatctgctaaaatttgatggttgtcactgtgacagtgtagtatccttggagaatggttataagtacatggccttggccttgaacaggacaggccgaagcattgtatactcctgtgagtggccactttatttgagaccctttcataagcccaattatacagatatccaatattactgcaatcattggagaaattttgatgatgtttatgattcctgggaaagcataaagaatatcttgtcttggacagtggtttaccagaaggagattgttgaagtcgctggaccaggcagctggaatgacccagacatgttagtgatcggcaactttggcctcagttgggaccagcaggtgacacagatggccctctgggctatcatggccgctcccctactcatgtccaacgatctccgacaaatcagctctcaagccaaagctctgcttcagaataaggatgtaattgccatcaaccaagaccccttgggcaagcagggctactgtttcagaaaggaaaaccacattgaggtttgggaacggccactctccaacctagcctgggctgtggctgtgagaaacctgcaggagattggtggaccttgtccttataccatccagatttcttccctgggtagaggactagcctgcaatcctggctgcatcattactcagcttctccccgagaaagtacacctaggcttctatgaatggactttgaccttaaaaactcgagtaaacccctcaggcactgttttgtttcggttagaaagataatttttttccggtcggtagcatcccttgctccgcgtggaccactttcctgaggcacagtgataggaacagagccactaatctgaagagaacagagatgtgacagactacactaatgtgagaaaaacaaggaaagggtgacttattggagatttcagaaataaaatgcatttattattatattcccttattttaattttctattagggaattagaaagggcataaactgctttatccagtgttatattaaaagcttaatgtatataatcttttagaggtaaaatctacagccagcaaaagtcatggtaaatattctttgactgaactctcactaaactcctctaaattatatgtcatattaactggttaaattaatataaatttgtgacatgaccttaactggttaggtaggatatttttcttcatgcaaaaatatgactaataataatttagcacaaaaatatttcccaatactttaattctgtgatagaaaaatgtttaactcagctactatgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgccagctggcgtaatagcgaagaggcccgcaccgatcgcccttcccaacagttgcgcagcctgaatggcgaatggcgattccgttgcaatggctggcggtaatattgttctggatattaccagcaaggccgatagtttgagttcttctactcaggcaagtgatgttattactaatcaaagaagtattgcgacaacggttaatttgcgtgatggacagactcttttactcggtggcctcactgattataaaaacacttctcaggattctggcgtaccgttcctgtctaaaatccctttaatcggcctcctgtttagctcccgctctgattctaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttaaatatttgcttatacaatcttcctgtttttggggcttttctgattatcaaccggggtacatatgattgacatgctagttttacgattaccgttcatcgattctcttgtttgctccagactctcaggcaatgacctgatagcctttgtagagacctctcaaaaatagctaccctctccggcatgaatttatcagctagaacggttgaatatcatattgatggtgatttgactgtctccggcctttctcacccgtttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgttggaatcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatg

SEQ ID NO: 305 - DNA, 6499 bp, Synthetic construct
  source 1..6499: mol_type = other DNA
cagctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctaccctaagtttttatgactacaaaaatcagtagtatgtcctgaaataatcattaagctgtttgaaagtatgactgcttgccatgtagataccatggcttgctgaataatcagaagaggtgtgactcttattctaaaatttgtcacaaaatgtcaaaatgagagactctgtaggaacgagtccttgacagacagctcaaggggtttttttcctttgtctcatttctacatgaaagtaaatttgaaatgatcttttttattataagagtagaaatacagttgggtttgaactatatgttttaatggccacggttttgtaagacatttggtcctttgttttcccagttattactcgattgtaattttatatcgccagcaatggactgaaacggtccgcaacctcttctttacaactgggtgacctcgcggctgtgccagccatttggcgttcaccctgccgctaagggccatgtgaacccccggaacagagaaacaggagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctctatataagcagagctcgtttagtgaaccgtcagatcgccgccaccatgtataggatgcagctgctgagctgcatcgccctgagcctggccctggtgaccaacagcttggacaatggcttggcgcggactcctactatgggctggctgcattgggaacgtttcatgtgcaaccttgactgccaagaagagcctgatgcctgcataagtgagcaactgttcatgcagatggcagagctcatggtctctgatggctggcgggatgcaggttatgactatctctgcatagatgactgttggatggctcccgagagggattcaaagggcaggcttcaggcagatccccaacgctttcctagtgggatcaaacacctcgcaaattacgtccacagcaaaggattgaagctagggatttatgcagatgttgggaataaaacctgtgcaggtttccccgggagttttggatcctatgacattgatgcgcagacatttgctgactggggcgtagatctgctaaaatttgatggttgtcactgtgacagtgtagtatccttggagaatggttataagtacatggccttggccttgaacaggacaggccgaagcattgtatactcctgtgagtggccactttatttgagaccctttcataagcccaattatacagatatccaatattactgcaatcattggagaaattttgatgatgtttatgattcctgggaaagcataaagaatatcttgtcttggacagtggtttaccagaaggagattgttgaagtcgctggaccaggcagctggaatgacccagacatgttagtgatcggcaactttggcctcagttgggaccagcaggtgacacagatggccctctgggctatcatggccgctcccctactcatgtccaacgatctccgacaaatcagctctcaagccaaagctctgcttcagaataaggatgtaattgccatcaaccaagaccccttgggcaagcagggctactgtttcagaaaggaaaaccacattgaggtttgggaacggccactctccaacctagcctgggctgtggctgtgagaaacctgcaggagattggtggaccttgtccttataccatccagatttcttccctgggtagaggactagcctgcaatcctggctgcatcattactcagcttctccccgagaaagtacacctaggcttctatgaatggactttgaccttaaaaactcgagtaaacccctcaggcactgttttgtttcggttagaaagataatttttttccggtcggtagcatcccttgctccgcgtggaccactttcctgaggcacagtgataggaacagagccactaatctgaagagaacagagatgtgacagactacactaatgtgagaaaaacaaggaaagggtgacttattggagatttcagaaataaaatgcatttattattatattcccttattttaattttctattagggaattagaaagggcataaactgctttatccagtgttatattaaaagcttaatgtatataatcttttagaggtaaaatctacagccagcaaaagtcatggtaaatattctttgactgaactctcactaaactcctctaaattatatgtcatattaactggttaaattaatataaatttgtgacatgaccttaactggttaggtaggatatttttcttcatgcaaaaatatgactaataataatttagcacaaaaatatttcccaatactttaattctgtgatagaaaaatgtttaactcagctactatgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgccagctggcgtaatagcgaagaggcccgcaccgatcgcccttcccaacagttgcgcagcctgaatggcgaatggcgattccgttgcaatggctggcggtaatattgttctggatattaccagcaaggccgatagtttgagttcttctactcaggcaagtgatgttattactaatcaaagaagtattgcgacaacggttaatttgcgtgatggacagactcttttactcggtggcctcactgattataaaaacacttctcaggattctggcgtaccgttcctgtctaaaatccctttaatcggcctcctgtttagctcccgctctgattctaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttaaatatttgcttatacaatcttcctgtttttggggcttttctgattatcaaccggggtacatatgattgacatgctagttttacgattaccgttcatcgattctcttgtttgctccagactctcaggcaatgacctgatagcctttgtagagacctctcaaaaatagctaccctctccggcatgaatttatcagctagaacggttgaatatcatattgatggtgatttgactgtctccggcctttctcacccgtttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgttggaatcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatg

SEQ ID NO: 306 - DNA, 6504 bp, Synthetic construct
  source 1..6504: mol_type = other DNA
cagctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctaccctaagtttttatgactacaaaaatcagtagtatgtcctgaaataatcattaagctgtttgaaagtatgactgcttgccatgtagataccatggcttgctgaataatcagaagaggtgtgactcttattctaaaatttgtcacaaaatgtcaaaatgagagactctgtaggaacgagtccttgacagacagctcaaggggtttttttcctttgtctcatttctacatgaaagtaaatttgaaatgatcttttttattataagagtagaaatacagttgggtttgaactatatgttttaatggccacggttttgtaagacatttggtcctttgttttcccagttattactcgattgtaattttatatcgccagcaatggactgaaacggtccgcaacctcttctttacaactgggtgacctcgcggctgtgccagccatttggcgttcaccctgccgctaagggccatgtgaacccccggaacagagaaacaggagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctctatataagcagagctcgtttagtgaaccgtcagatcaccgggccgccaccatgtataggatgcagctgctgagctgcatcgccctgagcctggccctggtgaccaacagcttggacaatggcttggcgcggactcctactatgggctggctgcattgggaacgtttcatgtgcaaccttgactgccaagaagagcctgatgcctgcataagtgagcaactgttcatgcagatggcagagctcatggtctctgatggctggcgggatgcaggttatgactatctctgcatagatgactgttggatggctcccgagagggattcaaagggcaggcttcaggcagatccccaacgctttcctagtgggatcaaacacctcgcaaattacgtccacagcaaaggattgaagctagggatttatgcagatgttgggaataaaacctgtgcaggtttccccgggagttttggatcctatgacattgatgcgcagacatttgctgactggggcgtagatctgctaaaatttgatggttgtcactgtgacagtgtagtatccttggagaatggttataagtacatggccttggccttgaacaggacaggccgaagcattgtatactcctgtgagtggccactttatttgagaccctttcataagcccaattatacagatatccaatattactgcaatcattggagaaattttgatgatgtttatgattcctgggaaagcataaagaatatcttgtcttggacagtggtttaccagaaggagattgttgaagtcgctggaccaggcagctggaatgacccagacatgttagtgatcggcaactttggcctcagttgggaccagcaggtgacacagatggccctctgggctatcatggccgctcccctactcatgtccaacgatctccgacaaatcagctctcaagccaaagctctgcttcagaataaggatgtaattgccatcaaccaagaccccttgggcaagcagggctactgtttcagaaaggaaaaccacattgaggtttgggaacggccactctccaacctagcctgggctgtggctgtgagaaacctgcaggagattggtggaccttgtccttataccatccagatttcttccctgggtagaggactagcctgcaatcctggctgcatcattactcagcttctccccgagaaagtacacctaggcttctatgaatggactttgaccttaaaaactcgagtaaacccctcaggcactgttttgtttcggttagaaagataatttttttccggtcggtagcatcccttgctccgcgtggaccactttcctgaggcacagtgataggaacagagccactaatctgaagagaacagagatgtgacagactacactaatgtgagaaaaacaaggaaagggtgacttattggagatttcagaaataaaatgcatttattattatattcccttattttaattttctattagggaattagaaagggcataaactgctttatccagtgttatattaaaagcttaatgtatataatcttttagaggtaaaatctacagccagcaaaagtcatggtaaatattctttgactgaactctcactaaactcctctaaattatatgtcatattaactggttaaattaatataaatttgtgacatgaccttaactggttaggtaggatatttttcttcatgcaaaaatatgactaataataatttagcacaaaaatatttcccaatactttaattctgtgatagaaaaatgtttaactcagctactatgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgccagctggcgtaatagcgaagaggcccgcaccgatcgcccttcccaacagttgcgcagcctgaatggcgaatggcgattccgttgcaatggctggcggtaatattgttctggatattaccagcaaggccgatagtttgagttcttctactcaggcaagtgatgttattactaatcaaagaagtattgcgacaacggttaatttgcgtgatggacagactcttttactcggtggcctcactgattataaaaacacttctcaggattctggcgtaccgttcctgtctaaaatccctttaatcggcctcctgtttagctcccgctctgattctaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttaaatatttgcttatacaatcttcctgtttttggggcttttctgattatcaaccggggtacatatgattgacatgctagttttacgattaccgttcatcgattctcttgtttgctccagactctcaggcaatgacctgatagcctttgtagagacctctcaaaaatagctaccctctccggcatgaatttatcagctagaacggttgaatatcatattgatggtgatttgactgtctccggcctttctcacccgtttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgttggaatcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatg

SEQ ID NO: 307 - DNA, 7308 bp, Synthetic construct
  source 1..7308: mol_type = other DNA
gacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgagtacatttatattggctcatgtccaatatgaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtcagatcctcactctcttccgcatcgctgtctgcgagggccagctgttgggctcgcggttgaggacaaactcttcgcggtctttccagtactcttggatcggaaacccgtcggcctccgaacggtactccgccaccgagggacctgagcgagtccgcatcgaccggatcggaaaacctctcgagaaaggcgtctaaccagtcacagtcgcaaggtaggctgagcaccgtggcgggcggcagcgggtggcggtcggggttgtttctggcggaggtgctgctgatgatgtaattaaagtaggcggtcttgagacggcggatggtcgaggtgaggtgtggcaggcttgagatccagctgttggggtgagtactccctctcaaaagcgggcattacttctgcgctaagattgtcagtttccaaaaacgaggaggatttgatattcacctggcccgatctggccatacacttgagtgacaatgacatccactttgcctttctctccacaggtgtccactcccaggtccaagtttaaactttaatacgactcactataggggccgccaccaagcttatgtacagaatgcagctgctgagctgcatcgccctgagcctggccctggtgaccaacagcctggacaatggattggcaaggacgcctaccatgggctggctgcactgggagcgcttcatgtgcaaccttgactgccaggaagagccagattcctgcatcagtgagaagctcttcatggagatggcagagctcatggtctcagaaggctggaaggatgcaggttatgagtacctctgcattgatgactgttggatggctccccaaagagattcagaaggcagacttcaggcagaccctcagcgctttcctcatgggattcgccagctagctaattatgttcacagcaaaggactgaagctagggatttatgcagatgttggaaataaaacctgcgcaggcttccctgggagttttggatactacgacattgatgcccagacctttgctgactggggagtagatctgctaaaatttgatggttgttactgtgacagtttggaaaatttggcagatggttataagcacatgtccttggccctgaataggactggcagaagcattgtgtactcctgtgagtggcctctttatatgtggccctttcaaaagcccaattatacagaaatccgacagtactgcaatcactggcgaaattttgctgacattgatgattcctggaaaagtataaagagtatcttggactggacatcttttaaccaggagagaattgttgatgttgctggaccagggggttggaatgacccagatatgttagtgattggcaactttggcctcagctggaatcagcaagtaactcagatggccctctgggctatcatggctgctcctttattcatgtctaatgacctccgacacatcagccctcaagccaaagctctccttcaggataaggacgtaattgccatcaatcaggaccccttgggcaagcaagggtaccagcttagacagggagacaactttgaagtgtgggaacgacctctctcaggcttagcctgggctgtagctatgataaaccggcaggagattggtggacctcgctcttataccatcgcagttgcttccctgggtaaaggagtggcctgtaatcctgcctgcttcatcacacagctcctccctgtgaaaaggaagctagggttctatgaatggacttcaaggttaagaagtcacataaatcccacaggcactgttttgcttcagctagaaaatacaatgcagatgtcattaaaagacttactttaatttttttctagagcggccgccgaattcgggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatagcacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 308 - DNA, 7308 bp, Synthetic construct
  source 1..7308: mol_type = other DNA
gacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgagtacatttatattggctcatgtccaatatgaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtcagatcctcactctcttccgcatcgctgtctgcgagggccagctgttgggctcgcggttgaggacaaactcttcgcggtctttccagtactcttggatcggaaacccgtcggcctccgaacggtactccgccaccgagggacctgagcgagtccgcatcgaccggatcggaaaacctctcgagaaaggcgtctaaccagtcacagtcgcaaggtaggctgagcaccgtggcgggcggcagcgggtggcggtcggggttgtttctggcggaggtgctgctgatgatgtaattaaagtaggcggtcttgagacggcggatggtcgaggtgaggtgtggcaggcttgagatccagctgttggggtgagtactccctctcaaaagcgggcattacttctgcgctaagattgtcagtttccaaaaacgaggaggatttgatattcacctggcccgatctggccatacacttgagtgacaatgacatccactttgcctttctctccacaggtgtccactcccaggtccaagtttaaactttaatacgactcactataggggccgccaccaagcttatgagaagaatgcagctgctgctgctgatcgccctgagcctggccctggtgaccaacagcctggacaatggattggcaaggacgcctaccatgggctggctgcactgggagcgcttcatgtgcaaccttgactgccaggaagagccagattcctgcatcagtgagaagctcttcatggagatggcagagctcatggtctcagaaggctggaaggatgcaggttatgagtacctctgcattgatgactgttggatggctccccaaagagattcagaaggcagacttcaggcagaccctcagcgctttcctcatgggattcgccagctagctaattatgttcacagcaaaggactgaagctagggatttatgcagatgttggaaataaaacctgcgcaggcttccctgggagttttggatactacgacattgatgcccagacctttgctgactggggagtagatctgctaaaatttgatggttgttactgtgacagtttggaaaatttggcagatggttataagcacatgtccttggccctgaataggactggcagaagcattgtgtactcctgtgagtggcctctttatatgtggccctttcaaaagcccaattatacagaaatccgacagtactgcaatcactggcgaaattttgctgacattgatgattcctggaaaagtataaagagtatcttggactggacatcttttaaccaggagagaattgttgatgttgctggaccagggggttggaatgacccagatatgttagtgattggcaactttggcctcagctggaatcagcaagtaactcagatggccctctgggctatcatggctgctcctttattcatgtctaatgacctccgacacatcagccctcaagccaaagctctccttcaggataaggacgtaattgccatcaatcaggaccccttgggcaagcaagggtaccagcttagacagggagacaactttgaagtgtgggaacgacctctctcaggcttagcctgggctgtagctatgataaaccggcaggagattggtggacctcgctcttataccatcgcagttgcttccctgggtaaaggagtggcctgtaatcctgcctgcttcatcacacagctcctccctgtgaaaaggaagctagggttctatgaatggacttcaaggttaagaagtcacataaatcccacaggcactgttttgcttcagctagaaaatacaatgcagatgtcattaaaagacttactttaatttttttctagagcggccgccgaattcgggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatagcacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 309 - DNA, 7347 bp, Synthetic construct
  source 1..7347: mol_type = other DNA
gacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgagtacatttatattggctcatgtccaatatgaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtcagatcctcactctcttccgcatcgctgtctgcgagggccagctgttgggctcgcggttgaggacaaactcttcgcggtctttccagtactcttggatcggaaacccgtcggcctccgaacggtactccgccaccgagggacctgagcgagtccgcatcgaccggatcggaaaacctctcgagaaaggcgtctaaccagtcacagtcgcaaggtaggctgagcaccgtggcgggcggcagcgggtggcggtcggggttgtttctggcggaggtgctgctgatgatgtaattaaagtaggcggtcttgagacggcggatggtcgaggtgaggtgtggcaggcttgagatccagctgttggggtgagtactccctctcaaaagcgggcattacttctgcgctaagattgtcagtttccaaaaacgaggaggatttgatattcacctggcccgatctggccatacacttgagtgacaatgacatccactttgcctttctctccacaggtgtccactcccaggtccaagtttaaactttaatacgactcactataggggccgccaccaagcttggtaccatgcagctgaggaacccagaactacatctgggctgcgcgcttgcgcttcgcttcctggccctcgtttcctgggacatccctggggctagagcactggacaatggattggcaaggacgcctaccatgggctggctgcactgggagcgcttcatgtgcaaccttgactgccaggaagagccagattcctgcatcagtgagaagctcttcatggagatggcagagctcatggtctcagaaggctggaaggatgcaggttatgagtacctctgcattgatgactgttggatggctccccaaagagattcagaaggcagacttcaggcagaccctcagcgctttcctcatgggattcgccagctagctaattatgttcacagcaaaggactgaagctagggatttatgcagatgttggaaataaaacctgcgcaggcttccctgggagttttggatactacgacattgatgcccagacctttgctgactggggagtagatctgctaaaatttgatggttgttactgtgacagtttggaaaatttggcagatggttataagcacatgtccttggccctgaataggactggcagaagcattgtgtactcctgtgagtggcctctttatatgtggccctttcaaaagcccaattatacagaaatccgacagtactgcaatcactggcgaaattttgctgacattgatgattcctggaaaagtataaagagtatcttggactggacatcttttaaccaggagagaattgttgatgttgctggaccagggggttggaatgacccagatatgttagtgattggcaactttggcctcagctggaatcagcaagtaactcagatggccctctgggctatcatggctgctcctttattcatgtctaatgacctccgacacatcagccctcaagccaaagctctccttcaggataaggacgtaattgccatcaatcaggaccccttgggcaagcaagggtaccagcttagacagggagacaactttgaagtgtgggaacgacctctctcaggcttagcctgggctgtagctatgataaaccggcaggagattggtggacctcgctcttataccatcgcagttgcttccctgggtaaaggagtggcctgtaatcctgcctgcttcatcacacagctcctccctgtgaaaaggaagctagggttctatgaatggacttcaaggttaagaagtcacataaatcccacaggcactgttttgcttcagctagaaaatacaatgcagatgtcattaaaagacttactttaatttttttctagagcggccgccgaattcgggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatagcacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 310 - DNA, 7087 bp, Synthetic construct
  source 1..7087: mol_type = other DNA
cagctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacccaggggtgattctagtcagactctggggtttttgtcgggtatagaggaaaaatccactattgtgattactatgctatggactactggggtcaaggaacctcagtcaccgtctcctcaggtaagaatggcctctccaggtctttatttttaacctttgttatggagttttctgagcattgcagactaatcttggatatttgtccctgagggagccggctgagagaagttgggaaataaactgtctagggatctcagagcctttaggacagattatctccacatctttgaaaaactaagaatctgtgtgatggtgttggtggagtccctggatgatgggatagggactttggaggctcatttgagggagatgctaaaacaatcctatggctggagggatagttggggctgtagttggagattttcagtttttagaataaaagtattagttgtggaatatacttcaggaccacctctgtgacagcatttatacagtatccgatggatgacaagtgagtgtctcaggttaggattctattttaagattgagatattaggctttgatactacatctaaatggtctgtacatgtctcgaagaaagttcttcagacagagttaggacttggatccaggagttaggacttggactgactcaggaggactctagtttcttcttctccagctggaatgtccttatgtaagaaaagccttgcctcatgagtatgcaaatcatgtgcgactgtgatgattaatatagggatatccacaccaaacatcatatgagccctatcttctctacagacactgaatctcaaggtccttacagccgccaccatggaaaccgacacactgctgctgtgggtgctgcttctttgggtgcccggaagcacaggcgacatggtcatgtctcaatctccatcctctcttgctgttagcgccggggaaaaggtatctatgagttgcaagtcttctcaaaccttgctgaacagccgcactagaaaaaattacctggcttggtatcaacaaaaaccaggacagagtcctaaactgctcatttactgggccagcacccgagagtctggagtacctgatcgatttactggctcaggcagcggcacagatttcacacttacaatctcttctgtccaagccgaagacttggctgtttattactgcaaacaatcctataatctgtggacattcgggggcggaaccaagcttgaaattaaacgcgccgatgccgctcctaccgtgtctatctttccacctagcagcgagcagctgacatctggcggagcctctgtcgtgtgcttcctgaacaacttctaccctaaggacatcaacgtcaagtggaagatcgacggctccgagagacagaacggcgtgctgaactcttggaccgaccaggacagcaaggatagcacctacagcatgagcagcactctgaccctgacaaaggacgagtacgagaggcacaactcctacacatgcgaggccacacacaagaccagcacatccccaatcgtgaagtccttcaaccggaacgagtgcggaggaagtagtggcagcgggagtgggtccaattggagtcatcctcaatttgagaaaggagggggagggtccaattggtctcatccgcagtttgagaagggcggcggcggctccaattggtcccatccccagtttgaaaaaggctctggtggaggtggtagtgctggtgggcaggtccagttgcagcagagtggcgccgagcttgcaaaaccaggtgctagtgttaaaatgagctgtaaggcatccggttacacttttacccgatattggatgcactgggtgaagcaaaggccaggtcaaggacttgagtggataggatacattaatccttcaactggctacactgagtataatcaaaagtttaaggacaaagctacactcaccgctgataagagttccagcactgtatatatgcagttgtctagtctcacatccgaggattccgctgtatactattgtgctcgaactactgtagacggatatgattttgcatactgggggcagggaactctcgttacagtctcatccggtgagtcctaacttctcccattctaaatgcatgttggggggattctgggccttcaggaccaccatgtaccaaaagccataacgatcggtgggagtattcattgtggtcaagatccatagggacaaagagtggagtggggcactttctttagatttgtgaggaatgttccgcactagattgtttaaaacttcatttgttggaaggagagctgtcttagtgattgagtcaagggagaaaggcatctagcctcggtctcaaaagggtagttgctgtctagagaggtctggtggagcctgcaaaagtccagctttcaaaggaacacagaagtatgtgtatggaatattagaagatgttgcttttactcttaagttggttcctaggaaaaatagttaaatactgtgactttaaaatgtgagagggttttcaagtactcatttttttaaatgtccaaaatttttgtcaatcagtttgaggtcttgtttgtgtagaactgatattacttaaagtttaaccgaggaatgggagtgaggctctctcataacctattcagaactgacttttaacaataataaattaagtttaaaatatttttaaatgaattgagcaatgttgagttggagtcaagatggccgatcagaaccagaacacctgcagcagctggcaggaagcaggtcatgtggcaaggctatttggggaagggaaaataaaaccactaggtaaacttgtagctgtggtttgaagaagtggttttgaaacactctgtccagccccaccaaaccgaaagtccaggctgagcaaaacaccacctgggtaatttgcatttctaaaataagttgaggattcagccgaaactggagaggtcctcttttaacttattgagttcaaccttttaattttagcttgagtagttctagtttccccaaacttaagtttatcgacttctaaaatgtatttagaattcattttcaaaattaggttatgtaagaaattgaaggactttagtgtctttaatttctaatatatttagaaaacttcttaaaattactctattattcttccctctgattattggtctccattcagtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgccagctggcgtaatagcgaagaggcccgcaccgatcgcccttcccaacagttgcgcagcctgaatggcgaatggcgattccgttgcaatggctggcggtaatattgttctggatattaccagcaaggccgatagtttgagttcttctactcaggcaagtgatgttattactaatcaaagaagtattgcgacaacggttaatttgcgtgatggacagactcttttactcggtggcctcactgattataaaaacacttctcaggattctggcgtaccgttcctgtctaaaatccctttaatcggcctcctgtttagctcccgctctgattctaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttaaatatttgcttatacaatcttcctgtttttggggcttttctgattatcaaccggggtacatatgattgacatgctagttttacgattaccgttcatcgattctcttgtttgctccagactctcaggcaatgacctgatagcctttgtagagacctctcaaaaatagctaccctctccggcatgaatttatcagctagaacggttgaatatcatattgatggtgatttgactgtctccggcctttctcacccgtttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgttggaatcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatg

SEQ ID NO: 311 - DNA, 8264 bp, Synthetic construct
  source 1..8264: mol_type = other DNA
agatctatacattgaatcaatattggcaattagccatattagtcattggttatatagcataaatcaatattggctattggccattgcatacgttgtatctatatcataatatgtacatttatattggctcatgtccaatatgaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtcagatcctcactctcttccgcatcgctgtctgcgagggccagctgttgggctcgcggttgaggacaaactcttcgcggtctttccagtactcttggatcggaaacccgtcggcctccgaacggtactccgccaccgagggacctgagcgagtccgcatcgaccggatcggaaaacctctcgagaaaggcgtctaaccagtcacagtcgcaaggtaggctgagcaccgtggcgggcggcagcgggtggcggtcggggttgtttctggcggaggtgctgctgatgatgtaattaaagtaggcggtcttgagacggcggatggtcgaggtgaggtgtggcaggcttgagatccagctgttggggtgagtactccctctcaaaagcgggcattacttctgcgctaagattgtcagtttccaaaaacgaggaggatttgatattcacctggcccgatctggccatacacttgagtgacaatgacatccactttgcctttctctccacaggtgtccactcccaggtccaagtttaaacggatctctagcgaattcgccgccaccatggacgccatgaagagagggctgtgctgcgtgctgctgctgtgcggagcagtgttcgtcagcccttccgctagcgacatggtcatgtctcaatctccatcctctcttgctgttagcgccggggaaaaggtatctatgagttgcaagtcttctcaaaccttgctgaacagccgcactagaaaaaattacctggcttggtatcaacaaaaaccaggacagagtcctaaactgctcatttactgggccagcacccgagagtctggagtacctgatcgatttactggctcaggcagcggcacagatttcacacttacaatctcttctgtccaagccgaagacttggctgtttattactgcaaacaatcctataatctgtggacattcgggggcggaaccaagcttgaaattaaacgcgccgatgccgctcctaccgtgtctatctttccacctagcagcgagcagctgacatctggcggagcctctgtcgtgtgcttcctgaacaacttctaccctaaggacatcaacgtcaagtggaagatcgacggctccgagagacagaacggcgtgctgaactcttggaccgaccaggacagcaaggatagcacctacagcatgagcagcactctgaccctgacaaaggacgagtacgagaggcacaactcctacacatgcgaggccacacacaagaccagcacatccccaatcgtgaagtccttcaaccggaacgagtgcggaggaagtagtggcagcgggagtgggtccaattggagtcatcctcaatttgagaaaggagggggagggtccaattggtctcatccgcagtttgagaagggcggcggcggctccaattggtcccatccccagtttgaaaaaggctctggtggaggtggtagtgctggtgggcaggtccagttgcagcagagtggcgccgagcttgcaaaaccaggtgctagtgttaaaatgagctgtaaggcatccggttacacttttacccgatattggatgcactgggtgaagcaaaggccaggtcaaggacttgagtggataggatacattaatccttcaactggctacactgagtataatcaaaagtttaaggacaaagctacactcaccgctgataagagttccagcactgtatatatgcagttgtctagtctcacatccgaggattccgctgtatactattgtgctcgaactactgtagacggatatgattttgcatactgggggcagggaactctcgttacagtctcatccgccaagaccacacctcctagcgtgtaccctctggctcctggatctgccgctcagaccaacagcatggtcacactgggctgtctggtcaagggctacttccctgagcctgtgaccgtgacatggaacagcggatctctgagcagcggagtgcacacattccctgccgtgctgcagagcgacctgtacacactgagcagcagcgtgacagtgcccagctctacatggcctagcgagacagtgacctgcaacgtggcccatcctgccagcagtaccaaggtggacaagaaaatcgtgcccagagactgcggctgcaagccctgcatctgtacagtgcctgaagtgtccagcgtgttcatcttcccacctaagcctaaggacgtgctgacaatcaccctgacacctaaagtgacctgtgtggtggtggacatctccaaggacgaccccgaggtgcagttcagttggttcgtggacgacgtggaagtgcacactgcccagacacagcccagagaggaacagttcaacagcaccttcagaagcgtgtccgagctgcccatcatgcaccaggattggctgaacggcaaagaattcaagtgcagagtgaacagcgccgcctttcctgctcctatcgaaaagacaatcagcaagaccaagggcagacccaaggctccccaggtgtacacaatccctccacctaaagaacagatggccaaggacaaggtgtccctcacctgtatgatcaccgatttcttcccagaggacatcaccgtggaatggcagtggaacggacagcccgccgagaactacaagaacacccagcctatcatggacaccgacggcagctacttcgtgtacagcaagctgaacgtgcagaagtccaactgggaggccggcaacaccttcacctgttctgtgctgcacgagggcctgcacaatcaccacaccgagaagtccctgtctcacagccctggcaaaggatccggttccggtcaccatcaccatcaccatggcctgaatgatatttttgaggcccagaagattgagtggcatgaatgacccgacctcgacctctggctaataaaggaaatttattttcattgcaatagtgtgttggaattttttgtgtctctcactcggaaggacatatgggagggcaaatcatttggtcgagatccctcggagatctctagctagaggatcgatccccgccccggacgaactaaacctgactacgacatctctgccccttcttcgcggggcagtgcatgtaatcccttcagttggttggtacaacttgccaactgggccctgttccacatgtgacacggggggggaccaaacacaaaggggttctctgactgtagttgacatccttataaatggatgtgcacatttgccaacactgagtggctttcatcctggagcagactttgcagtctgtggactgcaacacaacattgcctttatgtgtaactcttggctgaagctcttacaccaatgctgggggacatgtacctcccaggggcccaggaagactacgggaggctacaccaacgtcaatcagaggggcctgtgtagctaccgataagcggaccctcaagagggcattagcaatagtgtttataaggcccccttgttaaccctaaacgggtagcatatgcttcccgggtagtagtatatactatccagactaaccctaattcaatagcatatgttacccaacgggaagcatatgctatcgaattagggttagtaaaagggtcctaaggaacagcgatatctcccaccccatgagctgtcacggttttatttacatggggtcaggattccacgagggtagtgaaccattttagtcacaagggcagtggctgaagatcaaggagcgggcagtgaactctcctgaatcttcgcctgcttcttcattctccttcgtttagctaatagaataactgctgagttgtgaacagtaaggtgtatgtgaggtgctcgaaaacaaggtttcaggtgacgcccccagaataaaatttggacggggggttcagtggtggcattgtgctatgacaccaatataaccctcacaaaccccttgggcaataaatactagtgtaggaatgaaacattctgaatatctttaacaatagaaatccatggggtggggacaagccgtaaagactggatgtccatctcacacgaatttatggctatgggcaacacataatcctagtgcaatatgatactggggttattaagatgtgtcccaggcagggaccaagacaggtgaaccatgttgttacactctatttgtaacaaggggaaagagagtggacgccgacagcagcggactccactggttgtctctaacacccccgaaaattaaacggggctccacgccaatggggcccataaacaaagacaagtggccactcttttttttgaaattgtggagtgggggcacgcgtcagcccccacacgccgccctgcggttttggactgtaaaataagggtgtaataacttggctgattgtaaccccgctaaccactgcggtcaaaccacttgcccacaaaaccactaatggcaccccggggaatacctgcataagtaggtgggcgggccaagataggggcgcgattgctgcgatctggaggacaaattacacacacttgcgcctgagcgccaagcacagggttgttggtcctcatattcacgaggtcgctgagagcacggtgggctaatgttgccatgggtagcatatactacccaaatatctggatagcatatgctatcctaatctatatctgggtagcataggctatcctaatctatatctgggtagcatatgctatcctaatctatatctgggtagtatatgctatcctaatttatatctgggtagcataggctatcctaatctatatctgggtagcatatgctatcctaatctatatctgggtagtatatgctatcctaatctgtatccgggtagcatatgctatcctaatagagattagggtagtatatgctatcctaatttatatctgggtagcatatactacccaaatatctggatagcatatgctatcctaatctatatctgggtagcatatgctatcctaatctatatctgggtagcataggctatcctaatctatatctgggtagcatatgctatcctaatctatatctgggtagtatatgctatcctaatttatatctgggtagcataggctatcctaatctatatctgggtagcatatgctatcctaatctatatctgggtagtatatgctatcctaatctgtatccgggtagcatatgctatcctcatgataagctgtcaaacatgagaattttcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctctagctagaggtcgaccaattctcatgtttgacagcttatcatcgcagatccgggcaacgttgttgccattgctgcaggcgcagaactggtaggtatggc

SEQ ID NO: 312 - protein, 310 aa, Vaccinia virus
MKTISVVTLLCVLPAVVYSTCTVPTMNNAKLTSTETSFNNNQKVTFTCDQGYHSSDPNAVCETDKWKYENPCKKMCTVSDYISELYNKPLYEVNSTMTLSCNGETKYFRCEEKNGNTSWNDTVTCPNAECQPLQLEHGSCQPVKEKYSFGEYITINCDVGYEVIGASYISCTANSWNVIPSCQQKCDIPSLSNGLISGSTFSIGGVIHLSCKSGFILTGSPSSTCIDGKWNPILPTCVRSNEKFDPVDDGPDDETDLSKLSKDVVQYEQEIESLEATYHIIIVALTIMGVIFLISVIVLVCSCDKNNDQY

SEQ ID NO: 313 - protein, 185 aa, Vaccinia virus
MMTPENDEEQTSVFSATVYRDKIQGKNKRKRVIGLCIRISMVISLLSMITMSAFLIVRLNQCMSANEAAITDAAVAVAAASSTHRKVASSTTQYDHKESCNGLYYQGSCYILHSDYQLFSDAKANCTAESSTLPNKSDVLTTWLIDYVKDTWGSDGNPITKTTSDYQDSDVSQEVRKYFCVKTMN

SEQ ID NO: 314 - protein, 392 aa, Enterovirus C
MARAMAAAWPLLLVALLVLSWPPPGTGDVVVQAPTQVPGFLGDSVTLPCYLQVPNMEVTHVSQLTWARHGESGSMAVFHQTQGPSYSESKRLEFVAARLGAELRNASLRMFGLRVEDEGNYTCLFVTFPQGSRSVDIWLRVLAKPQNTAEVQKVQLTGEPVPMARCVSTGGRPPAQITWHSDLGGMPNTSQVPGFLSGTVTVTSLWILVPSSQVDGKNVTCKVEHESFEKPQLLTVNLTVYYPPEVSISGYDNNWYLGQNEATLTCDARSNPEPTGYNWSTTMGPLPPFAVAQGAQLLIRPVDKPINTTLICNVTNALGARQAELTVQVKEGPPSEHSGISRNAIIFLVLGILVFLILLGIGIYFYWSKCSREVLWHCHLCPSSEHHQSCRN

SEQ ID NO: 315 - protein, 120 aa, Synthetic construct
EVQLLESGGGLIKPGGSLRLSCAASGFIFRDYNINWVRQAPGKGLEWLGFIRTRASGRSTEYSASVKGRFTISRDDSKNIAYLHINSLKMEDTAVYYCAKKGDSYYYMDFWGKGTAVTVS

SEQ ID NO: 316 - protein, 112 aa, Synthetic construct
ELALTQPASVSGSPGQSITISCTGGRSDLGDSNFVSWYQQYPGKAPKLLIYQVNKRPSGVPDRFSASKSANTASLTISGLQTEDEADYFCSSYTTTSTYVFGIGTKVVVLGQ

SEQ ID NO: 317 - protein, 5 aa, Synthetic construct
DYNIN

SEQ ID NO: 318 - protein, 12 aa, Synthetic construct
FIRTRASGRSTE

SEQ ID NO: 319 - protein, 10 aa, Synthetic construct
KGDSYYYMDF

SEQ ID NO: 320 - protein, 14 aa, Synthetic construct
TGGRSDLGDSNFVS

SEQ ID NO: 321 - protein, 7 aa, Synthetic construct
QVNKRPS

SEQ ID NO: 322 - protein, 10 aa, Synthetic construct
SSYTTTSTYV

SEQ ID NO: 323 - protein, 123 aa, Synthetic construct
EVQLEQSGSEVKKPGASVKLSCKASGYTFTSYSLGWVRQAPGQGLEWMGWINTKTGNPTYAQGFTGRFVFSLDTSVNTAYLQITSLKAEDTAVYFCAKGTFYYGWGPYYNWFDPWGQGALVTV

SEQ ID NO: 324 - protein, 113 aa, Synthetic construct
AELVLTQPPSVSAAPGQKITISCSGSGSNIGRHYVSWYQQFPGTAPKILIYDNDKRPSGISDRFSGSKSGASATLDITGLQTGDEADYYCATWDTNLSGGVFGGGTKVTVLGQ

SEQ ID NO: 325 - protein, 5 aa, Synthetic construct
SYSLG

SEQ ID NO: 326 - protein, 10 aa, Synthetic construct
WINTKTGNPT

SEQ ID NO: 327 - protein, 16 aa, Synthetic construct
GTFYYGWGPYYNWFDP

SEQ ID NO: 328 - protein, 13 aa, Synthetic construct
SGSGSNIGRHYVS

SEQ ID NO: 329 - protein, 7 aa, Synthetic construct
DNDKRPS

SEQ ID NO: 330 - protein, 11 aa, Synthetic construct
ATWDTNLSGGV

SEQ ID NO: 331 - protein, 119 aa, Synthetic construct
QVQLQQSGAELMKPGASVKISCKATGYTFSNYWIEWIKQRPGHGLEWIGEIFPGSGRINFNEKFKGKATFTADTSSDTTYMQLSSLTSADSAVYYCARTKIYGNSFDYWGQGTTLTVSP

SEQ ID NO: 332 - protein, 107 aa, Synthetic construct
DIMMTQSHKFMSTSVGDRVNITCKASQDVGTAVVWYQQKPGQSPKLLIYWASSRHNGVPDRFTGSGSGTDFTLTISNVQSEDLSDYFCQQYSRYPLTFGAGTKLELK

SEQ ID NO: 333 - protein, 8 aa, Synthetic construct
GFDFSRYW

SEQ ID NO: 334 - protein, 15 aa, Synthetic construct
EIHPDSSKINYTPSQ

SEQ ID NO: 335 - protein, 11 aa, Synthetic construct
PDGNYNALDYW

SEQ ID NO: 336 - protein, 11 aa, Synthetic construct
KASQDVGTAVT

SEQ ID NO: 337 - protein, 7 aa, Synthetic construct
WASTRHT

SEQ ID NO: 338 - protein, 9 aa, Synthetic construct
QQYSRYPYT

SEQ ID NO: 339 - protein, 21 aa, Synthetic construct
  REGION 1..3: note = GSG residues can be added to the 5' end of the peptide to improve cleavage efficiency
GSGEGRGSLLTCGDVEENPGP

SEQ ID NO: 340 - protein, 22 aa, Synthetic construct
  REGION 1..3: note = GSG residues can be added to the 5' end of the peptide to improve cleavage efficiency
GSGATNFSLLKQAGDVEENPGP

SEQ ID NO: 341 - protein, 24 aa, Synthetic construct
  REGION 1..3: note = GSG residues can be added to the 5' end of the peptide to improve cleavage efficiency
GSGQCTNYALLKLAGDVESNPGPP

SEQ ID NO: 342 - protein, 25 aa, Synthetic construct
  REGION 1..3: note = GSG residues can be added to the 5' end of the peptide to improve cleavage efficiency
GSGVKQTLNFDLLKLAGDVESNPGP

SEQ ID NO: 343 - skipped

SEQ ID NO: 344 - protein, 4 aa, Synthetic construct
  REPEAT 1..4: note = (GGGS) is repeated n times wherein n is an integer from 1 to 100
GGGS

SEQ ID NO: 345 - protein, 5 aa, Synthetic construct
  REPEAT 1..5: note = wherin (GGGGS) is repeated n times wherein n is an integer from 1 to 100
GGGGS

SEQ ID NO: 346 - DNA, 171 bp, Synthetic construct
  source 1..171: mol_type = other DNA
ggaggaagtagtggcagcgggagtgggtccaattggagtcatcctcaatttgagaaaggagggggagggtccaattggtctcatccgcagtttgagaagggcggcggcggctccaattggtcccatccccagtttgaaaaaggctctggtggaggtggtagtgctggtggg

SEQ ID NO: 347 - protein, 57 aa, Synthetic construct
GGSSGSGSGSNWSHPQFEKGGGGSNWSHPQFEKGGGGSNWSHPQFEKGSGGGGSAGG

SEQ ID NO: 348 - protein, 14 aa, Synthetic construct
  REPEAT 1..14: note = sequence is repeated n times, wherein n is an integer from 1 to 100
EGKSSGSGSESKST

SEQ ID NO: 349 - DNA, 67 bp, Synthetic construct
  source 1..67: mol_type = other DNA
caggtaagtctgctgtctggggatagcggggagccaggtgtactgggccaggcaagggctttggatc

SEQ ID NO: 350 - DNA, 87 bp, Synthetic construct
  source 1..87: mol_type = other DNA
caggtgagttggctttccttctgcctcctttctctgggcccagcgtcctctgacctggagctgggagataatgtccgggggctcctt

SEQ ID NO: 351 - protein, 19 aa, Synthetic construct
MELGLSWIFLLAILKGVQC

SEQ ID NO: 352 - protein, 19 aa, Synthetic construct
MELGLRWVFLVAILEGVQC

SEQ ID NO: 353 - protein, 19 aa, Synthetic construct
MKHLWFFLLLVAAPRWVLS

SEQ ID NO: 354 - protein, 19 aa, Synthetic construct
MDWTWRILFLVAAATGAHS

SEQ ID NO: 355 - protein, 19 aa, Synthetic construct
MDWTWRFLFVVAAATGVQS

SEQ ID NO: 356 - protein, 19 aa, Synthetic construct
MEFGLSWLFLVAILKGVQC

SEQ ID NO: 357 - protein, 19 aa, Synthetic construct
MEFGLSWVFLVALFRGVQC

SEQ ID NO: 358 - protein, 26 aa, Synthetic construct
MDLLHKNMKHLWFFLLLVAAPRWVLS

SEQ ID NO: 359 - protein, 22 aa, Synthetic construct
MDMRVPAQLLGLLLLWLSGARC

SEQ ID NO: 360 - protein, 22 aa, Synthetic construct
MKYLLPTAAAGLLLLAAQPAMA

SEQ ID NO: 361 - DNA, 93 bp, Homo sapiens
  source 1..93: mol_type = genomic DNA
atgcagctgaggaacccagaactacatctgggctgcgcgcttgcgcttcgcttcctggccctcgtttcctgggacatccctggggctagagca

SEQ ID NO: 362 - DNA, 60 bp, Synthetic construct
  source 1..60: mol_type = other DNA
atgtataggatgcagctgctgagctgcatcgccctgagcctggccctggtgaccaacagc

SEQ ID NO: 363 - DNA, 60 bp, Synthetic construct
  source 1..60: mol_type = other DNA
atgaggaggatgcagctgctgctgctgatcgccctgagcctggccctggtgaccaacagc

SEQ ID NO: 364 - DNA, 93 bp, Mus musculus
  source 1..93: mol_type = genomic DNA
atgaagcttttgagcagagatacgcggctggtctgtgagcttgcgctttgtcccctggctttagttttctggagcattcttggggtcagagca

SEQ ID NO: 365 - protein, 8 aa, Synthetic construct
WSHPQFEK

SEQ ID NO: 366 - protein, 8 aa, Synthetic construct
DYKDDDDK

SEQ ID NO: 367 - protein, 8 aa, Synthetic construct
DLYDDDDK

SEQ ID NO: 368 - protein, 15 aa, Synthetic construct
GLNDIFEAQKIEWHE

SEQ ID NO: 369 - protein, 26 aa, Synthetic construct
KRRWKKNFIAVSAANRFKKISSSGAL

SEQ ID NO: 370 - protein, 9 aa, Synthetic construct
YPYDVPDYA

SEQ ID NO: 371 - protein, 10 aa, Synthetic construct
EQKLISEEDL

SEQ ID NO: 372 - protein, 13 aa, Synthetic construct
SLAELLNAGLGGS

SEQ ID NO: 373 - protein, 8 aa, Synthetic construct
TQDPSRVG

SEQ ID NO: 374 - protein, 14 aa, Synthetic construct
GKPIPNPLLGLDST

SEQ ID NO: 375 - protein, 123 aa, Synthetic construct
QVQLVQSGAEVKKPGSSVKVSCKASGGTFSSYTINWVRQAPGQGLEWMGGIIPIFGIPNYAQKFQGRVTITADESTNTAYMELSSLRAEDTAVYYCARASGGSADYSYGMDVWGQGTAVTVSS

SEQ ID NO: 376 - protein, 107 aa, Synthetic construct
DIQMTQSPSSLSASVGDRVTITCRASQGISSWLAWYQQKPEKAPKSLIYAASSLQSGVPSRFSGSGSGTDFTLTISSLQPEDFATYYCQQYKRYPYTFGQGTKLEIK

SEQ ID NO: 377 - protein, 10 aa, Synthetic construct
GYAFSSYWMN

SEQ ID NO: 378 - protein, 17 aa, Synthetic construct
QIWPGDGDTNYNGKFKG

SEQ ID NO: 379 - protein, 15 aa, Synthetic construct
RETTTVGRYYYAMDY

SEQ ID NO: 380 - protein, 15 aa, Synthetic construct
KASQSVDYDGDSYLN

SEQ ID NO: 381 - protein, 7 aa, Synthetic construct
DASNLVS

SEQ ID NO: 382 - protein, 9 aa, Synthetic construct
QQSTEDPWT

SEQ ID NO: 383 - protein, 121 aa, Synthetic construct
QVQLLESGAELVRPGSSVKISCKASGYAFSSYWMNWVKQRPGQGLEWIGQIWPGDGDTNYNGKFKGKATLTADESSSTAYMQLSSLRSEDSAVYSCARRETTTVGRYYYAMDYWGQGTTVT

SEQ ID NO: 384 - protein, 114 aa, Synthetic construct
ELVLTQSPASLAVSLGQRATISCKASQSVDYDGDSYLNWYQQIPGQPPKLLIYDASNLVSGIPPRFSGSGSGTDFTLNIHPVEKVDAATYHCQQSTEDPWTFGGGTKLEIKRRS

SEQ ID NO: 385 - protein, 5 aa, Synthetic construct
SYTMS

SEQ ID NO: 386 - protein, 17 aa, Synthetic construct
YISSGGGNTYYPDSVKG

SEQ ID NO: 387 - protein, 8 aa, Synthetic construct
HRGNPFDY

SEQ ID NO: 388 - protein, 15 aa, Synthetic construct
RASKSVSTSGYSFMH

SEQ ID NO: 389 - protein, 7 aa, Synthetic construct
LASNLES

SEQ ID NO: 390 - skipped

SEQ ID NO: 391 - protein, 10 aa, Synthetic construct
GFTFSSYTMS

SEQ ID NO: 392 - protein, 17 aa, Synthetic construct
YISSGGGNTYYPDSVKG

SEQ ID NO: 393 - protein, 19 aa, Synthetic construct
  REPEAT 1..19: note = sequence is repeated n times, wherein n is an integer from 1 to 100
KESGSVSSEQLAQFRSLDN

SEQ ID NO: 394 - protein, 15 aa, Synthetic construct
RASKSVSAWGYSFMH

SEQ ID NO: 395 - protein, 7 aa, Synthetic construct
VASRLES

SEQ ID NO: 396 - protein, 9 aa, Synthetic construct
QHSRELPWT

SEQ ID NO: 397 - protein, 10 aa, Synthetic construct
GFTFSSYGMS

SEQ ID NO: 398 - protein, 17 aa, Synthetic construct
YIDEGGGQTIYPDSVKG

SEQ ID NO: 399 - DNA, 6532 bp, Synthetic construct
  source 1..6532: mol_type = other DNA
cagctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctaccctaagtttttatgactacaaaaatcagtagtatgtcctgaaataatcattaagctgtttgaaagtatgactgcttgccatgtagataccatggcttgctgaataatcagaagaggtgtgactcttattctaaaatttgtcacaaaatgtcaaaatgagagactctgtaggaacgagtccttgacagacagctcaaggggtttttttcctttgtctcatttctacatgaaagtaaatttgaaatgatcttttttattataagagtagaaatacagttgggtttgaactatatgttttaatggccacggttttgtaagacatttggtcctttgttttcccagttattactcgattgtaattttatatcgccagcaatggactgaaacggtccgcaacctcttctttacaactgggtgacctcgcggctgtgccagccatttggcgttcaccctgccgctaagggccatgtgaacccccggaacagagaaacaggagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctctatataagcagagctcgtttagtgaaccgtcagatcgccgccaccatgaagcttttgagcagagatacgcggctggtctgtgagcttgcgctttgtcccctggctttagttttctggagcattcttggggtcagagcattggacaatggcttggcgcggactcctactatgggctggctgcattgggaacgtttcatgtgcaaccttgactgccaagaagagcctgatgcctgcataagtgagcaactgttcatgcagatggcagagctcatggtctctgatggctggcgggatgcaggttatgactatctctgcatagatgactgttggatggctcccgagagggattcaaagggcaggcttcaggcagatccccaacgctttcctagtgggatcaaacacctcgcaaattacgtccacagcaaaggattgaagctagggatttatgcagatgttgggaataaaacctgtgcaggtttccccgggagttttggatcctatgacattgatgcgcagacatttgctgactggggcgtagatctgctaaaatttgatggttgtcactgtgacagtgtagtatccttggagaatggttataagtacatggccttggccttgaacaggacaggccgaagcattgtatactcctgtgagtggccactttatttgagaccctttcataagcccaattatacagatatccaatattactgcaatcattggagaaattttgatgatgtttatgattcctgggaaagcataaagaatatcttgtcttggacagtggtttaccagaaggagattgttgaagtcgctggaccaggcagctggaatgacccagacatgttagtgatcggcaactttggcctcagttgggaccagcaggtgacacagatggccctctgggctatcatggccgctcccctactcatgtccaacgatctccgacaaatcagctctcaagccaaagctctgcttcagaataaggatgtaattgccatcaaccaagaccccttgggcaagcagggctactgtttcagaaaggaaaaccacattgaggtttgggaacggccactctccaacctagcctgggctgtggctgtgagaaacctgcaggagattggtggaccttgtccttataccatccagatttcttccctgggtagaggactagcctgcaatcctggctgcatcattactcagcttctccccgagaaagtacacctaggcttctatgaatggactttgaccttaaaaactcgagtaaacccctcaggcactgttttgtttcggttagaaagataatttttttccggtcggtagcatcccttgctccgcgtggaccactttcctgaggcacagtgataggaacagagccactaatctgaagagaacagagatgtgacagactacactaatgtgagaaaaacaaggaaagggtgacttattggagatttcagaaataaaatgcatttattattatattcccttattttaattttctattagggaattagaaagggcataaactgctttatccagtgttatattaaaagcttaatgtatataatcttttagaggtaaaatctacagccagcaaaagtcatggtaaatattctttgactgaactctcactaaactcctctaaattatatgtcatattaactggttaaattaatataaatttgtgacatgaccttaactggttaggtaggatatttttcttcatgcaaaaatatgactaataataatttagcacaaaaatatttcccaatactttaattctgtgatagaaaaatgtttaactcagctactatgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgccagctggcgtaatagcgaagaggcccgcaccgatcgcccttcccaacagttgcgcagcctgaatggcgaatggcgattccgttgcaatggctggcggtaatattgttctggatattaccagcaaggccgatagtttgagttcttctactcaggcaagtgatgttattactaatcaaagaagtattgcgacaacggttaatttgcgtgatggacagactcttttactcggtggcctcactgattataaaaacacttctcaggattctggcgtaccgttcctgtctaaaatccctttaatcggcctcctgtttagctcccgctctgattctaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttaaatatttgcttatacaatcttcctgtttttggggcttttctgattatcaaccggggtacatatgattgacatgctagttttacgattaccgttcatcgattctcttgtttgctccagactctcaggcaatgacctgatagcctttgtagagacctctcaaaaatagctaccctctccggcatgaatttatcagctagaacggttgaatatcatattgatggtgatttgactgtctccggcctttctcacccgtttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgttggaatcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatg

SEQ ID NO: 400 - protein, 15 aa, Synthetic construct
RASKSVSHVRWSFMH

SEQ ID NO: 401 - protein, 7 aa, Synthetic construct
LASKLES

SEQ ID NO: 402 - protein, 119 aa, Synthetic construct
QVQLQQSGAELAKPGASVKMSCKASGYTFTRYWMHWVKQRPGQGLEWIGYINPSTGYTEYNQKFKDKATLTADKSSSTVYMQLSSLTSEDSAVYYCARTTVDGYDFAYWGQGTLVTVSS

SEQ ID NO: 403 - protein, 15 aa, Synthetic construct
RMWPSSTVNLSAGRR

SEQ ID NO: 404 - protein, 470 aa, Synthetic construct
MAVLVLFLCLVAFPSCVLSQVQLKESGPGLVAPSQSLSITCTVSGFSLTNYGVHWVRQPPGKGLEWLGMIWAGGSTNYNSALMSRLNINKDNSKSQVFLEMNSLQTDDTALYFCAREATSGYVDYAVDYWGQGTSVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 405 - protein, 235 aa, Synthetic construct
MDFQVQIFSFLLISASVIISRGQIILNQSPAIMSAFPGEKVTMTCSASSSVSYMHWYQQKSGTSPKRWIYDTSKLASGVPARFSGSGSGTSYSLTISSMEAEDAATYYCQQWSSSPLTFGAGTKLELKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 406 - protein, 7 aa, Synthetic construct
GYTFTSN

SEQ ID NO: 407 - protein, 6 aa, Synthetic construct
SPGDGD

SEQ ID NO: 408 - protein, 13 aa, Synthetic construct
GDSSGWGPNWFDS

SEQ ID NO: 409 - protein, 11 aa, Synthetic construct
LASQDIGNYLS

SEQ ID NO: 410 - protein, 7 aa, Synthetic construct
DVNNLED

SEQ ID NO: 411 - protein, 9 aa, Synthetic construct
QQYYEYPLT

SEQ ID NO: 412 - protein, 7 aa, Synthetic construct
GYTFTTN

SEQ ID NO: 413 - protein, 6 aa, Synthetic construct
NPGDGN

SEQ ID NO: 414 - protein, 13 aa, Synthetic construct
GDYSGWGPNWFDY

SEQ ID NO: 415 - protein, 11 aa, Synthetic construct
LASQDIGDYLS

SEQ ID NO: 416 - protein, 7 aa, Synthetic construct
GATNLED

SEQ ID NO: 417 - protein, 9 aa, Synthetic construct
HQYYQYPLT

SEQ ID NO: 418 - DNA, 357 bp, Synthetic construct
  source 1..357: mol_type = other DNA
caggtccagttgcagcagagtggcgccgagcttgcaaaaccaggtgctagtgttaaaatgagctgtaaggcatccggttacacttttacccgatattggatgcactgggtgaagcaaaggccaggtcaaggacttgagtggataggatacattaatccttcaactggctacactgagtataatcaaaagtttaaggacaaagctacactcaccgctgataagagttccagcactgtatatatgcagttgtctagtctcacatccgaggattccgctgtatactattgtgctcgaactactgtagacggatatgattttgcatactgggggcagggaactctcgttacagtctcatcc

SEQ ID NO: 419 - DNA, 336 bp, Synthetic construct
  source 1..336: mol_type = other DNA
gacatggtcatgtctcaatctccatcctctcttgctgttagcgccggggaaaaggtatctatgagttgcaagtcttctcaaaccttgctgaacagccgcactagaaaaaattacctggcttggtatcaacaaaaaccaggacagagtcctaaactgctcatttactgggccagcacccgagagtctggagtacctgatcgatttactggctcaggcagcggcacagatttcacacttacaatctcttctgtccaagccgaagacttggctgtttattactgcaaacaatcctataatctgtggacattcgggggcggaaccaagcttgaaattaaa

SEQ ID NO: 420 - DNA, 15 bp, Synthetic construct
  source 1..15: mol_type = other DNA
cgatattggatgcac

SEQ ID NO: 421 - DNA, 51 bp, Synthetic construct
  source 1..51: mol_type = other DNA
tacattaatccttcaactggctacactgagtataatcaaaagtttaaggac

SEQ ID NO: 422 - DNA, 30 bp, Synthetic construct
  source 1..30: mol_type = other DNA
actactgtagacggatatgattttgcatac

SEQ ID NO: 423 - DNA, 51 bp, Synthetic construct
  source 1..51: mol_type = other DNA
aagtcttctcaaaccttgctgaacagccgcactagaaaaaattacctggct

SEQ ID NO: 424 - DNA, 21 bp, Synthetic construct
  source 1..21: mol_type = other DNA
tgggccagcacccgagagtct

SEQ ID NO: 425 - DNA, 24 bp, Synthetic construct
  source 1..24: mol_type = other DNA
aaacaatcctataatctgtggaca

SEQ ID NO: 426 - DNA, 64 bp, Synthetic construct
  source 1..64: mol_type = other DNA
caggtgagtcctaacttctcccattctaaatgcatgttggggggattctgggccttcaggacca

SEQ ID NO: 427 - DNA, 509 bp, Synthetic construct
  source 1..509: mol_type = other DNA
cgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctc

SEQ ID NO: 428 - DNA, 587 bp, Synthetic construct
  source 1..587: mol_type = other DNA
gttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagct

SEQ ID NO: 429 - DNA, 508 bp, Synthetic construct
  source 1..508: mol_type = other DNA
cgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagct

SEQ ID NO: 430 - DNA, 347 bp, Synthetic construct
  source 1..347: mol_type = other DNA
gaacagagaaacaggagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctctatataagcagagctcgtttagtgaaccgtcagatc

SEQ ID NO: 431 - DNA, 188 bp, Synthetic construct
  source 1..188: mol_type = other DNA
aaaaatgtatgacccttggcatggataatatatgtatttagaagggtcctttaagacacatgtgatgctctagtaagttttctgctgacaccaactccctgtgggtgaaacaggagcaggtgccctttgaccaacagtacacactctgctgagctgatttgcatatggagtcattatacaacatccca

SEQ ID NO: 432 - DNA, 319 bp, Synthetic construct
  source 1..319: mol_type = other DNA
gacaagtgagtgtctcaggttaggattctattttaagattgagatattaggctttgatactacatctaaatggtctgtacatgtctcgaagaaagttcttcagacagagttaggacttggatccaggagttaggacttggactgactcaggaggactctagtttcttcttctccagctggaatgtccttatgtaagaaaagccttgcctcatgagtatgcaaatcatgtgcgactgtgatgattaatatagggatatccacaccaaacatcatatgagccctatcttctctacagacactgaatctcaaggtccttaca

SEQ ID NO: 433 - DNA, 548 bp, Synthetic construct
  source 1..548: mol_type = other DNA
gtttagtgaaccgtcagatcctcactctcttccgcatcgctgtctgcgagggccagctgttgggctcgcggttgaggacaaactcttcgcggtctttccagtactcttggatcggaaacccgtcggcctccgaacggtactccgccaccgagggacctgagcgagtccgcatcgaccggatcggaaaacctctcgagaaaggcgtctaaccagtcacagtcgcaaggtaggctgagcaccgtggcgggcggcagcgggtggcggtcggggttgtttctggcggaggtgctgctgatgatgtaattaaagtaggcggtcttgagacggcggatggtcgaggtgaggtgtggcaggcttgagatccagctgttggggtgagtactccctctcaaaagcgggcattacttctgcgctaagattgtcagtttccaaaaacgaggaggatttgatattcacctggcccgatctggccatacacttgagtgacaatgacatccactttgcctttctctccacaggtgtccactcccaggtccaagtttaaactt

SEQ ID NO: 434 - DNA, 478 bp, Mus musculus
  source 1..478: mol_type = genomic DNA
cctaagtttttatgactacaaaaatcagtagtatgtcctgaaataatcattaagctgtttgaaagtatgactgcttgccatgtagataccatggcttgctgaataatcagaagaggtgtgactcttattctaaaatttgtcacaaaatgtcaaaatgagagactctgtaggaacgagtccttgacagacagctcaaggggtttttttcctttgtctcatttctacatgaaagtaaatttgaaatgatcttttttattataagagtagaaatacagttgggtttgaactatatgttttaatggccacggttttgtaagacatttggtcctttgttttcccagttattactcgattgtaattttatatcgccagcaatggactgaaacggtccgcaacctcttctttacaactgggtgacctcgcggctgtgccagccatttggcgttcaccctgccgctaagggccatgtgaacccccg

SEQ ID NO: 435 - DNA, 500 bp, Mus musculus
  source 1..500: mol_type = genomic DNA
cggtagcatcccttgctccgcgtggaccactttcctgaggcacagtgataggaacagagccactaatctgaagagaacagagatgtgacagactacactaatgtgagaaaaacaaggaaagggtgacttattggagatttcagaaataaaatgcatttattattatattcccttattttaattttctattagggaattagaaagggcataaactgctttatccagtgttatattaaaagcttaatgtatataatcttttagaggtaaaatctacagccagcaaaagtcatggtaaatattctttgactgaactctcactaaactcctctaaattatatgtcatattaactggttaaattaatataaatttgtgacatgaccttaactggttaggtaggatatttttcttcatgcaaaaatatgactaataataatttagcacaaaaatatttcccaatactttaattctgtgatagaaaaatgtttaactcagctactat

SEQ ID NO: 436 - DNA, 502 bp, Mus musculus
  source 1..502: mol_type = genomic DNA
ccaggggtgattctagtcagactctggggtttttgtcgggtatagaggaaaaatccactattgtgattactatgctatggactactggggtcaaggaacctcagtcaccgtctcctcaggtaagaatggcctctccaggtctttatttttaacctttgttatggagttttctgagcattgcagactaatcttggatatttgtccctgagggagccggctgagagaagttgggaaataaactgtctagggatctcagagcctttaggacagattatctccacatctttgaaaaactaagaatctgtgtgatggtgttggtggagtccctggatgatgggatagggactttggaggctcatttgagggagatgctaaaacaatcctatggctggagggatagttggggctgtagttggagattttcagtttttagaataaaagtattagttgtggaatatacttcaggaccacctctgtgacagcatttatacagtatccgatg

SEQ ID NO: 437 - DNA, 968 bp, Mus musculus
  source 1..968: mol_type = genomic DNA
catagggacaaagagtggagtggggcactttctttagatttgtgaggaatgttccgcactagattgtttaaaacttcatttgttggaaggagagctgtcttagtgattgagtcaagggagaaaggcatctagcctcggtctcaaaagggtagttgctgtctagagaggtctggtggagcctgcaaaagtccagctttcaaaggaacacagaagtatgtgtatggaatattagaagatgttgcttttactcttaagttggttcctaggaaaaatagttaaatactgtgactttaaaatgtgagagggttttcaagtactcatttttttaaatgtccaaaatttttgtcaatcagtttgaggtcttgtttgtgtagaactgatattacttaaagtttaaccgaggaatgggagtgaggctctctcataacctattcagaactgacttttaacaataataaattaagtttaaaatatttttaaatgaattgagcaatgttgagttggagtcaagatggccgatcagaaccagaacacctgcagcagctggcaggaagcaggtcatgtggcaaggctatttggggaagggaaaataaaaccactaggtaaacttgtagctgtggtttgaagaagtggttttgaaacactctgtccagccccaccaaaccgaaagtccaggctgagcaaaacaccacctgggtaatttgcatttctaaaataagttgaggattcagccgaaactggagaggtcctcttttaacttattgagttcaaccttttaattttagcttgagtagttctagtttccccaaacttaagtttatcgacttctaaaatgtatttagaattcattttcaaaattaggttatgtaagaaattgaaggactttagtgtctttaatttctaatatatttagaaaacttcttaaaattactctattattcttccctctgattattggtctccattca

SEQ ID NO: 438 - DNA, 60 bp, Synthetic construct
  source 1..60: mol_type = other DNA
atgtacagaatgcagctgctgagctgcatcgccctgagcctggccctggtgaccaacagc

SEQ ID NO: 439 - DNA, 60 bp, Mus musculus
  source 1..60: mol_type = genomic DNA
atggaaaccgacacactgctgctgtgggtgctgcttctttgggtgcccggaagcacaggc

SEQ ID NO: 440 - DNA, 75 bp, Synthetic construct
  source 1..75: mol_type = other DNA
atggacgccatgaagagagggctgtgctgcgtgctgctgctgtgcggagcagtgttcgtcagcccttccgctagc

SEQ ID NO: 441 - DNA, 136 bp, Synthetic construct
  source 1..136: mol_type = other DNA
gaaggtaccccattgtatgggatctgatctggggcctcggtgcacatgctttacatgtgtttagtcgaggttaaaaaacgtctaggccccccgaaccacggggacgtggttttcctttgaaaaacacgataatacc

SEQ ID NO: 442 - DNA, 1242 bp, Synthetic construct
  source 1..1242: mol_type = other DNA
atgctcgagggagtgcaggtggagactatctccccaggagacgggcgcaccttccccaagcgcggccagacctgcgtggtgcactacaccgggatgcttgaagatggaaagaaagttgattcctcccgggacagaaacaagccctttaagtttatgctaggcaagcaggaggtgatccgaggctgggaagaaggggttgcccagatgagtgtgggtcagagagccaaactgactatatctccagattatgcctatggtgccactgggcacccaggcatcatcccaccacatgccactctcgtcttcgatgtggagcttctaaaactggaatctggcggtggatccggagtcgacggatttggtgatgtcggtgctcttgagagtttgaggggaaatgcagatttggcttacatcctgagcatggagccctgtggccactgcctcattatcaacaatgtgaacttctgccgtgagtccgggctccgcacccgcactggctccaacatcgactgtgagaagttgcggcgtcgcttctcctcgctgcatttcatggtggaggtgaagggcgacctgactgccaagaaaatggtgctggctttgctggagctggcgcggcaggaccacggtgctctggactgctgcgtggtggtcattctctctcacggctgtcaggccagccacctgcagttcccaggggctgtctacggcacagatggatgccctgtgtcggtcgagaagattgtgaacatcttcaatgggaccagctgccccagcctgggagggaagcccaagctctttttcatccaggcctgtggtggggagcagaaagaccatgggtttgaggtggcctccacttcccctgaagacgagtcccctggcagtaaccccgagccagatgccaccccgttccaggaaggtttgaggaccttcgaccagctggacgccatatctagtttgcccacacccagtgacatctttgtgtcctactctactttcccaggttttgtttcctggagggaccccaagagtggctcctggtacgttgagaccctggacgacatctttgagcagtgggctcactctgaagacctgcagtccctcctgcttagggtcgctaatgctgtttcggtgaaagggatttataaacagatgcctggttgctttaatttcctccggaaaaaacttttctttaaaacatcagtcgactatccgtacgacgtaccagactacgcactcgactaa

SEQ ID NO: 443 - DNA, 937 bp, Synthetic construct
  source 1..937: mol_type = other DNA
atggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctctctgcaggacggcgagttcatctacaaggtgaagctgcgcggcaccaacttcccctccgacggccccgtaatgcagaagaagactatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtgagcggccgcttaattaaaagcttgtcgacggatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttt

SEQ ID NO: 444 - DNA, 727 bp, Synthetic construct
  source 1..727: mol_type = other DNA
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaattttttt

SEQ ID NO: 445 - DNA, 1204 bp, Homo sapiens
  source 1..1204: mol_type = genomic DNA
ctggacaatggattggcaaggacgcctaccatgggctggctgcactgggagcgcttcatgtgcaaccttgactgccaggaagagccagattcctgcatcagtgagaagctcttcatggagatggcagagctcatggtctcagaaggctggaaggatgcaggttatgagtacctctgcattgatgactgttggatggctccccaaagagattcagaaggcagacttcaggcagaccctcagcgctttcctcatgggattcgccagctagctaattatgttcacagcaaaggactgaagctagggatttatgcagatgttggaaataaaacctgcgcaggcttccctgggagttttggatactacgacattgatgcccagacctttgctgactggggagtagatctgctaaaatttgatggttgttactgtgacagtttggaaaatttggcagatggttataagcacatgtccttggccctgaataggactggcagaagcattgtgtactcctgtgagtggcctctttatatgtggccctttcaaaagcccaattatacagaaatccgacagtactgcaatcactggcgaaattttgctgacattgatgattcctggaaaagtataaagagtatcttggactggacatcttttaaccaggagagaattgttgatgttgctggaccagggggttggaatgacccagatatgttagtgattggcaactttggcctcagctggaatcagcaagtaactcagatggccctctgggctatcatggctgctcctttattcatgtctaatgacctccgacacatcagccctcaagccaaagctctccttcaggataaggacgtaattgccatcaatcaggaccccttgggcaagcaagggtaccagcttagacagggagacaactttgaagtgtgggaacgacctctctcaggcttagcctgggctgtagctatgataaaccggcaggagattggtggacctcgctcttataccatcgcagttgcttccctgggtaaaggagtggcctgtaatcctgcctgcttcatcacacagctcctccctgtgaaaaggaagctagggttctatgaatggacttcaaggttaagaagtcacataaatcccacaggcactgttttgcttcagctagaaaatacaatgcagatgtcattaaaagacttactttaattttttt

SEQ ID NO: 446 - DNA, 1174 bp, Mus musculus
  source 1..1174: mol_type = genomic DNA
ttggacaatggcttggcgcggactcctactatgggctggctgcattgggaacgtttcatgtgcaaccttgactgccaagaagagcctgatgcctgcataagtgagcaactgttcatgcagatggcagagctcatggtctctgatggctggcgggatgcaggttatgactatctctgcatagatgactgttggatggctcccgagagggattcaaagggcaggcttcaggcagatccccaacgctttcctagtgggatcaaacacctcgcaaattacgtccacagcaaaggattgaagctagggatttatgcagatgttgggaataaaacctgtgcaggtttccccgggagttttggatcctatgacattgatgcgcagacatttgctgactggggcgtagatctgctaaaatttgatggttgtcactgtgacagtgtagtatccttggagaatggttataagtacatggccttggccttgaacaggacaggccgaagcattgtatactcctgtgagtggccactttatttgagaccctttcataagcccaattatacagatatccaatattactgcaatcattggagaaattttgatgatgtttatgattcctgggaaagcataaagaatatcttgtcttggacagtggtttaccagaaggagattgttgaagtcgctggaccaggcagctggaatgacccagacatgttagtgatcggcaactttggcctcagttgggaccagcaggtgacacagatggccctctgggctatcatggccgctcccctactcatgtccaacgatctccgacaaatcagctctcaagccaaagctctgcttcagaataaggatgtaattgccatcaaccaagaccccttgggcaagcagggctactgtttcagaaaggaaaaccacattgaggtttgggaacggccactctccaacctagcctgggctgtggctgtgagaaacctgcaggagattggtggaccttgtccttataccatccagatttcttccctgggtagaggactagcctgcaatcctggctgcatcattactcagcttctccccgagaaagtacacctaggcttctatgaatggactttgaccttaaaaactcgagtaaacccctcaggcactgttttgtttcggttagaaagataattttttt

SEQ ID NO: 447 - DNA, 321 bp, Synthetic construct
  source 1..321: mol_type = other DNA
cgcgccgatgccgctcctaccgtgtctatctttccacctagcagcgagcagctgacatctggcggagcctctgtcgtgtgcttcctgaacaacttctaccctaaggacatcaacgtcaagtggaagatcgacggctccgagagacagaacggcgtgctgaactcttggaccgaccaggacagcaaggatagcacctacagcatgagcagcactctgaccctgacaaaggacgagtacgagaggcacaactcctacacatgcgaggccacacacaagaccagcacatccccaatcgtgaagtccttcaaccggaacgagtgc

SEQ ID NO: 448 - protein, 112 aa, Synthetic construct
DMVMSQSPSSLAVSAGEKVSMSCKSSQTLLNSRTRKNYLAWYQQKPGQSPKLLIYWASTRESGVPDRFTGSGSGTDFTLTISSVQAEDLAVYYCKQSYNLWTFGGGTKLEIK

SEQ ID NO: 449 - DNA, 61 bp, Synthetic construct
  source 1..61: mol_type = other DNA
gtgagtcctaacttctcccattctaaatgcatgttggggggattctgggccttcaggacca

SEQ ID NO: 450 - DNA, 972 bp, Mus musculus
  source 1..972: mol_type = genomic DNA
gccaagaccacacctcctagcgtgtaccctctggctcctggatctgccgctcagaccaacagcatggtcacactgggctgtctggtcaagggctacttccctgagcctgtgaccgtgacatggaacagcggatctctgagcagcggagtgcacacattccctgccgtgctgcagagcgacctgtacacactgagcagcagcgtgacagtgcccagctctacatggcctagcgagacagtgacctgcaacgtggcccatcctgccagcagtaccaaggtggacaagaaaatcgtgcccagagactgcggctgcaagccctgcatctgtacagtgcctgaagtgtccagcgtgttcatcttcccacctaagcctaaggacgtgctgacaatcaccctgacacctaaagtgacctgtgtggtggtggacatctccaaggacgaccccgaggtgcagttcagttggttcgtggacgacgtggaagtgcacactgcccagacacagcccagagaggaacagttcaacagcaccttcagaagcgtgtccgagctgcccatcatgcaccaggattggctgaacggcaaagaattcaagtgcagagtgaacagcgccgcctttcctgctcctatcgaaaagacaatcagcaagaccaagggcagacccaaggctccccaggtgtacacaatccctccacctaaagaacagatggccaaggacaaggtgtccctcacctgtatgatcaccgatttcttcccagaggacatcaccgtggaatggcagtggaacggacagcccgccgagaactacaagaacacccagcctatcatggacaccgacggcagctacttcgtgtacagcaagctgaacgtgcagaagtccaactgggaggccggcaacaccttcacctgttctgtgctgcacgagggcctgcacaatcaccacaccgagaagtccctgtctcacagccctggcaaa

SEQ ID NO: 451 - DNA, 81 bp, Synthetic construct
  source 1..81: mol_type = other DNA
ggatccggttccggtcaccatcaccatcaccatggcctgaatgatatttttgaggcccagaagattgagtggcatgaatga

SEQ ID NO: 452 - protein, 107 aa, Homo sapiens [sequence omitted]

SEQ ID NO: 453 - protein, 106 aa, Homo sapiens
GQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 454 - protein, 330 aa, Homo sapiens [sequence omitted]

SEQ ID NO: 455 - protein, 225 aa, Homo sapiens
THTCPPCPAPEFFGGPSVFFFPPKPKDTFMISRTPEVTCVVVDVSHEDPEVKFNWYVDGVEVHNAKTKPREEQYNSTYRVVSVETVFHQDWENGKEYKCKVSNKAFPVPIEKTISKAKGQPREPQVYTLPPSREEMTKNQVSLTCLVKGFYPSDIAVEWESNGQPENNYKTTPPVLDSDGPFFLYSKLTVDKSRWQQGNVFSCSVMHEALHNHYTQKSLSLSPGK

SEQ ID NO: 456 - protein, 326 aa, Homo sapiens
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGK

SEQ ID NO: 457 - protein, 217 aa, Homo sapiens
PAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 458 - protein, 377 aa, Homo sapiens
ASTKGPSVFPLAPCSRSTSGGTAALGCLVKDYFPEPVTVSWNSGALTSGVHTFPAVLQSSGLYSLSSVVTVPSSSLGTQTYTCNVNHKPSNTKVDKRVELKTPLGDTTHTCPRCPEPKSCDTPPPCPRCPEPKSCDTPPPCPRCPEPKSCDTPPPCPRCPAPELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 459 - protein, 218 aa, Homo sapiens
PAPELLGGPSVFLFPPKPKDTLMISRTPEVTCVVVDVSHEDPEVQFKWYVDGVEVHNAKTKPREEQFNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 460 - protein, 327 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 461 - protein, 218 aa, Homo sapiens
PAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

(19) United States
(12) Patent Application Publication
(10) Pub. No.: US 2026/0048123 A1
(43) Pub. Date: Feb. 19, 2026

(54) SYSTEMS AND METHODS TO PRODUCE B CELLS THAT EXPRESS SELECTED ANTIBODIES AND GENE PRODUCTS

(71) Applicant: Fred Hutchinson Cancer Center, Seattle, WA (US)

(72) Inventors: Justin J. Taylor, Seattle, WA (US); Julia Luna McKechnie, Renton, WA (US); Carson Harms, Seattle, WA (US)

(73) Assignee: Fred Hutchinson Cancer Center, Seattle, WA (US)

(21) Appl. No.: 18/852,997

(22) PCT Filed: Mar. 30, 2023
(86) PCT No.: PCT/US23/65142

§ 371 Date: Sep. 30, 2024

Related U.S. Application Data
(60) Provisional application No. 63/325,537, filed on Mar. 30, 2022.

(51) Int. Cl. A61K 40/13; A61K 40/33; C12N 9/22; C12N 15/11; C12N 15/90

(52) U.S. Cl. CPC A61K 40/13; A61K 40/33; C12N 9/226; C12N 15/11; C12N 15/907; C12N 2310/20; C12N 2830/15; C12N 2840/002

(57) ABSTRACT

A number of medical disorders are caused by either an insufficiency of a gene product or a defective gene product. Gene therapy can be used to provide a sufficient amount of a gene product when a disorder is caused by an insufficiency and can also be used to inactivate genes that produce defective gene products. Examples of disorders that can be treated by providing a sufficient amount of a gene product include lysosomal storage diseases, clotting disorders, diabetes, and alpha-1 antitrypsin deficiency. Systems and methods to produce B cells that express selected antibodies and gene products are described. The systems and methods can be used to provide prolonged and tunable expression of the gene products for the treatment of diseases such as lysosomal storage diseases, clotting disorders, diabetes, or other protein deficiencies.

### CROSS-REFERENCE TO RELATED APPLICATIONS

This application is a U.S. National Phase application based on International Patent Application No. PCT/US2023/065142, filed on Mar. 30, 2023, which claims priority to U.S. Provisional Patent Application No. 63/325,537 filed Mar. 30, 2022, each of which is incorporated herein by reference in its entirety as if fully set forth herein.

### REFERENCE TO SEQUENCE LISTING

The Sequence Listing associated with this application is provided in XML format in lieu of a paper copy and is hereby incorporated by reference into the specification. The name of the file containing the Sequence Listing is 3B46477.XML. The file is 575,003 bytes, was created on Sep. 30, 2024, and is being submitted electronically via Patent Center.

### FIELD OF THE DISCLOSURE

The current disclosure provides systems and methods to produce B cells that express selected antibodies and gene products. The systems and methods can be used to provide prolonged and tunable expression of the gene products for the treatment of diseases such as lysosomal storage diseases.

### BACKGROUND OF THE DISCLOSURE

A number of medical disorders are caused by either an insufficiency of a gene product or a defective gene product. Gene therapy can be used to provide a sufficient amount of a gene product when a disorder is caused by an insufficiency and can also be used to inactivate genes that produce defective gene products. Examples of disorders that can be treated by providing a sufficient amount of a gene product include lysosomal storage diseases, clotting disorders, diabetes, and alpha-1 antitrypsin deficiency.

Lysosomal storage diseases are inherited metabolic diseases characterized by a lack of sufficient enzymatic activity to prevent the accumulation of specific macromolecules in various tissues. The progressive accumulation of the specific macromolecules cause the cells containing the lysosomes to become engorged and ultimately leads to tissue damage and organ dysfunction and/or failure. There are nearly 50 of these disorders altogether, and they affect different parts of the body, including the skeleton, brain, skin, heart, and central nervous system.

Enzyme replacement therapy (ERT) can be used as a treatment for lysosomal storage diseases and has significantly elevated treatment options from symptom management and comfort care to actual therapeutic interventions which address the underlying metabolic defect. ERT is not a cure for lysosomal storage diseases but can be very beneficial in attenuating symptoms and disease progression. In particular ERT regimens, patients are required to have bi-weekly (i.e., every other week) intravenous infusion of the enzyme they lack for their entire life. Furthermore, each infusion takes 3 to 4 hours depending on the enzyme and the dose. The bolus nature of these administrations results in high levels of the enzyme following administration that significantly wane between treatments. Thus, despite the many benefits and improved quality of life ERT has provided for patients with lysosomal storage diseases, better treatment options are still needed. A variety of other disorders are similarly in need of improved treatment options.

### SUMMARY OF THE DISCLOSURE

The current disclosure provides systems and methods to provide prolonged and tunable expression of gene products within subjects. In relation to lysosomal storage diseases, the prolonged expression obviates the need to receive bi-weekly infusions and overcomes drawbacks associated with the bolus nature of bi-weekly treatments. Moreover, if expression of a gene product wanes over time, the disclosed systems and methods provide fast, safe, and efficient mechanisms to amplify expression of the gene product.

The disclosed systems and methods provide these significant benefits by engineering B cells to express a selected antibody and a gene product. If expression of the gene product falls below a threshold, an antigen that binds the selected antibody can be administered. Binding of the antigen to the selected antibody activates the B cell, such that expression of the gene product will be amplified. Particular embodiments also include a suicide gene so that expression of the gene product can be reduced if expression levels were to exceed a threshold or otherwise cause adverse effects in a subject. Thus, the systems and methods provide for tunable in vivo expression of gene products in subjects.

In particular embodiments, the current disclosure provides these benefits through the targeted insertion of a first genetic construct encoding the selected antibody in an intronic region upstream or downstream of an Eμ enhancer within the native antibody heavy chain gene of B cells.

In particular embodiments, the current disclosure provides these benefits through the targeted insertion of a second genetic construct encoding the gene product in an intronic region upstream or downstream of an iE_{κ} enhancer within the native antibody light chain gene of B cells.

Particular embodiments utilize a selected antibody that binds an antigen that the subject would rarely, if ever, encounter in the natural environment. This feature reduces the likelihood of unintended amplified expression of the gene product.

### BRIEF DESCRIPTION OF THE FIGURES

Some of the drawings submitted herewith may be better understood in color. Applicant considers the color versions of the drawings as part of the original submission and reserves the right to present color images of the drawings in later proceedings.

FIG. 1. Schematic of engineering B cells to mimic vaccination.

FIG. 2. Schematic of an exemplary method of engineering a B cell's genome to express an engineered monoclonal antibody. In this exemplary method, a single cut is made using Cas9 upstream of the Eμ enhancer and a gene encoding the engineered monoclonal antibody is inserted.

FIGS. 3A, 3B. (3A) Schematic for engineering B cells to express engineered monoclonal antibody. At day 0, the B cells are stimulated. At day 2, the cells are engineered using Cas9 and a viral vector including the genetic construct encoding the engineered monoclonal antibody. At day 5, the cells are analyzed for expression of the engineered monoclonal antibody. (3B) Flow cytometry shows efficient engineering of primary human B cells.

FIGS. 4A, 4B. (4A) Schematic showing how engineered B cells can be used as a source of gene products. (4B) Schematic showing approach for genetically engineering cells to express a protein of interest. Using Cas9, the B cell's genome can be cut upstream of the iE_{κ} and a genetic construct encoding the protein of interest is inserted operably before the light chain constant region gene of the B cell.

FIGS. 5A, 5B. Efficient cutting of the (5A) human and (5B) murine antibody light chain loci. Human or murine B cells were stimulated in culture for 1-2 days prior to electroporation of the listed guide RNA (gRNA) targeting the antibody kappa light chain loci precomplexed with Cas9. 48 hours later DNA from cells was assessed for the presence of insertions or deletions at the gRNA cut site compared to control cells using Tracking of Indels by Decomposition (TIDE). The total % of insertions and deletions at this region is reported as “% editing”.

FIG. 6. Efficient engineering of target locus using mCherry construct. Murine B cells were stimulated in culture for 1 day prior to electroporation of mIgK1045 gRNA targeting the antibody kappa light chain loci precomplexed with Cas9 prior to incubation with an AAV encoding mCherry driven by a constitutive CMV promoter flanked by IgK homology regions. 48 hours later cells were assessed for mCherry expression by flow cytometry.

FIG. 7. Simultaneous engineering of both the heavy chain and light chain loci. Murine B cells were stimulated in culture for 1 day prior to electroporation of mIgK1045 gRNA targeting the antibody kappa light chain loci precomplexed with Cas9 and mIgH367 gRNA (sgRNA-mIgH_3, SEQ ID NO: 181) targeting the murine antibody heavy chain loci precomplexed to Cas9 prior to incubation with an AAV encoding mCherry driven by a constitutive CMV promoter flanked by IgK homology regions and/or an AAV encoding an engineered monoclonal antibody (emAb) construct encoding an RSV-specific antibody. 48 hours later cells were assessed by flow cytometry for dual expression of mCherry and expression of the RSV-specific antibody, which is detected based upon binding to a fluorescent RSV F antigen and Strep-Tactin binding to StrepII tag in the emAb linker.

FIG. 8. Transfection of enzyme-encoding construct results in production of functional enzyme. A plasmid containing murine alpha-galactosidase A (alpha-GlaA) was transfected into 293E cells 24 hours prior to the assessment of alpha-GlaA concentration in culture supernatant. Three different signal sequences were assessed to determine which led to the highest enzyme secretion. The endogenous alpha-GlaA signal sequence and an IL2 signal sequence that has been reported to induce increased secretion were superior in this assay.

FIGS. 9A, 9B. (9A) Enzyme-encoding construct produces functional enzyme and inserts into the kappa locus. 293E cells were transfected in culture with a plasmid containing human alpha-GlaA using the endogenous human alpha-GlaA signal peptide. 24 hours post transfection, culture supernatants were collected and assessed for alpha-GlaA activity. (9B) Murine B cells were stimulated in culture for 1 day prior to electroporation of mIgK1045 gRNA targeting the antibody kappa light chain loci precomplexed with Cas9 prior to incubation with an AAV encoding alpha-GlaA driven by a constitutive CMV promoter flanked by IgK homology regions (HR). 48 hours later cells were assessed for the presence of alpha-GlaA DNA in the kappa light chain gene.

FIGS. 10A-10I. Constructs for (10A) pCH24-CMV-mCherry-Version 1 sequence, (10B) pCH24-MND-mCherry-with-Kozak sequence, (10C) pCH24-MND-Casp9IRESGFP sequence, (10D) pCH24-MND-Casp9IRESGFP-wo-ACCGG sequence, (10E) pCH24-Vk21E-Casp9IRESGFP-wo-ACCGG sequence, (10F) pCH24-CMV-aGlaA-WTsp sequence, (10G) pCH24-MND-aGlaAwt (wo accgg) sequence, (10H) pCH24-MND-MutantIL2-aGLA (wo ACCGG) sequence, and (10I) pCH24-MND-WTIL2-aGLA (wo ACCGG) sequence.

FIG. 11. Sequences supporting the disclosure.

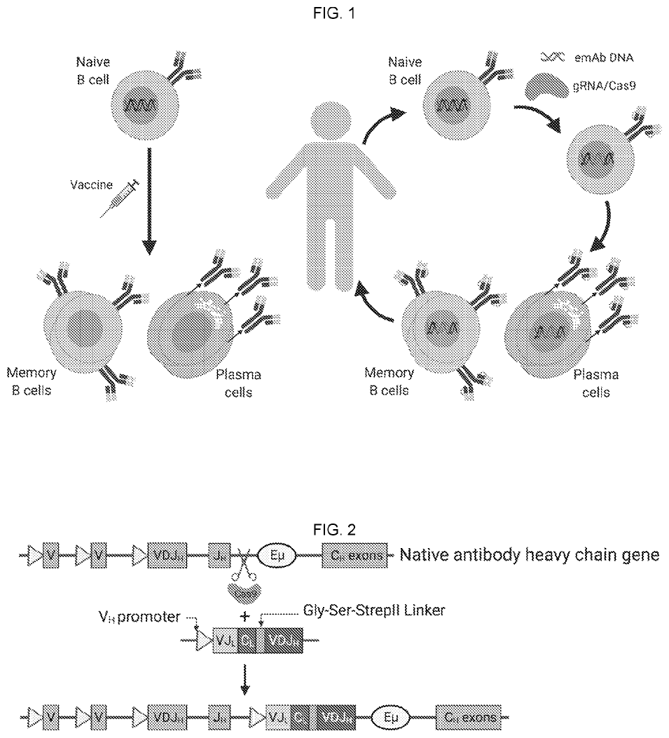

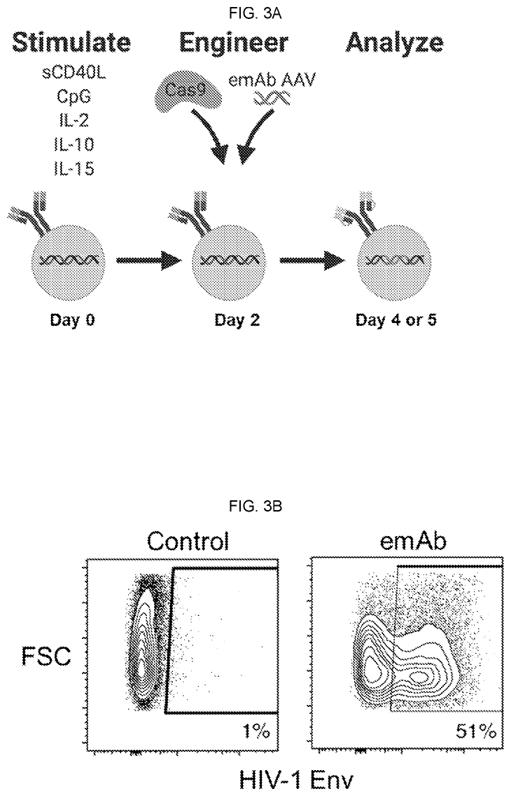

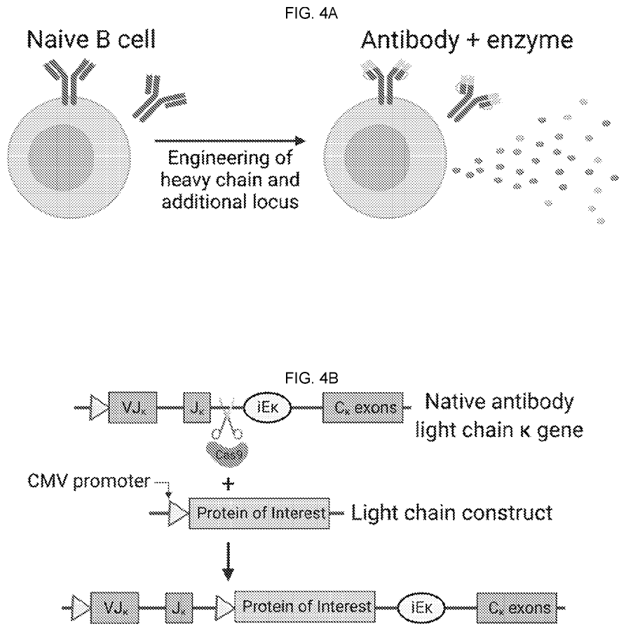

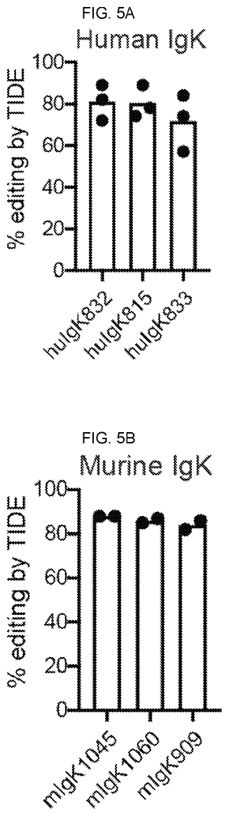

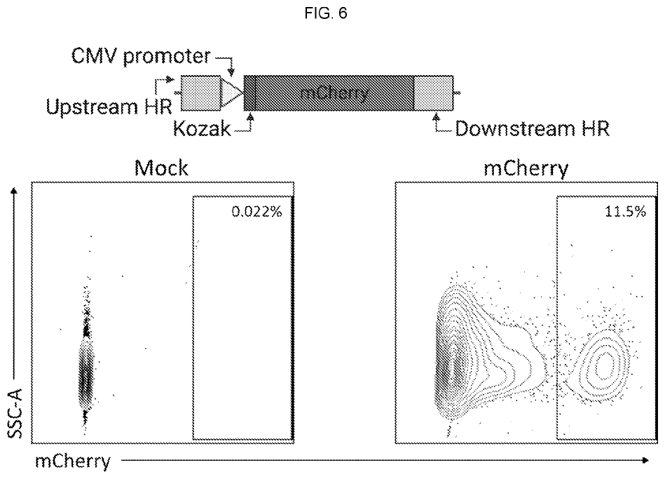

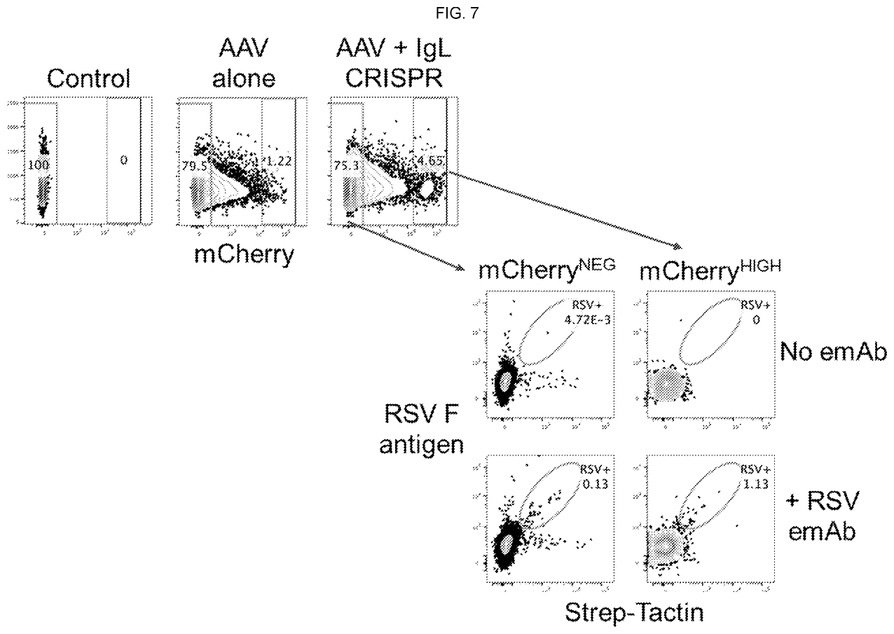

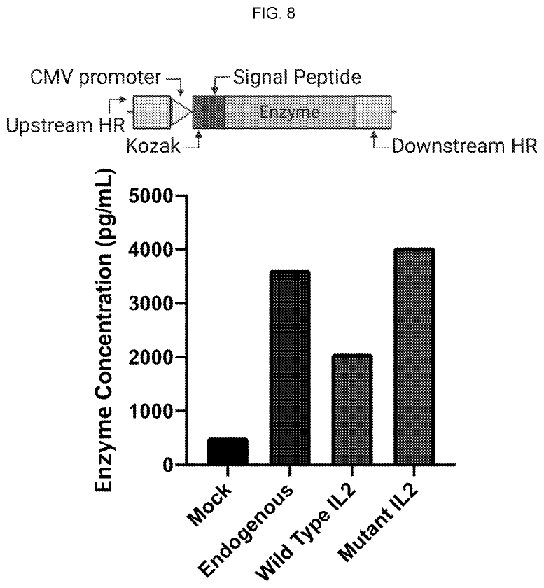

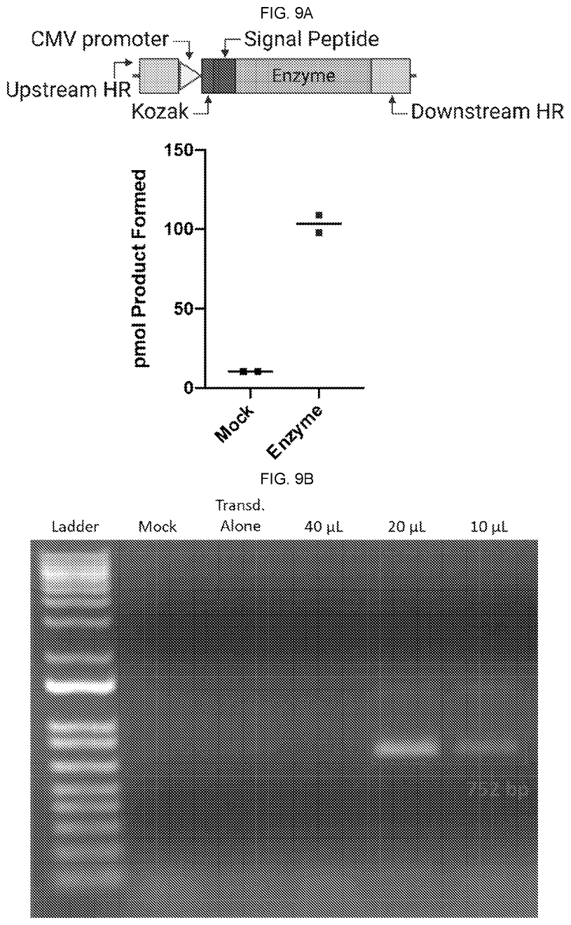

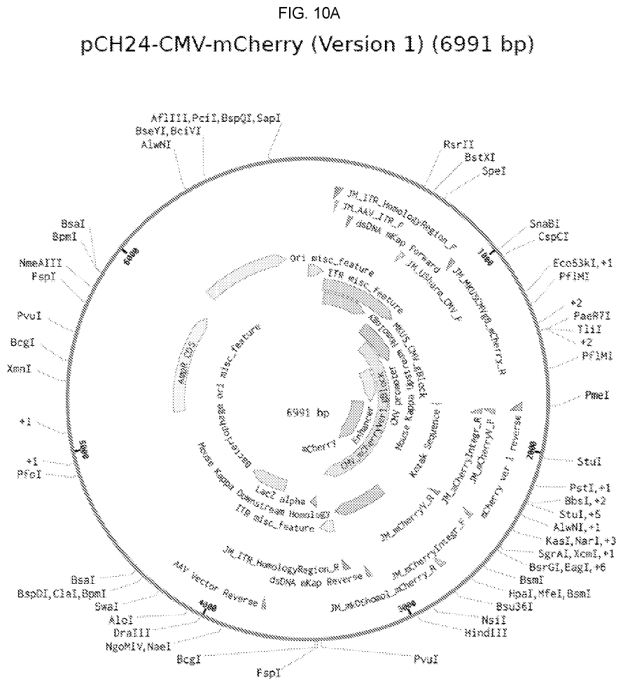

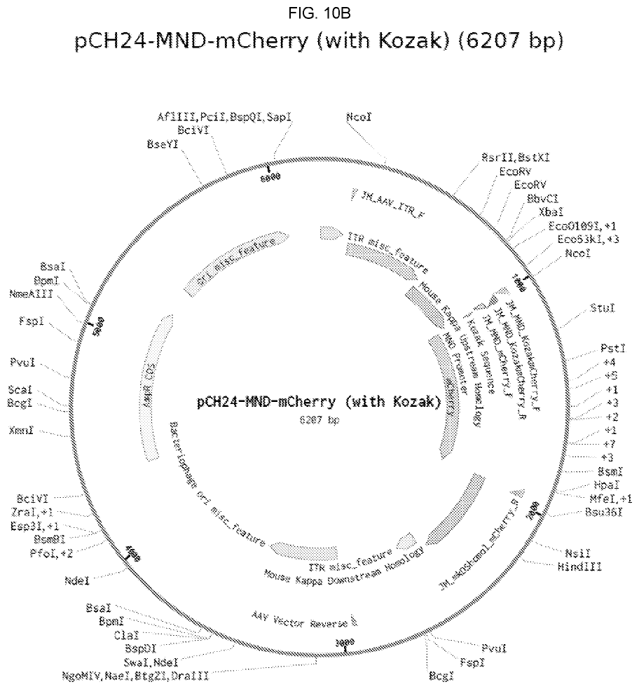

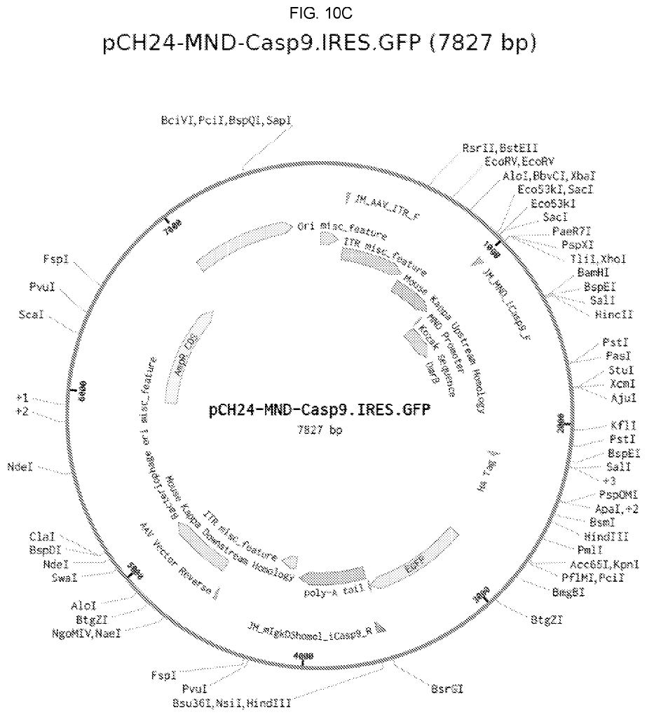

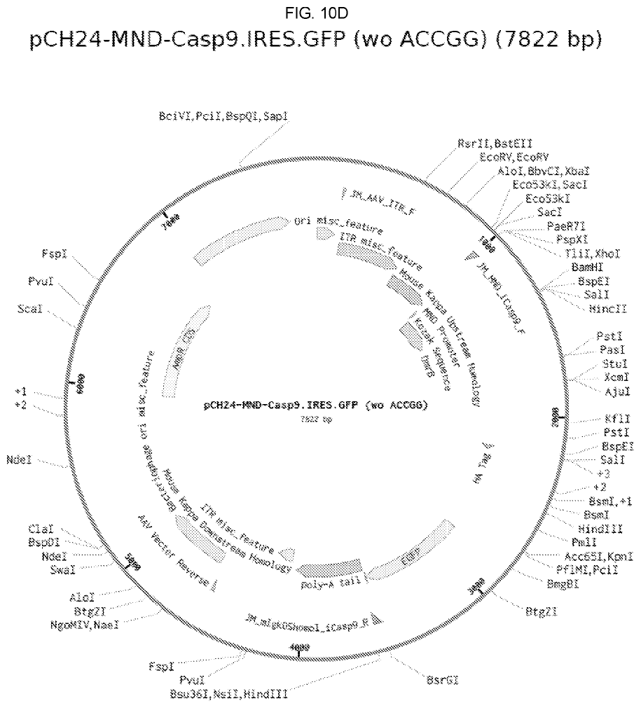

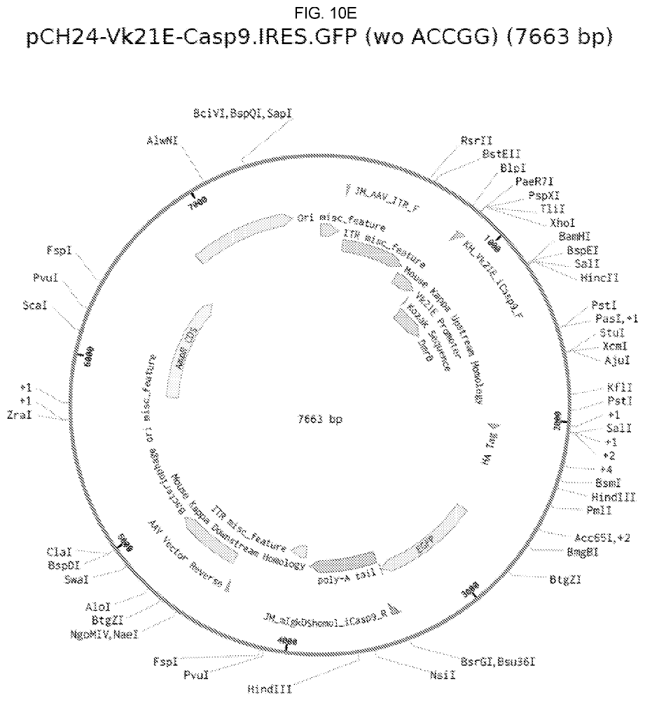

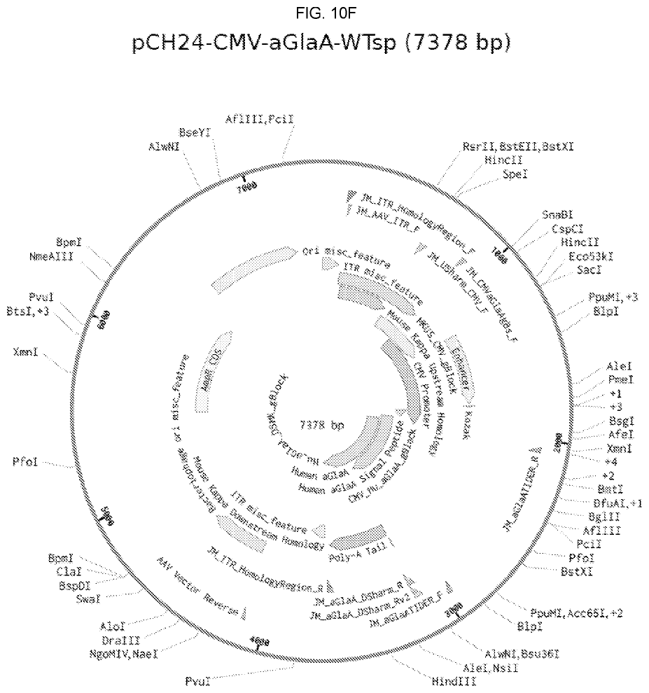

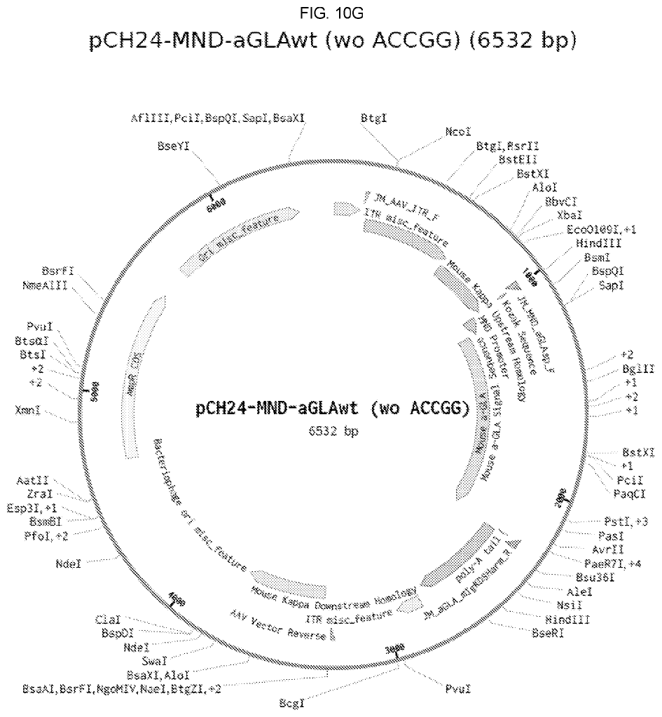

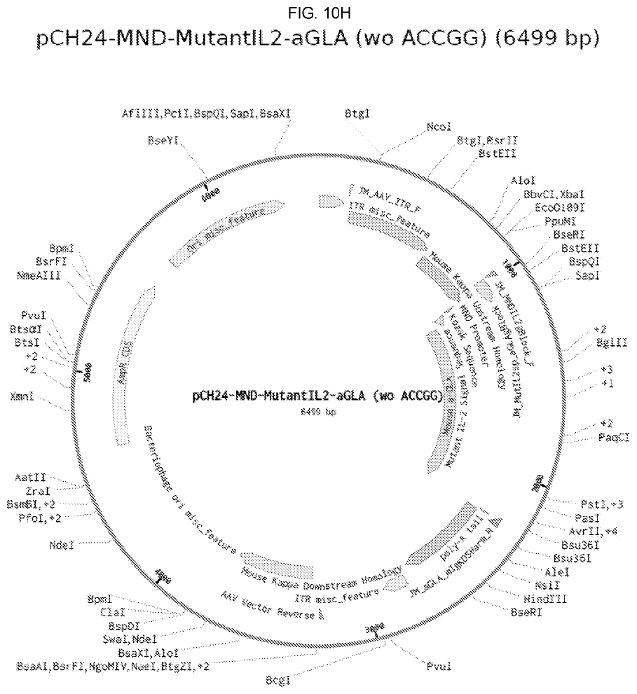

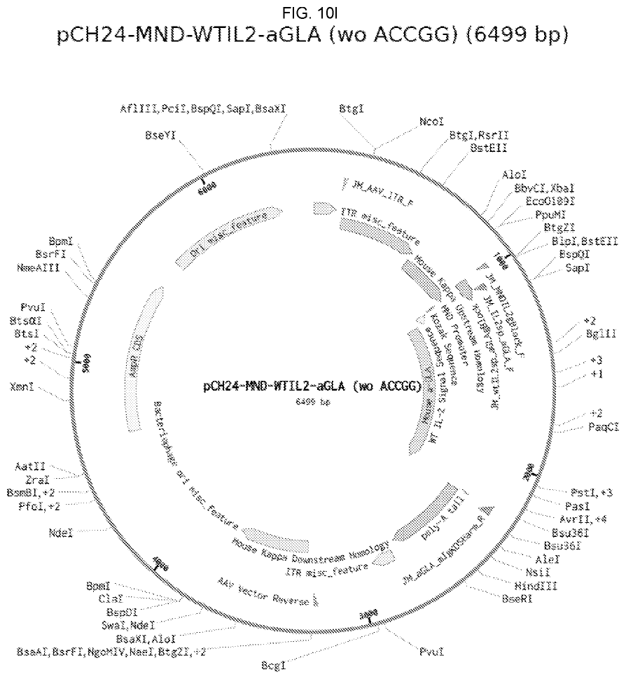

### DETAILED DESCRIPTION

A number of medical disorders are caused by either an insufficiency of a gene product or a defective gene product. Gene therapy can be used to provide a sufficient amount of a gene product when a disorder is caused by an insufficiency and can also be used to inactivate genes that produce defective gene products. Examples of disorders that can be treated by providing a sufficient amount of a gene product include lysosomal storage diseases, clotting disorders, diabetes, and alpha-1 antitrypsin deficiency.

Lysosomal storage diseases are inherited metabolic diseases characterized by a lack of sufficient enzymatic activity to prevent the accumulation of specific macromolecules in various tissues. The progressive accumulation of the specific macromolecules cause the cells containing the lysosomes to become engorged and ultimately leads to tissue damage and organ dysfunction and/or failure. There are nearly 50 of these disorders altogether, and they affect different parts of the body, including the skeleton, brain, skin, heart, and central nervous system.

Enzyme replacement therapy (ERT) can be used as a treatment for lysosomal storage diseases and has significantly elevated treatment options from symptom management and comfort care to actual therapeutic interventions which address the underlying metabolic defect. ERT is not a cure for lysosomal storage diseases but can be very beneficial in attenuating symptoms and disease progression. In particular ERT regimens, patients are required to have bi-weekly intravenous infusion of the enzyme they lack for their entire life. Furthermore, each infusion takes 3 to 4 hours depending on the enzyme and the dose. The bolus nature of these administrations results in high levels of the enzyme following administration that significantly wanes between treatments. Thus, despite the many benefits and improved quality of life ERT has provided for patients with lysosomal storage diseases, better treatment options are still needed. A variety of other disorders are similarly in need of improved treatment options.

The current disclosure provides systems and methods to provide prolonged and tunable expression of gene products within subjects. In relation to lysosomal storage diseases, the prolonged expression obviates the need to receive weekly infusions and overcomes drawbacks associated with the bolus nature of weekly treatments. Moreover, if expression of a gene product wanes over time, the disclosed systems and methods provide fast, safe, and efficient mechanisms to amplify expression of the gene product.

The disclosed systems and methods provide these significant benefits by engineering B cells to express a selected antibody and a gene product. If expression of the gene product falls below a threshold, an antigen that binds the selected antibody can be administered. Binding of the antigen to the selected antibody activates the B cell, such that expression of the gene product will be amplified. Particular embodiments also include a suicide gene so that expression of the gene product can be reduced if expression levels were to exceed a threshold or otherwise cause adverse effects in a subject. Thus, the systems and methods provide for tunable in vivo expression of gene products in subjects.

In particular embodiments, the current disclosure provides these benefits through the targeted insertion of a first genetic construct encoding the selected antibody into an intronic region upstream or downstream of an Eμ enhancer within the native antibody heavy chain gene of B cells.

In particular embodiments, the first genetic construct is inserted into one of SEQ ID NOs: 14, 15, 16, and 17 and includes (i) a promoter; (ii) a signal peptide; (iii) a transgene encoding an entire light chain of a selected antibody; (iv) a flexible linker or a skipping element; (v) the variable portion of the heavy chain of a selected antibody; and (vi) a splice junction that results in expression of the B cell's endogenous heavy chain constant region. In these embodiments, expressing the selected antibody as a single construct overcomes challenges associated with portions of antibodies being encoded by different areas of the endogenous B cell genome. Inclusion of a flexible linker physically links the light chain portion and the heavy chain portion of the expressed selected antibody in a manner that allows them to form a functional unit and at the same time reduces the risk of the antibody portions binding with other potentially expressed antibody chains from the B cell's endogenous genome. Use of a skipping element does not physically link the light chain portion and the heavy chain portion, but their expression in close proximity also results in association to form a functional unit. Inclusion of a splice junction results in the selected antibody including a heavy chain constant region appropriate for the B cell's current activation and/or maturation state. In other words, the selected expressed antibodies can be expressed having any of the B cell's endogenous heavy chain constant regions, and the heavy chain constant region expressed with the selected antibody can naturally change over time.

In particular embodiments, the current disclosure provides the described benefits through the targeted insertion of a second genetic construct encoding the gene product into an intronic region upstream or downstream of an iE_{κ} enhancer within the native antibody light chain gene of B cells. Similar to the selected insertion site for the heavy chain locus, the insertion within the light chain locus results in expression of the gene product instead of the endogenous light chain gene, without relying on excision of the light chain genome.

In particular embodiments, the second genetic construct is inserted into one of SEQ ID NO: 18 or SEQ ID NO: 19 and includes: (i) a promoter and (ii) encodes a gene product.

Particular embodiments utilize a selected antibody that binds an antigen the subject would rarely, if ever, encounter in the natural environment. This feature reduces the likelihood of unintended amplified expression of the gene product. In certain examples, a rare antigen is one that—in relation to a particular subject—has not been detected within 100 miles of where the subject resides in the last 50 years and has not been detected within 100 miles of any location the subject intends to visit in the last 50 years. In certain examples, the antigen is derived from an eradicated virus that is no longer encountered in the natural world. For example, the antigen can be derived from the smallpox virus or the polio virus. Particular examples utilize an antigen of an FDA-approved vaccine, such as an FDA-approved rare antigen. In certain examples a synthetic antigen is used. In this context, synthetic means that the antigen does not naturally occur and will not be encountered in the natural environment. Antigens can be provided in protein form, mRNA form, DNA form, or any other form that results in in vivo presence of the antigen within an intended subject.

As indicated, in addition to amplifying expression of a gene product by administering an antigen that binds the selected antibody, expression of the gene product can also be reduced or eliminated based on the inclusion of a suicide gene encoding a suicide switch.

In particular embodiments, a suicide gene encodes a suicide switch such as an induced caspase protein wherein the induced caspase protein is truncated to delete its physiological dimerization domain and modified to include a chemically induced dimerization (CID) domain. In particular embodiments, the CID provides for dimerization only in the presence of a suicide switch activating agent. In particular embodiments, the induced caspase protein is an induced caspase 9 (iCasp9) with an FRB or FKBP domain of mTor which is activated with rapamycin or analogs thereof. In particular embodiments, administration of rapamycin or analogs thereof leads to dimerization of the suicide switch thereby killing the cell and reducing expression of the gene product.

Aspects of the current disclosure are now described in more supporting detail as follows (i) Disorders and Associated Gene Products; (ii) Antigens and Selected Antibodies; (iii) Targeted Genetic Engineering and Components of Genetic Constructs Encoding Gene Products and Selected Antibodies; (iv) Introduction of Genetic Engineering Components into B Cells; (v) B Cell Markers for Cell Sorting and Targeting; (vi) Formulations & Compositions for Administration; (vii) Methods of Use; (viii) Exemplary Embodiments; and (ix) Closing Paragraphs. These headings are provided for organizational purposes only and do not limit the scope or interpretation of the disclosure.

(i) Disorders and Associated Gene Products. In particular embodiments, B cells are genetically modified to express a gene product. In particular embodiments, the gene product elicits a therapeutic effect. In particular embodiments, the gene product elicits a therapeutic effect in subjects with lysosomal storage diseases, clotting disorders, diabetes, or alpha-1 antitrypsin deficiency. In particular embodiments, the gene product includes a secreted protein, a non-secreted protein, or an inhibitory nucleic acid molecule.

Lysosomal storage diseases (LSD) include over forty genetic disorders, many of which involve genetic defects in select enzymes including various lysosomal hydrolases. Lysosomal storage diseases include Gaucher disease (GD), Fabry disease, mucopolysaccharidosis (MPS, Hurler-Scheie) type I, MPS type II (Hunter Syndrome), MPS type VI, Pompe disease, Aspartylglucosaminuria, Infantile Batten Disease (CNL1), Classic Late Infantile Batten Disease (CNL2), Juvenile Batten Disease (CNL3), other forms of Batten Disease (CNL4-CNL8), Cystinosis, Fucosidosis, Galactosidosialidosis, GM1 gangliosidosis, Krabbe, α-Mannosidosis, β-Mannosidosis, Maroteaux-Lamy, Metachromatic leukodystrophy, Morquio A, Morquio B, Mucolipidosis II/III, Niemann-Pick, Sandhoff, Sanfilippo, Schindler Disease, Schindler-Kanzaki, Sialidosis, Sly, Tay-Sachs, and Wolman Disease. In particular embodiments, a lysosomal storage disease includes Gaucher disease (GD), Fabry disease, mucopolysaccharidosis (MPS) type I, MPS type II, MPS type VI, and Pompe disease.

Gaucher disease results as a consequence of an inherited deficiency of the lysosomal hydrolase glucocerebrosidase (GC), leading to the accumulation of its substrate, glucosylceramide (GL-1), in the lysosomes of histiocytes. The progressive accumulation of GL-1 in tissue macrophages (Gaucher cells) occurs in various tissues. The extent of the accumulation is dependent in part on the genotype. Clinically, three different Gaucher phenotypes are recognized, the non-neuropathic type 1, which is the most common with onset ranging from early childhood to adulthood, and the neuropathic types 2 and 3, presenting in infancy and early childhood, respectively. The primary clinical manifestations common to all forms of Gaucher disease are hepatosplenomegaly, cytopenia, pathological bone fractures and, occasionally, pulmonary failure. A detailed discussion of Gaucher disease may be found in the Online Metabolic & Molecular Bases of Inherited Diseases, Part 16, Chapter 146 and 146.1 (2007).

Niemann-Pick disease (NPD) is a lysosomal storage disease and is an inherited neurometabolic disorder characterized by a genetic deficiency in acid sphingomyelinase (ASM; sphingomyelin cholinephosphohydrolase, EC 3.1.3.12). The lack of functional ASM protein results in the accumulation of sphingomyelin substrate within the lysosomes of neurons and glia throughout the brain. This leads to the formation of large numbers of distended lysosomes in the perikaryon, which are a hallmark feature and the primary cellular phenotype of type A NPD. The presence of distended lysosomes correlates with the loss of normal cellular function and a progressive neurodegenerative course that leads to death of the affected individual in early childhood (The Metabolic and Molecular Bases of Inherited Diseases, eds. Scriver et al., McGraw-Hill, New York, 2001, pp. 3589-3610). Secondary cellular phenotypes (e.g., additional metabolic abnormalities) are also associated with this disease, notably the high-level accumulation of cholesterol in the lysosomal compartment. Sphingomyelin has strong affinity for cholesterol, which results in the sequestering of large amounts of cholesterol in the lysosomes of ASMKO mice and human patients (Leventhal et al. (2001) J. Biol. Chem., 276:44976-44983; Slotte (1997) Subcell. Biochem., 28:277-293; and Viana et al. (1990) J. Med. Genet., 27:499-504.) A detailed discussion of NPD disease may be found in the Online Metabolic & Molecular Bases of Inherited Diseases, Part 16, Chapter 144 (2007).

Mucopolysaccharidoses (MPS) are a group of lysosomal storage diseases caused by deficiencies of enzymes catalyzing the degradation of glycosaminoglycans (mucopolysaccharides). There are 11 known enzyme deficiencies that give rise to 7 distinct MPS, including MPS I (Hurler, Scheie, and Hurler-Scheie Syndromes) and MPS II (Hunter Syndrome). A detailed discussion of MPS may be found in the Online Metabolic & Molecular Bases of Inherited Diseases, Part 16, Chapter 136 (2007).

Pompe disease, or glycogen storage disease type II (GSDII), also termed acid maltase deficiency (AMD) is an inherited disorder of glycogen metabolism resulting from defects in activity of the lysosomal hydrolase acid alpha-glucosidase in all tissues of affected individuals. The enzyme deficiency results in intralysosomal accumulation of glycogen of normal structure in numerous tissues. The accumulation is most marked in cardiac and skeletal muscle and in hepatic tissues of infants with the generalized disorder. In late-onset GSDII, intralysosomal accumulation of glycogen is virtually limited to skeletal muscle and is of lesser magnitude. Electromyographic abnormalities suggestive of the diagnosis include pseudomyotonic discharges and irritability, but in juvenile- and adult-onset patients, the abnormalities can vary in different muscles. CAT scans can reveal the site(s) of affected muscles. Most patients have elevated blood plasma levels of creatine kinase (CK) and elevations in hepatic enzymes, particularly in adult-onset patients, can be found.

B cells can be genetically modified to express an appropriate gene product or active fragment thereof to treat the selected lysosomal storage disease. In particular embodiments, the gene product is an enzyme used to treat a lysosomal storage disease. In particular embodiments, the gene product used to treat a lysosomal storage disease includes glucocerebrosidase (GC), acid sphingomyelinase, mucopolysaccharides, acid alpha-glucosidase, aspartylglucosaminidase, alpha-galactosidase A, palmitoyl protein thioesterase, tripeptidyl peptidase, lysosomal transmembrane protein, cysteine transporter, acid ceramidase, acid alpha-L-fucosidase, cathepsin A, acid beta-glucosidase, acid beta-galactosidase, iduronate-2-sulfatase, alpha-L-iduronidase, galactocerebrosidase, acid alpha-mannosidase, acid beta-mannosidase, arylsulfatase B, arylsulfatase A, N-acetylgalactosamine-6-sulfate, N-acetylglucosamine-1-phosphotransferase, acid sphingomyelinase, NPC-1, alpha-glucosidase, beta-hexosaminidase B, heparan N-sulfatase, alpha-N-acetylglucosaminidase, acetyl-CoA: alpha-glucosaminide, N-acetylglucosamine-6-sulfate, alpha-N-acetylgalactosaminidase, alpha-neuramidase, beta-glucuronidase, beta-hexosaminidase A, and/or acid lipase. In particular embodiments, the gene product includes glucocerebrosidase (SEQ ID NO: 284), acid sphingomyelinase (SEQ ID NO: 285), acid maltase (SEQ ID NO: 286), and/or alpha-galactosidase A (SEQ ID NO: 287) or active fragments thereof. As used herein, “active fragments” are shortened forms of a reference gene product that retain an intended physiological activity.

Clotting disorders are fairly common genetic disorders where factors in the clotting cascade are aberrant in some manner, i.e., lack of expression or production of a mutant protein. Most clotting disorders result in hemophilias such as hemophilia A (factor VIII deficiency), hemophilia B (factor IX deficiency), or hemophilia C (factor XI deficiency). Treatment for these disorders is often related to the severity. For mild hemophilias, treatments can involve therapeutics designed to increase expression of the under-expressed factor, while for more severe hemophilias, therapy involves regular infusion of the missing clotting factor (often 2-3 times a week via enzyme replacement therapy (ERT)) to prevent bleeding episodes. In particular embodiments, the gene product elicits a therapeutic effect in subjects with clotting disorders. In particular embodiments, the gene product is factor VIII (SEQ ID NO: 288), factor IX (SEQ ID NO: 289), or factor XI (SEQ ID NO: 290) or active fragments thereof.

Type I diabetes is a disorder in which immune-mediated destruction of pancreatic beta cells results in a profound deficiency of insulin, which is the primary secreted product of these cells. Restoration of baseline insulin levels provide substantial relief from many of the more serious complications of this disorder which can include “macrovascular” complications involving the large vessels: ischemic heart disease (angina and myocardial infarction), stroke and peripheral vascular disease, as well as “microvascular” complications from damage to the small blood vessels. Microvascular complications may include diabetic retinopathy, which affects blood vessel formation in the retina of the eye, and can lead to visual symptoms, reduced vision, and potentially blindness, and diabetic nephropathy, which may involve scarring changes in the kidney tissue, loss of small or progressively larger amounts of protein in the urine, and eventually chronic kidney disease requiring dialysis. In particular embodiments, the gene product elicits a therapeutic effect in subjects with diabetes. In particular embodiments, the gene product is insulin or an active fragment thereof. In particular embodiments, the insulin sequence is set forth in SEQ ID NO: 291.

Alpha-1 antitrypsin (A1AT) deficiency is an autosomal recessive disease caused by defective production of alpha 1-antitrypsin which leads to inadequate AI AT levels in the blood and lungs. It can be associated with the development of chronic obstructive pulmonary disease (COPD) and liver disorders. Currently, treatment of the diseases associated with this deficiency can involve infusion of exogenous AI AT and lung or liver transplant. In particular embodiments, the gene product elicits a therapeutic effect in subjects with alpha-1 antitrypsin deficiency. In particular embodiments, the gene product is alpha-1 antitrypsin or an active fragment thereof. In particular embodiments, the alpha-1 antitrypsin sequence is set forth in SEQ ID NO: 292.

Other diseases or disorders that include an enzyme deficiency include (disease/enzyme): lactose intolerance/lactase, hemolytic anemia/glucose-6-phosphate dehydrogenase (G6PD) or pyruvate kinase; Gaucher disease/glucocerebrosidase; Wilson disease/ATPase7B; galactosemia/galactose-1-phosphate uridyl transferase (GALT); maple syrup urine disease/branched-chain α-ketoacid dehydrogenase (BCKD) complex; phenylketonuria (PKU)/phenylalanine hydroxylase (PAH); glycogen storage disease Type I (Gierke disease)/glucose-6-phosphatase (G6Pase); glycogen storage disease Type III (Cori disease, or Forbes disease)/debranching enzyme; glycogen storage disease Type IV (Andersen disease)/glycogen-branching enzyme; mitochondrial dysfunction and production of the toxins glutaric acid and 3-OH-glutaric acid/glutaryl-CoA dehydrogenase; Friedreich ataxia (FRDA)/frataxin; Zellweger spectrum disorders (ZSD)/peroxisome biogenesis disorders (PBDs) or peroxisomes; abnormal masculinity/5α-reductase; glucose phosphate isomerase deficiency/glucose phosphate isomerase; Tay-Sachs disease/hexosaminidase A; or fibrodysplasia ossificans progressive (FOP)/activin receptor type-1 (ACVR1) (also known as Activin receptor-like kinase-2 (ALK2)).

Other proteins useful as a gene product includes pramlinitide acetate, growth hormone (GH), insulin-like growth factor, protein C, α1-proteinase inhibitor, erythropoietin, granulocyte colony-stimulating factor (G-CSF), Interleukin 11, human follicle-stimulating hormone (FSH), human chorionic gonadotropin (HCG), Lutropin-α, Interleukin 2 (IL2), Denileukin diftitox (fusion of IL2 and Diphtheria toxin), Interferon-α2a, Interferon-α2b, Interferon-αn3, Interferon-β1a, Interferon-β1b, Interferon-γ1b, human parathyroid hormone, glucagon-like peptide 1, somatostatin, bone morphogenic protein 2, bone morphogenic protein 7, gonadotropin-releasing hormone (GnRH), keratinocyte growth factor (KGF), platelet-derived growth factor (PDGF), B-type natriuretic peptide, hirudin or fragments or mimetics thereof.

In particular embodiments, the gene product includes a secreted protein. In particular embodiments, the gene product includes a non-secreted protein that alters the activity of a B cell.

(ii) Antigens and Selected Antibodies. An antigen refers to any substance that specifically binds to a selected antibody to stimulate the B cell expressing the selected antibody. In particular embodiments, the antigen binds to the selected antibody and causes the B cell to be activated and proliferate, thereby upregulating expression of the gene product.

In particular embodiments, the antigen is selected such that the activation and proliferation of the B cell can be controlled. For example, in certain examples a synthetic antigen is used. In this context, synthetic means that the antigen does not naturally occur and will not be encountered in the natural environment. In certain examples, the antigen is derived from an eradicated virus that is no longer encountered in the natural world. For example, the antigen can be derived from the smallpox virus or the polio virus. In other examples, an antigen can be derived from a virus that is not eradicated, but that does not exist in a geographic area where a subject resides or will travel. In certain examples, the antigen is a rare antigen. A rare antigen is one that—in relation to a particular subject—has not been detected within 100 miles of where the subject resides in the last 50 years and has not been detected within 100 miles of any location the subject intends to visit in the last 50 years.

Particular examples utilize an antigen of an FDA-approved vaccine, such as an FDA-approved rare antigen. Antigens can be provided in protein form, DNA form, mRNA form, or any other form that results in in vivo presence of the antigen within an intended subject.

In particular embodiments, the antigen is the B5 protein of the vaccinia virus or a fragment thereof. In particular embodiments, B5 includes the sequence

(SEQ ID NO: 312)
MKTISVVTLLCVLPAVVYSTCTVPTMNNAKLTSTETSFNNNQKVTFTCD
QGYHSSDPNAVCETDKWKYENPCKKMCTVSDYISELYNKPLYEVNSTMT
LSCNGETKYFRCEEKNGNTSWNDTVTCPNAECQPLQLEHGSCQPVKEKY
SFGEYITINCDVGYEVIGASYISCTANSWNVIPSCQQKCDIPSLSNGLI
SGSTFSIGGVIHLSCKSGFILTGSPSSTCIDGKWNPILPTCVRSNEKFD
PVDDGPDDETDLSKLSKDVVQYEQEIESLEATYHIIIVALTIMGVIFLI
SVIVLVCSCDKNNDQY.

In particular embodiments, the antigen is the A33 protein of the vaccinia virus or a fragment thereof. In particular embodiments, A33 includes the sequence

(SEQ ID NO: 313)
MMTPENDEEQTSVFSATVYRDKIQGKNKRKRVIGLCIRISMVISLLSMI
TMSAFLIVRLNQCMSANEAAITDAAVAVAAASSTHRKVASSTTQYDHKE
SCNGLYYQGSCYILHSDYQLFSDAKANCTAESSTLPNKSDVLTTWLIDY
VKDTWGSDGNPITKTTSDYQDSDVSQEVRKYFCVKTMN.

In particular embodiments, the antigen is a poliovirus receptor protein or a fragment thereof. In particular embodiments, the poliovirus receptor includes the sequence

(SEQ ID NO: 314)
MARAMAAAWPLLLVALLVLSWPPPGTGDVVVQAPTQVPGFLGDSVTLPC
YLQVPNMEVTHVSQLTWARHGESGSMAVFHQTQGPSYSESKRLEFVAAR
LGAELRNASLRMFGLRVEDEGNYTCLFVTFPQGSRSVDIWLRVLAKPQN
TAEVQKVQLTGEPVPMARCVSTGGRPPAQITWHSDLGGMPNTSQVPGFL
SGTVTVTSLWILVPSSQVDGKNVTCKVEHESFEKPQLLTVNLTVYYPPE
VSISGYDNNWYLGQNEATLTCDARSNPEPTGYNWSTTMGPLPPFAVAQG
AQLLIRPVDKPINTTLICNVTNALGARQAELTVQVKEGPPSEHSGISRN
AIIFLVLGILVFLILLGIGIYFYWSKCSREVLWHCHLCPSSEHHQSCR
N.

The selected antibody binds the selected antigen. Naturally occurring antibody structural units include a tetramer. Each tetramer includes two pairs of polypeptide chains, each pair having one light chain and one heavy chain. The amino-terminal portion of each chain includes a variable region that is responsible for antigen recognition and epitope binding. The variable regions exhibit the same general structure of relatively conserved framework regions (FR) joined by three hyper variable regions, also called complementarity determining regions (CDRs). The CDRs from the two chains of each pair are aligned by the framework regions, which enables binding to a specific epitope. From N-terminal to C-terminal, both light and heavy chain variable regions include the domains FR1, CDR1, FR2, CDR2, FR3, CDR3 and FR4.

The assignment of amino acids to each domain can be in accordance with Kabat numbering (Kabat et al. (1991), “Sequences of Proteins of Immunological Interest,” 5th Ed. Public Health Service, National Institutes of Health, Bethesda, Md. (“Kabat” numbering scheme)); Chothia (Al-Lazikani et al., (1997) JMB 273, 927-948 (“Chothia” numbering scheme)), Martin (Abinandan et al., Mol Immunol. 45:3832-3839 (2008), “Analysis and improvements to Kabat and structurally correct numbering of antibody variable domains”), Gelfand, Contact (MacCallum et al., J. Mol. Biol. 262:732-745 (1996), “Antibody-antigen interactions: Contact analysis and binding site topography,” J. Mol. Biol. 262, 732-745.” (Contact numbering scheme)), IMGT (Lefranc M P et al., “IMGT unique numbering for immunoglobulin and T cell receptor variable domains and Ig superfamily V-like domains,” Dev Comp Immunol, 2003 January; 27(1):55-77 (“IMGT” numbering scheme)), AHo (Honegger A and Plückthun A, “Yet another numbering scheme for immunoglobulin variable domains: an automatic modeling and analysis tool,” J Mol Biol, 2001 Jun. 8; 309(3):657-70, (AHo numbering scheme)), North (North et al., J Mol Biol. 406(2):228-256 (2011), “A new clustering of antibody CDR loop conformations”), or other numbering schemes.

Software programs and bioinformatical tools, such as ABodyBuilder and Paratome can also be used to determine CDR sequences. Additionally, delineation of a CDR can be according to X-ray crystallography.

The carboxy-terminal portion of each chain defines a constant region, which can be responsible for effector function particularly in the heavy chain (the Fc). Examples of effector functions include: C1q binding and complement dependent cytotoxicity (CDC); antibody-dependent cell-mediated cytotoxicity (ADCC); phagocytosis; down regulation of cell surface receptors (e.g., B-cell receptors); and B-cell activation.

Within full-length light and heavy chains, the variable and constant regions are joined by a “J” region of amino acids, with the heavy chain also including a “D” region of amino acids. See, e.g., Fundamental Immunology, Ch. 7 (Paul, W., ed., 2nd ed. Raven Press, N.Y. (1989)).

Human light chains are classified as kappa and lambda light chains. In particular embodiments, a human Igκ Fc region includes the sequence:

(SEQ ID NO: 452)
RTVAAPSVFIFPPSDEQLKSGTASVVCLLNNFYPREAKVQWKVDNALQS
GNSQESVTEQDSKDSTYSLSSTLTLSKADYEKHKVYACEVTHQGLSSPV
TKSFNRGEC.

In particular embodiments, a human Igλ Fc region includes the sequence:

(SEQ ID NO: 453)
GQPKANPTVTLFPPSSEELQANKATLVCLISDFYPGAVTVAWKADGSPV
KAGVETTKPSKQSNNKYAASSYLSLTPEQWKSHRSYSCQVTHEGSTVEK
TVAPTECS.

Heavy chains are classified as mu, delta, gamma, alpha, or epsilon, and define the antibody's isotype as IgM, IgD, IgG, IgA, and IgE, respectively. IgG has several subclasses, including, IgG1, IgG2, IgG3, and IgG4. IgM has subclasses including IgM1 and IgM2. IgA is similarly subdivided into subclasses including IgA1 and IgA2.

In particular embodiments, a human IgG1 Fc region includes the sequence:

(SEQ ID NO: 454)
ASTKGPSVFPLAPSSKSTSGGTAALGCLVKDYFPEPVTVSWNSGALTSG
VHTFPAVLQSSGLYSLSSVVTVPSSSLGTQTYICNVNHKPSNTKVDKKV
EPKSCDKTHTCPPCPAPELLGGPSVFLFPPKPKDTLMISRTPEVTCVVV
DVSHEDPEVKFNWYVDGVEVHNAKTKPREEQYNSTYRVVSVLTVLHQDW
LNGKEYKCKVSNKALPAPIEKTISKAKGQPREPQVYTLPPSRDELTKNQ
VSLTCLVKGFYPSDIAVEWESNGQPENNYKTTPPVLDSDGSFFLYSKLT
VDKSRWQQGNVFSCSVMHEALHNHYTQKSLSLSPGK.

In particular embodiments, a human IgG1 Fc region includes the sequence:

(SEQ ID NO: 455)
THTCPPCPAPEFFGGPSVFFFPPKPKDTFMISRTPEVTCVVVDVSHEDP
EVKFNWYVDGVEVHNAKTKPREEQYNSTYRVVSVETVFHQDWENGKEYK
CKVSNKAFPVPIEKTISKAKGQPREPQVYTLPPSREEMTKNQVSLTCLV
KGFYPSDIAVEWESNGQPENNYKTTPPVLDSDGPFFLYSKLTVDKSRWQ
QGNVFSCSVMHEALHNHYTQKSLSLSPGK.

In particular embodiments, a human IgG2 Fc region includes the amino acid sequence:

(SEQ ID NO: 456)
ASTKGPSVFPLAPCSRSTSESTAALGCLVKDYFPEPVTVSWNSGALTSGVHTFPAVLQSSGLY
SLSSVVTVPSSNFGTQTYTCNVDHKPSNTKVDKTVERKCCVECPPCPAPPVAGPSVFLFPPKP
KDTLMISRTPEVTCVVVDVSHEDPEVQFNWYVDGVEVHNAKTKPREEQFNSTFRVVSVLTVVH
QDWLNGKEYKCKVSNKGLPAPIEKTISKTKGQPREPQVYTLPPSREEMTKNQVSLTCLVKGFY
PSDISVEWESNGQPENNYKTTPPMLDSDGSFFLYSKLTVDKSRWQQGNVFSCSVMHEALHNH
YTQKSLSLSPGK

In particular embodiments, a human IgG2 Fc region includes the amino acid sequence:

(SEQ ID NO: 457)
PAPPVAGPSVFLFPPKPKDTLMISRTPEVTCVVVDVSHEDPEVQFNWYVDGVEVHNAKTKPRE
EQFNSTFRVVSVLTVVHQDWLNGKEYKCKVSNKGLPAPIEKTISKTKGQPREPQVYTLPPSRE
EMTKNQVSLTCLVKGFYPSDIAVEWESNGQPENNYKTTPPMLDSDGSFFLYSKLTVDKSRWQ
QGNVFSCSVMHEALHNHYTQKSLSLSPGK

In particular embodiments, a human IgG3 Fc region includes the amino acid sequence:

(SEQ ID NO: 458)
ASTKGPSVFPLAPCSRSTSGGTAALGCLVKDYFPEPVTVSWNSGALTSGVHTFPAVLQSSGLY
SLSSVVTVPSSSLGTQTYTCNVNHKPSNTKVDKRVELKTPLGDTTHTCPRCPEPKSCDTPPPC
PRCPEPKSCDTPPPCPRCPEPKSCDTPPPCPRCPAPELLGGPSVFLFPPKPKDTLMISRTPEV
TCVVVDVSHEDPEVQFKWYVDGVEVHNAKTKPREEQYNSTFRVVSVLTVLHQDWLNGKEYK
CKVSNKALPAPIEKTISKTKGQPREPQVYTLPPSREEMTKNQVSLTCLVKGFYPSDIAVEWESS
GQPENNYNTTPPMLDSDGSFFLYSKLTVDKSRWQQGNIFSCSVMHEALHNRFTQKSLSLSPG
K

In particular embodiments, a human IgG3 Fc region includes the amino acid sequence:

(SEQ ID NO: 459)
PAPELLGGPSVFLFPPKPKDTLMISRTPEVTCVVVDVSHEDPEVQFKWYVDGVEVHNAKTKPR
EEQFNSTFRVVSVLTVLHQDWLNGKEYKCKVSNKALPAPIEKTISKTKGQPREPQVYTLPPSRE
EMTKNQVSLTCLVKGFYPSDIAVEWESSGQPENNYNTTPPMLDSDGSFFLYSKLTVDKSRWQ
QGNIFSCSVMHEALHNRFTQKSLSLSPGK.

In particular embodiments, a human IgG4 Fc region includes the amino acid sequence:

(SEQ ID NO: 460)
ASTKGPSVFPLAPCSRSTSESTAALGCLVKDYFPEPVTVSWNSGALTSGVHTFPAVLQSSGLY
SLSSVVTVPSSSLGTKTYTCNVDHKPSNTKVDKRVESKYGPPCPSCPAPEFLGGPSVFLFPPK
PKDTLMISRTPEVTCVVVDVSQEDPEVQFNWYVDGVEVHNAKTKPREEQFNSTYRVVSVLTVL
HQDWLNGKEYKCKVSNKGLPSSIEKTISKAKGQPREPQVYTLPPSQEEMTKNQVSLTCLVKGF
YPSDIAVEWESNGQPENNYKTTPPVLDSDGSFFLYSRLTVDKSRWQEGNVFSCSVMHEALHN
HYTQKSLSLSLGK.

In particular embodiments, a human IgG4 Fc region includes the amino acid sequence:

(SEQ ID NO: 461)
PAPEFLGGPSVFLFPPKPKDTLMISRTPEVTCVVVDVSQEDPEVQFNWYVDGVEVHNAKTKPR
EEQFNSTYRVVSVLTVLHQDWLNGKEYKCKVSNKGLPSSIEKTISKAKGQPREPQVYTLPPSQ
EEMTKNQVSLTCLVKGFYPSDIAVEWESNGQPENNYKTTPPVLDSDGSFFLYSRLTVDKSRW
QEGNVFSCSVMHEALHNHYTQKSLSLSLGK.

Unless otherwise indicated, the term “antibody” includes (in addition to antibodies having two full-length heavy chains and two full-length light chains as described above) variants, derivatives, and fragments thereof, examples of which are described below. Furthermore, unless explicitly excluded, antibodies can include monoclonal antibodies, human or humanized antibodies, bispecific antibodies, trispecific antibodies, tetraspecific antibodies, multi-specific antibodies, polyclonal antibodies, linear antibodies, minibodies, nanobodies, domain antibodies, synthetic antibodies, chimeric antibodies, antibody fusions, and fragments thereof, respectively. In particular embodiments, antibodies can include oligomers or multiplexed versions of antibodies.

A monoclonal antibody refers to an antibody obtained from a population of substantially homogeneous antibodies, i.e., the individual antibodies including the population are identical and/or bind the same epitope, except for possible variant antibodies, e.g., containing naturally occurring mutations or arising during production of a monoclonal antibody preparation, such variants generally being present in minor amounts. In contrast to polyclonal antibody preparations, which include different antibodies directed against different epitopes, each monoclonal antibody of a monoclonal antibody preparation is directed against a single epitope on an antigen. Thus, the modifier “monoclonal” indicates the character of the antibody as being obtained from a substantially homogeneous population of antibodies and is not to be construed as requiring production of the antibody by any particular method. For example, monoclonal antibodies can be made by a variety of techniques, including the hybridoma method, recombinant DNA methods, phage-display methods, and methods utilizing transgenic animals containing all or part of the human immunoglobulin loci.

A “human antibody” is one which includes an amino acid sequence which corresponds to that of an antibody produced by a human or a human cell or derived from a non-human source that utilizes human antibody repertoires or other human antibody-encoding sequences.

A “human consensus framework” is a framework that represents the most commonly occurring amino acid residues in a selection of human immunoglobulin V_{L} or V_{H} framework sequences. Generally, the selection of human immunoglobulin V_{L} or V_{H} sequences is from a subgroup of variable domain sequences. The subgroup of sequences can be a subgroup as in Kabat et al., Sequences of Proteins of Immunological Interest, Fifth Edition, NIH Publication 91-3242, Bethesda Md. (1991), vols. 1-3. In particular embodiments, for the V_{L}, the subgroup is subgroup kappa I as in Kabat et al. (supra). In particular embodiments, for the V_{H}, the subgroup is subgroup III as in Kabat et al. (supra).

A “humanized” antibody refers to an engineered antibody including amino acid residues from non-human CDRs and amino acid residues from human FRs. In particular embodiments, a humanized antibody will include substantially all of at least one, and typically two, variable domains, in which all or substantially all of the CDRs correspond to those of a non-human antibody, and all or substantially all of the FRs correspond to those of a human antibody. A humanized antibody optionally may include at least a portion of an antibody constant region derived from a human antibody. A “humanized form” of an antibody, e.g., a non-human antibody, refers to an antibody that has undergone humanization.

Humanized antibodies and methods of making them are reviewed, e.g., in Almagro and Fransson, Front. Biosci. 13:1619-1633, 2008, and are further described, e.g., in Riechmann et al., Nature 332:323-329, 1988; Queen et al., Proc. Nat'l Acad. Sci. USA 86:10029-10033, 1989; U.S. Pat. Nos. 5,821,337, 7,527,791, 6,982,321, and 7,087,409; Kashmiri et al., Methods 36:25-34, 2005 (describing SDR (a-CDR) grafting); Padlan, Mol. Immunol. 28:489-498, 1991 (describing “resurfacing”); Dall'Acqua et al., Methods 36:43-60, 2005 (describing “FR shuffling”); and Osbourn et al., Methods 36:61-68, 2005 and Klimka et al., Br. J. Cancer, 83:252-260, 2000 (describing the “guided selection” approach to FR shuffling). EP-B-0239400 provides additional description of “CDR-grafting”, in which one or more CDR sequences of a first antibody is/are placed within a framework of sequences not of that antibody, for instance of another antibody.

Human framework regions that may be used for humanization include: framework regions selected using the “best-fit” method (see, e.g., Sims et al. J. Immunol. 151:2296, 1993); framework regions derived from the consensus sequence of human antibodies of a particular subgroup of light or heavy chain variable regions (see, e.g., Carter et al., Proc. Nati. Acad. Sci. USA, 89:4285, 1992; and Presta et al., J. Immunol., 151:2623, 1993); human mature (somatically mutated) framework regions or human germline framework regions (see, e.g., Almagro and Fransson, Front. Biosci. 13:1619-1633, 2008); and framework regions derived from screening FR libraries (see, e.g., Baca et al., J. Biol. Chem. 272:10678-10684, 1997; and Rosok et al., J. Biol. Chem. 271:22611-22618, 1996).

In particular embodiments, B cells express chimeric antibodies. In particular embodiments, and as used herein, chimeric antibodies refer to a synthetic antibody that includes: (i) at least one portion that is encoded by a B cell's endogenous genome, and (ii) at least one portion that is encoded by an inserted genetic construct. In particular embodiments, the chimeric antibody includes an endogenous heavy chain constant domain, an exogenous immunoglobulin variable and constant light chain, and an exogenous variable heavy chain.

In particular embodiments, the selected antibody binds a rare antigen. In certain examples, the rare antigen is a synthetic antigen, a smallpox virus antigen, or a poliovirus antigen.

In particular embodiments, a smallpox antibody is 7D11. In particular embodiments, the 7D11 antibody includes a variable heavy chain sequence encoded by CAGGTCCAGTTGCAGCAGAGTGGCGCCGAGCTTGCAAAACCAGGTGCTAGTGTTAAAATG AGCTGTAAGGCATCCGGTTACACTTTTACCCGATATTGGATGCACTGGGTGAAGCAAAGGC CAGGTCAAGGACTTGAGTGGATAGGATACATTAATCCTTCAACTGGCTACACTGAGTATAA TCAAAAGTTTAAGGACAAAGCTACACTCACCGCTGATAAGAGTTCCAGCACTGTATATATGC AGTTGTCTAGTCTCACATCCGAGGATTCCGCTGTATACTATTGTGCTCGAACTACTGTAGAC GGATATGATTTTGCATACTGGGGGCAGGGAACTCTCGTTACAGTCTCATCC (SEQ ID NO: 418); and a variable light chain sequence encoded by

(SEQ ID NO: 419)
GACATGGTCATGTCTCAATCTCCATCCTCTCTTGCTGTTAGCGCCGGGGAAAAGGTATCTA
TGAGTTGCAAGTCTTCTCAAACCTTGCTGAACAGCCGCACTAGAAAAAATTACCTGGCTTG
GTATCAACAAAAACCAGGACAGAGTCCTAAACTGCTCATTTACTGGGCCAGCACCCGAGAG
TCTGGAGTACCTGATCGATTTACTGGCTCAGGCAGCGGCACAGATTTCACACTTACAATCT
CTTCTGTCCAAGCCGAAGACTTGGCTGTTTATTACTGCAAACAATCCTATAATCTGTGGACA
TTCGGGGGCGGAACCAAGCTTGAAATTAAA.

In particular embodiments, a 7D11 antibody includes a variable heavy chain including a CDRH1 sequence encoded by CGATATTGGATGCAC (SEQ ID NO: 420), a CDRH2 sequence encoded by TACATTAATCCTTCAACTGGCTACACTGAGTATAATCAAAAGTTTAAGGAC (SEQ ID NO: 421), and a CDRH3 sequence encoded by ACTACTGTAGACGGATATGATTTTGCATAC (SEQ ID NO: 422); and a variable light chain sequence including a CDRL1 sequence encoded by AAGTCTTCTCAAACCTTGCTGAACAGCCGCACTAGAAAAAATTACCTGGCT (SEQ ID NO: 423), a CDRL2 sequence encoded by TGGGCCAGCACCCGAGAGTCT (SEQ ID NO: 424), and a CDRL3 sequence encoded by AAACAATCCTATAATCTGTGGACA (SEQ ID NO: 425).

In particular embodiments, the 7D11 antibody includes a variable heavy chain sequence including QVQLQQSGAELAKPGASVKMSCKASGYTFTRYWMHWVKQRPGQGLEWIGYINPSTGYTEYN QKFKDKATLTADKSSSTVYMQLSSLTSEDSAVYYCARTTVDGYDFAYWGQGTLVTVSS (SEQ ID NO: 402); and a variable light chain including

(SEQ ID NO: 448)
DMVMSQSPSSLAVSAGEKVSMSCKSSQTLLNSRTRKNYLAWYQQKPGQS
PKLLIYWASTRESGVPDRFTGSGSGTDFTLTISSVQAEDLAVYYCKQSY
NLWTFGGGTKLEIK.

In particular embodiments, a smallpox antibody is anti-B5 which binds the B5 protein of smallpox. In particular embodiments, an anti-B5 antibody (8AH8AL) includes a variable heavy chain sequence including EVQLLESGGGLIKPGGSLRLSCAASGFIFRDYNINWVRQAPGKGLEWLGFIRTRASGRSTEYSA SVKGRFTISRDDSKNIAYLHINSLKMEDTAVYYCAKKGDSYYYMDFWGKGTAVTVS (SEQ ID NO: 315); and a variable light chain sequence including ELALTQPASVSGSPGQSITISCTGGRSDLGDSNFVSWYQQYPGKAPKLLIYQVNKRPSGVPDR FSASKSANTASLTISGLQTEDEADYFCSSYTTTSTYVFGIGTKVVVLGQ (SEQ ID NO: 316).

In particular embodiments, an anti-smallpox antibody derived from anti-B5 includes a variable heavy chain including a CDRH1 sequence including DYNIN (SEQ ID NO: 317), a CDRH2 sequence including FIRTRASGRSTE (SEQ ID NO: 318), and a CDRH3 sequence including KGDSYYYMDF (SEQ ID NO: 319); and a variable light chain sequence including a CDRL1 sequence including TGGRSDLGDSNFVS (SEQ ID NO: 320), a CDRL2 sequence including QVNKRPS (SEQ ID NO: 321), and a CDRL3 sequence including SSYTTTSTYV (SEQ ID NO: 322).

In particular embodiments, a smallpox antibody is anti-A33 which binds the A33 protein of smallpox. In particular embodiments, an anti-A33 antibody includes a variable heavy chain sequence including EVQLEQSGSEVKKPGASVKLSCKASGYTFTSYSLGWVRQAPGQGLEWMGWINTKTGNPTYA QGFTGRFVFSLDTSVNTAYLQITSLKAEDTAVYFCAKGTFYYGWGPYYNWFDPWGQGALVTV (SEQ ID NO: 323); and a variable light chain sequence including AELVLTQPPSVSAAPGQKITISCSGSGSNIGRHYVSWYQQFPGTAPKILIYDNDKRPSGISDRFS GSKSGASATLDITGLQTGDEADYYCATWDTNLSGGVFGGGTKVTVLGQ (SEQ ID NO: 324).

In particular embodiments, an anti-smallpox antibody derived from anti-A33 includes a variable heavy chain including a CDRH1 sequence including SYSLG (SEQ ID NO: 325), a CDRH2 sequence including WINTKTGNPT (SEQ ID NO: 326), and a CDRH3 sequence including GTFYYGWGPYYNWFDP (SEQ ID NO: 327); and a variable light chain sequence including a CDRL1 sequence including SGSGSNIGRHYVS (SEQ ID NO: 328), a CDRL2 sequence including DNDKRPS (SEQ ID NO: 329), and a CDRL3 sequence including ATWDTNLSGGV (SEQ ID NO: 330).

In particular embodiments, an anti-poliovirus antibody binds to a poliovirus receptor. In particular embodiments, an anti-polio antibody includes a variable heavy chain sequence including QVQLQQSGAELMKPGASVKISCKATGYTFSNYWIEWIKQRPGHGLEWIGEIFPGSGRINFNEKF KGKATFTADTSSDTTYMQLSSLTSADSAVYYCARTKIYGNSFDYWGQGTTLTVSP (SEQ ID NO: 331); and a variable light chain sequence including DIMMTQSHKFMSTSVGDRVNITCKASQDVGTAVVWYQQKPGQSPKLLIYWASSRHNGVPDRF TGSGSGTDFTLTISNVQSEDLSDYFCQQYSRYPLTFGAGTKLELK (SEQ ID NO: 332).

In particular embodiments, an anti-poliovirus antibody includes a variable heavy chain including a CDRH1 sequence including GFDFSRYW (SEQ ID NO: 333), a CDRH2 sequence including EIHPDSSKINYTPSQ (SEQ ID NO: 334), and a CDRH3 sequence including PDGNYNALDYW (SEQ ID NO: 335); and a variable light chain sequence including a CDRL1 sequence including KASQDVGTAVT (SEQ ID NO: 336), a CDRL2 sequence including WASTRHT (SEQ ID NO: 337), and a CDRL3 sequence including QQYSRYPYT (SEQ ID NO: 338).

In particular embodiments, a selected antibody binds to a synthetic antigen. Methods to generate antibodies against synthetic antigens are well known to those of ordinary skill in the art. In particular embodiments, a synthetic antigen includes a fluorescent protein. In particular embodiments, a fluorescent protein includes R-Phycoerythrin (PE) or allophycocyanin (APC).

Numerous additional antibody sequences are available and known to those of ordinary skill in the art that can be used within the teachings of the current disclosure. Sequence information for commercially available antibodies may be found in the Drug Bank database, the CAS Registry, and/or the RSCB Protein Data Bank. Moreover, nucleic acid sequences encoding portions of selected antibodies described herein can be easily derived by one of ordinary skill in the art.

(iii) Targeted Genetic Engineering and Components of Genetic Constructs Encoding Gene Products and Selected Antibodies. Targeted genetic engineering allows control over the target sites of genetic therapies. Within the teachings of the current disclosure, any gene editing system capable of precise sequence targeting and modification can be used. These systems typically include a targeting element for precise targeting and a cutting element for cutting the targeted genetic site. Guide RNA is one example of a targeting element while various nucleases provide examples of cutting elements. Targeting elements and cutting elements can be separate molecules or linked, for example, by a nanoparticle. In particular embodiments, the targeting elements and/or cutting elements can be associated with a nanoparticle. Herein, “associated with” means functionally linked such that targeted genetic insertion occurs at an intended genomic site. A functional linkage to a nanoparticle can be through covalent linkage, through electrostatic attraction or through an intervening molecule or layer. Alternatively, a targeting element and a cutting element can be linked together into one dual purpose molecule. When insertion of a genetic construct is intended, systems can also include homology-directed repair templates (i.e., homology regions also commonly referred to as homology arms) associated with the genetic constructs. As detailed further below, however, different gene editing systems can adopt different components and configurations while maintaining the ability to precisely target, cut, and modify selected genomic sites.

Particular embodiments utilize zinc finger nucleases (ZFNs) as gene editing agents. ZFNs are a class of site-specific nucleases engineered to bind and cleave DNA at specific positions. ZFNs are used to introduce double stranded breaks (DSBs) at a specific site in a DNA sequence which enables the ZFNs to target unique sequences within a genome in a variety of different cells. Moreover, subsequent to double-stranded breakage, homology-directed repair (HDR) or non-homologous end joining (NHEJ) takes place to repair the DSB, thus enabling genome editing.

ZFNs are synthesized by fusing a zinc finger DNA-binding domain to a DNA cleavage domain. The DNA-binding domain includes three to six zinc finger proteins which are similar to those found in transcription factors. The DNA cleavage domain includes the catalytic domain of, for example, Fokl endonuclease. The Fokl domain functions as a dimer requiring two constructs with unique DNA binding domains for sites on either side of the target site cleavage sequence. The Fokl cleavage domain cleaves within a five or six base pair spacer sequence separating the two inverted half-sites.

For additional information regarding ZFNs and ZFNs useful within the teachings of the current disclosure, see, e.g., U.S. Pat. Nos. 6,534,261; 6,607,882; 6,746,838; 6,794,136; 6,824,978; 6,866,997; 6,933,113; 6,979,539; 7,013,219; 7,030,215; 7,220,719; 7,241,573; 7,241,574; 7,585,849; 7,595,376; 6,903,185; 6,479,626; and U.S. Application Publication Nos. 2003/0232410 and 2009/0203140 as well as Gaj et al., Nat Methods, 2012, 9(8):805-7; Ramirez et al., Nucl Acids Res, 2012, 40(12):5560-8; Kim et al., Genome Res, 2012, 22(7): 1327-33; Urnov et al., Nature Reviews Genetics, 2010, 11:636-646; Miller, et al. Nature biotechnology 25, 778-785 (2007); Bibikova, et al. Science 300, 764 (2003); Bibikova, et al. Genetics 161, 1169-1175 (2002); Wolfe, et al. Annual review of biophysics and biomolecular structure 29, 183-212 (2000); Kim, et al. Proceedings of the National Academy of Sciences of the United States of America 93, 1156-1160 (1996); and Miller, et al. The EMBO journal 4, 1609-1614 (1985).

Particular embodiments can use transcription activator like effector nucleases (TALENs) as gene editing agents. TALENs refer to fusion proteins including a transcription activator-like effector (TALE) DNA binding protein and a DNA cleavage domain. TALENs are used to edit genes and genomes by inducing DSBs in the DNA, which induce repair mechanisms in cells. Generally, two TALENs must bind and flank each side of the target DNA site for the DNA cleavage domain to dimerize and induce a DSB. The DSB is repaired in the cell by NHEJ or HDR if an exogenous double-stranded donor DNA fragment is present.

As indicated, TALENs have been engineered to bind a target sequence of, for example, an endogenous genome, and cut DNA at the location of the target sequence. The TALEs of TALENs are DNA binding proteins secreted by Xanthomonas bacteria. The DNA binding domain of TALEs include a highly conserved 33 or 34 amino acid repeat, with divergent residues at the 12^{th} and 13^{th} positions of each repeat. These two positions, referred to as the Repeat Variable Diresidue (RVD), show a strong correlation with specific nucleotide recognition. Accordingly, targeting specificity can be improved by changing the amino acids in the RVD and incorporating nonconventional RVD amino acids.

Examples of DNA cleavage domains that can be used in TALEN fusions are wild-type and variant Fokl endonucleases. For additional information regarding TALENs, see U.S. Pat. Nos. 8,440,431; 8,440,432; 8,450,471; 8,586,363; and 8,697,853; as well as Joung and Sander, Nat Rev Mol Cell Biol, 2013, 14(1):49-55; Beurdeley et al., Nat Commun, 2013, 4: 1762; Scharenberg et al., Curr Gene Ther, 2013, 13(4):291-303; Gaj et al., Nat Methods, 2012, 9(8):805-7; Miller, et al. Nature biotechnology 29, 143-148 (2011); Christian, et al. Genetics 186, 757-761 (2010); Boch, et al. Science 326, 1509-1512 (2009); and Moscou & Bogdanove, Science 326, 1501 (2009)).

Particular embodiments can utilize MegaTALs as gene editing agents. MegaTALs have a single chain rare-cleaving nuclease structure in which a TALE is fused with the DNA cleavage domain of a meganuclease. Meganucleases, also known as homing endonucleases, are single peptide chains that have both DNA recognition and nuclease function in the same domain. In contrast to the TALEN, the megaTAL only requires the delivery of a single peptide chain for functional activity.

Exemplary meganucleases include I-SceI, I-SceII, I-SceIII, I-SceIV, I-SceV, I-SceVI, I-SceVII, I-CeuI, I-CeuAIIP, I-CreI, I-CrepsbIP, I-CrepsbIIP, I-CrepsbIIIP, I-CrepsbIVP, I-TliI, I-PpoI, PI-PspI, F-SceI, F-SceII, F-SuvI, F-TevI, F-TevII, I-AmaI, I-AniI, I-ChuI, I-CmoeI, I-CpaI, I-CpaII, I-CsmI, I-CvuI, I-CvuAIP, I-DdiI, I-DdiII, I-DirI, I-DmoI, I-HmuI, I-HmuII, I-HsNIP, I-LlaI, I-MsoI, I-NaaI, I-NanI, I-NcIIP, I-NgrIP, I-NitI, I-NjaI, I-Nsp236IP, I-PakI, I-PboIP, I-PcuIP, I-PcuAI, I-PcuVI, I-PgrIP, 1-PobIP, I-PorI, I-PorIIP, I-PbpIP, I-SpBetaIP, I-ScaI, I-SexIP, 1-SneIP, I-SpomI, I-SpomCP, I-SpomIP, I-SpomlIP, I-SquIP, I-Ssp6803I, I-SthPhiJP, I-SthPhiST3P, I-SthPhiSTe3bP, I-TdeIP, I-TevI, I-TevII, I-TevlII, I-UarAP, 1-UarHGPAIP, 1-UarHGPA13P, I-VinIP, 1-ZbiIP, PI-MtuI, PI-MtuHIP PI-MtuHIIP, PI-PfuI, PI-PfuII, PI-PkoI, PI-PkoII, PI-Rma43812IP, PI-SpBetaIP, PI-SceI, PI-TfuI, PI-TfuII, PI-ThyI, PI-TliI, and PI-THII.

In particular embodiments, different areas of the endogenous B cell genome can be targeted using CRISPR gene editing systems. The CRISPR nuclease system is a prokaryotic immune system that confers resistance to foreign genetic elements such as plasmids and phages and provides a form of acquired immunity. CRISPRs are DNA loci containing short repetitions of base sequences. In the context of a prokaryotic immune system, each repetition is followed by short segments of spacer DNA belonging to foreign genetic elements that the prokaryote was exposed to. This CRISPR array of repeats interspersed with spacers can be transcribed into RNA. The RNA can be processed to a mature form and associate with a nuclease, such as cas (CRISPR-associated) nuclease. A CRISPR-Cas system including an RNA having a sequence that can hybridize to the foreign genetic elements and Cas nuclease can then recognize and cut these exogenous genetic elements in the genome.

A CRISPR-Cas system does not require the generation of customized proteins to target specific sequences, but rather a single Cas enzyme can be programmed by a short guide RNA molecule to recognize a specific DNA target. The CRISPR-Cas systems of bacterial and archaeal adaptive immunity show extreme diversity of protein composition and genomic loci architecture. The CRISPR-Cas system loci have more than 50 gene families and there are no strictly universal genes, indicating fast evolution and extreme diversity of loci architecture. So far, adopting a multi-pronged approach, there is comprehensive cas gene identification of 395 profiles for 93 Cas proteins. Classification includes signature gene profiles plus signatures of locus architecture. A new classification of CRISPR-Cas systems is proposed in which these systems are broadly divided into two classes, Class 1 with multi-subunit effector complexes and Class 2 with single-subunit effector modules exemplified by the Cas9 protein.

At least three different Cas9 nucleases have been developed for genome editing. The first is the wild type Cas9 which introduces double strand breaks (DSBs) at a specific DNA site, resulting in the activation of DSB repair machinery. DSBs can be repaired by non-homologous end joining (NHEJ), homology-directed repair (HDR), or microhomology mediated repair (MMEJ). NHEJ can involve repair of a DSB with no homology (<5 bp) between the two ends joined during repair; HDR can involve repair of a DSB with a large region of homology between the ends joined during repair (100 or more nucleotides); and MMEJ can involve repair of a DSB with a small (5 to 50 bp) region of homology between the ends joined during repair. Another type of Cas9 includes a mutant Cas9, known as the Cas9D10A, with only nickase activity, which means that it only cleaves one DNA strand and does not activate NHEJ. Thus, the DNA repairs proceed via the HDR pathway only. The third is a nuclease-deficient Cas9 (dCas9) which does not have cleavage activity but is able to bind DNA. Therefore, dCas9 is able to target specific sequences of a genome without cleavage. By fusing dCas9 with various effector domains, dCas9 can be used either as a gene silencing or activation tool.

In addition to the Class 1 and Class 2 CRISPR-Cas systems, more recently a putative Class 2, Type V CRISPR-Cas class exemplified by Cpf1 has been identified Zetsche et al. (2015) Cell 163(3): 759-771. The Cpf1 nuclease particularly can provide added flexibility in target site selection by means of a short, three base pair recognition sequence (TTN), known as the protospacer-adjacent motif or PAM. Cpf1's cut site is at least 18 bp away from the PAM sequence, thus the enzyme can repeatedly cut a specified locus after indel (insertion and deletion) formation, increasing the efficiency of HDR. Moreover, staggered DSBs with sticky ends permit orientation-specific donor template insertion.

Additional information regarding CRISPR-Cas systems and components thereof are described in, U.S. Pat. Nos. 8,697,359, 8,771,945, 8,795,965, 8,865,406, 8,871,445, 8,889,356, 8,889,418, 8,895,308, 8,906,616, 8,932,814, 8,945,839, 8,993,233 and 8,999,641 and applications related thereto; and WO2014/018423, WO2014/093595, WO2014/093622, WO2014/093635, WO2014/093655, WO2014/093661, WO2014/093694, WO2014/093701, WO2014/093709, WO2014/093712, WO2014/093718, WO2014/145599, WO2014/204723, WO2014/204724, WO2014/204725, WO2014/204726, WO2014/204727, WO2014/204728, WO2014/204729, WO2015/065964, WO2015/089351, WO2015/089354, WO2015/089364, WO2015/089419, WO2015/089427, WO2015/089462, WO2015/089465, WO2015/089473 and WO2015/089486, WO2016205711, WO2017/106657, WO2017/127807 and applications related thereto.

Particular embodiments combine CRISPR RNA (crRNA) and trans-activating CRISPR RNA (tracrRNA) into a guide RNA (gRNA) or synthetic single guide RNA (sgRNA). In particular embodiments, a gRNA or sgRNA are the RNA molecules used to specify a particular target area for cleavage by a nuclease. In particular embodiments, gRNA includes two parts: crRNA, a nucleotide sequence (e.g., 17-20 nucleotides) complementary to the target DNA, and a tracrRNA sequence, which serves as a binding scaffold for the Cas nuclease. When the crRNA and tracrRNA elements are combined into a single RNA molecule, the molecule is referred to as sgRNA, though gRNA and sgRNA are often used interchangeably. In particular embodiments, gRNA includes sgRNA. For certain gene editing systems, the target sequence may be adjacent to a PAM (e.g., 5′-20 nt target—NGG-3′) or can include a PAM (SEQ ID NOs: 20-139). In particular embodiments, guide RNA (gRNA) includes a target site adjacent to the PAM targeted by the genome editing complex. The gRNA can include at least the 16, 17, 18, 19, 20, 21, or 22 nucleotides adjacent to the PAM.

As indicated, within the disclosed systems and methods, genetic constructs are inserted at two areas of the B cell's endogenous genome: an area upstream or downstream of the Eμ enhancer and an area upstream or downstream of the iE_{κ} enhancer.

In particular embodiments, the targeted area upstream or downstream of the Eμ enhancer includes the sequence as set forth in SEQ ID NO: 14, SEQ ID NO: 15, SEQ ID NO: 16, or SEQ ID NO: 17. In particular embodiments, more discrete target areas upstream or downstream of the Eμ enhancer including or adjacent to the PAM sites include SEQ ID NOs: 20-99. Sequences particularly capable of targeting these sites for genetic modification are described as guide RNA (gRNA) provided as SEQ ID NOs: 140-219. In particular embodiments, a target site as set forth in SEQ ID NOs: 20-99 can be targeted by a gRNA sequence as set forth in one of SEQ ID NOs: 140-219.

In particular embodiments, sgRNA targeting the human or mouse IgH of each endogenous antibody targets the region 100 bp downstream of the J region. In particular embodiments, this region is targeted to express a version of the selected antibody containing the C region from the endogenous genome.

sgRNA targeting the heavy chain genome for genetic modification upstream or downstream of the Eμ enhancer include SEQ ID NO: 181 and/or SEQ ID NO: 142 in humans. In particular embodiments, sgRNA targeting the heavy chain genome upstream or downstream of the Eμ enhancer for genetic modification include SEQ ID NO: 145 in mice.

In particular embodiments, the targeted area upstream or downstream of the iE_{κ} enhancer includes the sequence as set forth in SEQ ID NO: 18 or 19. In particular embodiments, more discrete target areas upstream or downstream of the iE_{κ} enhancer including or adjacent to the PAM sites include SEQ ID NOs: 100-139. Sequences particularly capable of targeting these sites for genetic modification are described as gRNA provided as SEQ ID NOs: 220-259. In particular embodiments, a target site as set forth in SEQ ID NOs: 100-139 can be targeted by a gRNA sequence as set forth in one of SEQ ID NOs: 220-259.

In particular embodiments, sgRNA targeting the light chain genome for genetic modification include SEQ ID NO: 225, SEQ ID NO: 224, and/or SEQ ID NO: 221 in humans. In particular embodiments, sgRNA targeting the light chain sequence include SEQ ID NO: 244, SEQ ID NO: 242, and/or SEQ ID NO: 241 in mice.

In particular embodiments, a cutting element is directed to the targeted DNA location with the assistance of engineered gRNAs (Sternberg et al., Mol Cell. 2015; 58(4):568-74), such as the sgRNAs described above. Genetic constructs with homology regions flanking the cut genomic region are efficiently inserted into this location by the homology-directed DNA repair mechanism (Elliott et al., Mol Cell Biol. 1998; 18(1):93-101). Using this approach expression of the endogenous variable region of the heavy chain and entirety of the light chain will be eliminated and genes encoding the selected antibody and gene product will be inserted into the targeted genetic locations. This targeted insertion eliminates or significantly reduces the possibility of off-target effects resulting from random genetic insertion.

Genetic constructs inserted in the heavy chain or light chain locus of the B cell endogenous genome share certain features. For example, each inserted construct includes homology regions, a promoter, and a coding sequence encoding the selected antibody or gene product.

Homology regions cause the genetic construct to align and hybridize to a cut targeted genetic region, resulting in integration of the genetic construct into the endogenous genome. In particular embodiments, a genetic construct may include an upstream genome homology end with 20 to 1,500 bp of genome homology, and a downstream genome homology end with 20 to 1,500 bp of genome homology. The regions of homology may, for example, provide “homology stitches”, which can mediate insertion of the genetic construct into the targeted insertion site. In particular embodiments, regions of homology may particularly include 20-50 base pairs; 300-500 base pairs; 350-550 base pairs; 900-1,000 base pairs, or 400-600 base pairs. In particular embodiments, regions of homology may particularly include 30-40 base pairs (e.g., 36 base pairs); 445-455 base pairs (e.g., 450 base pairs); 495-510 base pairs (e.g., 503 base pairs); and/or 960-980 base pairs (e.g., 968 base pairs).

In particular embodiments, the upstream genome homology end and the downstream genome homology end may include sequences with homology to genome sequences between a light chain VDJ region and a light chain Eμ enhancer.

Exemplary homology regions for the heavy chain endogenous genome include upstream (SEQ ID NO: 260) and downstream (SEQ ID NO: 261) genome homology regions for mouse and upstream (SEQ ID NO: 262 and SEQ ID NO: 264) and downstream (SEQ ID NO: 263 and SEQ ID NO: 265) genome homology regions for human.

In particular embodiments, the upstream genome homology end and the downstream genome homology end may include sequences with homology to genome sequences between a light chain VJ region and a light chain iEκ enhancer.

Exemplary homology regions for the light chain endogenous genome include upstream (SEQ ID NO: 266, SEQ ID NO: 268, and SEQ ID NO: 270) and downstream (SEQ ID NO: 267, SEQ ID NO: 269, and SEQ ID NO: 271) genome homology regions for mouse and upstream (SEQ ID NO: 272, SEQ ID NO: 274, and SEQ ID NO: 276) and downstream (SEQ ID NO: 273, SEQ ID NO: 275, and SEQ ID NO: 277) genome homology regions for human.

Promoters of genetic constructs disclosed herein can include general promoters, tissue-specific promoters, cell-specific promoters, and/or promoters specific for the cytoplasm. Promoters may include strong promoters, weak promoters, constitutive expression promoters, and/or inducible promoters. Inducible promoters direct expression in response to certain conditions, signals or cellular events. For example, the promoter may be an inducible promoter that requires a particular ligand, small molecule, transcription factor or hormone protein in order to effect transcription from the promoter. Particular examples of promoters include V_{H} promoter, V_{L} promoter, minBglobin promoter, CMV promoter, MND promoter, Vk21E promoter, minCMV promoter, minRho promoter, SV40 immediately early promoter, the Hsp68 minimal promoter (proHSP68), and the Rous Sarcoma Virus (RSV) long-terminal repeat (LTR) promoter. Minimal promoters have no activity to drive gene expression on their own but can be activated to drive gene expression when linked to a proximal enhancer. In particular embodiments, the genetic construct encoding the selected antibody is under the control of a V_{H} promoter. In particular embodiments, the genetic construct encoding the gene product is under the control of a CMV promoter. In particular embodiments, the genetic construct encoding the gene product is under the control of an MND promoter or Vk21E promoter.

Coding sequences encode the selected antibody or gene product. Exemplary coding sequences are provided within the disclosure and numerous additional coding sequences are known, or can be generated, by those of ordinary skill in the art.

Coding sequences for selected antibodies can be provided as a single genetic construct. In particular embodiments, this is achieved by including a skipping element within the genetic construct. One example of a skipping element is a self-cleaving peptide, such as a self-cleaving “2A” peptide. 2A peptides function by causing the ribosome to skip the synthesis of a peptide bond at a defined location, leading to production of two proteins from one mRNA. The 2A sequences are short (e.g., 20 amino acids), facilitating use in size-limited constructs, and proteins are produced at a 1:1 ratio. Particular examples include T2A (GSG)EGRGSLLTCGDVEENPGP (SEQ ID NO: 339); P2A (GSG)ATNFSLLKQAGDVEENPGP (SEQ ID NO: 340); E2A (GSG)QCTNYALLKLAGDVESNPGPP (SEQ ID NO: 341); and F2A (GSG)VKQTLNFDLLKLAGDVESNPGP (SEQ ID NO: 342).

In particular embodiments, the genetic construct encoding the selected antibody includes an internal ribosome entry site (IRES) sequence. The IRES can be positioned upstream of the heavy chain VDJ of the genetic construct. IRES are non-coding structured RNA sequences that allow ribosomes to initiate translation at a second internal site on a mRNA molecule, leading to production of two proteins from one mRNA. However, IRES driven translation is less efficient than 2A driven translation, leading to lower expression of the second protein in the transcript.

In particular embodiments, the genetic construct encoding the selected antibody encodes a flexible linker between the light chain portion of the selected antibody and the heavy chain portion of the selected antibody. A linker can be a series of amino acids that flexibly link one protein domain to another protein domain in a way that allows the linked sequences to interact to form a functional unit.

In particular sequences, flexible linkers can include one or more series of combinations of glycine and serine, which provide flexibility to the linker sequence. Exemplary Gly-Ser linkers include (GGS)n (SEQ ID NO: 343), (GGGS)n (SEQ ID NO: 344), and (GGGGS)n (SEQ ID NO: 345) wherein n=1 to 100 and every integer therebetween. In particular embodiments, n=10, 11, 12, 13, 14, 15, 16, 17, 18, 19, 20, 21, 22, 23, 24, 25, 26, 27, 28, 29, or 30. In particular embodiments, a linker includes 50 to 80 amino acids. In particular embodiments, the linker includes 54, 57, or 60 amino acids. In particular embodiments, the linker is encoded by GGAGGAAGTAGTGGCAGCGGGAGTGGGTCCAATTGGAGTCATCCTCAATTTGAGAAAGGA GGGGGAGGGTCCAATTGGTCTCATCCGCAGTTTGAGAAGGGCGGCGGCGGCTCCAATTG GTCCCATCCCCAGTTTGAAAAAGGCTCTGGTGGAGGTGGTAGTGCTGGTGGG (SEQ ID NO: 346). In particular embodiments, the linker includes GGSSGSGSGSNWSHPQFEKGGGGSNWSHPQFEKGGGGSNWSHPQFEKGSGGGGSAGG (SEQ ID NO: 347).

Additional examples of flexible linkers include (KESGSVSSEQLAQFRSLD)n (SEQ ID NO: 393) and (EGKSSGSGSESKST)n (SEQ ID NO: 348). In these linkers the Gly and Ser residues in the linker were designed to provide flexibility, whereas Glu and Lys were added to improve the solubility. Bird, R E et al. Science, 1988; 242:423-426. In particular embodiments, n=1 to 100 and every integer therebetween. In particular embodiments, n=10, 11, 12, 13, 14, 15, 16, 17, 18, 19, 20, 21, 22, 23, 24, 25, 26, 27, 28, 29, or 30. In particular embodiments, these linkers include 50-80 amino acids.

Particular embodiments include a splice junction that allows splicing between RNA encoded by the genetic construct and RNA encoded by the endogenous heavy chain constant region. In particular embodiments, the genetic constructs include a splice junction sequence at the 3′ end. Splicing can refer to the removal of introns and joining together of exons by an RNA/protein complex known as the spliceosome. A splice junction refers to an intronic sequence directly flanking an exon. A splice junction at the 3′ end of an exon can include a splice donor site. Splice donor site sequences typically begin with “GU”. In particular embodiments, the splice junction may include 40-80 bp of an intron following the last exon of a VDJ. In particular embodiments, the splice junction includes 40-80 bp of the intron flaking the 3′ end of the human IGHJ1 gene segment or the mouse IGHJ3 gene. In particular embodiments, the splice junction includes CAG/gtaagt, with the cut and splice taking place after the uppercase G (indicated by the “splice” annotation). In particular embodiments, the splice junction includes CAG/gtgagt. The CA form the end of a serine codon, and the G begins the first codon from the constant region. In particular embodiments, a splice junction with flanking sequence includes CAGGTAAGTCTGCTGTCTGGGGATAGCGGGGAGCCAGGTGTACTGGGCCAGGCAAGGGC TTTGGATC (SEQ ID NO: 349) or CAGGTGAGTTGGCTTTCCTTCTGCCTCCTTTCTCTGGGCCCAGCGTCCTCTGACCTGGAG CTGGGAGATAATGTCCGGGGGCTCCTT (SEQ ID NO: 350) in genetic constructs for insertion into a human locus. In particular embodiments, a splice junction with flanking sequence includes CAGGTGAGTCCTAACTTCTCCCATTCTAAATGCATGTTGGGGGGATTCTGGGCCTTCAGGA CCA (SEQ ID NO: 426) in genetic constructs for insertion into a mouse locus.

Genetic constructs can include additional components. For example, in particular embodiments, the genetic constructs encode a signal peptide. In certain examples, genetic constructs encoding the selected antibody can also encode signal peptides derived from human IgH heavy chains (e.g, MELGLSWIFLLAILKGVQC (SEQ ID NO: 351); MELGLRWVFLVAILEGVQC (SEQ ID NO: 352); MKHLWFFLLLVAAPRWVLS (SEQ ID NO: 353); MDWTWRILFLVAAATGAHS (SEQ ID NO: 354); MDWTWRFLFVVAAATGVQS (SEQ ID NO: 355); MEFGLSWLFLVAILKGVQC (SEQ ID NO: 356); MEFGLSWVFLVALFRGVQC (SEQ ID NO: 357); and MDLLHKNMKHLWFFLLLVAAPRWVLS (SEQ ID NO: 358) or signal peptides derived from human lgL light chains (e.g., MDMRVPAQLLGLLLLWLSGARC (SEQ ID NO: 359) and MKYLLPTAAAGLLLLAAQPAMA (SEQ ID NO: 360)).

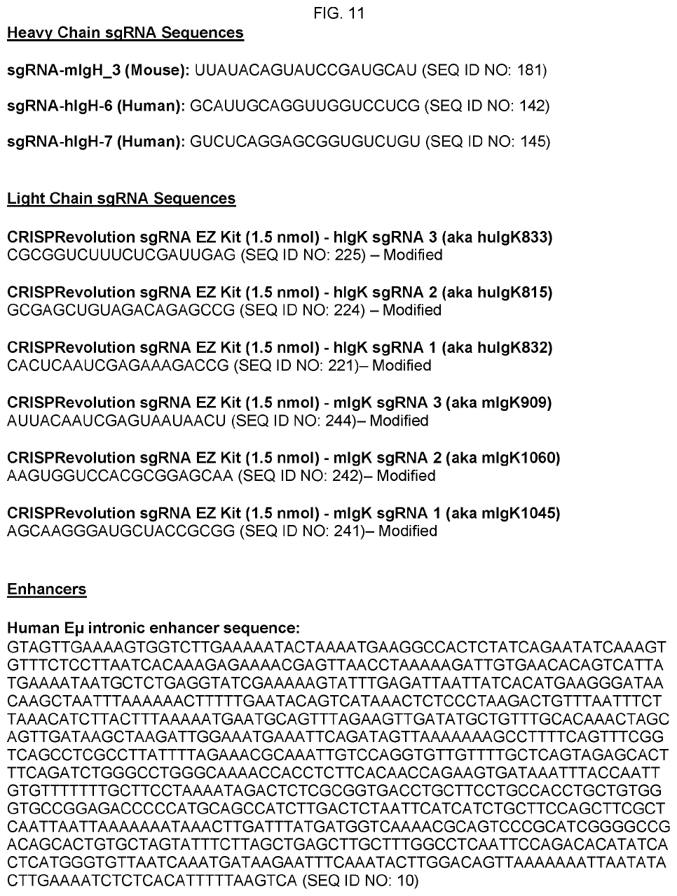

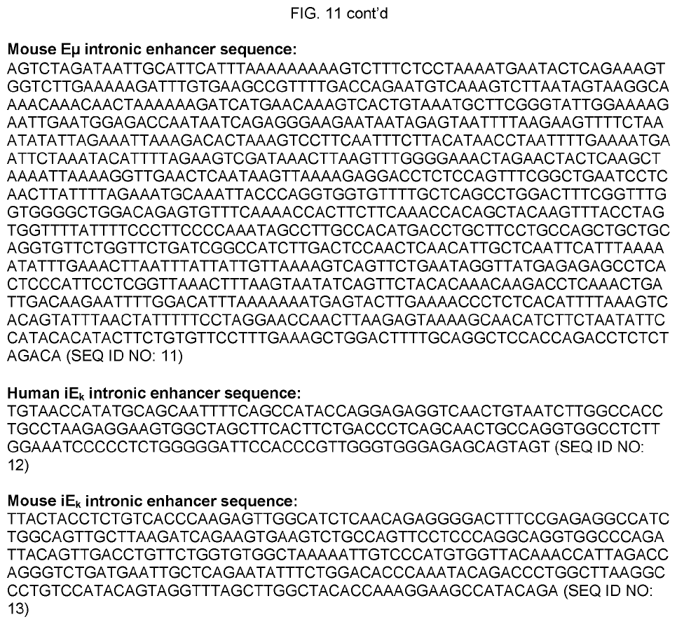

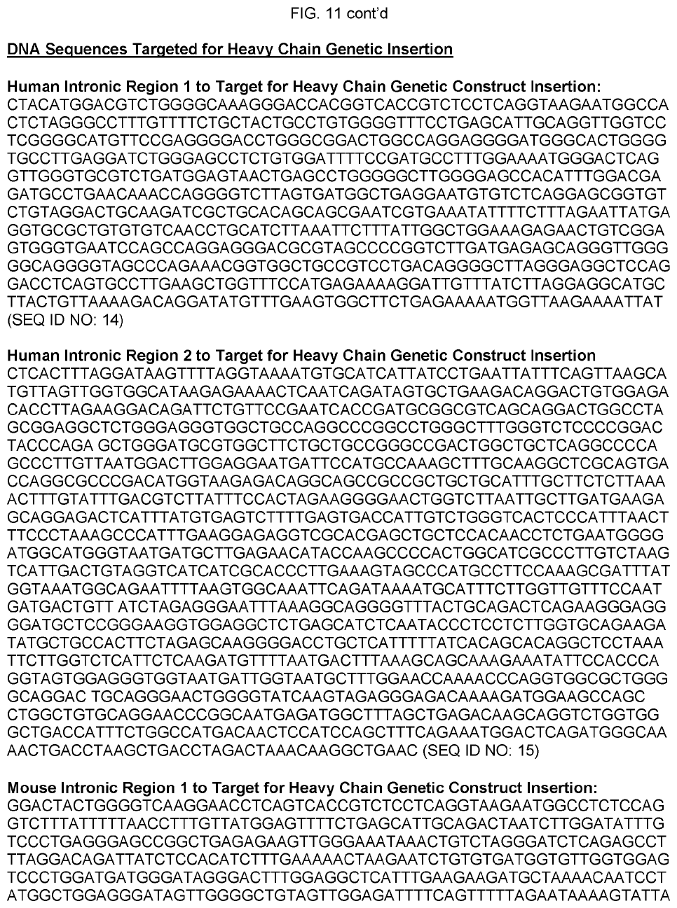

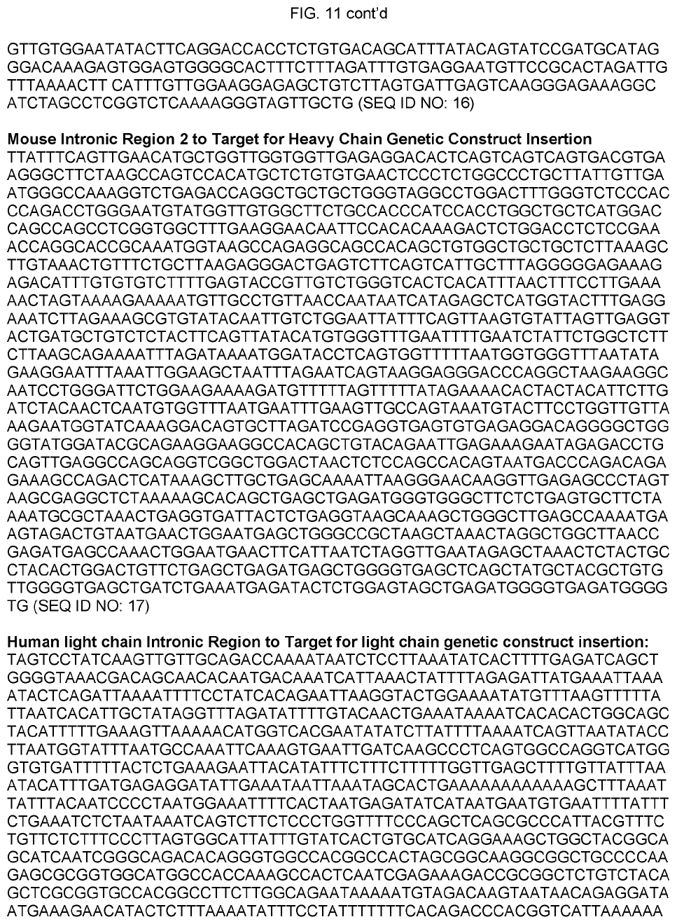

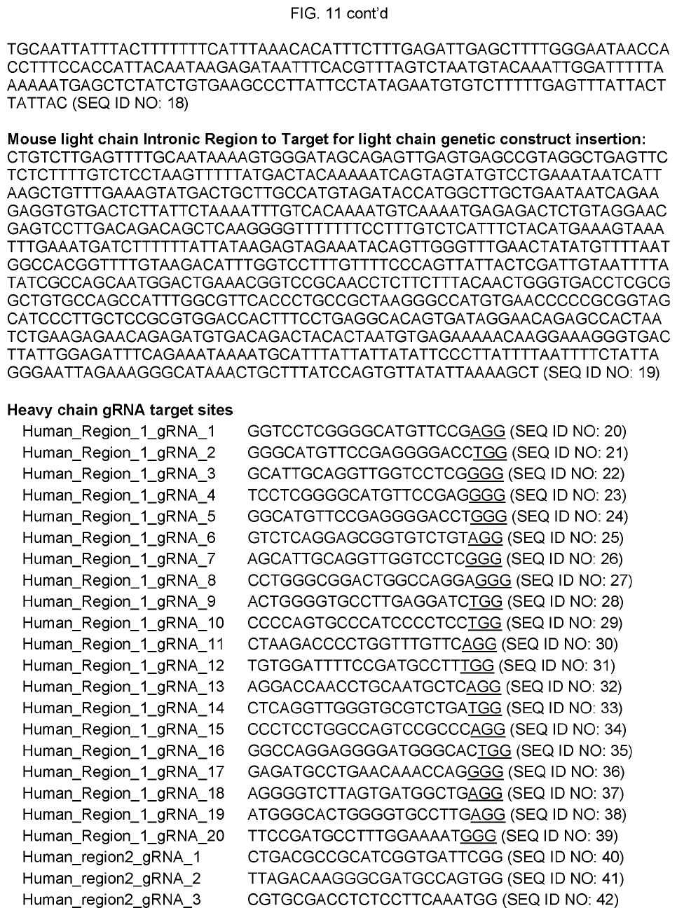

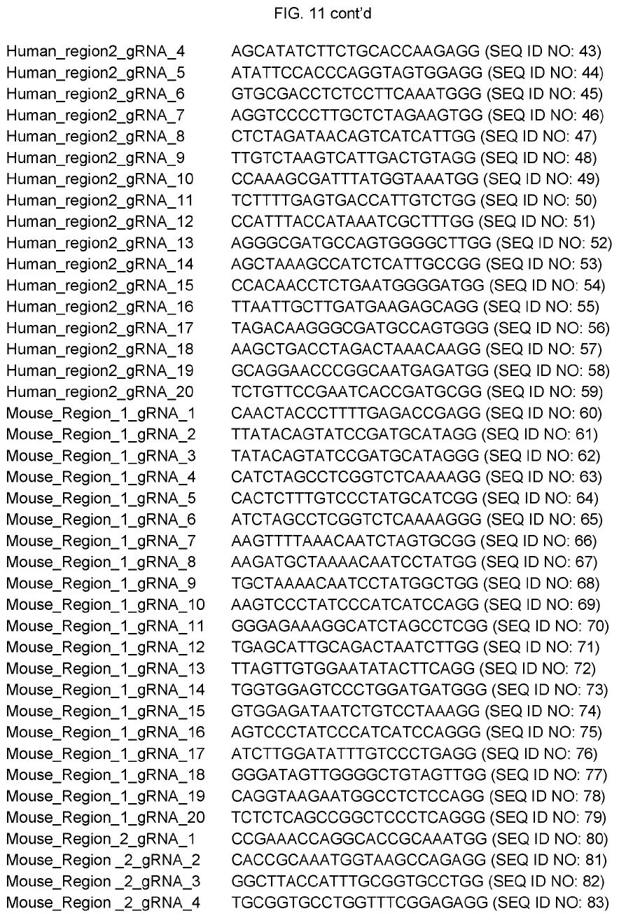

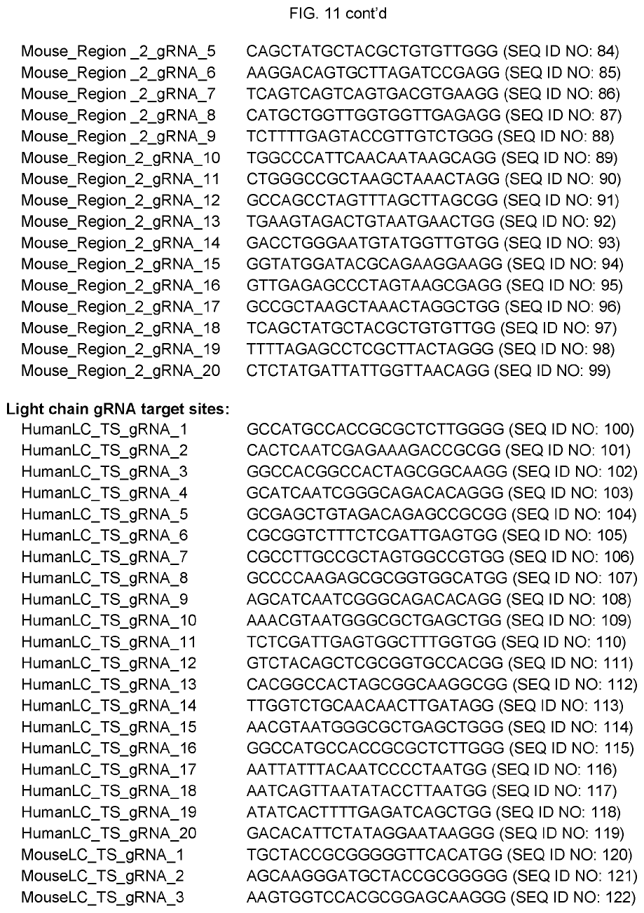

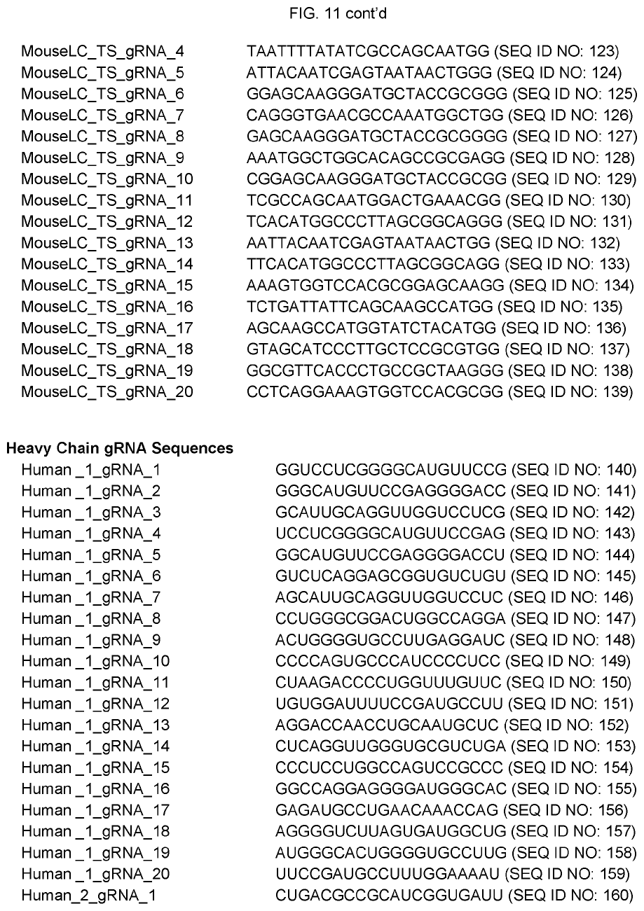

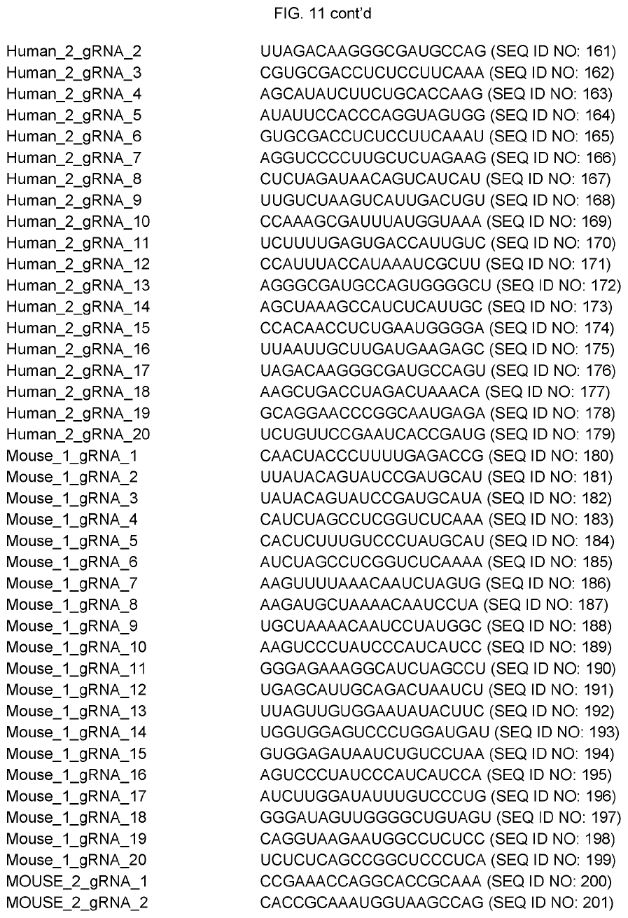

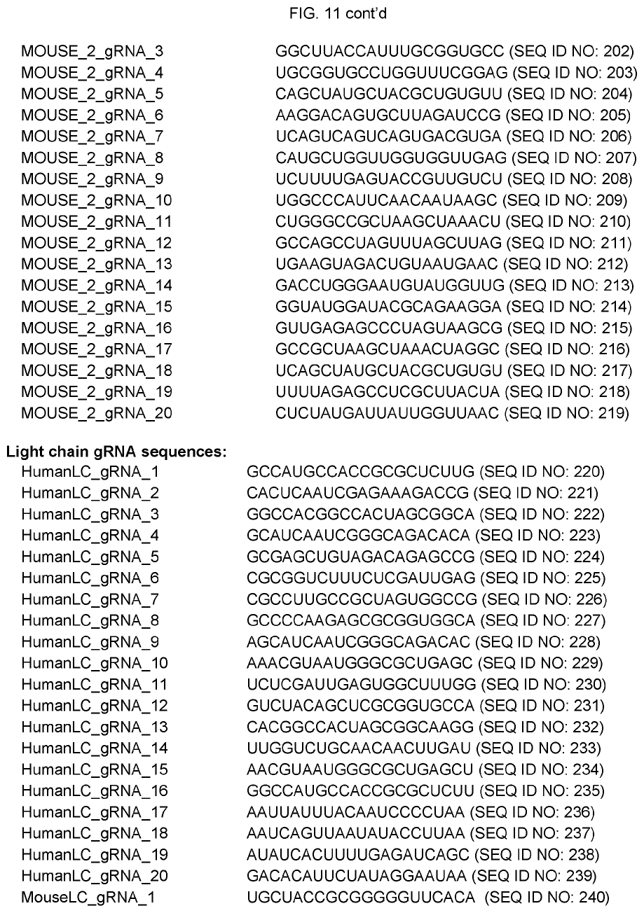

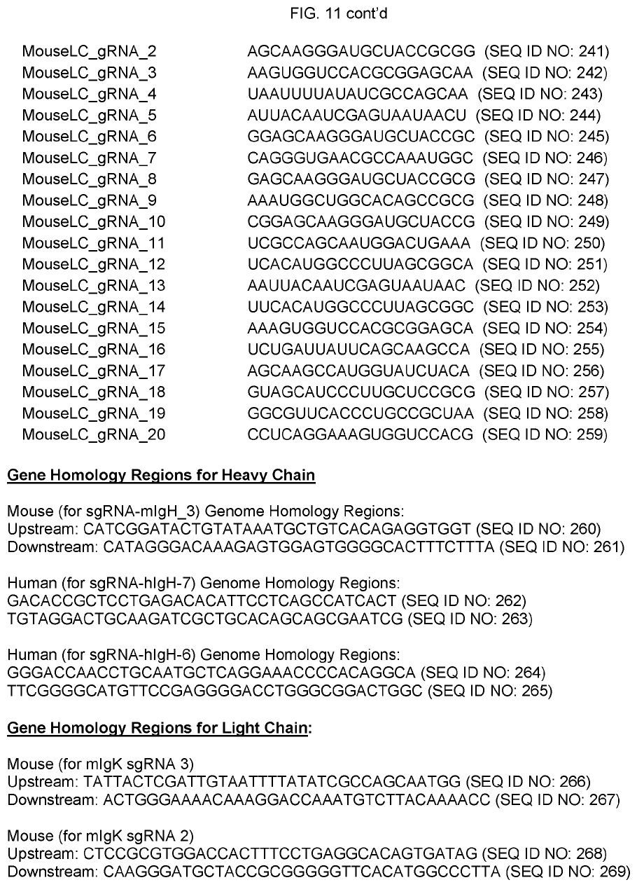

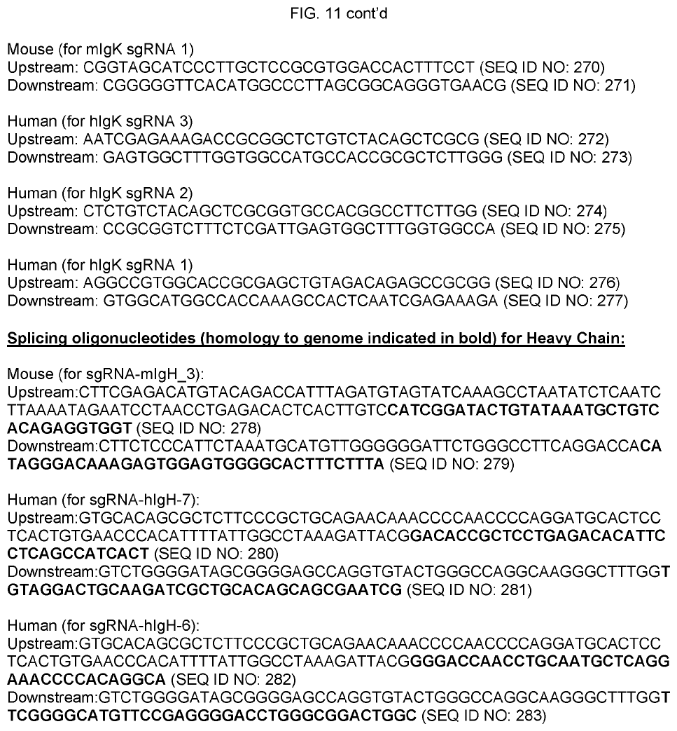

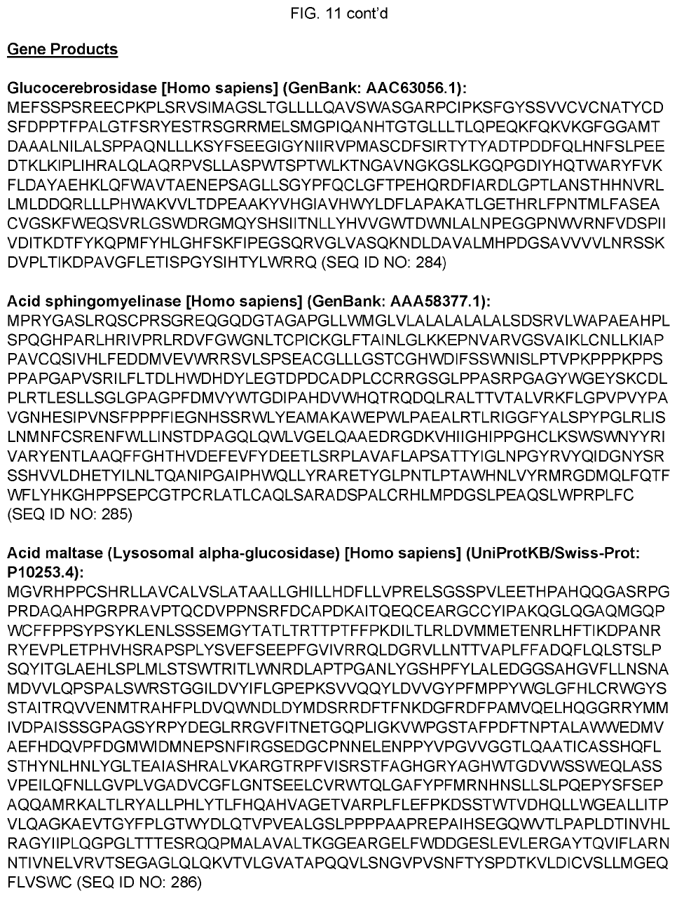

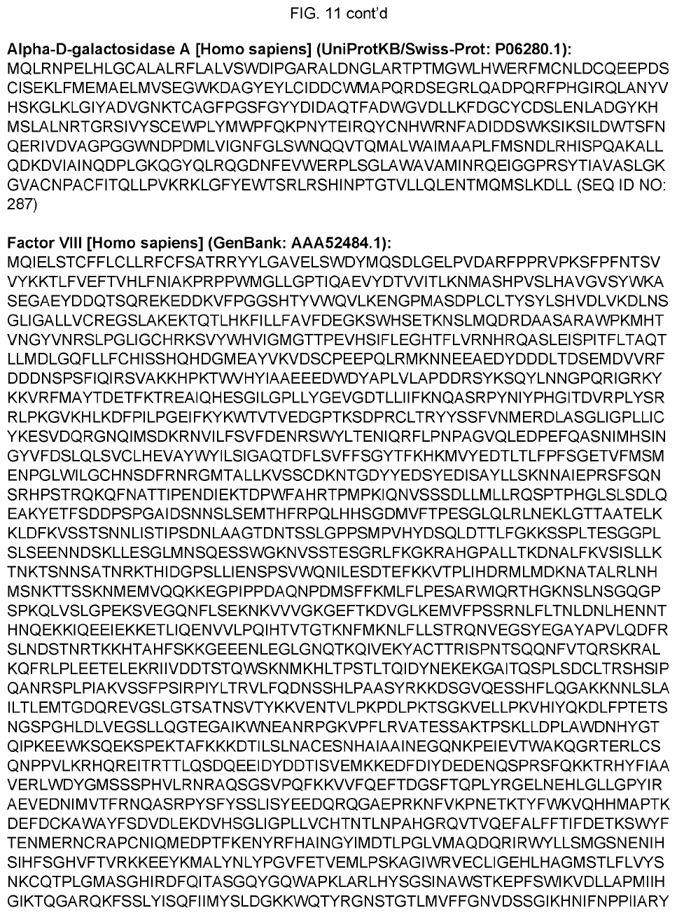

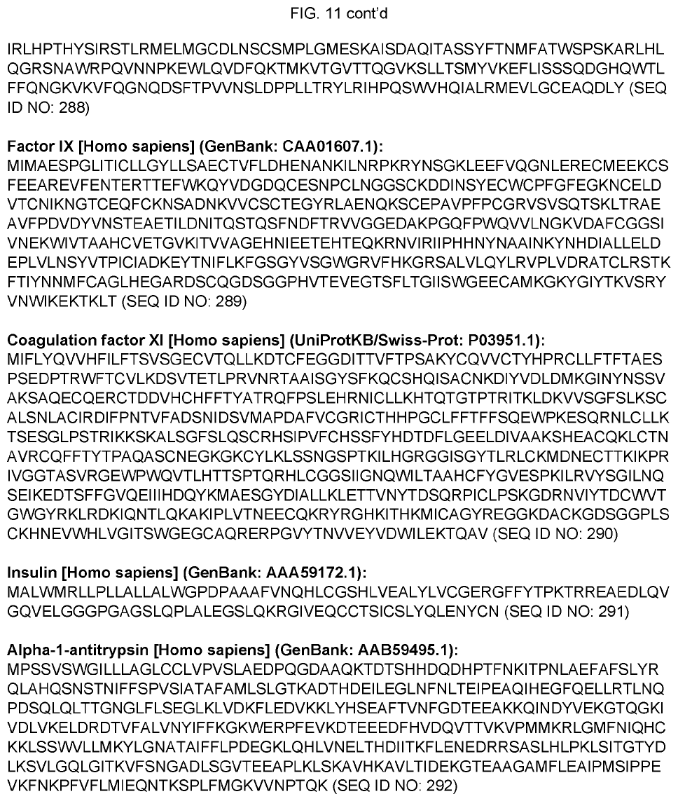

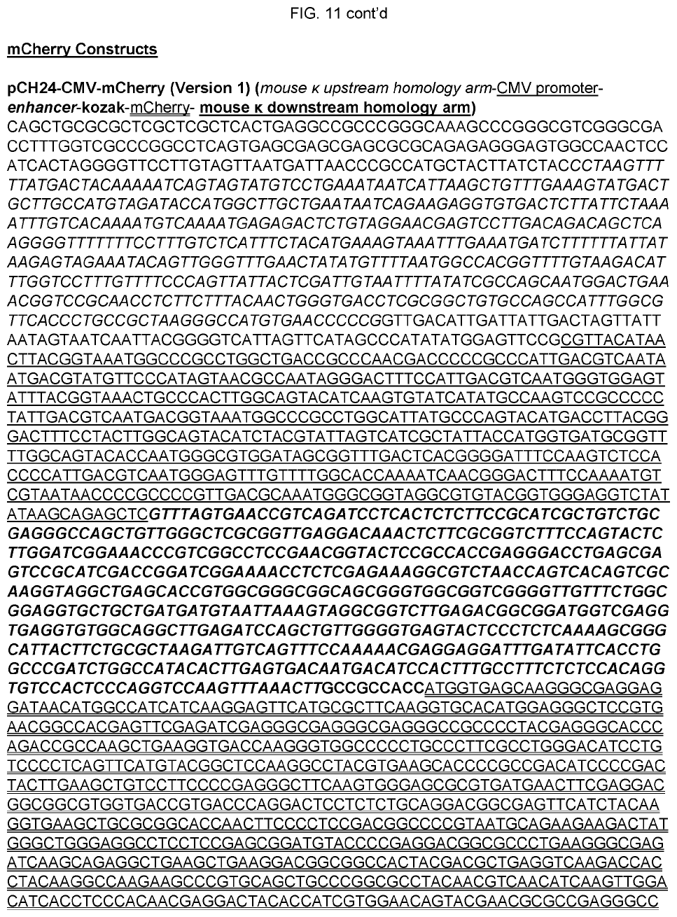

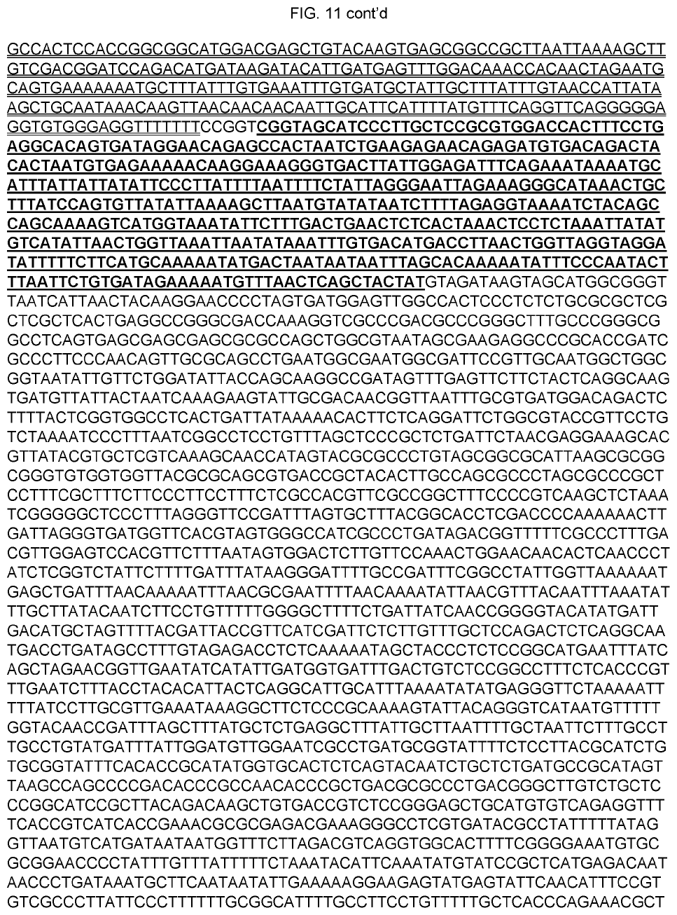

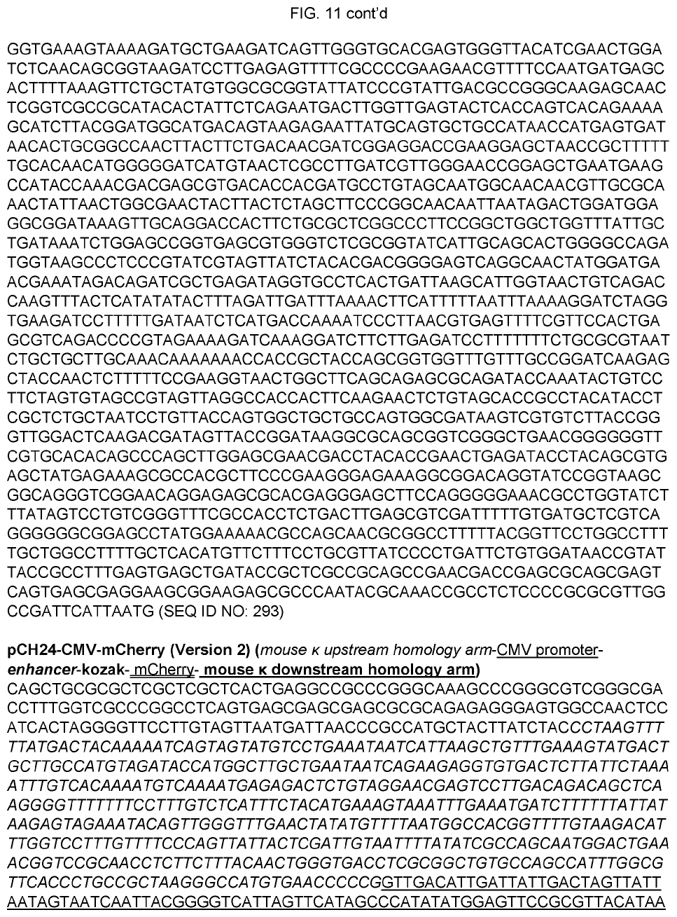

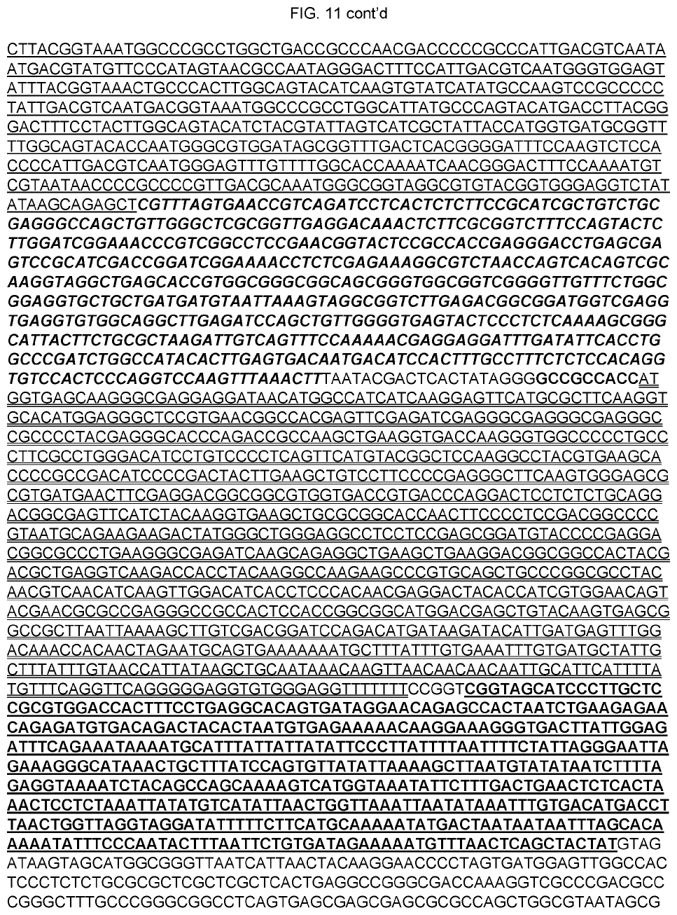

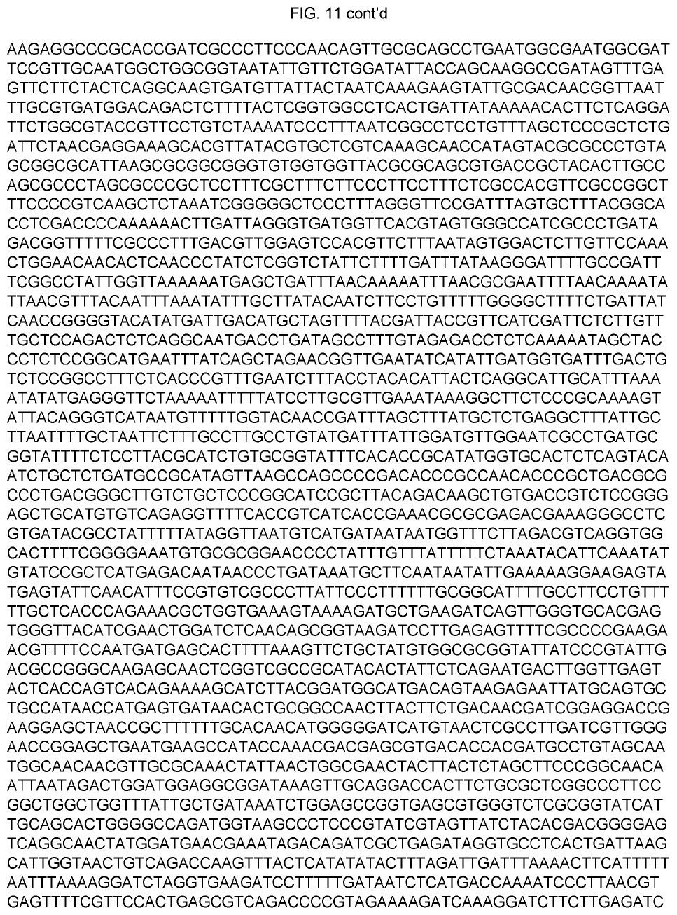

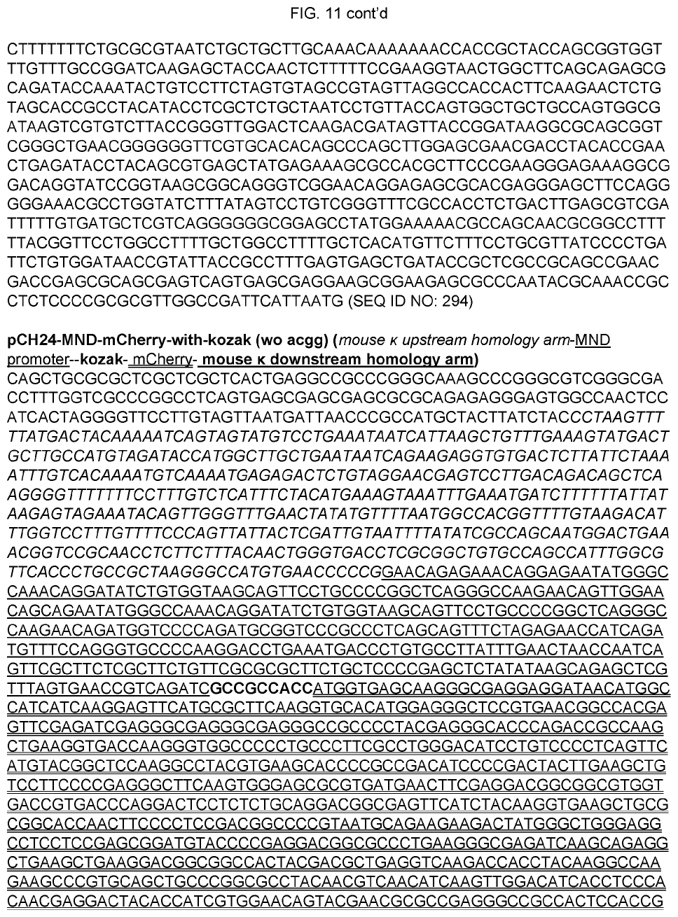

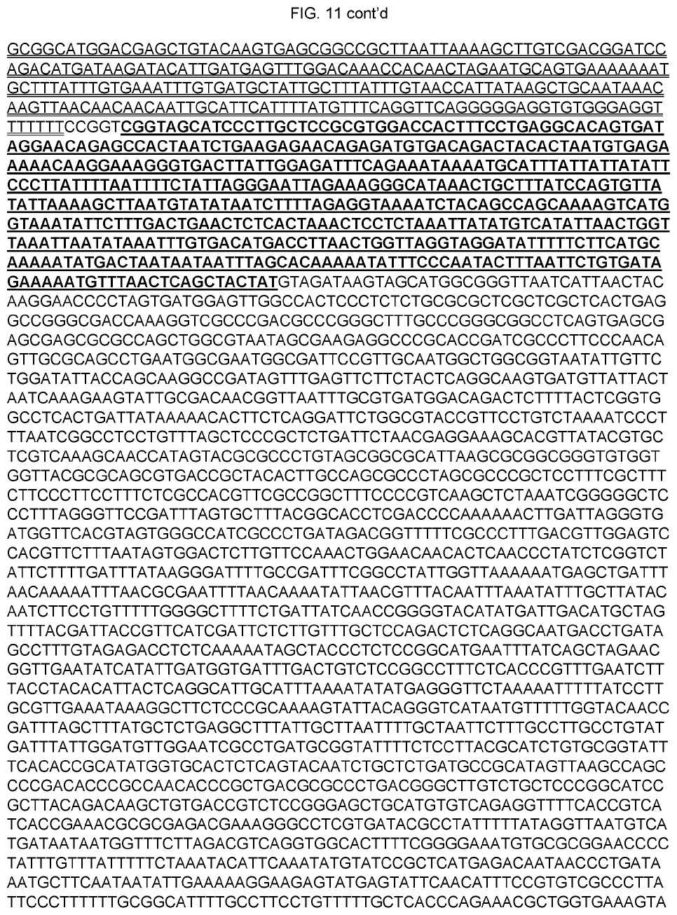

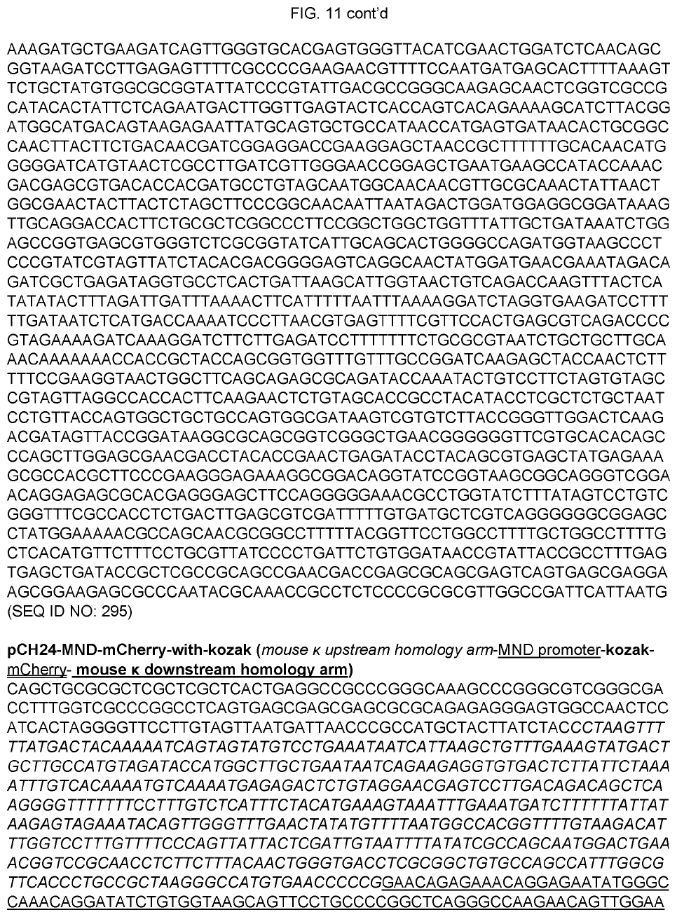

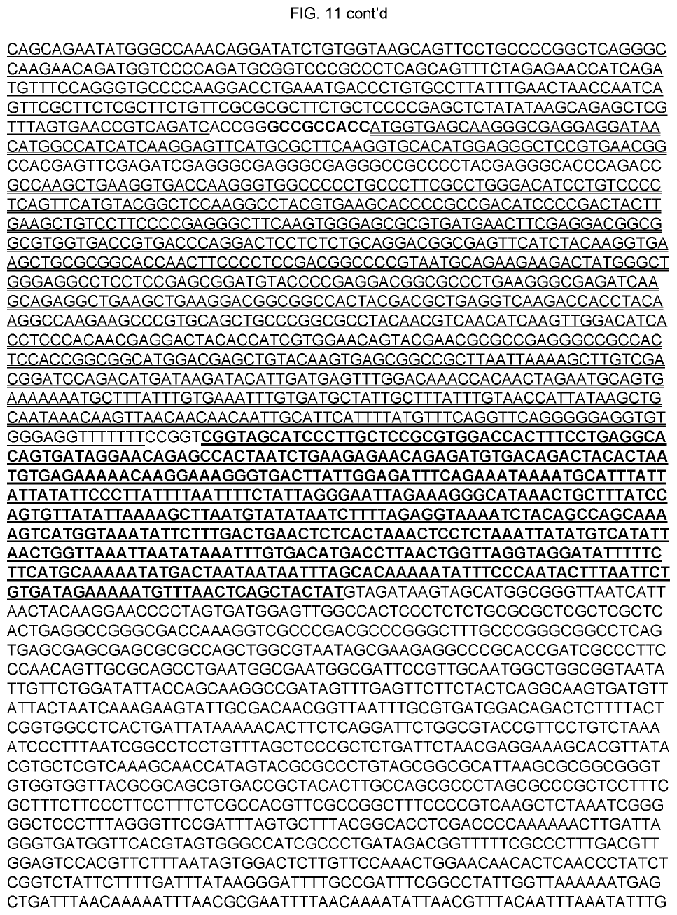

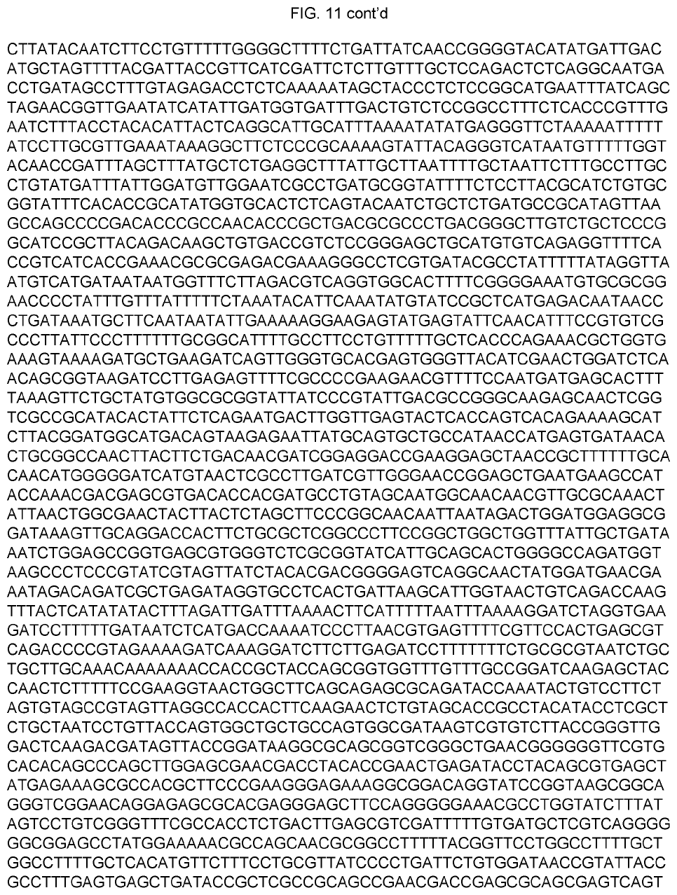

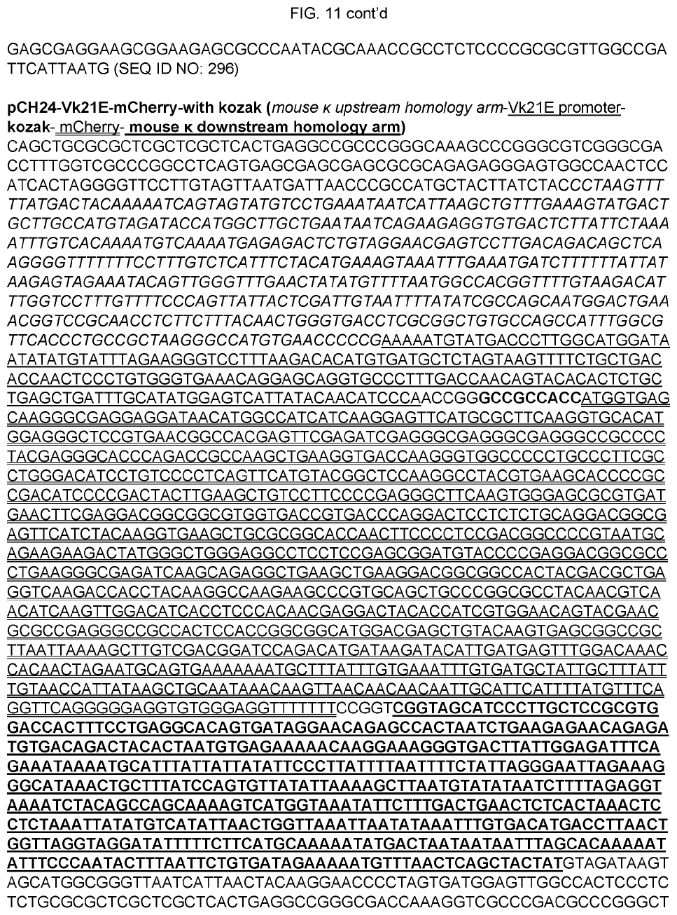

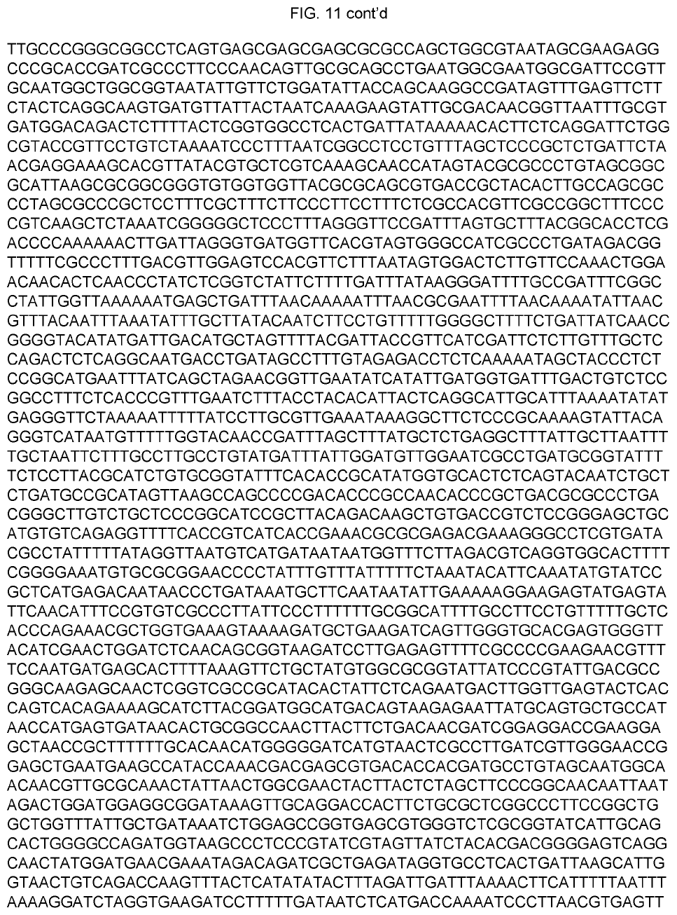

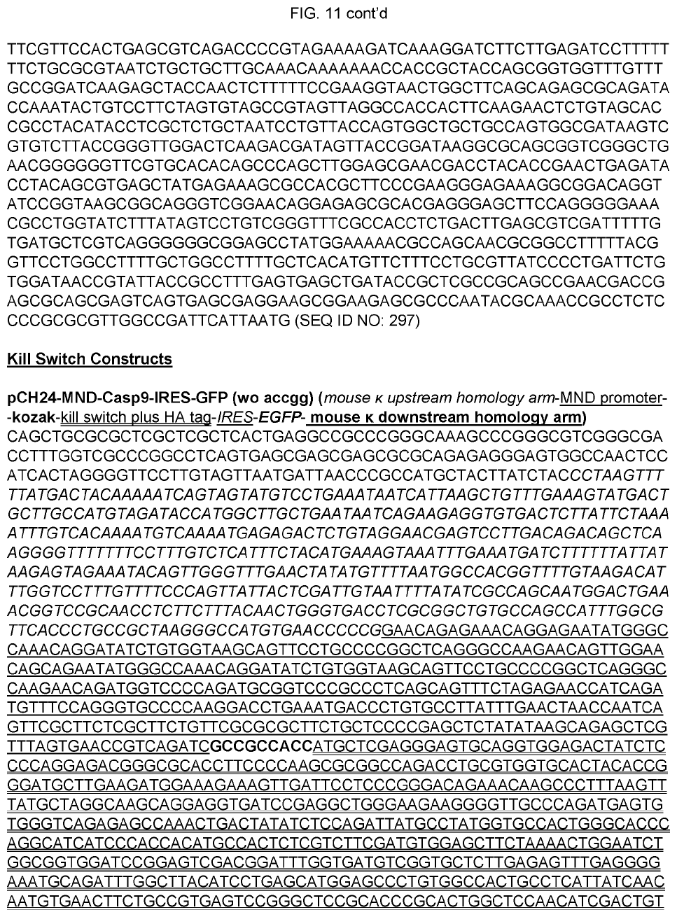

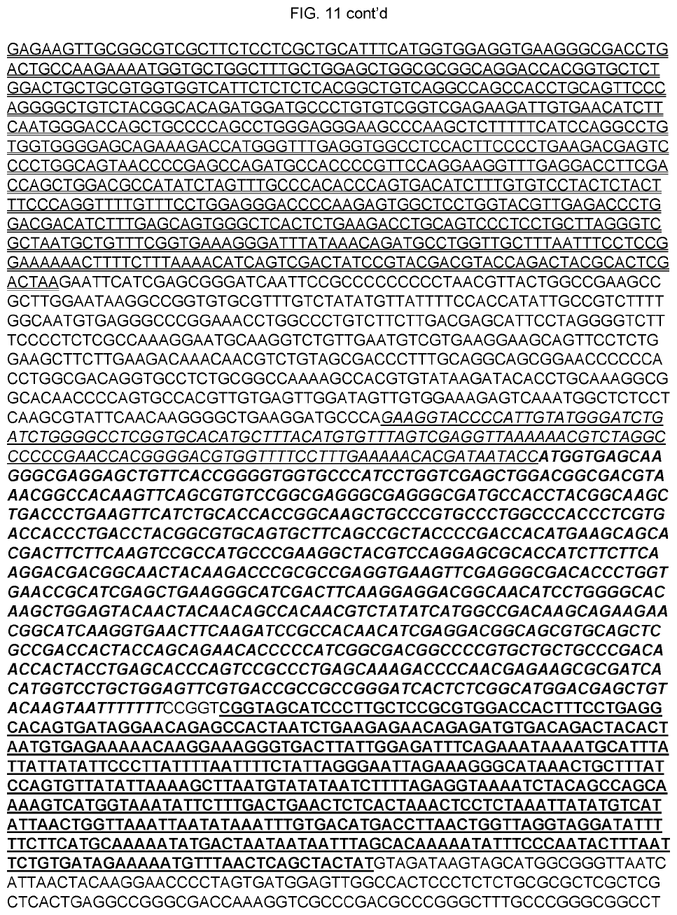

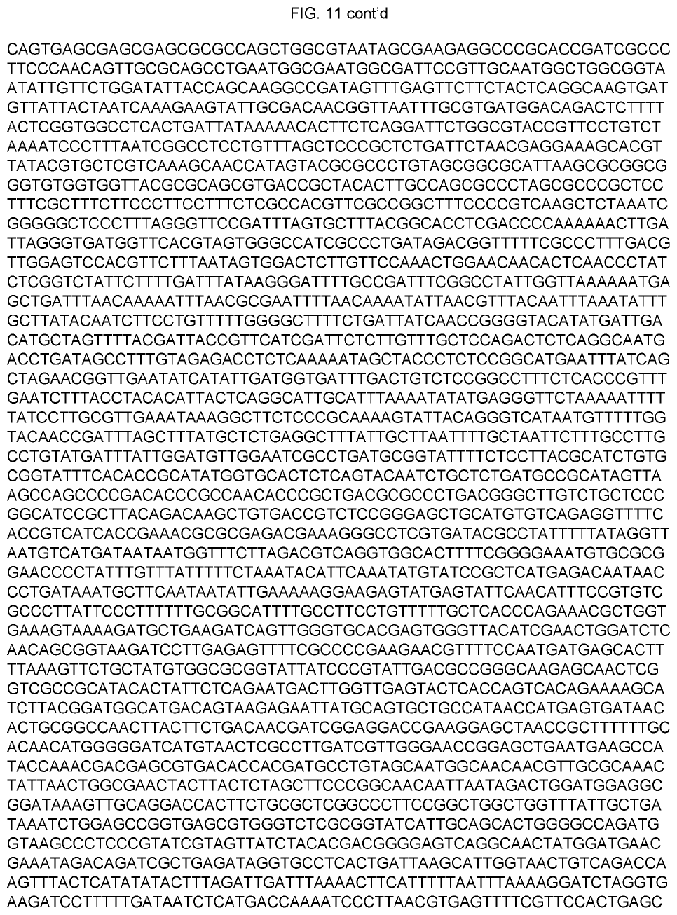

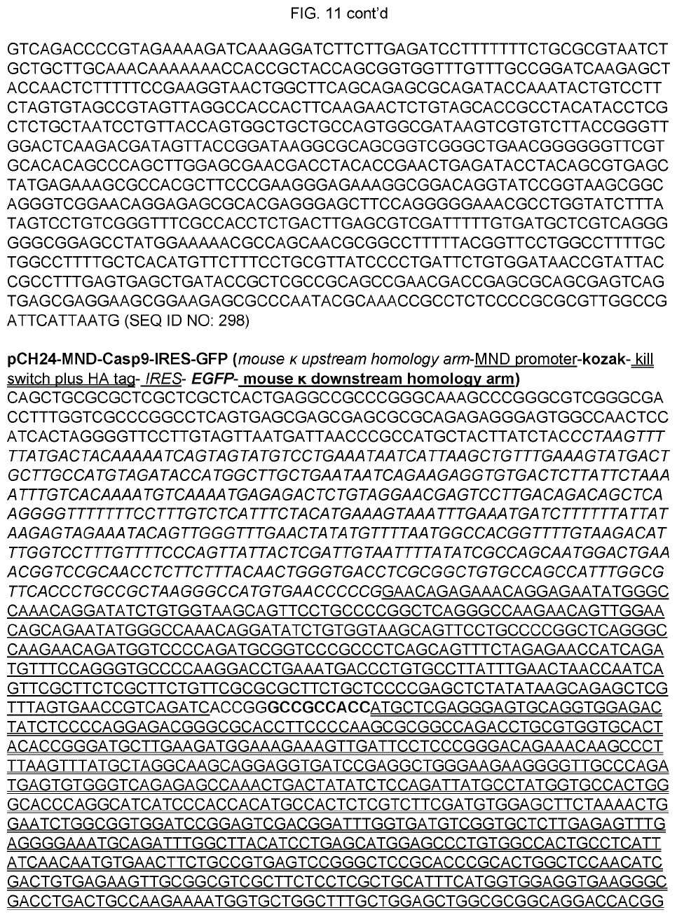

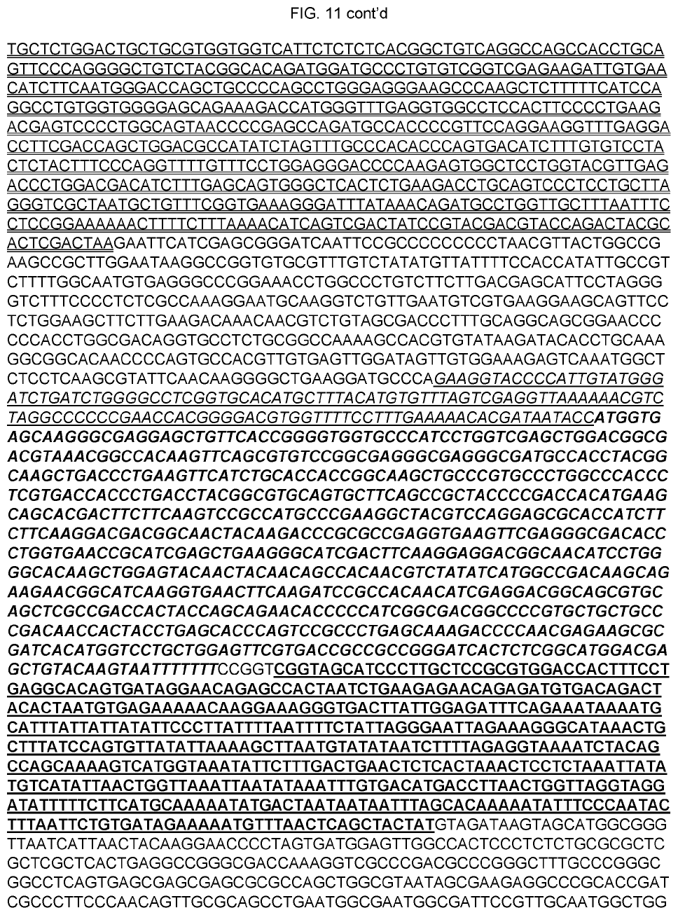

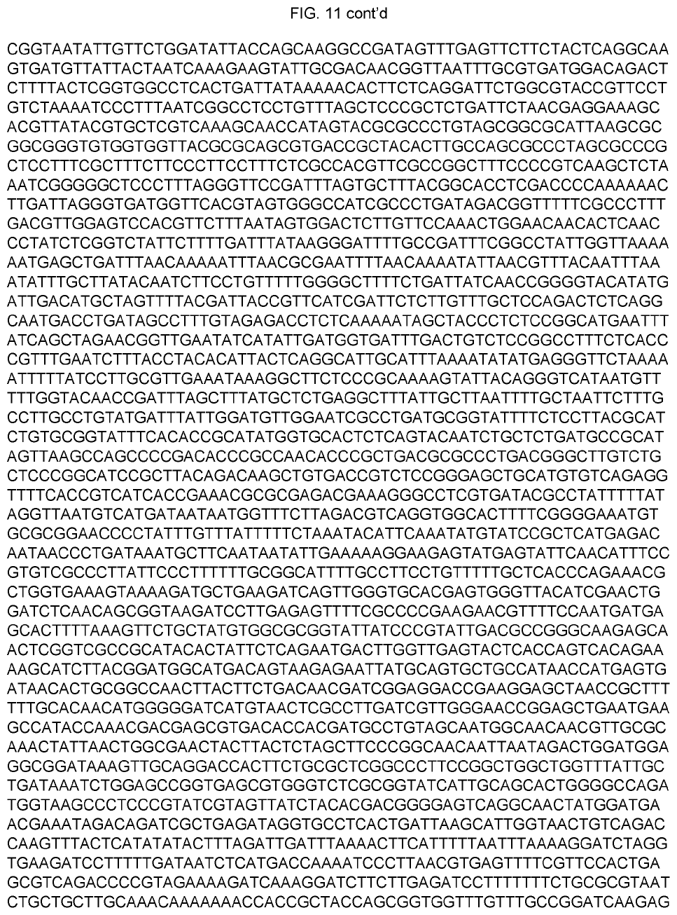

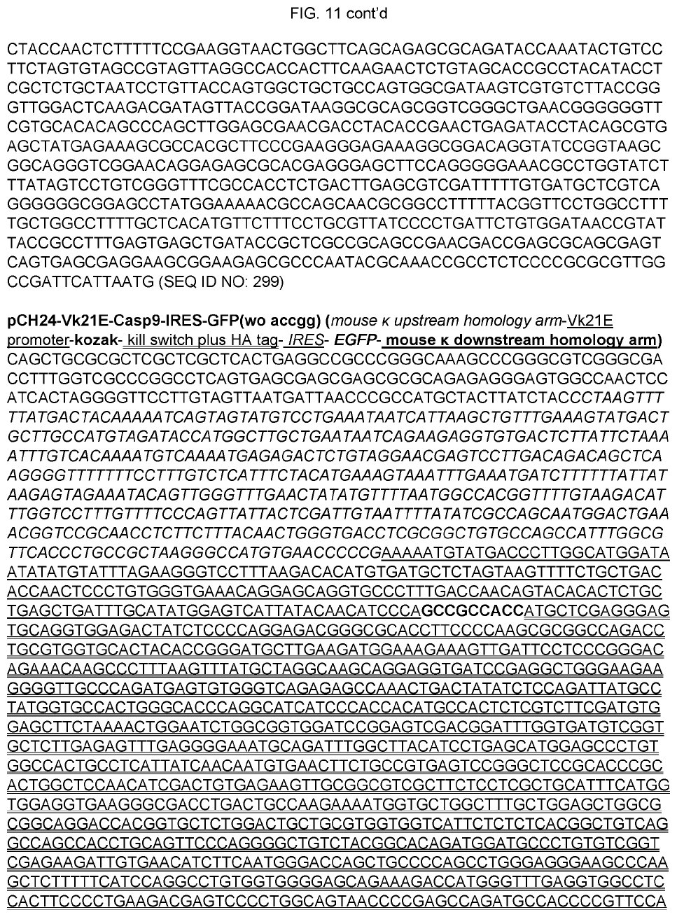

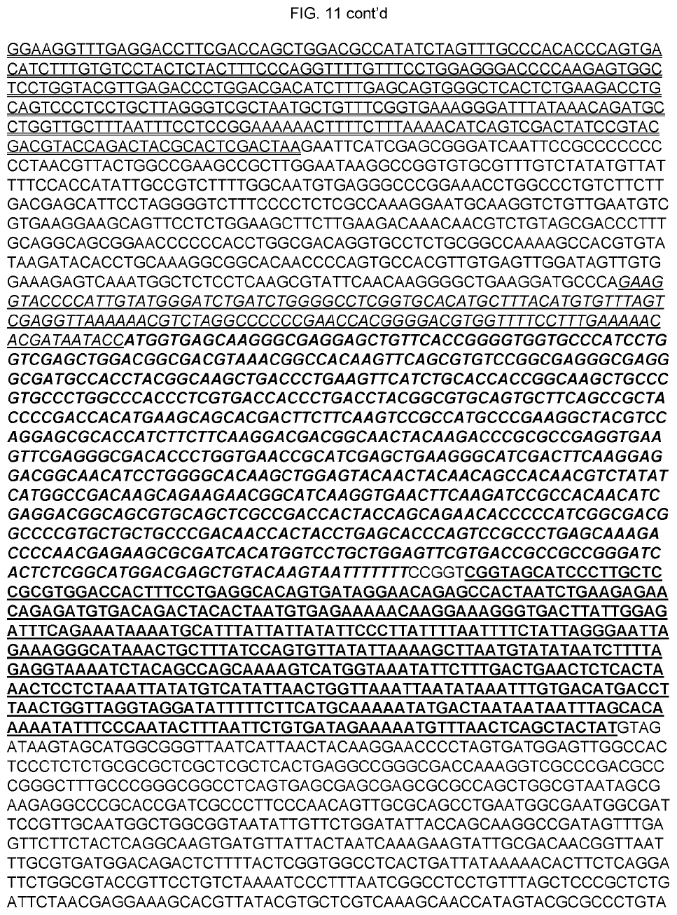

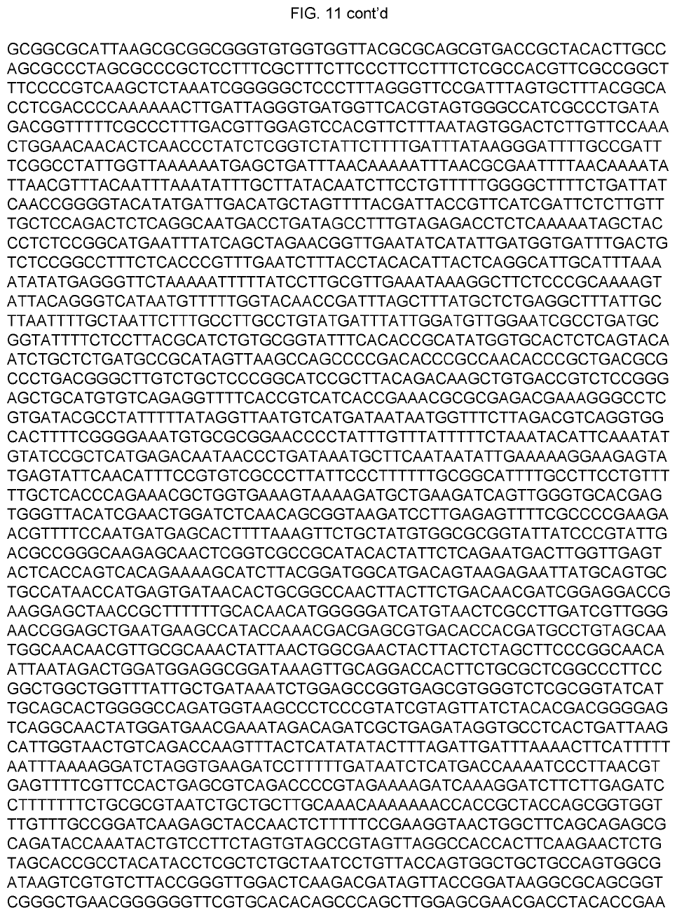

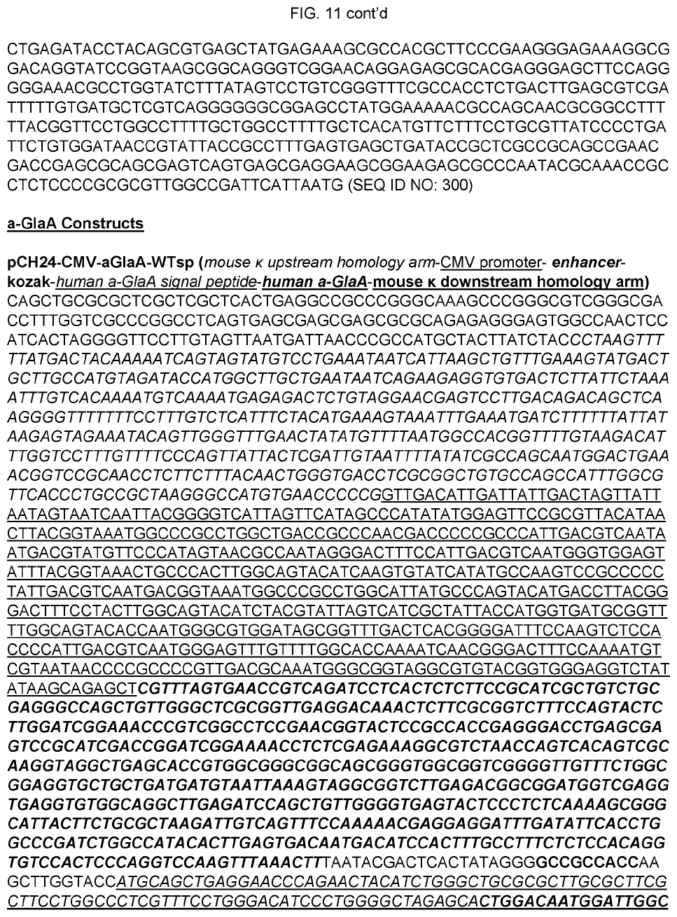

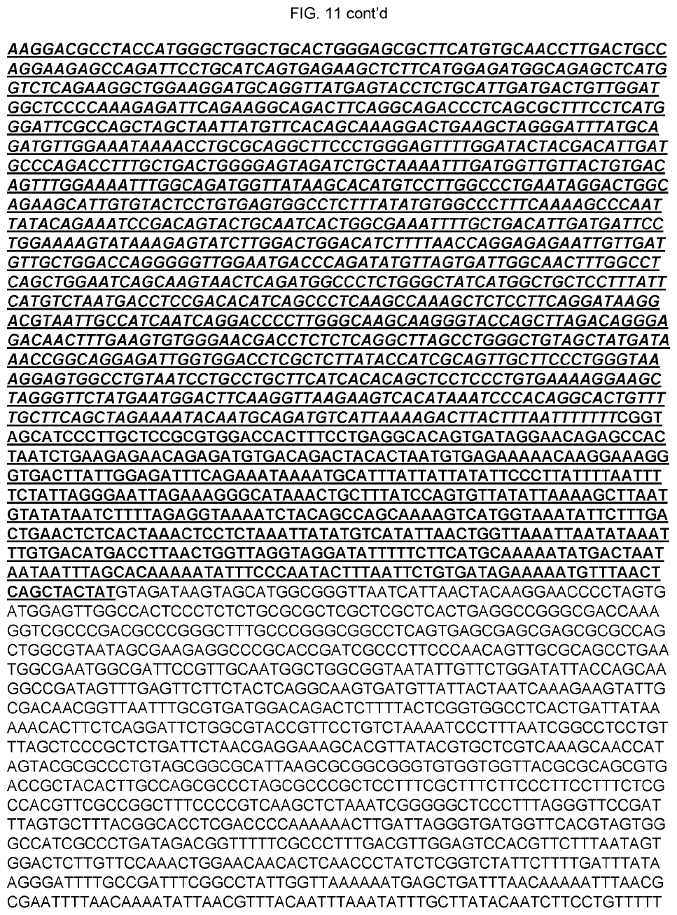

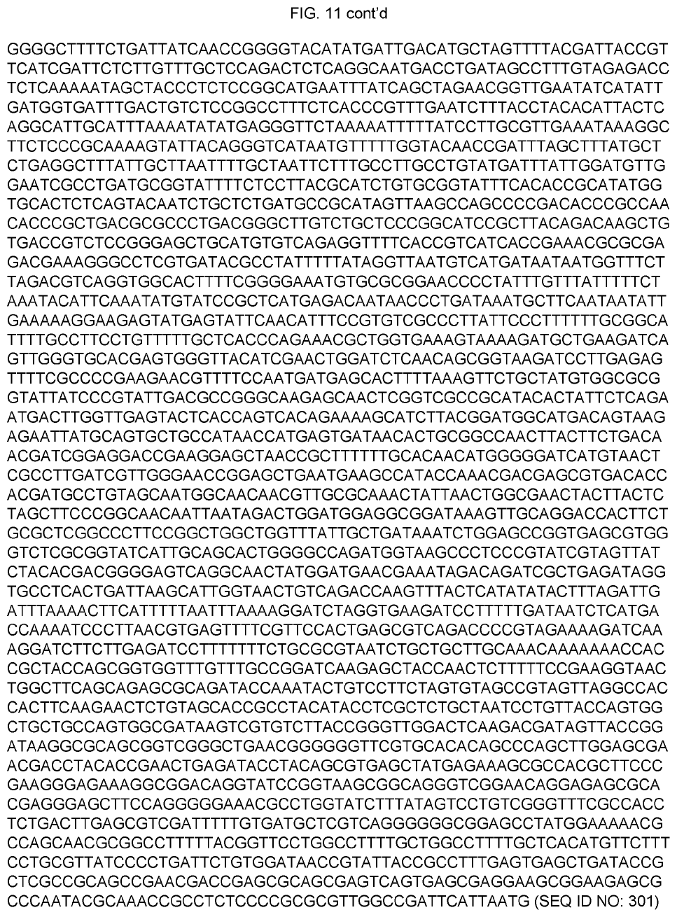

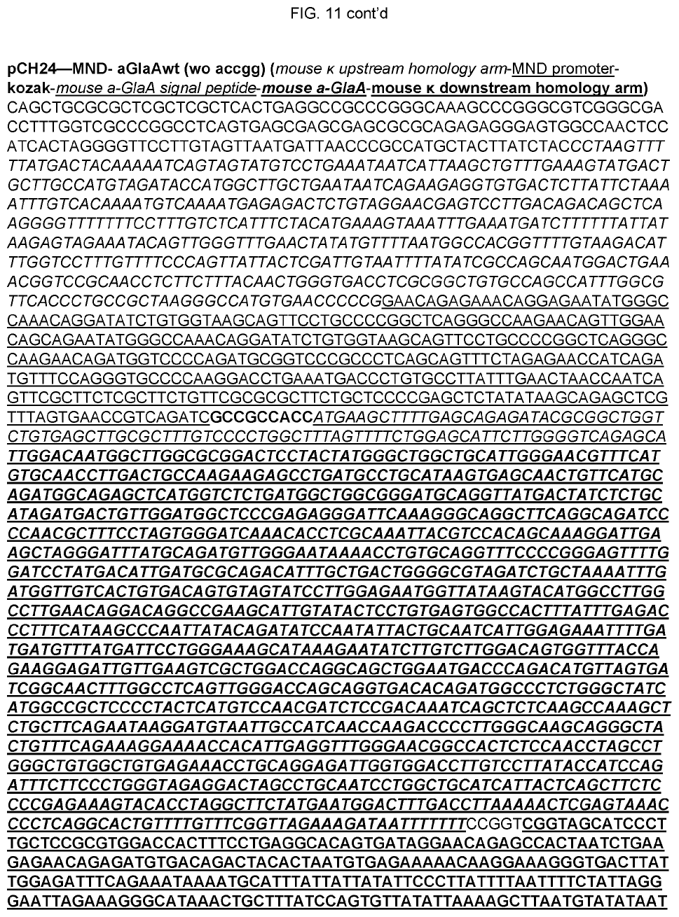

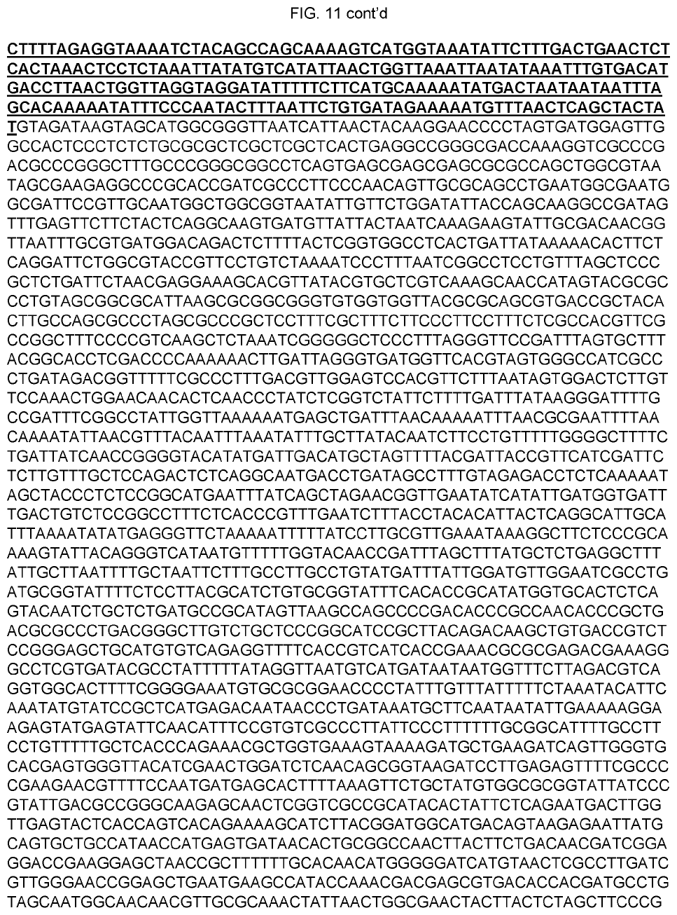

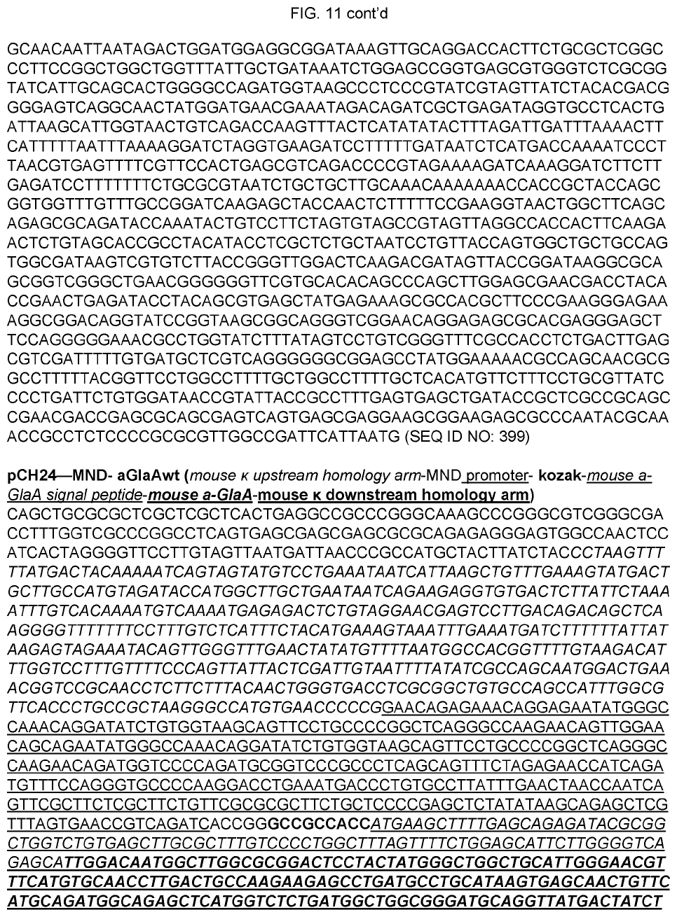

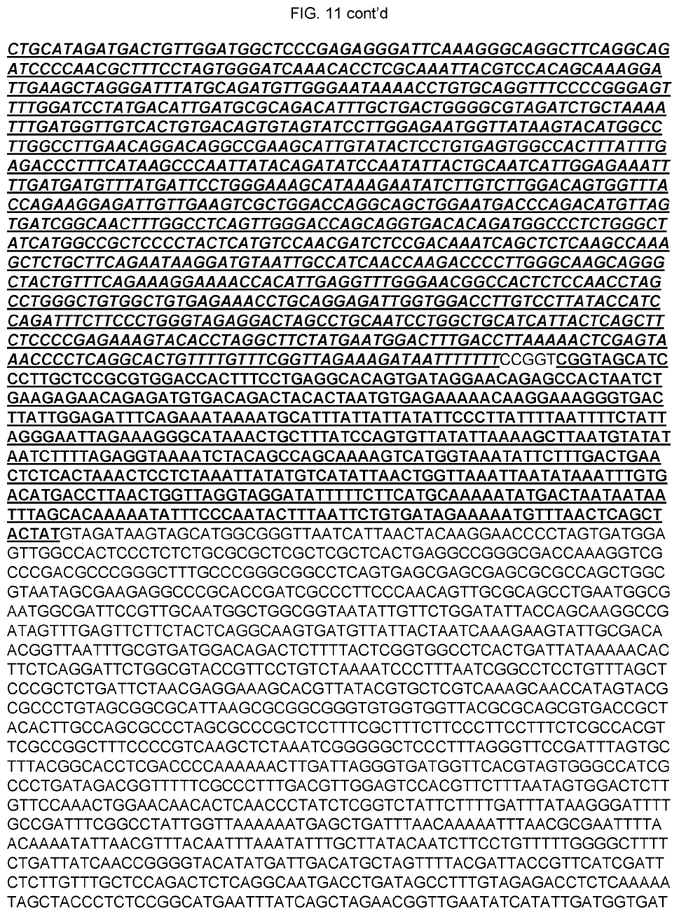

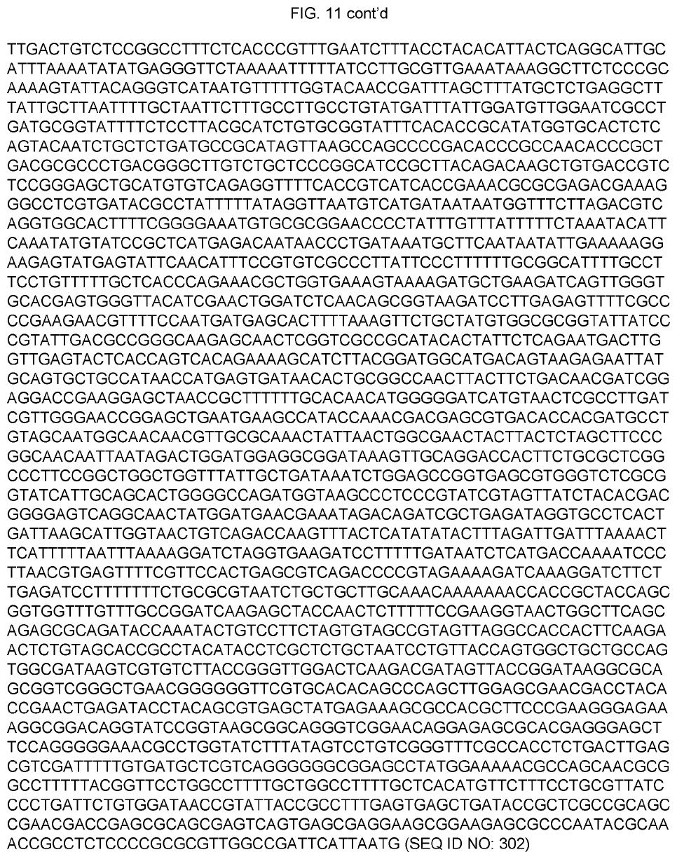

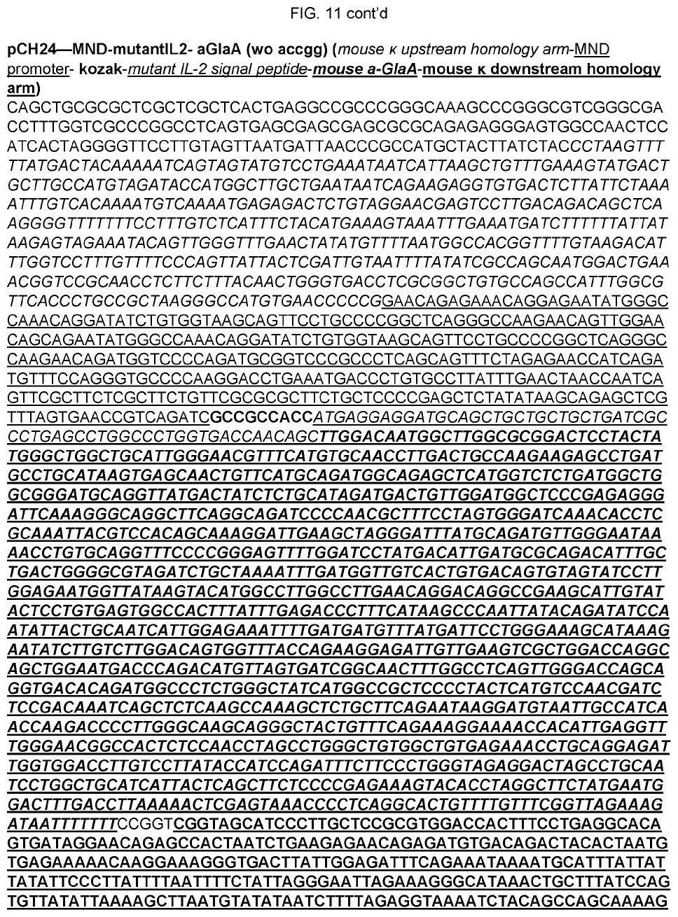

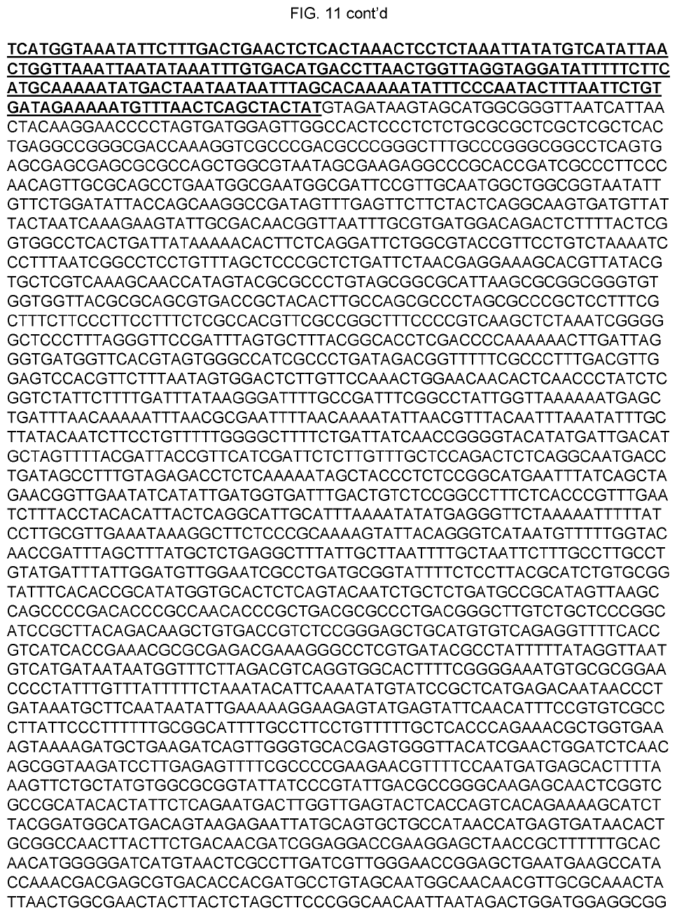

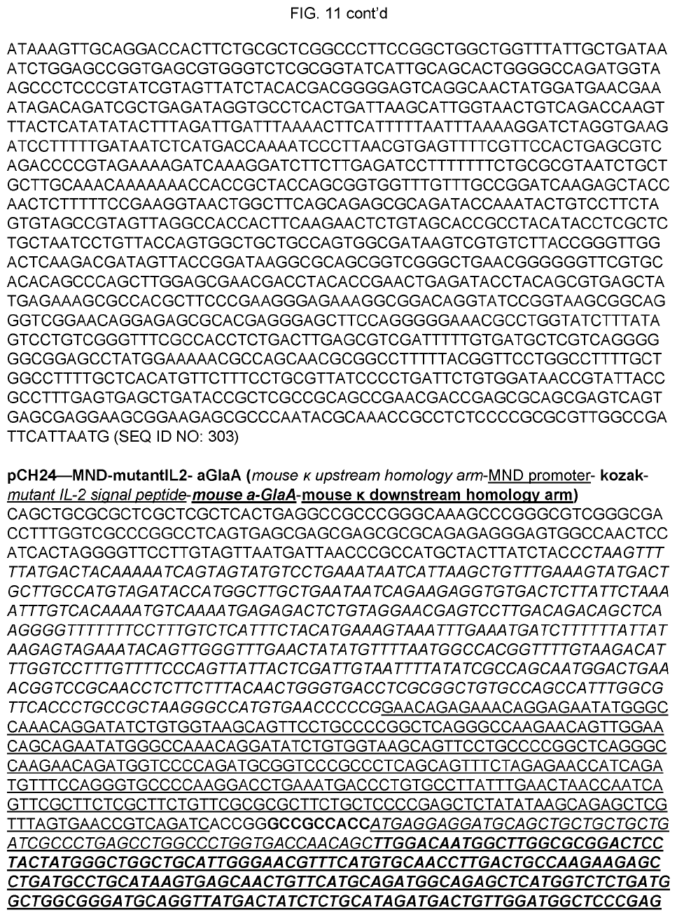

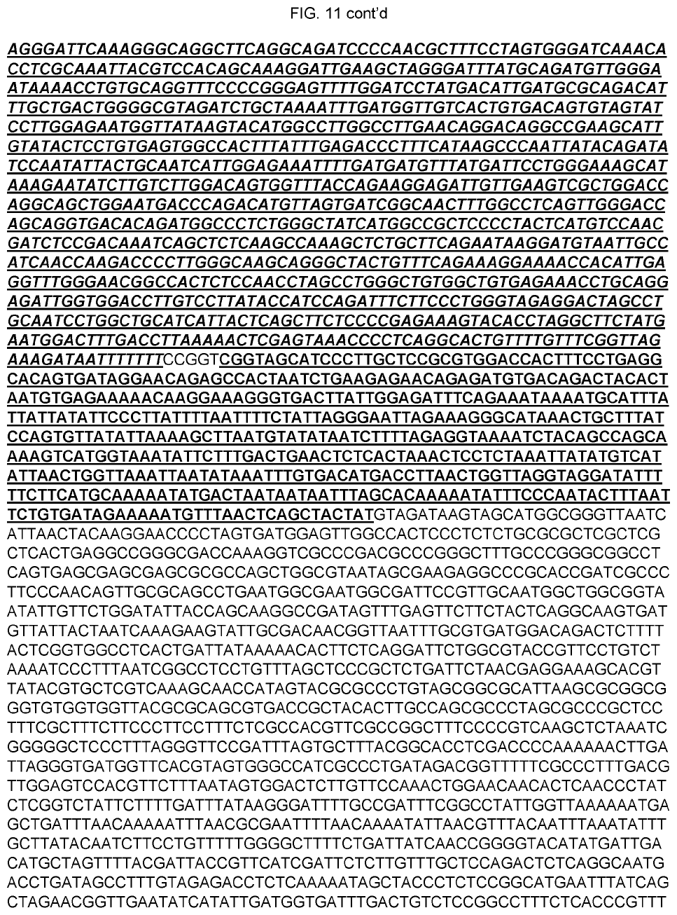

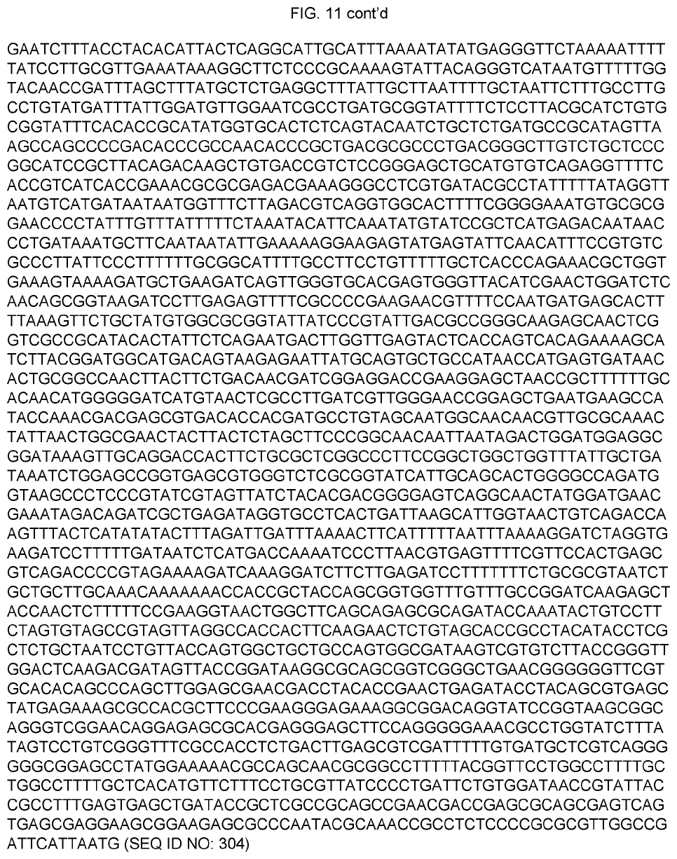

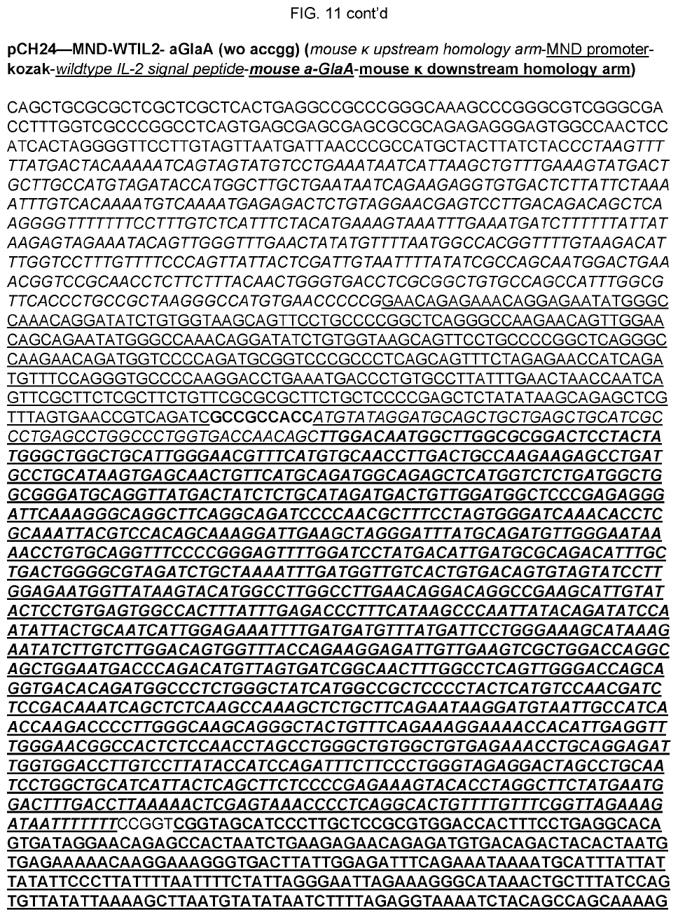

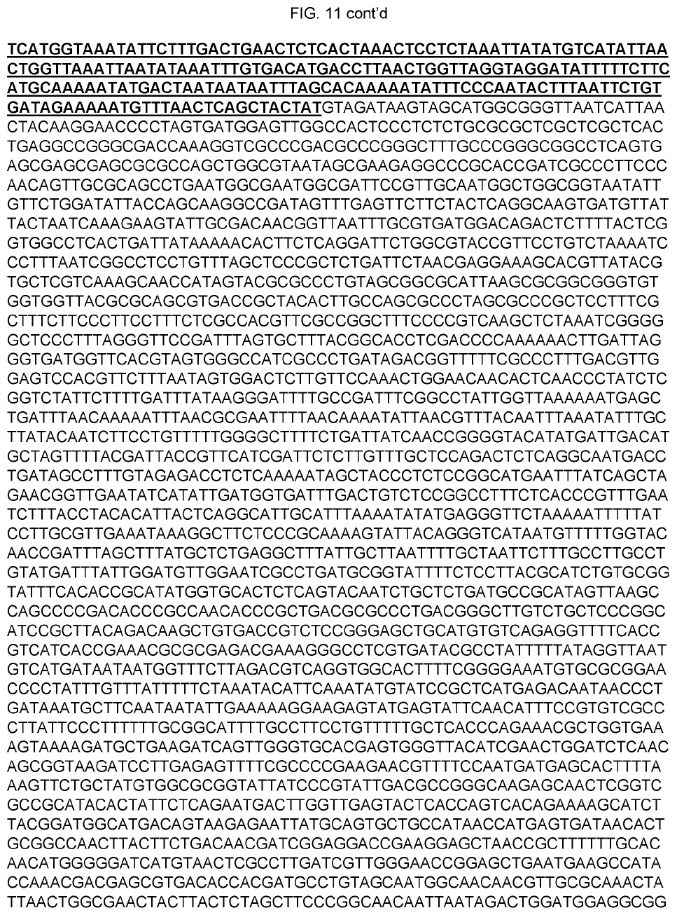

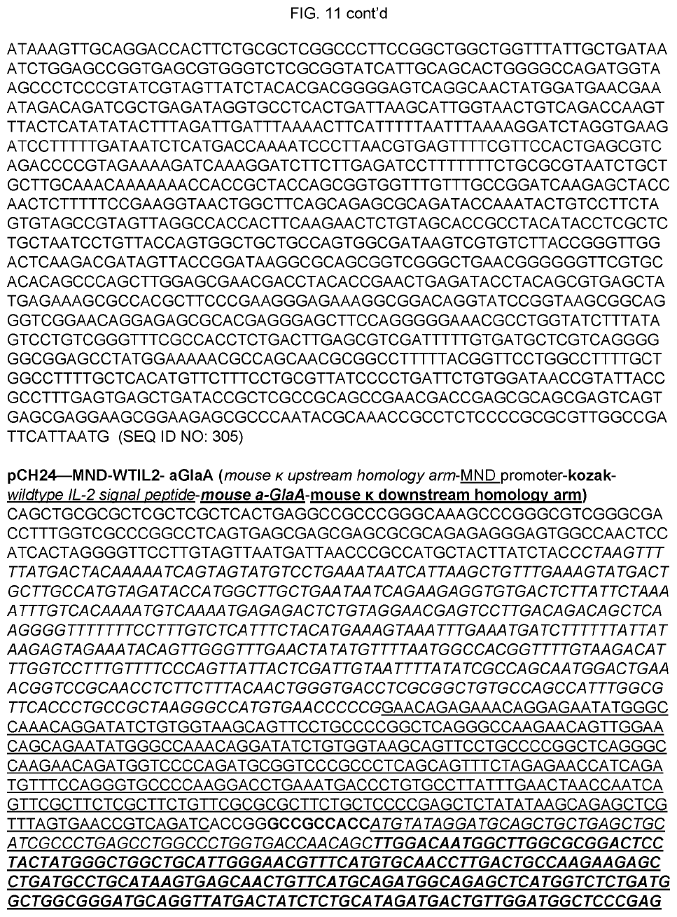

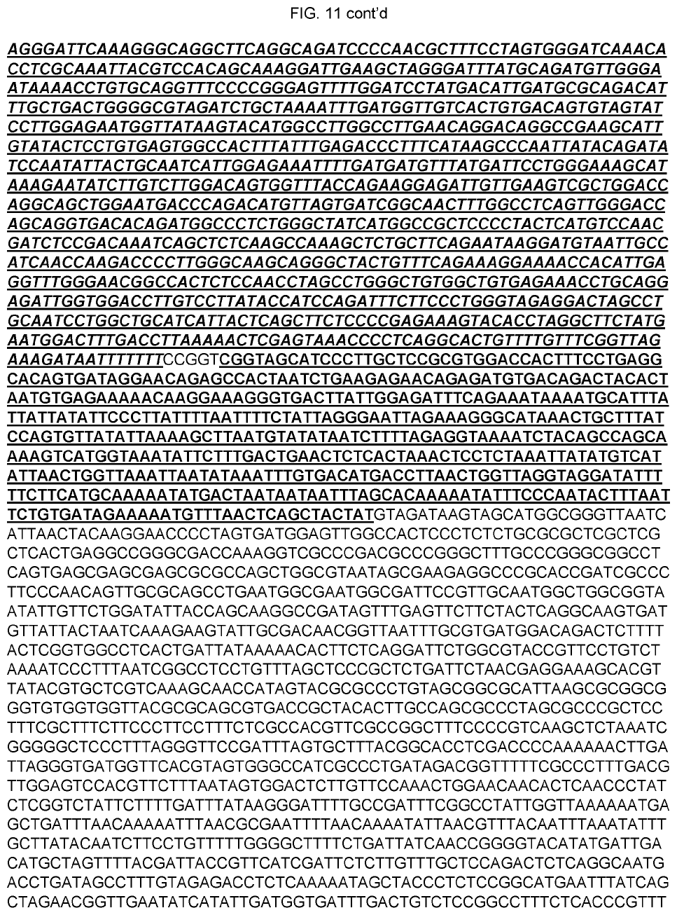

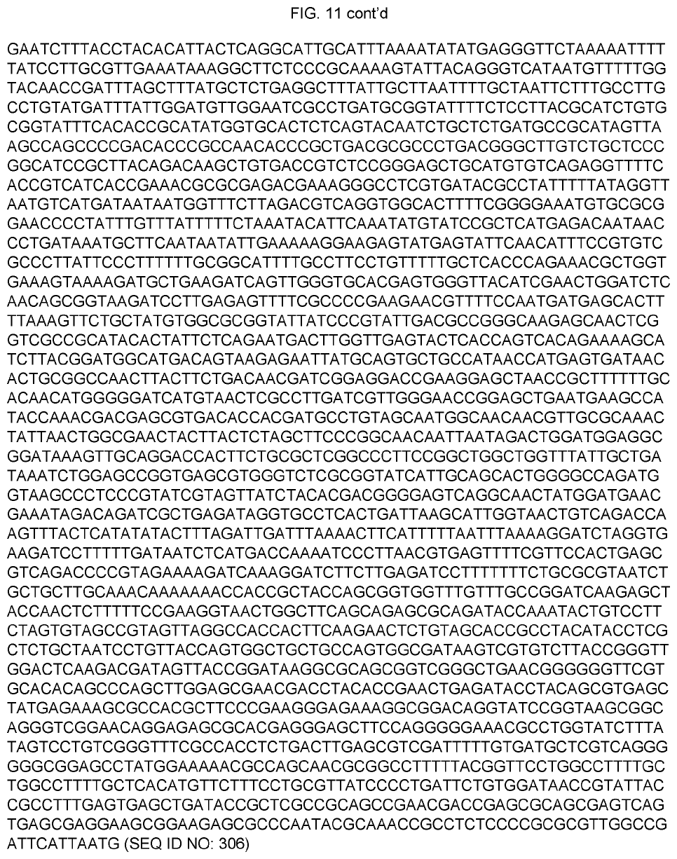

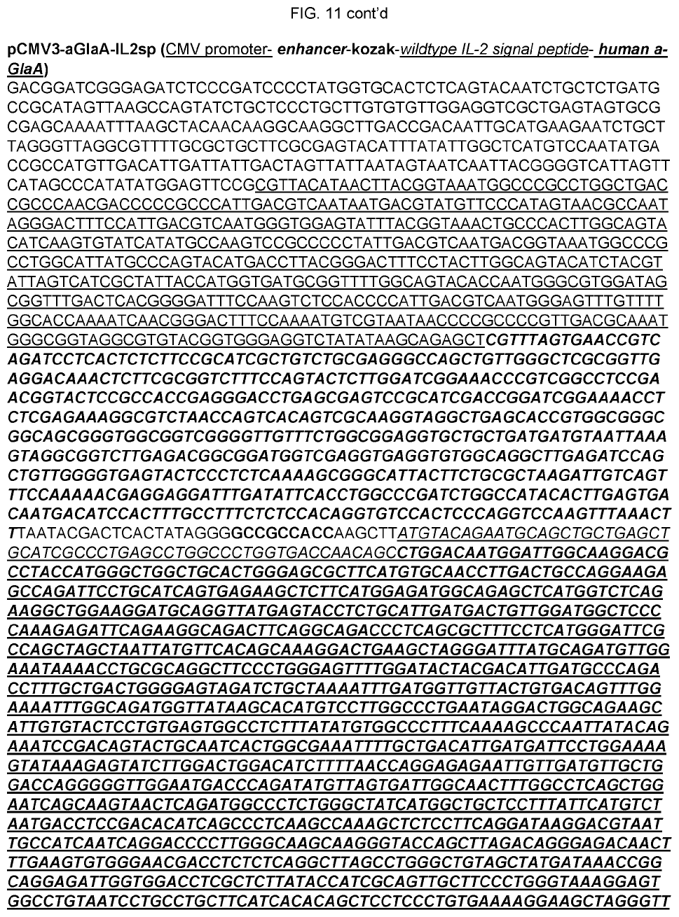

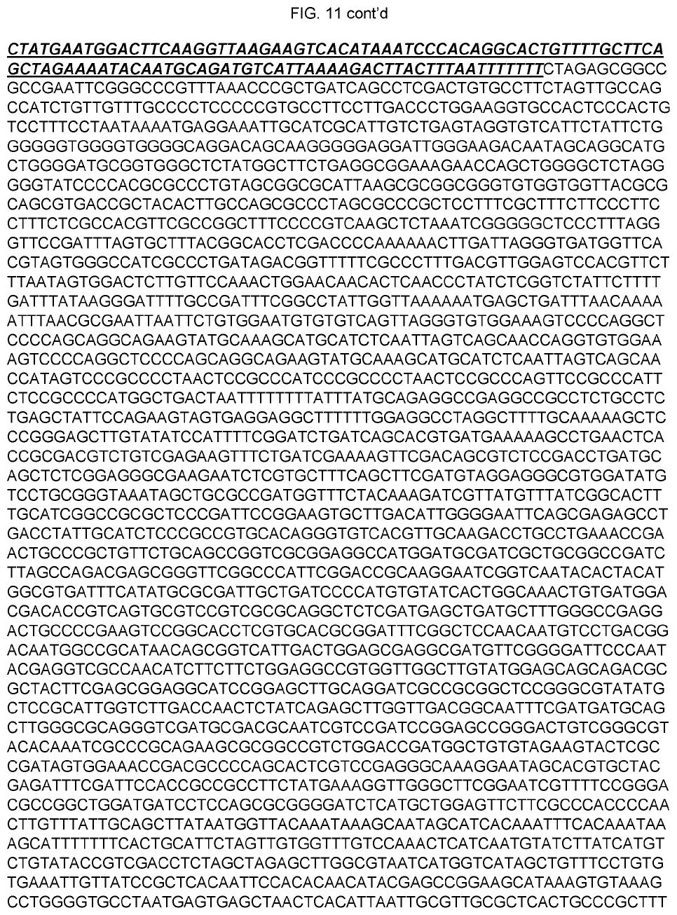

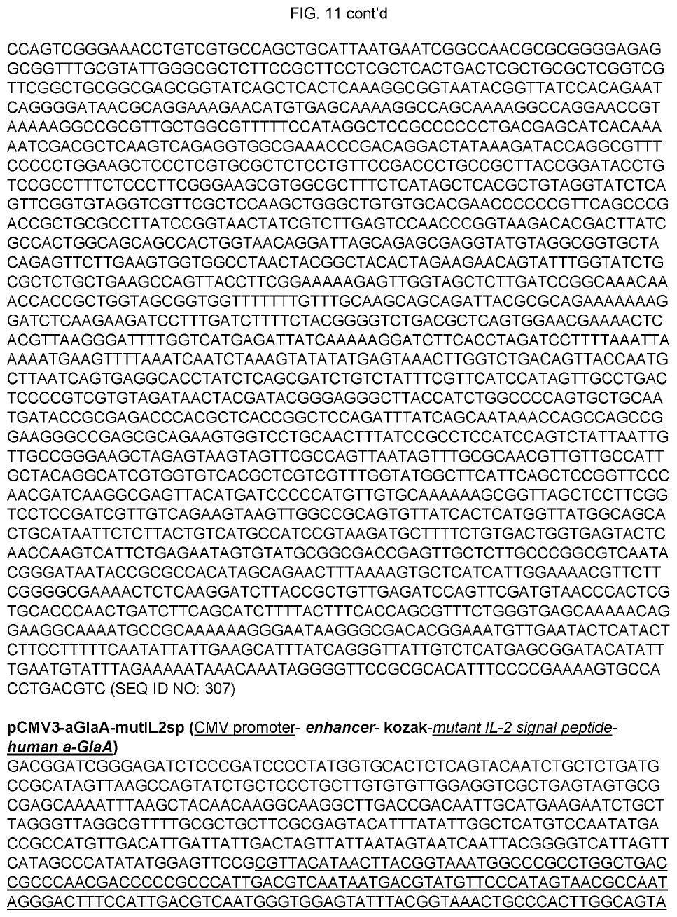

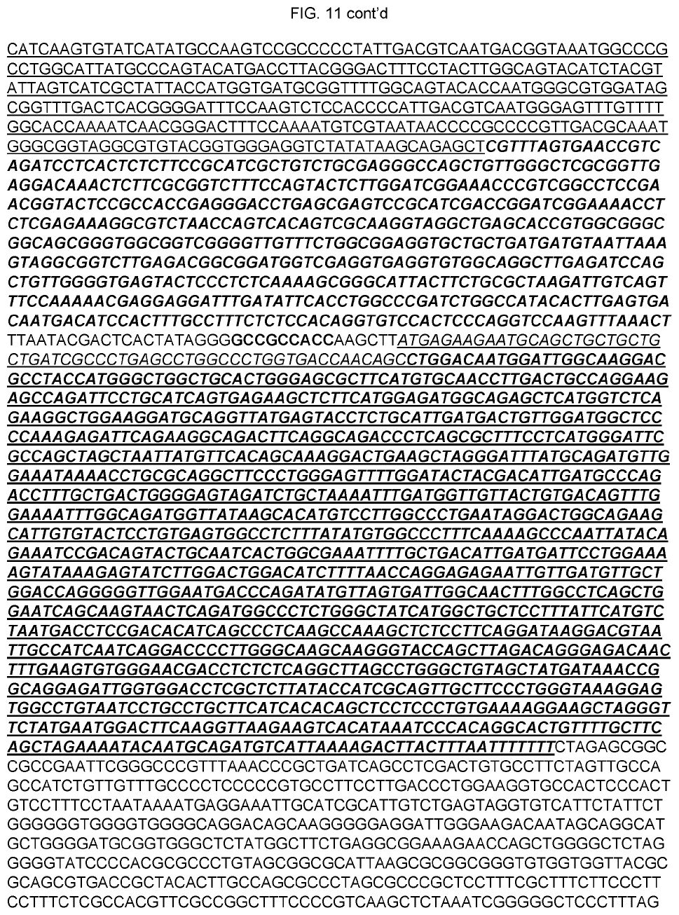

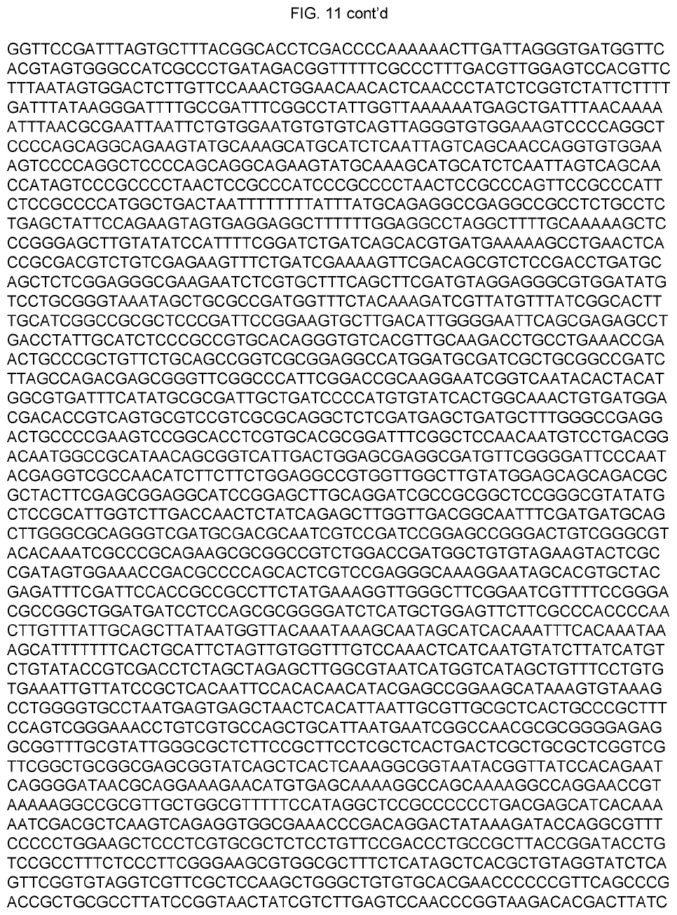

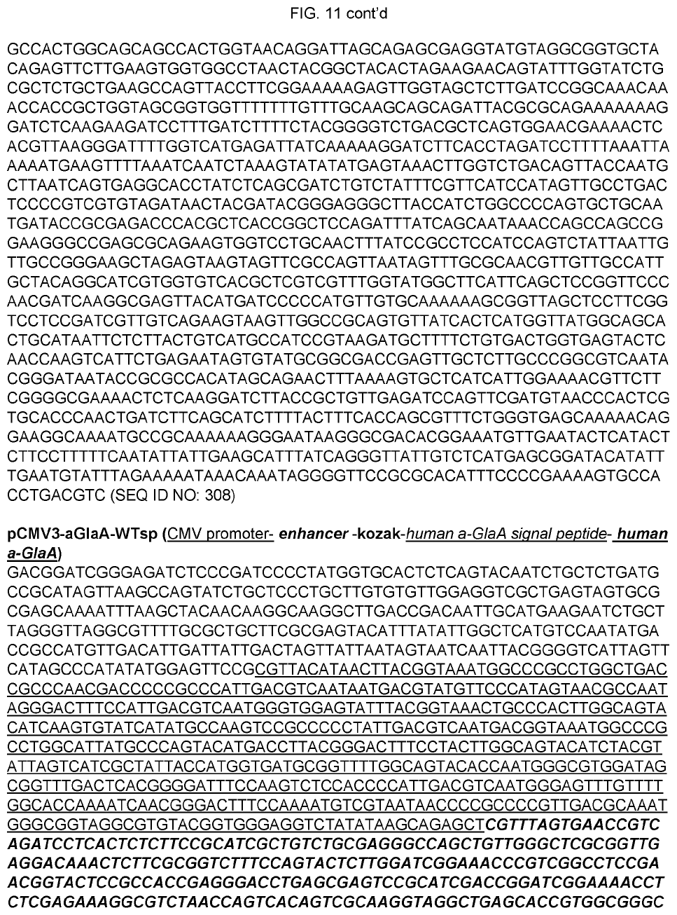

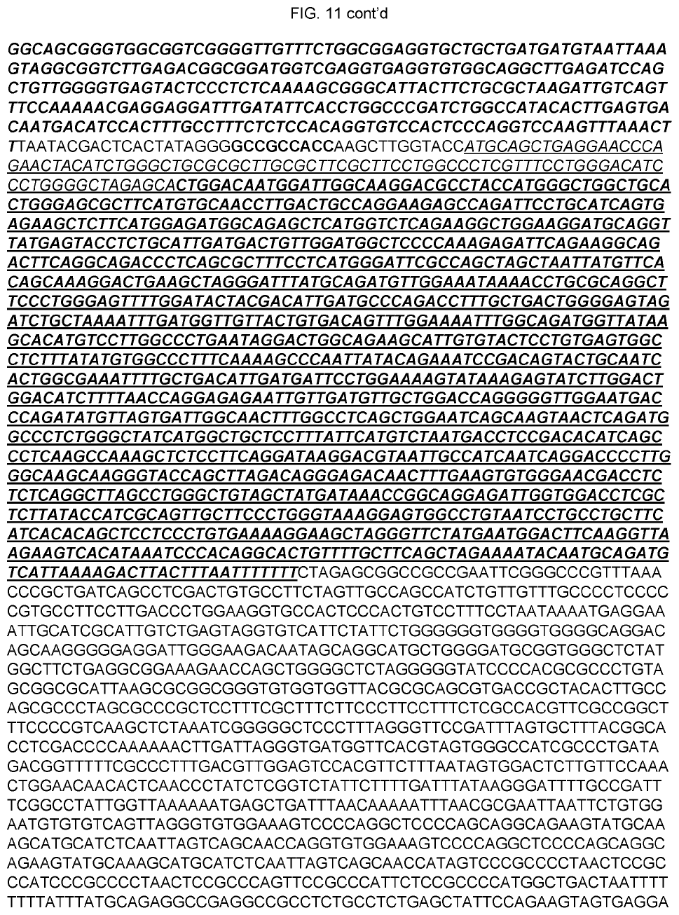

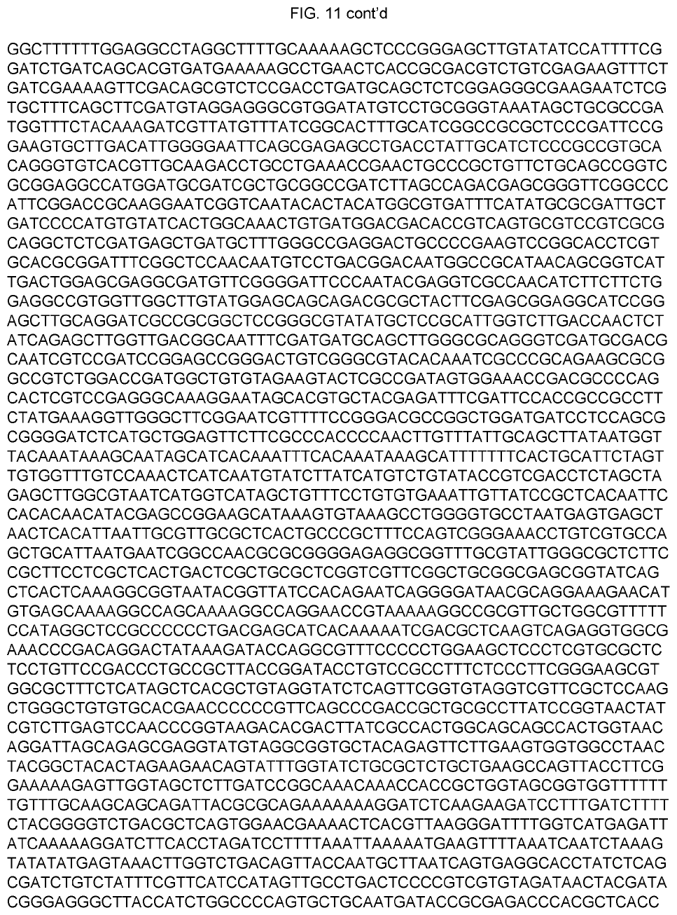

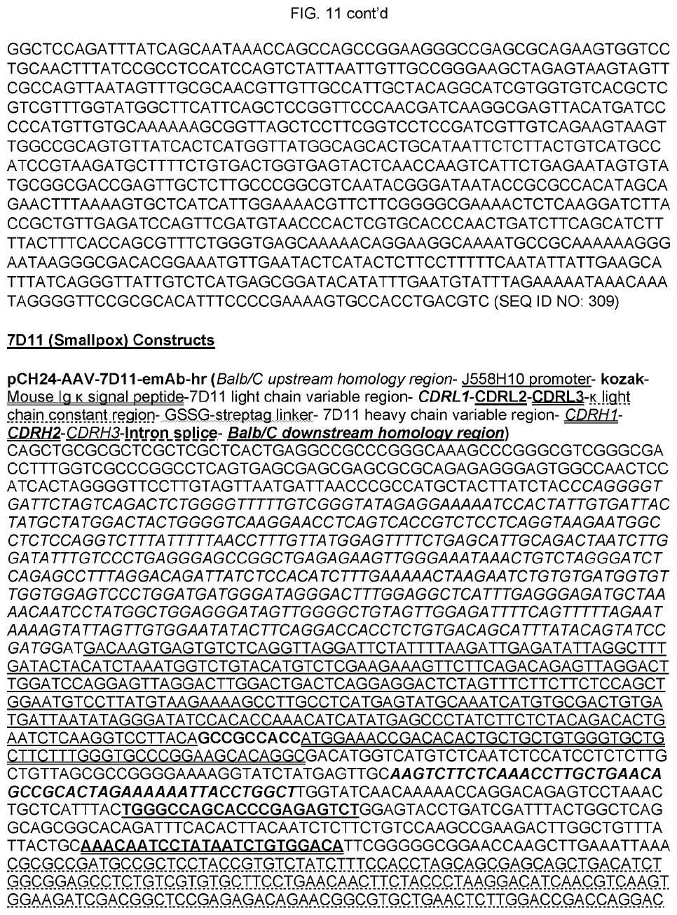

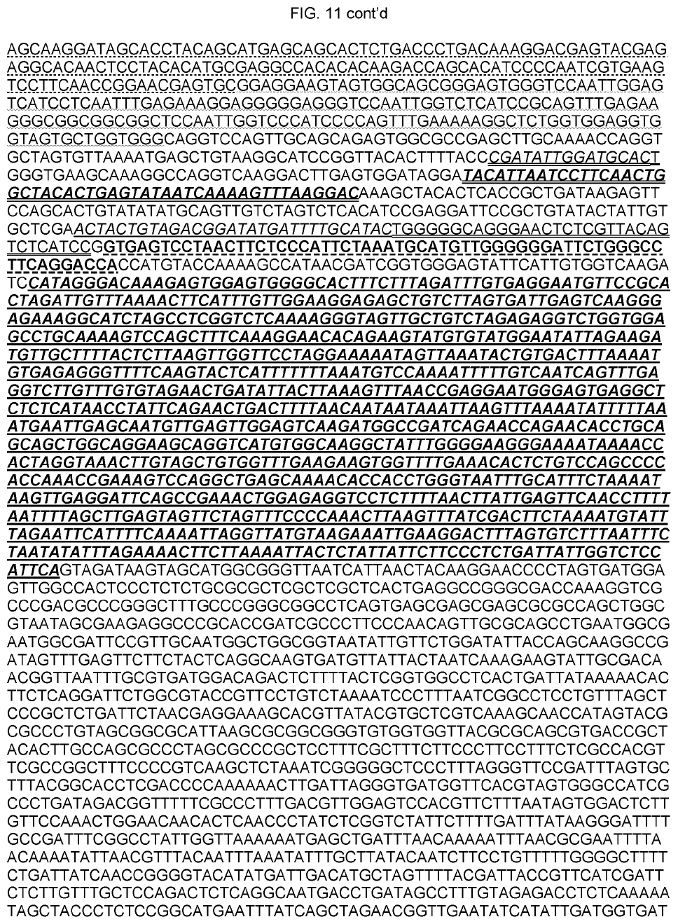

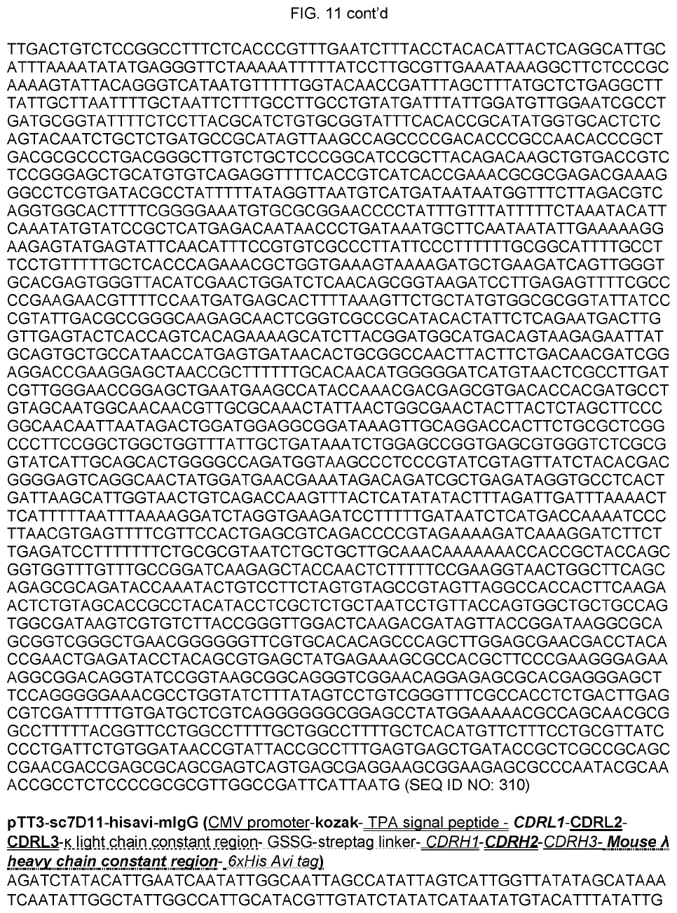

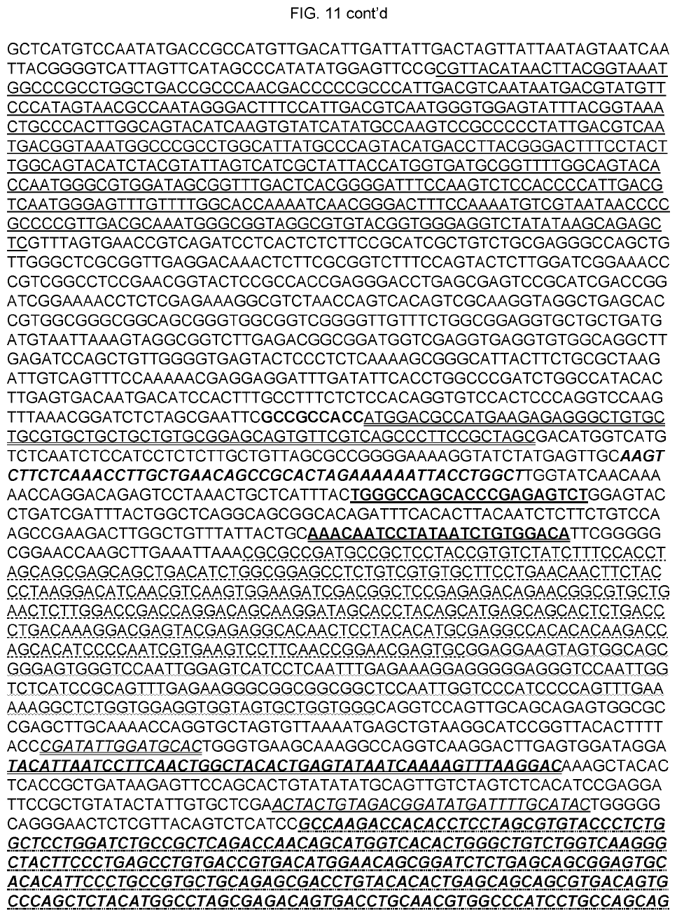

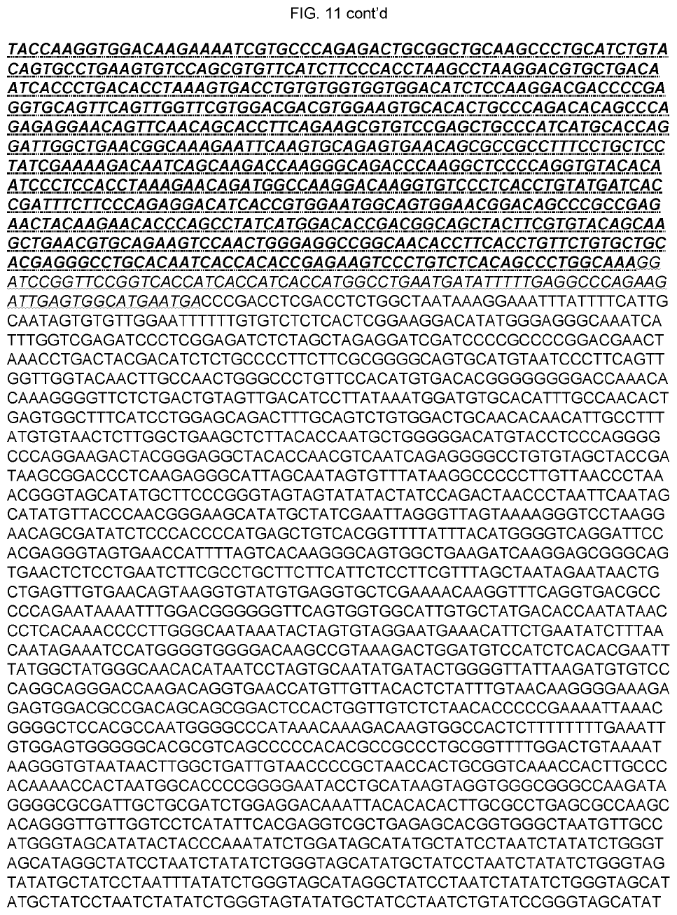

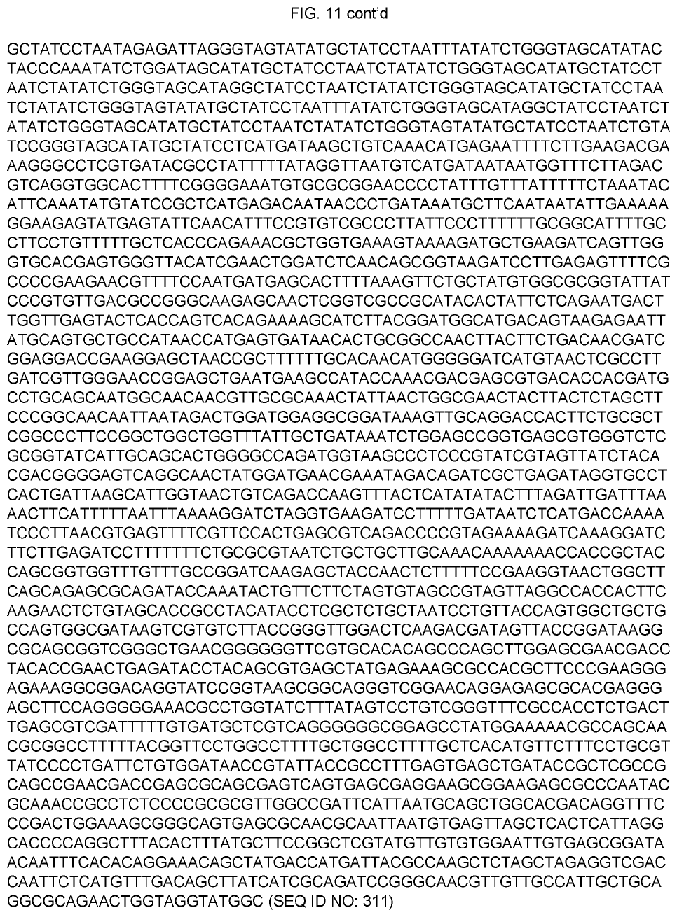

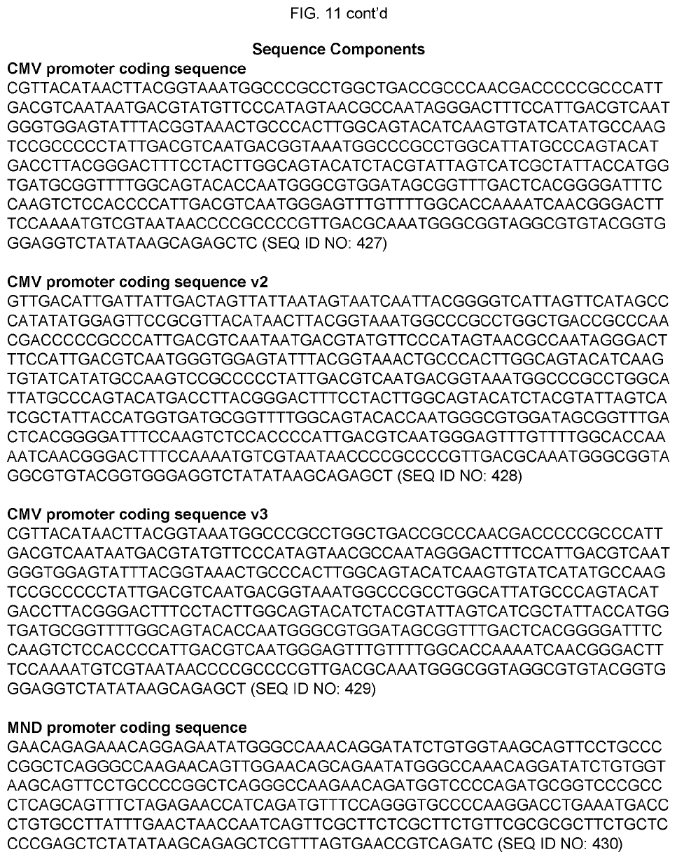

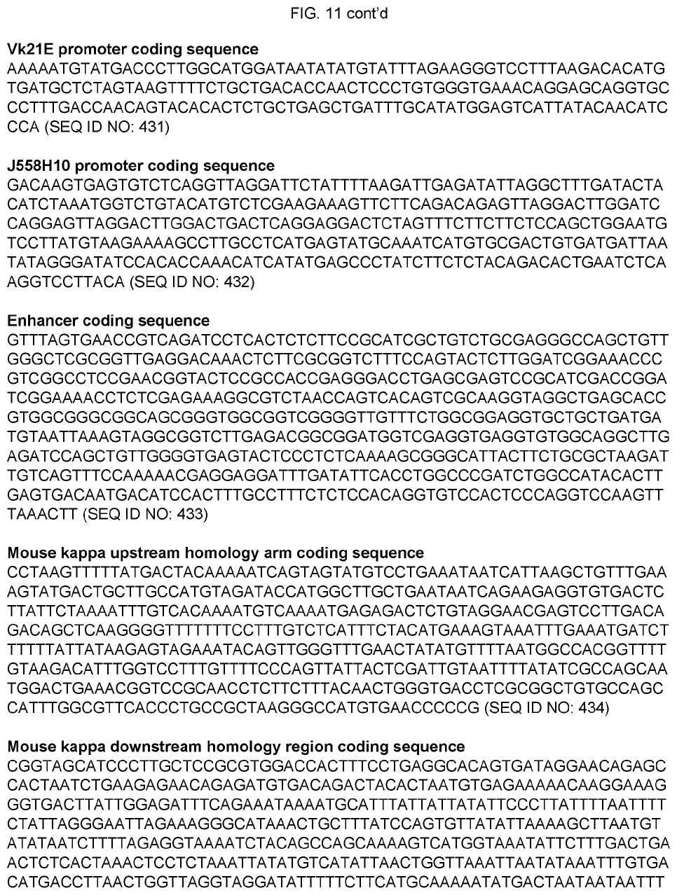

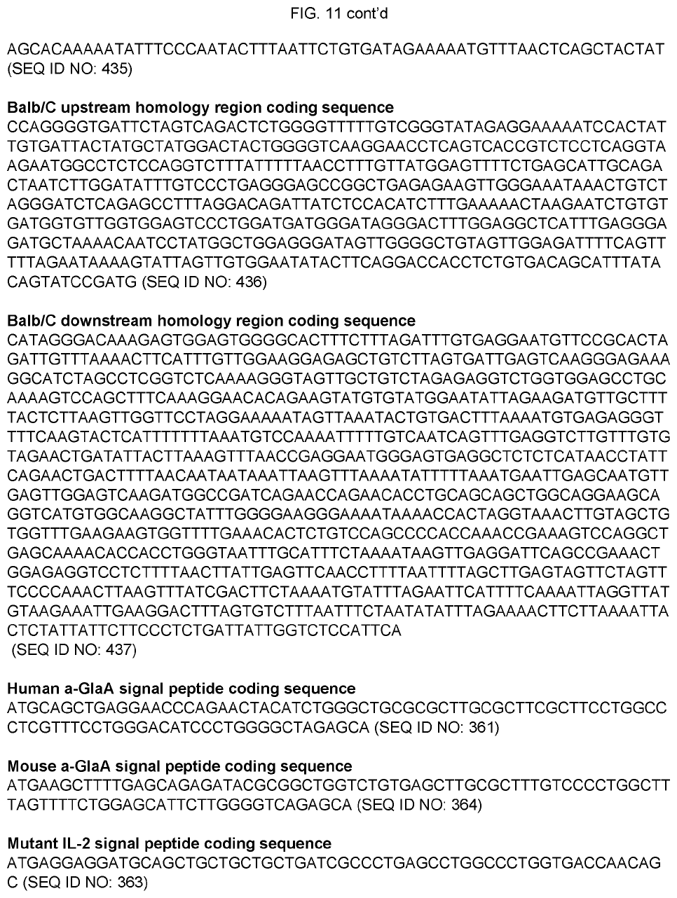

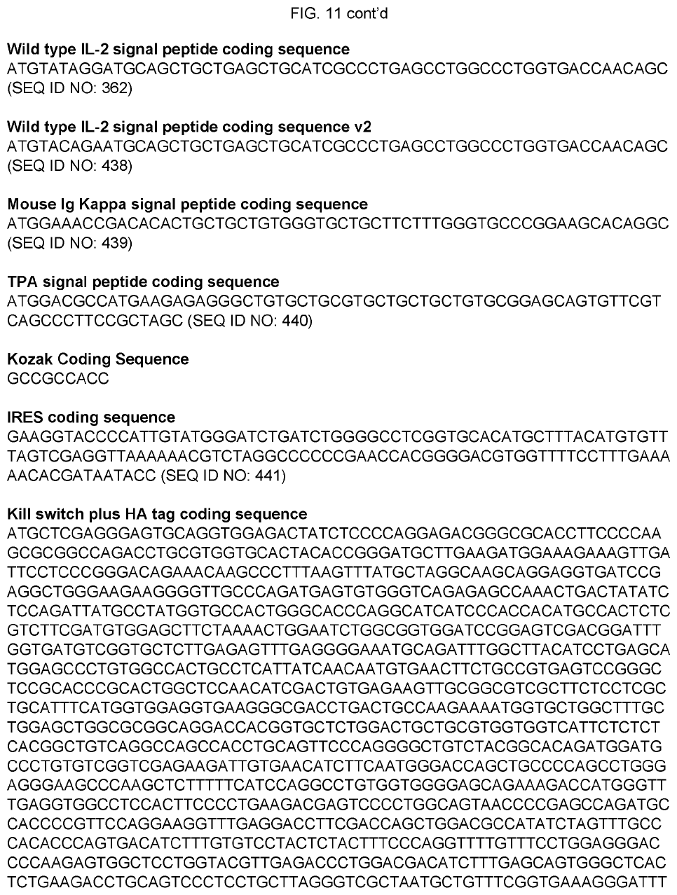

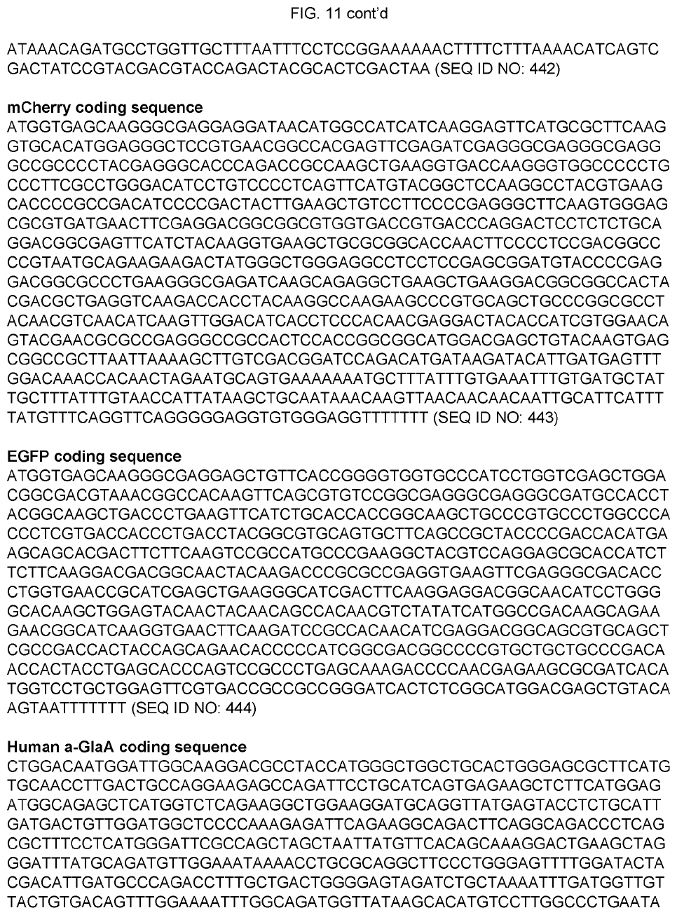

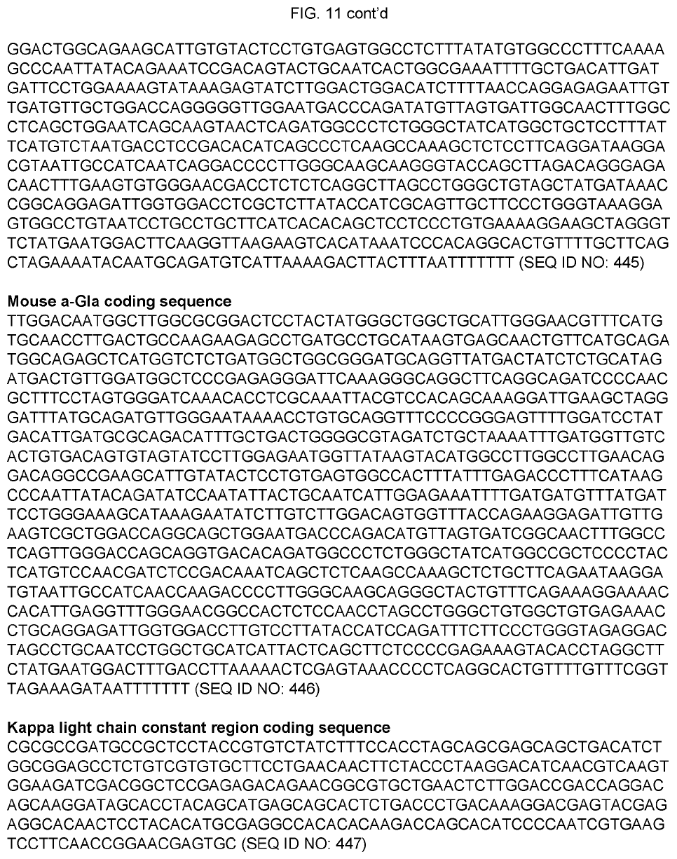

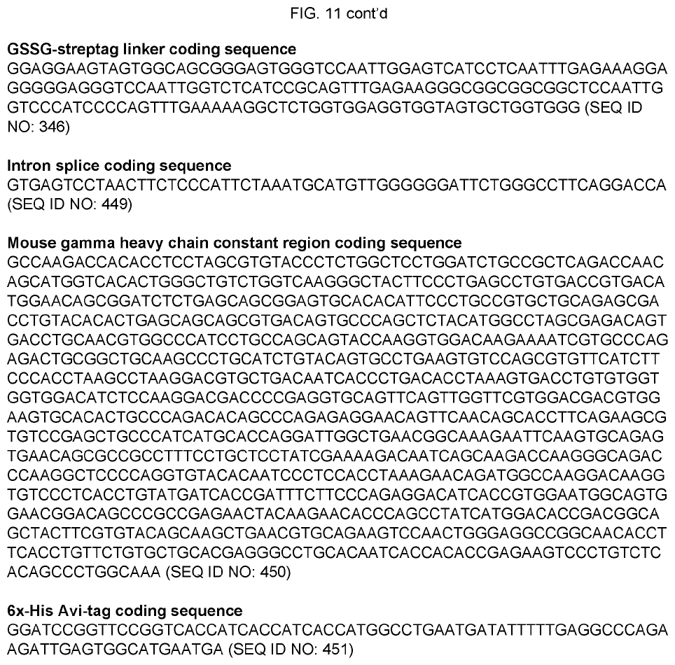

In particular embodiments, the genetic construct encoding the gene product can include a sequence encoding a signal peptide. In particular embodiments, a signal peptide for wild type human a-GlaA is encoded by the sequence set forth in SEQ ID NO: 361. In particular embodiments, a signal peptide for wild type IL-2 is encoded by the sequence set forth in SEQ ID NO: 362. In particular embodiments, a signal peptide for mutant IL-2 is encoded by the sequence set forth in SEQ ID NO: 363. In particular embodiments, a signal peptide for mouse a-GlaA is encoded by the sequence set forth in SEQ ID NO: 364. Additional signal peptide encoding sequences can be found in FIG. 11.

In particular embodiments, genetic constructs encoding the selected antibody or gene product can also encode a tag sequence. Tag sequences may be useful, for example, so that cells expressing the genetic construct may be identified and/or sorted during genetic modification processes and/or so that they can be controlled following administration to a subject. For example, in particular embodiments, it may be useful to track and/or terminate genetically modified cells following administration to a subject. Exemplary tags include STREPTAG® (GmbH, LLC, Gottingen, DE), STREP® tag II (WSHPQFEK (SEQ ID NO: 365)), or any variant thereof; see, e.g., U.S. Pat. No. 7,981,632), His tag, Flag tag (DYKDDDDK (SEQ ID NO: 366)), Xpress tag (DLYDDDDK (SEQ ID NO: 367)), Avi tag (GLNDIFEAQKIEWHE (SEQ ID NO: 368)), Calmodulin tag (KRRWKKNFIAVSAANRFKKISSSGAL (SEQ ID NO: 369)), Polyglutamate tag, HA tag (YPYDVPDYA (SEQ ID NO: 370)), Myc tag (EQKLISEEDL (SEQ ID NO: 371)), Nus tag, S tag, SBP tag, Softag 1 (SLAELLNAGLGGS (SEQ ID NO: 372)), Softag 3 (TQDPSRVG (SEQ ID NO: 373)), and V5 tag (GKPIPNPLLGLDST (SEQ ID NO: 374)).

In particular embodiments, a genetic construct encoding the gene product can further include a suicide gene. A suicide gene encodes a suicide switch. Suicide switches include mechanisms which result in selective destruction of a genetically modified cell in the face of, for example, unacceptable toxicity. The earliest clinical experience with suicide switches is with the Herpes Virus Thymidine Kinase (HSV-TK) which renders genetically modified cells with the suicide switch susceptible to Ganciclovir. In particular embodiments, the suicide switch is a protein that induces apoptosis upon dimerization. In particular embodiments the protein that induces apoptosis upon dimerization is a human caspase protein, e.g., caspase 1, caspase 2, caspase 3, caspase 4, caspase 5, caspase 6, caspase 7, caspase 8, caspase 9, caspase 10, caspase 14, etc. In certain embodiments the protein is human caspase 9. For use in a safety switch, the caspase should only dimerize upon activation with an activation agent, and thus the sequence of an inducible caspase is mutated to delete the native dimerization domain. In particular embodiments, the suicide switch includes an induced caspase.

Under physiological conditions, caspase 9 is activated by the release of cytochrome C from damaged mitochondria. Activated caspase 9 then activates caspase 3, which triggers terminal effector molecules leading to apoptosis. An inducible caspase 9 protein is truncated to delete its physiological dimerization domain (caspase activation domain (CARD), referred to as D caspase 9. D caspase 9 has low dimerizer-independent basal activity. In a suicide switch construct, an inducible caspase protein is linked to a chemically induced dimerization (CID) domain.

Chemically induced dimerization (CID) domains provide for dimerization only in the presence of the orthologous suicide switch activating agent. One or more CID domains may be fused to the inducible caspase protein, e.g., one or two different CID domains may be fused to the caspase protein. Examples of CID domains include, without limitation, FKBP and mTOR domains, which can be dimerized with FK102, FK506, AP21, AP20, FKCsA, rapamycin, etc. Other CID domains include GyrB dimerized by Coumermycin; GID1 (gibberellin insensitive dwarf 1) and gibberellin; SNAP-tag and HaXS; Bcl-xL and ABT-737, etc. In some embodiments the CID domain is a dimerization domain of FKBP or FRB (FKBP—rapamycin-binding) domain of mTOR, which are activated with rapamycin analogs. In particular embodiments a suicide switch includes a sequence encoding an iCaspase 9 construct (iCasp9) with an FRB or FKBP CID domain. In particular embodiments, the suicide switch activating agent is rapamycin or an analog thereof.

Based on the foregoing, in particular embodiments, a genetic construct encoding a selected antibody includes or encodes (i) a promoter, and/or (ii) an immunoglobulin light chain, and/or (iii) a heavy chain variable region, and/or (iv) a stop codon, and/or (v) a skipping element, and/or (vi) a splice junction, and/or (vii) homology regions, and/or (viii) a linker, and/or (ix) a tag, and/or (x) a suicide switch.

In particular embodiments, a genetic construct encoding a selected antibody includes or encodes: (i) a promoter, (ii) a signal peptide, (iii) an entire light chain of a selected antibody, (iv) a flexible linker or a skipping element, (v) the variable region of a selected antibody heavy chain, and (vi) a splice junction. Herein, “entire” or “full length” as it refers to a light chain means that the light chain protein or the sequence encoding it includes or encodes the variable light chain and the constant light chain.

In particular embodiments, a genetic construct encoding a selected antibody includes or encodes: (i) a promoter, (ii) a signal peptide, (iii) an entire light chain of a selected antibody, (iv) a flexible linker or a skipping element, (v) the variable region of a selected antibody heavy chain, (vi) a splice junction, and (vii) homology regions.

In particular embodiments, a genetic construct encoding a selected antibody includes or encodes: (i) a promoter, (ii) a signal peptide, (iii) an entire light chain of a selected antibody, (iv) a flexible linker or a skipping element, (v) the variable region of a selected antibody heavy chain, (vi) a splice junction, (vii) homology regions, and (viii) a tag.

In particular embodiments, a genetic construct encoding a selected antibody includes or encodes: (i) a promoter, (ii) a signal peptide, (iii) an entire light chain of a selected antibody, (iv) a flexible linker or a skipping element, (v) the variable region of a selected antibody heavy chain, (vi) a splice junction, (vii) homology regions, and (viii) a suicide switch.

In particular embodiments, a genetic construct encoding a selected antibody includes or encodes: (i) a promoter, (ii) a signal peptide, (iii) an entire light chain of a selected antibody, (iv) a flexible linker or a skipping element, (v) the variable region of a selected antibody heavy chain, (vi) a splice junction, (vii) homology regions, (viii) a suicide switch, and (ix) a tag.

In particular embodiments, a genetic construct encoding a gene product includes or encodes (i) a promoter, and/or (ii) a gene product, and/or (iii) a suicide switch, and/or (iv) a tag, and/or (v) homology regions.

In particular embodiments, a genetic construct encoding a gene product includes or encodes (i) a promoter and (ii) a gene product.

In particular embodiments, a genetic construct encoding a gene product includes or encodes (i) a promoter, (ii) a gene product, and/or (iii) a suicide switch.

In particular embodiments, a genetic construct encoding a gene product includes or encodes (i) a promoter, (ii) a gene product, and/or (iii) a tag.

In particular embodiments, a genetic construct encoding a gene product includes or encodes (i) a promoter, (ii) a gene product, (iii) a suicide switch, and/or (iv) a tag.

In particular embodiments, a genetic construct encoding a gene product includes or encodes (i) a promoter, (ii) a gene product, and/or (iii) homology regions.

In particular embodiments, a genetic construct encoding a gene product includes or encodes (i) a promoter, (ii) a gene product, (iii) a suicide switch, (iv) a tag, and/or (v) homology regions.

(iv) Introduction of Genetic Engineering Components into B Cells. In particular embodiments, genome targeting and cutting elements and genetic constructs for insertion can be administered through electroporation, nanoparticle-mediated delivery and/or viral vector delivery. Electroporation can be useful, for example, to deliver targeting elements and/or cutting elements because the membrane of the cell does not normally allow such foreign molecules into the cell. Electroporation sends an electric shock to the cells that temporarily allows such foreign molecules to pass through the cell membrane.

In particular embodiments, genetic constructs for insertion can be administered through electroporation, nanoparticle-mediated delivery and/or viral vector delivery. Adeno-associated viral vectors include those derived from e.g., adenovirus 5 (Ad5), adenovirus 35 (Ad35), adenovirus 11 (Ad11), adenovirus 26 (Ad26), adenovirus 48 (Ad48) or adenovirus 50 (Ad50)), and adeno-associated virus (AAV; see, e.g., U.S. Pat. No. 5,604,090; Kay et al., Nat. Genet. 24:257 (2000); Nakai et al., Blood 91:4600 (1998)).

In particular embodiments, genome targeting and cutting elements can be administered through electroporation and genetic constructs for insertion can be administered through AAV-mediated delivery. In particular embodiments, genome targeting and cutting elements can be administered through nanoparticle-mediated delivery and genetic constructs for insertion can be administered through AAV-mediated delivery.

In particular embodiments, genetic constructs including a transgene can be mixed with a targeting element (e.g., sgRNA) and a cutting element (e.g., Cas9 or cpf1) immediately or shortly before electroporation. Selected antibody expression can be confirmed later (e.g., 3 days later) by measuring cell binding to fluorescently tagged target proteins by flow cytometry. Enrichment and analysis methodologies for detecting and analyzing epitope-specific B cells can be used. Pape et al., Science. 2011; 331(6021):1203-7; Taylor et al., J Exp Med. 2012; 209(3):597-606; Taylor et al., J Exp Med. 2012; 209(11):2065-77; Haasken et al., J Immunol. 2013; 191(3):1055-62; Taylor et al., J Immunol Methods. 2014; 405:74-86; Nanton et al., Eur J Immunol. 2015; 45(2):428-41; Hamilton et al., J Immunol. 2015; 194(10):5022-34; Taylor et al., Science. 2015; 347(6223):784-7). These methodologies allow detection of selected antibody-expressing B cells at frequencies as extraordinarily low as 0.00002% of the total B cell population (Taylor et al., Science. 2015; 347(6223):784-7). Gene product can also be assessed using methods well known to those of ordinary skill in the art. For example, if a gene product is a secreted protein, the protein can be detected using ELISA and/or a supernatant activity assay.

B cells can be genetically modified in vivo or ex vivo utilizing, for example, cell-targeted nanoparticles. Exemplary cell-targeted nanoparticles include a cell targeting ligand on the surface of the nanoparticle wherein the cell targeting ligand results in selective uptake of the nanoparticle by a selected cell type (e.g., B cell). Exemplary cell targeting ligands for B cells are described below. The nanoparticle then binds the B cell, delivering gene modifying components that result in expression of the selected antibody and gene product.

Exemplary nanoparticles include liposomes (microscopic vesicles including at least one concentric lipid bilayer surrounding an aqueous core), liposomal nanoparticles (a liposome structure used to encapsulate another smaller nanoparticle within its core); and lipid nanoparticles (liposome-like structures that lack the continuous lipid bilayer characteristic of liposomes). Other polymer-based nanoparticles can also be used as well as porous nanoparticles constructed from any material capable of forming a porous network. Exemplary materials include metals, transition metals and metalloids (e.g., lithium, magnesium, zinc, aluminum, and silica).

For in vivo delivery and cellular uptake, nanoparticles can have a neutral or negatively-charged coating and a size of 130 nm or less. Dimensions of the nanoparticles can be determined using, e.g., conventional techniques, such as dynamic light scattering and/or electron microscopy.

In particular embodiments, gene product expression can be confirmed using any method known to a person skilled in the art. In particular embodiments, a sample can be extracted from subject tissue or blood. Gene product expression within the sample can be measured using immunoblotting, immunoprecipitation, immunofluorescence, chemiluminescence, electro-chemiluminescence (ECL), enzyme-linked immunoassays (ELISA), chromatography, spectroscopy, capillary electrophoresis, mass spectrometry, and/or any other qualitative or quantitative assay known in the art.

(v) B Cell Markers for Cell Sorting and Targeting. In particular embodiments, B cells can be targeted and bound by a nanoparticle, identified, and/or sorted based on B cell marker expression (e.g., before or after delivering the genetic constructs). For example, it may be useful to isolate or target a particular type of B cell (e.g., memory B cell, antibody-secreting B cell, naïve B cell, B1 B cell, marginal zone B cell) for in vivo delivery of genetic engineering components.

CD19 is an example of a protein expressed by B cells but few other cells of the body. Thus, CD19 can be used to identify, isolate, and/or target B cells. B220 is a useful marker to identify, isolate, and/or target mouse B cells. Binding domains that bind CD19 can be derived from antibodies known in the art. For example, blinatumomab, tafasitamab, and loncastuximab tesirine are monoclonal antibodies that target CD19.

In particular embodiments, a binding domain that binds CD19 includes a heavy chain including the sequence: QVQLVQSGAEVKKPGSSVKVSCKASGGTFSSYTINWVRQAPGQGLEWMGGIPIIFGIPNYAQK FQGRVTITADESTNTAYMELSSLRAEDTAVYYCARASGGSADYSYGMDVWGQGTAVTVSS (SEQ ID NO: 375) and a variable light chain having the sequence DIQMTQSPSSLSASVGDRVTITCRASQGISSWLAWYQQKPEKAPKSLIYAASSLQSGVPSRFS GSGSGTDFTLTISSLQPEDFATYYCQQYKRYPYTFGQGTKLEIK (SEQ ID NO: 376).

In particular embodiments, a binding domain that binds CD19 includes a variable heavy chain including a CDRH1 sequence including GYAFSSYWMN (SEQ ID NO: 377), a CDRH2 sequence including QIWPGDGDTNYNGKFKG (SEQ ID NO: 378), a CDRH3 sequence including RETTTVGRYYYAMDY (SEQ ID NO: 379); and a variable light chain including a CDRL1 sequence including KASQSVDYDGDSYLN (SEQ ID NO: 380), a CDRL2 sequence including DASNLVS (SEQ ID NO: 381), and a CDRL3 sequence including QQSTEDPWT (SEQ ID NO: 382).

In particular embodiments, a binding domain that binds CD19 includes a heavy chain including the sequence: QVQLLESGAELVRPGSSVKISCKASGYAFSSYWMNWVKQRPGQGLEWIGQIWPGDGDTNYN GKFKGKATLTADESSSTAYMQLSSLRSEDSAVYSCARRETTTVGRYYYAMDYWGQGTTVT (SEQ ID NO: 383) and a variable light chain having the sequence ELVLTQSPASLAVSLGQRATISCKASQSVDYDGDSYLNWYQQIPGQPPKLLIYDASNLVSGIPP RFSGSGSGTDFTLNIHPVEKVDAATYHCQQSTEDPWTFGGGTKLEIKRRS (SEQ ID NO: 384).

CD27 is an example of a protein expressed by memory but not naive human B cells. CD27 is a useful marker to identify, isolate, and/or target memory B cells. CD27 binds to ligand CD70 and plays a role in regulating B cell activation and immunoglobulin synthesis. Binding domains that bind CD27 can be derived from antibodies known in the art. For example, mAb 2177 binds CD27.

In particular embodiments, a binding domain that binds CD27 includes a variable heavy chain including a CDRH1 sequence including SYTMS (SEQ ID NO: 385), a CDRH2 sequence including YISSGGGNTYYPDSVKG (SEQ ID NO: 386), a CDRH3 sequence including HRGNPFDY (SEQ ID NO: 387); and a variable light chain including a CDRL1 sequence including RASKSVSTSGYSFMH (SEQ ID NO: 388), a CDRL2 sequence including LASNLES (SEQ ID NO: 389), and a CDRL3 sequence including QHSRELPWT (SEQ ID NO: 396).

In particular embodiments, a binding domain that binds CD27 includes a variable heavy chain including a CDRH1 sequence including GFTFSSYTMS (SEQ ID NO: 391), a CDRH2 sequence including YISSGGGNTYYPDSVKG (SEQ ID NO: 392), a CDRH3 sequence including HRGNPFDY (SEQ ID NO: 387); and a variable light chain including a CDRL1 sequence including RASKSVSAWGYSFMH (SEQ ID NO: 394), a CDRL2 sequence including VASRLES (SEQ ID NO: 395), and a CDRL3 sequence including QHSRELPWT (SEQ ID NO: 396).

In particular embodiments, a binding domain that binds CD27 includes a variable heavy chain including a CDRH1 sequence including GFTFSSYGMS (SEQ ID NO: 397), a CDRH2 sequence including YIDEGGGQTIYPDSVKG (SEQ ID NO: 398), a CDRH3 sequence including HRGNPFDY (SEQ ID NO: 387); and a variable light chain including a CDRL1 sequence including RASKSVSHVRWSFMH (SEQ ID NO: 400), a CDRL2 sequence including LASKLES (SEQ ID NO: 401), and a CDRL3 sequence including QHSRELPWT (SEQ ID NO: 396).

CD21 is an example of a protein not expressed (or expressed to a low degree) by some memory human B cells with the capacity to quickly secrete antibody following infection. Low CD21 expression can be used to define B cells primed for plasma cell differentiation. CD21 can be a useful marker to identify, isolate, and/or target B cells primed for plasma differentiation, particularly through negative selection. Binding domains that bind CD21 can be derived from antibodies known in the art. For example, MA5-11417, AF4909, and LT21 bind CD21.

In particular embodiments, a CD21 targeting peptide includes the sequence RMWPSSTVNLSAGRR (SEQ ID NO: 403).

In particular embodiments, a binding domain that binds CD21 includes a heavy chain including the sequence: MAVLVLFLCLVAFPSCVLSQVQLKESGPGLVAPSQSLSITCTVSGFSLTNYGVHWVRQPPGKG LEWLGMIWAGGSTNYNSALMSRLNINKDNSKSQVFLEMNSLQTDDTALYFCAREATSGYVDY AVDYWGQGTSVTVSSAKTTAPSVYPLAPVCGDTTGSSVTLGCLVKGYFPEPVTLTWNSGSLS SGVHTFPAVLQSDLYTLSSSVTVTSSTWPSQSITCNVAHPASSTKVDKKIEPRGPTIKPCPPCK CPAPNLLGGPSVFIFPPKIKDVLMISLSPIVTCVVVDVSEDDPDVQISWFVNNVEVHTAQTQTHR EDYNSTLRVVSALPIQHQDWMSGKEFKCKVNNKDLPAPIERTISKPKGSVRAPQVYVLPPPEEE MTKKQVTLTCMVTDFMPEDIYVEWTNNGKTELNYKNTEPVLDSDGSYFMYSKLRVEKKNWVE RNSYSCSVVHEGLHNHHTTKSFSRTPGK (SEQ ID NO: 404) and a variable light chain having the sequence:

(SEQ ID NO: 405)
MDFQVQIFSFLLISASVIISRGQIILNQSPAIMSAFPGEKVTMTCSASSSVSYMHWYQQKSGTSP
KRWIYDTSKLASGVPARFSGSGSGTSYSLTISSMEAEDAATYYCQQWSSSPLTFGAGTKLELK
RADAAPTVSIFPPSSEQLTSGGASVVCFLNNFYPKDINVKWKIDGSERQNGVLNSWTDQDSKD
STYSMSSTLTLTKDEYERHNSYTCEATHKTSTSPIVKSFNRNEC.

In particular embodiments, a binding domain that binds CD21 includes a variable heavy chain including a CDRH1 sequence including GYTFTSN (SEQ ID NO: 406), a CDRH2 sequence including SPGDGD (SEQ ID NO: 407), a CDRH3 sequence including GDSSGWGPNWFDS (SEQ ID NO: 408); and a variable light chain including a CDRL1 sequence including LASQDIGNYLS (SEQ ID NO: 409), a CDRL2 sequence including DVNNLED (SEQ ID NO: 410), and a CDRL3 sequence including QQYYEYPLT (SEQ ID NO: 411).

In particular embodiments, a binding domain that binds CD21 includes a variable heavy chain including a CDRH1 sequence including GYTFTTN (SEQ ID NO: 412), a CDRH2 sequence including NPGDGN (SEQ ID NO: 413), a CDRH3 sequence including GDYSGWGPNWFDY (SEQ ID NO: 414); and a variable light chain including a CDRL1 sequence including LASQDIGDYLS (SEQ ID NO: 415), a CDRL2 sequence including GATNLED (SEQ ID NO: 416), and a CDRL3 sequence including HQYYQYPLT (SEQ ID NO: 417).

Human naïve B cells can be identified, isolated, and/or targeted by the marker profile IgM+ IgD+ CD27−. Mouse naïve B cells can be identified, isolated, and/or targeted by the marker profile CD38+ GL7− IgM+ IgD+. Human 1 B cells can be identified, isolated, and/or targeted by the marker profile CD5+ CD43+. Mouse 1 B cells can be identified, isolated, and/or targeted by the marker profile CD43+ B220^{lo}. Human marginal zone B cells can be identified, isolated, and/or targeted by the marker profile CD21+++ IgM++ IgD− CD27+. Mouse marginal zone B cells can be identified, isolated, and/or targeted by the marker profile CD21+++ IgM++ IgD−.

Particular embodiments may utilize the CD19⁺CD27⁺CD21^{lo} marker profile to identify, isolate, and/or target B cells.

In particular embodiments, CD45 is a marker used for identifying, isolating, and/or targeting cell types. Different mouse strains express different versions of the protein called CD45, termed CD45.1 and CD45.2. In particular embodiments, B cells from a mouse that expresses CD45.2 will be taken and transferred into a mouse that expresses CD45.1. By marking CD45.1 and CD45.2 with different fluorescent molecules, one can identify the cells that came from the donor animal because they express CD45.2 but not CD45.1.

Particular embodiments include sorting B cells after genetic modification based on expression of an exogenous light chain. For example, B cells that naturally express a kappa light chain can be modified to express a selected antibody that includes a lambda light chain. B cells that naturally express a lambda light chain can be modified to express a selected antibody that includes a kappa light chain. Sorting based on expression of an exogenous light chain will allow for isolation of only those B cells expressing the selected antibody. In particular embodiments, only those B cells that completely lack surface expression of their endogenous light chain are isolated for formulation and administration to a subject.

In particular embodiments, B cells may be identified and/or isolated using flow cytometry. Flow cytometry is a sensitive and powerful analysis approach that uses lasers to individually analyze the fluorescent molecules marking millions of individual cells. By analyzing the combination of fluorescent molecules each cell is marked with, different B cell subtypes can be identified. Flow cytometry can be used to identify B cell subsets and analyze the expression of selected antibodies or gene products by B cells.

In particular embodiments, methods of modifying B cells can include obtaining hematopoietic stem cells (HSC), and/or delivering the genetic constructs to HSC. HSC can refer to a type of stem cell that naturally produces B cells as well as all other cells of the immune system. HSC can be obtained, for example, from cord blood.

In particular embodiments, an immortalized B cell line can be used. In particular embodiments, an immortalized B cell line includes A20 cells.

In particular embodiments, B cells may be obtained from a human subject and obtained B cells or a subset thereof may be modified ex vivo.

(vi) Formulations & Compositions for Administration. The current disclosure provides formulations and compositions for administration. Formulations include ex vivo genetically modified B cells and a pharmaceutically acceptable carrier. Compositions include a nanoparticle that results in in vivo genetic modification of B cells and a pharmaceutically acceptable carrier (a nanoparticle composition); gene editing tools (e.g., Cas9, sgRNA, genetic constructs) that result in in vivo genetic modification of B cells; and/or an antigen that binds the selected antibody expressed by a B cell and a pharmaceutically acceptable carrier (an antigen composition); and/or a suicide switch activating agent and a pharmaceutically acceptable carrier (suicide switch activating agent composition).

The phrase “pharmaceutically acceptable” refers to those compounds, materials, and/or dosage forms which are, within the scope of sound medical judgment, suitable for use in contact with the tissues of human beings and animals without excessive toxicity, commensurate with a reasonable benefit/risk ratio. In certain instances, pharmaceutically-acceptable carriers have been approved by a relevant regulatory agency (e.g., the United States Food and Drug Administration (US FDA)).

In particular embodiments, exemplary pharmaceutically acceptable carriers for formulations and compositions include saline, buffered saline, physiological saline, water, Hanks' solution, Ringer's solution, Normosol-R (Abbott Labs), PLASMA-LYTE A® (Baxter Laboratories, Inc., Morton Grove, IL), glycerol, ethanol, and combinations thereof. In particular embodiments, carriers can be supplemented with human serum albumin (HSA) or other human serum components or fetal bovine serum. In particular embodiments, a carrier for infusion includes buffered saline with 5% hyaluronic acid sodium salt (HAS) or dextrose.

Depending on the context and compound for delivery, “pharmaceutically acceptable carriers” can also include any adjuvant, excipient, glidant, diluent, preservative, dye/colorant, surfactant, wetting agent, dispersing agent, suspending agent, stabilizer, isotonic agent, solvent, surfactant, or emulsifier which meets the requirements noted above. Exemplary pharmaceutically acceptable carriers are disclosed in Remington's Pharmaceutical Sciences, 18th Ed. Mack Printing Company, 1990. Moreover, formulations and compositions can be prepared to meet sterility, pyrogenicity, general safety, and purity standards as required by the US FDA Office of Biological Standards and/or other relevant foreign regulatory agencies.

Exemplary antioxidants include ascorbic acid, methionine, and vitamin E.

An exemplary chelating agent is EDTA.

Exemplary isotonic agents include polyhydric sugar alcohols including trihydric or higher sugar alcohols, such as glycerin, erythritol, arabitol, xylitol, sorbitol, or mannitol.

Carriers can include buffering agents, such as citrate buffers, succinate buffers, tartrate buffers, fumarate buffers, gluconate buffers, oxalate buffers, lactate buffers, acetate buffers, phosphate buffers, histidine buffers, and/or trimethylamine salts.

Exemplary preservatives include phenol, benzyl alcohol, meta-cresol, methyl paraben, propyl paraben, octadecyldimethylbenzyl ammonium chloride, benzalkonium halides, hexamethonium chloride, alkyl parabens such as methyl or propyl paraben, catechol, resorcinol, cyclohexanol, and 3-pentanol.

Stabilizers refer to a broad category of excipients which can range in function from a bulking agent to an additive which helps to prevent cell adherence to container walls. Typical stabilizers can include polyhydric sugar alcohols; amino acids, such as arginine, lysine, glycine, glutamine, asparagine, histidine, alanine, ornithine, L-leucine, 2-phenylalanine, glutamic acid, and threonine; organic sugars or sugar alcohols, such as lactose, trehalose, stachyose, mannitol, sorbitol, xylitol, ribitol, myoinisitol, galactitol, glycerol, and cyclitols, such as inositol; PEG; amino acid polymers; sulfur-containing reducing agents, such as urea, glutathione, thioctic acid, sodium thioglycolate, thioglycerol, alpha-monothioglycerol, and sodium thiosulfate; low molecular weight polypeptides (i.e., <10 residues); proteins such as HSA, bovine serum albumin, gelatin or immunoglobulins; hydrophilic polymers such as polyvinylpyrrolidone; monosaccharides such as xylose, mannose, fructose and glucose; disaccharides such as lactose, maltose and sucrose; trisaccharides such as raffinose, and polysaccharides such as dextran. Stabilizers are typically present in the range of from 0.1 to 10,000 parts by weight based on therapeutic weight.

Where necessary or beneficial, formulations and/or compositions can include a local anesthetic such as lidocaine to ease pain at a site of injection.

The formulations and compositions disclosed herein can be formulated for administration by, for example, injection. For injection, formulation can be formulated as aqueous solutions, such as in buffers including Hanks' solution, Ringer's solution, or physiological saline, or in culture media, such as Iscove's Modified Dulbecco's Medium (IMDM). The aqueous solutions can include formulatory agents such as suspending, stabilizing, and/or dispersing agents.

Compositions can be formulated for oral administration. For example, compositions can be formulated as tablets, pills, dragees, capsules, liquids, gels, syrups, slurries, suspensions and the like. For oral solid compositions such as powders, capsules and tablets, suitable excipients include binders (gum tragacanth, acacia, cornstarch, gelatin), fillers such as sugars, e.g. lactose, sucrose, mannitol and sorbitol; dicalcium phosphate, starch, magnesium stearate, sodium saccharine, cellulose, magnesium carbonate; cellulose preparations such as maize starch, wheat starch, rice starch, potato starch, gelatin, gum tragacanth, methyl cellulose, hydroxypropylmethyl-cellulose, sodium carboxy-methylcellulose, and/or polyvinylpyrrolidone (PVP); granulating agents; and binding agents. If desired, disintegrating agents can be added, such as corn starch, potato starch, alginic acid, cross-linked polyvinylpyrrolidone, agar, or alginic acid or a salt thereof such as sodium alginate. If desired, solid dosage forms can be sugar-coated or enteric-coated using standard techniques. Flavoring agents, such as peppermint, oil of wintergreen, cherry flavoring, orange flavoring, etc. can also be used.

Compositions can be formulated as an aerosol. In particular embodiments, the aerosol is provided as part of an anhydrous, liquid or dry powder inhaler. Aerosol sprays from pressurized packs or nebulizers can also be used with a suitable propellant, e.g., dichlorodifluoromethane, trichlorofluoromethane, dichlorotetrafluoroethane, carbon dioxide or other suitable gas. In the case of a pressurized aerosol, a dosage unit may be determined by providing a valve to deliver a metered amount. Capsules and cartridges of gelatin for use in an inhaler or insufflator may also be formulated including a powder mix of antigens and a suitable powder base such as lactose or starch.

Compositions can also be formulated as depot preparations. Depot preparations can be formulated with suitable polymeric or hydrophobic materials (for example as an emulsion in an acceptable oil) or ion exchange resins, or as sparingly soluble derivatives, for example, as a sparingly soluble salt.

Depot compositions can include a variety of bioerodible polymers including poly(lactide), poly(glycolide), poly(caprolactone) and poly(lactide)-co(glycolide) (PLG) of desirable lactide:glycolide ratios, average molecular weights, polydispersities, and terminal group chemistries. Blending different polymer types in different ratios using various grades can result in characteristics that borrow from each of the contributing polymers.

The use of different solvents (for example, dichloromethane, chloroform, ethyl acetate, triacetin, N-methyl pyrrolidone, tetrahydrofuran, phenol, or combinations thereof) can alter microparticle size and structure in order to modulate release characteristics. Other useful solvents include water, ethanol, dimethyl sulfoxide (DMSO), N-methyl-2-pyrrolidone (NMP), acetone, methanol, isopropyl alcohol (IPA), ethyl benzoate, and benzyl benzoate.

Exemplary release modifiers can include surfactants, detergents, internal phase viscosity enhancers, complexing agents, surface active molecules, co-solvents, chelators, stabilizers, derivatives of cellulose, (hydroxypropyl)methyl cellulose (HPMC), HPMC acetate, cellulose acetate, pluronics (e.g., F68/F127), polysorbates, Span® (Croda Americas, Wilmington, Delaware), poly(vinyl alcohol) (PVA), Brij® (Croda Americas, Wilmington, Delaware), sucrose acetate isobutyrate (SAIB), salts, and buffers.

Excipients that partition into the external phase boundary of microparticles such as surfactants including polysorbates, dioctylsulfosuccinates, poloxamers, PVA, can also alter properties including particle stability and erosion rates, hydration and channel structure, interfacial transport, and kinetics in a favorable manner.

Additional processing of the disclosed sustained release depot compositions can utilize stabilizing excipients including mannitol, sucrose, trehalose, and glycine with other components such as polysorbates, PVAs, and dioctylsulfosuccinates in buffers such as Tris, citrate, or histidine. A freeze-dry cycle can also be used to produce very low moisture powders that reconstitute to similar size and performance characteristics of the original suspension.

Compositions disclosed herein can be formulated for administration by, for example, injection, infusion, perfusion, or lavage. The compositions disclosed herein can further be formulated for intravenous, intradermal, intraarterial, intranodal, intralymphatic, intraperitoneal, intralesional, intraprostatic, intravaginal, intrarectal, topical, intrathecal, intratumoral, intramuscular, intravesicular, oral and/or subcutaneous administration and more particularly by intravenous, intradermal, intraarterial, intranodal, intralymphatic, intraperitoneal, intralesional, intraprostatic, intravaginal, intrarectal, intrathecal, intratumoral, intramuscular, intravesicular, and/or subcutaneous injection. In particular embodiments, formulations disclosed herein can be formulated for administration by infusion.

Any formulation or composition disclosed herein can advantageously include any other pharmaceutically acceptable carriers which include those that do not produce significantly adverse, allergic, or other untoward reactions that outweigh the benefit of administration. Exemplary pharmaceutically acceptable carriers and formulations are disclosed in Remington's Pharmaceutical Sciences, 18th Ed. Mack Printing Company, 1990. Moreover, formulations can be prepared to meet sterility, pyrogenicity, general safety, and purity standards as required by U.S. FDA Office of Biological Standards and/or other relevant foreign regulatory agencies.

Modified formulations can include, for example, greater than 10² modified B cells, greater than 10³ modified B cells, greater than 10⁴ modified B cells, greater than 10⁵ modified B cells, greater than 10⁶ modified B cells, greater than 10⁷ modified B cells, greater than 10⁸ modified B cells, greater than 10⁹ modified B cells, greater than 10¹⁰ modified B cells, or greater than 10¹¹ modified B cells.

Therapeutically effective amounts of nanoparticles and/or antigens and/or suicide switch activating agent within compositions can range from 0.1 to 5 μg/kg or from 0.5 to 1 μg/kg. In other examples, a dose can include 1 μg/kg, 30 μg/kg, 90 μg/kg, 150 μg/kg, 500 μg/kg, 750 μg/kg, 0.1 to 5 mg/kg or from 0.5 to 1 mg/kg. In other examples, a dose can include 1 mg/kg, 10 mg/kg, 30 mg/kg, 50 mg/kg, 70 mg/kg, 100 mg/kg, 300 mg/kg, 500 mg/kg, 700 mg/kg, 1000 mg/kg or more.

(vii) Methods of Use. Methods disclosed herein include treating subjects (e.g., humans, veterinary animals (dogs, cats, reptiles, birds) livestock (e.g., horses, cattle, goats, pigs, chickens) and research animals (e.g., monkeys, rats, mice, fish) with formulations and/or compositions disclosed herein. Treating subjects includes delivering therapeutically effective amounts. Therapeutically effective amounts include those that provide effective amounts, prophylactic treatments and/or therapeutic treatments.

An “effective amount” is the amount of a formulation and/or composition necessary to result in a desired physiological change in the subject. Effective amounts are often administered for research purposes. Effective amounts disclosed herein can cause a statistically-significant effect in an animal model or in vitro assay relevant to the assessment of a condition's development, progression, and/or resolution.

A “prophylactic treatment” includes a treatment administered to a subject who does not display signs or symptoms of a condition or displays only early signs or symptoms of a condition such that treatment is administered for the purpose of diminishing or decreasing the risk of developing the condition. Thus, a prophylactic treatment functions as a preventative treatment against a condition. In particular embodiments, prophylactic treatments reduce, delay, or prevent the worsening of a condition.

A “therapeutic treatment” includes a treatment administered to a subject who displays symptoms or signs of a condition and is administered to the subject for the purpose of diminishing or eliminating those signs or symptoms of the condition. The therapeutic treatment can reduce, control, or eliminate the presence or activity of the condition and/or reduce control or eliminate side effects of the condition.

Function as an effective amount, prophylactic treatment or therapeutic treatment are not mutually exclusive, and in particular embodiments, administered dosages may accomplish more than one treatment type.

In particular embodiments, the condition is a lysosomal storage disease, clotting disorders, diabetes, or alpha-1 antitrypsin deficiency. In particular embodiments, the lysosomal storage disease includes Gaucher disease (GD), Fabry disease, mucopolysaccharidosis (MPS) type I, MPS type II, MPS type VI, and Pompe disease (PD). In particular embodiments, the clotting disorder includes hemophilia A, hemophilia B, or hemophilia C.

Therapeutically effective amounts can be achieved by administering single or multiple doses during the course of a treatment regimen (e.g., daily, every other day, every 3 days, weekly, every 2 weeks, monthly, every 2 months, every 4 months, every 6 months, yearly, etc.).

As indicated, the formulations and/or compositions can be administered by injection, infusion, transfusion, implantation or transplantation. Compositions can also be administered via ingestion or inhalation. In particular embodiments, formulations and compositions are administered parenterally. The phrases “parenteral administration” and “administered parenterally” refer to modes of administration other than enteral and topical administration, usually by injection, and includes, intravascular, intravenous, intramuscular, intraarterial, intrathecal, intracapsular, intratumoral, intraperitoneal, and subcutaneous, injection and infusion. In particular embodiments, the formulations and compositions described herein are administered to a subject by infusion.

In certain examples the disclosure provides methods of amplifying expression of a gene product by a B cell including: administering a formulation or composition that results in in vivo expression by a B cell of a gene product and a selected antibody and administering an antigen composition that binds the selected antibody and activates the B cell upon antigen binding. In some alternatives, the antigen composition is delivered prior to, at the same time as the formulation or nanoparticle composition, or at later time points after the formulation or nanoparticle composition has been administered.

In particular embodiments, a lower threshold value is determined as the minimal amount of gene product a subject needs in order to avoid detrimental symptoms of a disease or disorder. In particular embodiments, an upper threshold value is determined to be the highest amount of gene product that a subject can have without toxicity or side effects of the treatment.

In particular embodiments, if the gene product is below a lower threshold value, the antigen composition is administered. In particular embodiments, if a subject experiences a symptom of a disorder, the antigen composition can be administered. In particular embodiments, a symptom of a lysosomal storage disease can include seizures, movement disorders, dementia, blindness, deafness, skin sores, body aches, fever, pain, anemia, joint stiffness, and swollen abdomen. In particular embodiments, a symptom of a clotting disorder can include fatigue, internal bleeding, blood in urine or stool, easily bruising, stroke, heart attack, and leg pain. In particular embodiments, a symptom of a diabetes can include frequent urination, weight change, hunger, blurry vision, fatigue, dry skin, tingling hands or feet, slow wound healing, and infections. In particular embodiments, a symptom of an alpha-trypsin deficiency can include shortness of breath, excessive cough, phlegm production, wheezing, fatigue, chest pain, jaundice, vomiting, easy bruising, and swelling in belly or legs from fluid.

In particular embodiments, if the gene product is above an upper threshold value, the antigen composition is withdrawn until the gene product expression is below the threshold value.

In some alternatives, the antigen composition is administered with the formulation or nanoparticle composition, and if a toxic effect of the formulation or nanoparticle composition is observed the antigen composition is withdrawn until the toxic effects diminish. After the symptoms of toxicity diminish, the antigen composition can be administered again.

In particular embodiments, if the gene product is above an upper threshold value, the suicide switch activating agent composition can be administered to activate the suicide switch.

In particular embodiments, if toxic effects of treatment are observed or retained, the suicide switch activating agent composition can be administered to activate the suicide switch. In particular embodiments, genetic constructs encode a suicide switch that can be activated by a suicide switch activating agent. In particular embodiments, administration of a composition including the suicide switch activating agent causes the destruction of the B cell.

In particular embodiments, modified B cells express a tag that allows, for example, tracking and/or elimination after administration to a subject.

For administration, therapeutically effective amounts (also referred to herein as doses) can be initially estimated based on results from in vitro assays and/or animal model studies. Such information can be used to more accurately determine useful doses in subjects of interest. The actual dose amount administered to a particular subject can be determined by a physician, veterinarian or researcher taking into account parameters such as physical and physiological factors including age, previous vaccinations (if any), target, body weight, severity of condition, type of condition, stage of condition, previous or concurrent therapeutic interventions, idiopathy of the subject and route of administration.

Exemplary therapeutically effective amounts can include greater than 10² modified B cells, greater than 10³ modified B cells, greater than 10⁴ modified B cells, greater than 10⁵ modified B cells, greater than 10⁶ modified B cells, greater than 10⁷ modified B cells, greater than 10⁸ modified B cells, greater than 10⁹ modified B cells, greater than 10¹⁰ modified B cells, or greater than 10¹¹ modified B cells.

In particular embodiments, therapeutically effective amounts of nanoparticles and/or antigen within compositions can range from, for example, 0.1 to 5 μg/kg or from 0.5 to 1 μg/kg. In other examples, a dose can include 1 μg/kg, 15 μg/kg, 30 μg/kg, 50 μg/kg, 55 μg/kg, 70 μg/kg, 90 μg/kg, 150 μg/kg, 350 μg/kg, 500 μg/kg, 750 μg/kg, 1000 μg/kg, 0.1 to 5 mg/kg or from 0.5 to 1 mg/kg. In other examples, a dose can include 1 mg/kg, 10 mg/kg, 30 mg/kg, 50 mg/kg, 70 mg/kg, 100 mg/kg, 300 mg/kg, 500 mg/kg, 700 mg/kg, 1000 mg/kg or more.

The Exemplary Embodiments and Example below are included to demonstrate particular embodiments of the disclosure. Those of ordinary skill in the art should recognize in light of the present disclosure that many changes can be made to the specific embodiments disclosed herein and still obtain a like or similar result without departing from the spirit and scope of the disclosure.

### (viii) Exemplary Embodiments

1. A B cell including a first genetic construct encoding an anti-poliovirus antibody at
a) an intronic region upstream or downstream of an Eμ enhancer within the native antibody heavy chain gene at a site targeted a gRNA sequence as set forth in SEQ ID NO: 145 and a second genetic construct encoding alpha-galactosidase A upstream or downstream of an iE_{κ} enhancer within the native antibody light chain gene at a site targeted by a gRNA sequence as set forth in SEQ ID NO: 221; or
b) an intronic region upstream or downstream of an Eμ enhancer within the native antibody heavy chain gene at a site targeted by a gRNA sequence as set forth in SEQ ID NO: 181 and a second genetic construct encoding alpha-galactosidase A upstream or downstream of an iE_{κ} enhancer within the native antibody light chain gene at a site targeted by a gRNA sequence as set forth in SEQ ID NO: 241.
   2. A B cell including a first genetic construct at a first particular area within the native antibody heavy chain gene of the B cell's genome and a second genetic construct at a second particular area within the native antibody light chain gene of the B cell's genome.
   3. The B cell of embodiment 2, wherein the first genetic construct encodes a selected antibody.
   4. The B cell of embodiments 2 or 3, wherein the first genetic construct includes a first promoter, a sequence encoding a signal peptide, a sequence encoding the variable light chain and constant light chain of the selected antibody, a sequence encoding a flexible linker or a skipping element, a sequence encoding the variable region of the heavy chain of the selected antibody, and/or a splice junction.
   5. The B cell of embodiments 3 or 4, wherein the selected antibody includes an anti-smallpox antibody or an anti-poliovirus antibody.
   6. The B cell of embodiments 4 or 5, wherein the variable light chain of the selected antibody includes the sequence as set forth in SEQ ID NO: 316, SEQ ID NO: 324, SEQ ID NO: 332, or SEQ ID NO: 428 or a sequence having at least 90% sequence identity to SEQ ID NO: 316, SEQ ID NO: 324, SEQ ID NO: 332, or SEQ ID NO: 428.
   7. The B cell of any of embodiments 4-6, wherein the sequence encoding the variable light chain of the selected antibody includes the sequence as set forth in SEQ ID NO: 419 or a sequence having at least 90% sequence identity to SEQ ID NO: 419.
   8. The B cell of any of embodiments 4-7, wherein the variable light chain of the selected antibody includes a variable light chain sequence having complementarity determining regions (CDRs) including a CDRL1 having the sequence as set forth in SEQ ID NO: 320, a CDRL2 having the sequence as set forth in SEQ ID NO: 321, and a CDRL3 having the sequence as set forth in SEQ ID NO: 322; a CDRL1 having the sequence as set forth in SEQ ID NO: 328, a CDRL2 having the sequence as set forth in SEQ ID NO: 329, and a CDRL3 having the sequence as set forth in SEQ ID NO: 330; or a CDRL1 having the sequence as set forth in SEQ ID NO: 336, a CDRL2 having the sequence as set forth in SEQ ID NO: 337, and a CDRL3 having the sequence as set forth in SEQ ID NO: 338.
   9. The B cell of any of embodiments 4-8, wherein the sequence encoding the variable light chain of the selected antibody includes a variable light chain sequence having complementarity determining regions (CDRs) including a CDRL1 having the sequence as set forth in SEQ ID NO: 423, a CDRL2 having the sequence as set forth in SEQ ID NO: 424, and a CDRL3 having the sequence as set forth in SEQ ID NO: 425.
   10. The B cell of any of embodiments 4-9, wherein the variable region of the heavy chain of the selected antibody includes the sequence as set forth in SEQ ID NO: 315, SEQ ID NO: 323, SEQ ID NO: 331, or SEQ ID NO: 427 or a sequence having at least 90% sequence identity to SEQ ID NO: 315, SEQ ID NO: 323, SEQ ID NO: 331, or SEQ ID NO: 42.
   11. The B cell of any of embodiments 4-10, wherein the sequence encoding the variable region of the heavy chain of the selected antibody includes the sequence as set forth in SEQ ID NO: 418 or a sequence having at least 90% sequence identity to SEQ ID NO: 418.
   12. The B cell of any of embodiments 4-11, wherein the variable region of the heavy chain of the selected antibody includes a variable heavy chain sequence having complementarity determining regions (CDRs) including a CDRH1 having the sequence as set forth in SEQ ID NO: 317, a CDRH2 having the sequence as set forth in SEQ ID NO: 318, and a CDRH3 having the sequence as set forth in SEQ ID NO: 319; a CDRH1 having the sequence as set forth in SEQ ID NO: 325, a CDRH2 having the sequence as set forth in SEQ ID NO: 326, and a CDRH3 having the sequence as set forth in SEQ ID NO: 327; or a CDRH1 having the sequence as set forth in SEQ ID NO: 333, a CDRH2 (SEQ ID NO: 334, and a CDRH3 having the sequence as set forth in SEQ ID NO: 335.
   13. The B cell of any of embodiments 4-12, wherein the sequence encoding the variable region of the heavy chain of the selected antibody includes a variable heavy chain sequence having complementarity determining regions (CDRs) including a CDRH1 having the sequence as set forth in SEQ ID NO: 420, a CDRH2 having the sequence as set forth in SEQ ID NO: 421, and a CDRH3 having the sequence as set forth in SEQ ID NO: 422.
   14. The B cell of any of embodiments 4-13, wherein the first promoter is a heavy chain (VH) promoter.
   15. The B cell of any of embodiments 4-14, wherein the flexible linker is between the variable light chain of the selected antibody and the variable region of the heavy chain of the selected antibody.
   16. The B cell of any of embodiments 4-15, wherein the flexible linker is a Gly-Ser linker.
   17. The B cell of embodiment 16, wherein the Gly-Ser linker includes (GGS)n (SEQ ID NO: 343), (GGGS)n (SEQ ID NO: 344), or (GGGGS)n (SEQ ID NO: 345).
   18. The B cell of any of embodiments 4-17, wherein the flexible linker includes the sequence as set forth in SEQ ID NO: 347 or a sequence having at least 90% sequence identity to SEQ ID NO: 347.
   19. The B cell of any of embodiments 4-18, wherein the flexible linker is encoded by the sequence as set forth in SEQ ID NO: 346 or a sequence having at least 90% sequence identity to SEQ ID NO: 346.
   20. The B cell of any of embodiments 2-19, wherein the first genetic construct further encodes a suicide switch.
   21. The B cell of embodiment 20, wherein the suicide switch includes an iCaspase 9 construct (iCasp9).
   22. The B cell of any of embodiments 2-21, wherein the first genetic construct further includes a sequence encoding a tag.
   23. The B cell of embodiment 22, wherein the tag includes the sequence as set forth in SEQ ID NO: 365, SEQ ID NO: 366, SEQ ID NO: 367, SEQ ID NO: 368, SEQ ID NO: 369, SEQ ID NO: 370, SEQ ID NO: 371, SEQ ID NO: 372, SEQ ID NO: 373, or SEQ ID NO: 374 or a sequence having at least 90% sequence identity to SEQ ID NO: 365, SEQ ID NO: 366, SEQ ID NO: 367, SEQ ID NO: 368, SEQ ID NO: 369, SEQ ID NO: 370, SEQ ID NO: 371, SEQ ID NO: 372, SEQ ID NO: 373, or SEQ ID NO: 374.
   24. The B cell of any of embodiments 4-23, wherein the skipping element includes a self-cleaving peptide.
   25. The B cell of embodiment 24, wherein the self-cleaving peptide includes a T2A self-cleaving peptide, a P2A self-cleaving peptide, an E2A self-cleaving peptide, or an F2A self-cleaving peptide.
   26. The B cell of any of embodiments 2-25, wherein the first genetic construct includes the sequence as set forth in SEQ ID NO: 310 or SEQ ID NO: 311 or a sequence having at least 90% sequence identity to SEQ ID NO: 310 or SEQ ID NO: 311.
   27. The B cell of any of embodiments 2-26, wherein the first particular area is an intronic region upstream or downstream of an Eμ enhancer within the native antibody heavy chain gene.
   28. The B cell of embodiment 27, wherein the Eμ enhancer includes the sequence set forth in SEQ ID NO: 10 or SEQ ID NO: 11.
   29. The B cell of any of embodiments 2-28, wherein the first particular area includes the sequence as set forth in SEQ ID NO: 14, SEQ ID NO: 15, SEQ ID NO: 16, or SEQ ID NO: 17.
   30. The B cell of any of embodiments 2-29, wherein the first particular area includes a gRNA target site including the sequence as set forth in one of SEQ ID NOs: 20-99.
   31. The B cell of embodiment 30, wherein a gRNA sequence targeting the gRNA target site includes the sequence as set forth in one of SEQ ID NOs: 140-219.
   32. The B cell of any of embodiments 2-31, wherein the first genetic construct further includes an upstream homology arm and a downstream homology arm.
   33. The B cell of embodiment 32, wherein the upstream homology arm of the first genetic construct includes a sequence as set forth in SEQ ID NO: 260 SEQ ID NO: 262, or SEQ ID NO: 264 or a sequence having at least 90% sequence identity to SEQ ID NO: 260 SEQ ID NO: 262, or SEQ ID NO: 264.
   34. The B cell of embodiments 321 or 33, wherein the downstream homology arm of the first genetic construct includes a sequence as set forth in SEQ ID NO: 261, SEQ ID NO: 263, or SEQ ID NO: 265 or a sequence having at least 90% sequence identity to SEQ ID NO: 261, SEQ ID NO: 263, or SEQ ID NO: 265.
   35. The B cell of any of embodiments 2-34, wherein the second genetic construct includes a second promoter and encodes a gene product.
   36. The B cell of embodiment 35, wherein the gene product includes a secreted protein, inhibitory nucleic acid molecule, or non-secreted protein.
   37. The B cell of embodiment 36, wherein the secreted protein includes glucocerebrosidase (GC), acid sphingomyelinase, mucopolysaccharides, acid alpha-glucosidase, aspartylglucosaminidase, alpha-galactosidase A, palmitoyl protein thioesterase, tripeptidyl peptidase, lysosomal transmembrane protein, cysteine transporter, acid ceramidase, acid alpha-L-fucosidase, cathepsin A, acid beta-glucosidase, acid beta-galactosidase, iduronate-2-sulfatase, alpha-L-iduronidase, galactocerebrosidase, acid alpha-mannosidase, acid beta-mannosidase, arylsulfatase B, arylsulfatase A, N-acetylgalactosamine-6-sulfate, N-acetylglucosamine-1-phosphotransferase, acid sphingomyelinase, NPC-1, alpha-glucosidase, beta-hexosaminidase B, heparan N-sulfatase, alpha-N-acetylglucosaminidase, acetyl-CoA: alpha-glucosaminide, N-acetylglucosamine-6-sulfate, alpha-N-acetylgalactosaminidase, alpha-neuramidase, beta-glucuronidase, beta-hexosaminidase A, or acid lipase.
   38. The B cell of embodiment 36, wherein the secreted protein includes factor VIII, factor IX, factor XI.
   39. The B cell of embodiment 36, wherein the secreted protein includes insulin, alpha-1 antitrypsin, lactase, glucose-6-phosphate dehydrogenase (G6PD), pyruvate kinase, glucocerebrosidase, ATPase7B, galactose-1-phosphate uridyl transferase (GALT), branched-chain α-ketoacid dehydrogenase (BCKD) complex, phenylalanine hydroxylase (PAH), glucose-6-phosphatase (G6Pase), debranching enzyme, glycogen-branching enzyme, glutaryl-CoA dehydrogenase, frataxin, peroxisome biogenesis disorders (PBDs) or peroxisomes, 5α-reductase, glucose phosphate isomerase, hexosaminidase A, activin receptor type-1 (ACVR1), pramlinitide acetate, growth hormone (GH), insulin-like growth factor, protein C, α1-proteinase inhibitor, erythropoietin, granulocyte colony-stimulating factor (G-CSF), Interleukin 11, human follicle-stimulating hormone (FSH), human chorionic gonadotropin (HCG), Lutropin-α, Interleukin 2 (IL2), Denileukin diftitox (fusion of IL2 and Diphtheria toxin), Interferon-α2a, Interferon-α2b, Interferon-αn3, Interferon-β1a, Interferon-β1b, Interferon-γ1b, human parathyroid hormone, glucagon-like peptide 1, somatostatin, bone morphogenic protein 2, bone morphogenic protein 7, gonadotropin-releasing hormone (GnRH), keratinocyte growth factor (KGF), platelet-derived growth factor (PDGF), B-type natriuretic peptide, or hirudin.
   40. The B cell of embodiment 36, wherein the secreted protein includes interleukin 2.
   41. The B cell of embodiment 36, wherein the secreted protein includes a fluorescent protein.
   42. The B cell of embodiment 41, wherein the fluorescent protein includes mCherry or green fluorescent protein (GFP).
   43. The B cell of any of embodiments 35-42, wherein the second promoter includes a CMV promoter, an MND promoter, or a Vk21E promoter.
   44. The B cell of any of embodiments 2-43, wherein the second genetic construct further encodes a suicide switch.
   45. The B cell of embodiment 44, wherein the suicide switch includes an iCaspase 9 construct (iCasp9).
   46. The B cell of any of embodiments 2-45, wherein the second genetic construct further includes a tag.
   47. The B cell of embodiment 46, wherein the tag includes the sequence as set forth in SEQ ID NO: 365, SEQ ID NO: 366, SEQ ID NO: 367, SEQ ID NO: 368, SEQ ID NO: 369, SEQ ID NO: 370, SEQ ID NO: 371, SEQ ID NO: 372, SEQ ID NO: 373, or SEQ ID NO: 374 or a sequence having at least 90% sequence identity to SEQ ID NO: 365, SEQ ID NO: 366, SEQ ID NO: 367, SEQ ID NO: 368, SEQ ID NO: 369, SEQ ID NO: 370, SEQ ID NO: 371, SEQ ID NO: 372, SEQ ID NO: 373, or SEQ ID NO: 374.
   48. The B cell of any of embodiments 2-47, wherein the second genetic construct includes the sequence as set forth in SEQ ID NO: 293, SEQ ID NO: 294, SEQ ID NO: 295, SEQ ID NO: 296, SEQ ID NO: 297, SEQ ID NO: 298, SEQ ID NO: 299, SEQ ID NO: 300, SEQ ID NO: 301, SEQ ID NO: 302, SEQ ID NO: 303, SEQ ID NO: 304, SEQ ID NO: 305, SEQ ID NO: 306, SEQ ID NO: 307, SEQ ID NO: 308, or SEQ ID NO: 309 or a sequence having at least 90% sequence identity to SEQ ID NO: 293, SEQ ID NO: 294, SEQ ID NO: 295, SEQ ID NO: 296, SEQ ID NO: 297, SEQ ID NO: 298, SEQ ID NO: 299, SEQ ID NO: 300, SEQ ID NO: 301, SEQ ID NO: 302, SEQ ID NO: 303, SEQ ID NO: 304, SEQ ID NO: 305, SEQ ID NO: 306, SEQ ID NO: 307, SEQ ID NO: 308, or SEQ ID NO: 309.
   49. The B cell of any of embodiments 2-48, wherein the second particular area is upstream or downstream of an iEκ enhancer within the native antibody light chain gene.
   50. The B cell of embodiment 49, wherein the iEκ enhancer includes the sequence as set forth in SEQ ID NO: 12 or SEQ ID NO: 13.
   51. The B cell of any of embodiments 2-50, wherein the second particular area includes the sequence as set forth in SEQ ID NO: 18 or SEQ ID NO: 19.
   52. The B cell of any of embodiments 2-51, wherein the second particular area includes a gRNA target site including the sequence as set forth in one of SEQ ID NOs: 100-139.
   53. The B cell of embodiment 52, wherein a gRNA sequence targeting the gRNA target site includes the sequence as set forth in one of SEQ ID NOs: 220-259.
   54. The B cell of any of embodiments 2-53, wherein the second genetic construct further includes an upstream homology arm and a downstream homology arm.
   55. The B cell of embodiment 54, wherein the upstream homology arm of the second genetic construct includes a sequence as set forth in SEQ ID NO: 266, SEQ ID NO: 268, SEQ ID NO: 270, SEQ ID NO: 272, SEQ ID NO: 274, or SEQ ID NO: 276 or a sequence having at least 90% sequence identity to SEQ ID NO: 266, SEQ ID NO: 268, SEQ ID NO: 270, SEQ ID NO: 272, SEQ ID NO: 274, or SEQ ID NO: 276.
   56. The B cell of embodiments 54 or 55, wherein the downstream homology arm of the second genetic construct includes a sequence as set forth in SEQ ID NO: 267, SEQ ID NO: 269, SEQ ID NO: 271, SEQ ID NO: 273, SEQ ID NO: 275, or SEQ ID NO: 277 or a sequence having at least 90% sequence identity to SEQ ID NO: 267, SEQ ID NO: 269, SEQ ID NO: 271, SEQ ID NO: 273, SEQ ID NO: 275, or SEQ ID NO: 277.
   57. The B cell of any of embodiments 2-56, wherein the B cell is used to treat a subject in need thereof.
   58. A formulation including the B cell of any of embodiments 1-57 and a pharmaceutically acceptable carrier.
   59. A kit for genetically modifying a B cell, the kit including a first genetic construct encoding a selected antibody, a second genetic construct encoding a gene product, a first gRNA targeting an intronic region upstream or downstream of an Eμ enhancer within the native antibody heavy chain gene of the B cell, and a second gRNA targeting a region upstream or downstream of an iEκ enhancer within the native antibody light chain gene.
   60. The kit of embodiment 59, wherein the first gRNA includes SEQ ID NO: 181, SEQ ID NO: 142, or SEQ ID NO: 145 or a sequence having at least 90% sequence identity to a sequence as set forth in SEQ ID NO: 181, SEQ ID NO: 142, or SEQ ID NO: 145.
   61. The kit of embodiments 59 or 60, wherein the first gRNA includes one of SEQ ID NOs: 140-219 or a sequence having at least 90% sequence identity to SEQ ID NOs: 140-219.
   62. The kit of embodiment 60, wherein the first gRNA is sgRNA.
   63. The kit of any of embodiments 59-62, wherein the first gRNA targets one of SEQ ID NOs: 20-99.
   64. The kit of any of embodiments 59-63, wherein the second gRNA includes the sequence as set forth in SEQ ID NO: 225, SEQ ID NO: 224, SEQ ID NO: 221, SEQ ID NO: 244, SEQ ID NO: 242, or SEQ ID NO: 241 or a sequence having at least 90% sequence identity to SEQ ID NO: 225, SEQ ID NO: 224, SEQ ID NO: 221, SEQ ID NO: 244, SEQ ID NO: 242, or SEQ ID NO: 241.
   65. The kit of any of embodiments 59-64, wherein the second gRNA includes one of SEQ ID NOs: 220-259 or a sequence having at least 90% sequence identity to of SEQ ID NOs: 220-259.
   66. The kit of embodiment 64, wherein the second gRNA is sgRNA.
   67. The kit of any of embodiments 59-66, wherein the second gRNA targets one of SEQ ID NOs: 100-139.
   68. The kit of any of embodiments 59-67, wherein the selected antibody includes a smallpox virus antibody or a poliovirus antibody.
   69. The kit of any of embodiments 59-68, wherein the selected antibody binds a synthetic antigen.
   70. The kit of any of embodiments 59-69, further including an antigen that binds the selected antibody.
   71. The kit of embodiment 70, wherein the antigen is a rare antigen.
   72. The kit of embodiments 70 or 71, wherein the antigen includes a smallpox virus antigen or a poliovirus antigen.
   73. The kit of embodiments 70 or 71, wherein the antigen is a synthetic antigen.
   74. The kit of embodiment 73, wherein the synthetic antigen includes R-phycoerythrin or allophycocyanin.
   75. The kit of any of embodiments 59-75, wherein the first genetic construct further includes a first promoter.
   76. The kit of embodiment 75, wherein the first promoter includes a VH promoter.
   77. The kit of any of embodiments 59-76, wherein the first genetic construct further includes a sequence encoding a tag.
   78. The kit of embodiment 77, wherein the tag includes the sequence as set forth in SEQ ID NO: 365, SEQ ID NO: 366, SEQ ID NO: 367, SEQ ID NO: 368, SEQ ID NO: 369, SEQ ID NO: 370, SEQ ID NO: 371, SEQ ID NO: 372, SEQ ID NO: 373, or SEQ ID NO: 374 or a sequence having at least 90% sequence identity to SEQ ID NO: 365, SEQ ID NO: 366, SEQ ID NO: 367, SEQ ID NO: 368, SEQ ID NO: 369, SEQ ID NO: 370, SEQ ID NO: 371, SEQ ID NO: 372, SEQ ID NO: 373, or SEQ ID NO: 374.
   79. The kit of any of embodiments 59-78, wherein the first genetic construct further includes a skipping element, homology regions, and/or a splice junction.
   80. The kit of any of embodiments 59-79, wherein the first genetic construct further encodes a suicide switch.
   81. The kit of embodiment 80, wherein the suicide switch includes an iCaspase 9 construct (iCasp9).
   82. The kit of any of embodiments 59-81, wherein the first genetic construct includes the sequence as set forth in SEQ ID NO: 310 or SEQ ID NO: 311 or a sequence having at least 90% sequence identity to SEQ ID NO: 310 or SEQ ID NO: 311.
   83. The kit of any of embodiments 59-82, wherein the gene product is a secreted protein, non-secreted protein, or inhibitory nucleic acid molecule.
   84. The kit of embodiment 83, wherein the secreted protein includes glucocerebrosidase (GC), acid sphingomyelinase, mucopolysaccharides, acid alpha-glucosidase, aspartylglucosaminidase, alpha-galactosidase A, palmitoyl protein thioesterase, tripeptidyl peptidase, lysosomal transmembrane protein, cysteine transporter, acid ceramidase, acid alpha-L-fucosidase, cathepsin A, acid beta-glucosidase, acid beta-galactosidase, iduronate-2-sulfatase, alpha-L-iduronidase, galactocerebrosidase, acid alpha-mannosidase, acid beta-mannosidase, arylsulfatase B, arylsulfatase A, N-acetylgalactosamine-6-sulfate, N-acetylglucosamine-1-phosphotransferase, acid sphingomyelinase, NPC-1, alpha-glucosidase, beta-hexosaminidase B, heparan N-sulfatase, alpha-N-acetylglucosaminidase, acetyl-CoA: alpha-glucosaminide, N-acetylglucosamine-6-sulfate, alpha-N-acetylgalactosaminidase, alpha-neuramidase, beta-glucuronidase, beta-hexosaminidase A, or acid lipase.
   85. The kit of embodiment 83, wherein the secreted protein includes factor VIII, factor IX, or factor XI.
   86. The kit of embodiment 83, wherein the secreted protein includes insulin, alpha-1 antitrypsin, lactase, glucose-6-phosphate dehydrogenase (G6PD), pyruvate kinase, glucocerebrosidase, ATPase7B, galactose-1-phosphate uridyl transferase (GALT), branched-chain α-ketoacid dehydrogenase (BCKD) complex, phenylalanine hydroxylase (PAH), glucose-6-phosphatase (G6Pase), debranching enzyme, glycogen-branching enzyme, glutaryl-CoA dehydrogenase, frataxin, peroxisome biogenesis disorders (PBDs) or peroxisomes, 5α-reductase, glucose phosphate isomerase, hexosaminidase A, activin receptor type-1 (ACVR1), pramlinitide acetate, growth hormone (GH), insulin-like growth factor, protein C, α1-proteinase inhibitor, erythropoietin, granulocyte colony-stimulating factor (G-CSF), Interleukin 11, human follicle-stimulating hormone (FSH), human chorionic gonadotropin (HCG), Lutropin-α, Interleukin 2 (IL2), Denileukin diftitox (fusion of IL2 and Diphtheria toxin), Interferon-α2a, Interferon-α2b, Interferon-αn3, Interferon-β1a, Interferon-β1b, Interferon-γ1b, human parathyroid hormone, glucagon-like peptide 1, somatostatin, bone morphogenic protein 2, bone morphogenic protein 7, gonadotropin-releasing hormone (GnRH), keratinocyte growth factor (KGF), platelet-derived growth factor (PDGF), B-type natriuretic peptide, or hirudin.
   87. The kit of embodiment 83, wherein the secreted protein includes interleukin 2.
   88. The kit of embodiment 83, wherein the secreted protein includes a fluorescent protein.
   89. The kit of embodiment 88, wherein the fluorescent protein includes mCherry or green fluorescent protein (GFP).
   90. The kit of any of embodiments 59-89, wherein the second genetic construct further includes a second promoter.
   91. The kit of embodiment 90, wherein the second promoter includes a CMV promoter, an MND promoter, or a Vk21E promoter.
   92. The kit of any of embodiments 59-91, wherein the second genetic construct further includes a sequence encoding a tag.
   93. The kit of embodiment 92, wherein the tag includes the sequence as set forth in SEQ ID NO: 365, SEQ ID NO: 366, SEQ ID NO: 367, SEQ ID NO: 368, SEQ ID NO: 369, SEQ ID NO: 370, SEQ ID NO: 371, SEQ ID NO: 372, SEQ ID NO: 373, or SEQ ID NO: 374 or a sequence having at least 90% sequence identity to SEQ ID NO: 365, SEQ ID NO: 366, SEQ ID NO: 367, SEQ ID NO: 368, SEQ ID NO: 369, SEQ ID NO: 370, SEQ ID NO: 371, SEQ ID NO: 372, SEQ ID NO: 373, or SEQ ID NO: 374.
   94. The kit of any of embodiments 59-93, wherein the second genetic construct further encodes a suicide switch.
   95. The kit of embodiment 94, wherein the suicide switch includes an iCasp9 construct.
   96. The kit of any of embodiments 59-95, further including a suicide switch activating agent.
   97. The kit of any of embodiments 59-96, further including a nuclease.
   98. The kit of embodiment 97, wherein the nuclease is Cas9 or Cpf1.
   99. The kit of any of embodiments 59-98, wherein the second genetic construct includes the sequence as set forth in SEQ ID NO: 293, SEQ ID NO: 294, SEQ ID NO: 295, SEQ ID NO: 296, SEQ ID NO: 297, SEQ ID NO: 298, SEQ ID NO: 299, SEQ ID NO: 300, SEQ ID NO: 301, SEQ ID NO: 302, SEQ ID NO: 303, SEQ ID NO: 304, SEQ ID NO: 305, SEQ ID NO: 306, SEQ ID NO: 307, SEQ ID NO: 308, or SEQ ID NO: 309 or a sequence having at least 90% sequence identity to SEQ ID NO: 293, SEQ ID NO: 294, SEQ ID NO: 295, SEQ ID NO: 296, SEQ ID NO: 297, SEQ ID NO: 298, SEQ ID NO: 299, SEQ ID NO: 300, SEQ ID NO: 301, SEQ ID NO: 302, SEQ ID NO: 303, SEQ ID NO: 304, SEQ ID NO: 305, SEQ ID NO: 306, SEQ ID NO: 307, SEQ ID NO: 308, or SEQ ID NO: 309.
   100. The kit of any of embodiments 59-99, further including a nanoparticle or adeno-associated viral vector.
   101. The kit of embodiment 100, wherein the gRNA and nuclease are associated with a nanoparticle.
   102. The kit of embodiments 100 or 101, wherein the nanoparticle is a B-cell targeted nanoparticle.
   103. The kit of any of embodiments 59-102, wherein the first genetic construct and second genetic construct are part of an adeno-associated viral vector.
   104. The kit of any of embodiments 59-103, wherein the genetic modification is performed in vivo or ex vivo.
   105. A method of genetically modifying a B cell including introducing into the B cell
a first targeting element, a first cutting element, and a first genetic construct encoding a selected antibody and
a second targeting element, a second cutting element, and a second genetic construct encoding a gene product
wherein
the first targeting element and the first cutting element result in insertion of the first genetic construct at a first particular area within the native antibody heavy chain gene of the B cell's genome and
the second targeting element and the second cutting element result in insertion of the second genetic construct at a second particular area within the native antibody light chain gene of the B cell's genome.
   106. The method of embodiment 105, wherein the first targeting element targets a first particular area including an intronic region upstream or downstream of an Eμ enhancer within the native antibody heavy chain gene.
   107. The method of embodiments 105 or 106, wherein the first particular area includes the sequence as set forth in SEQ ID NO: 14, SEQ ID NO: 15, SEQ ID NO: 16, or SEQ ID NO: 17.
   108. The method of any of embodiments 105-107, wherein the first particular area includes a gRNA target site including the sequence as set forth in one of SEQ ID NOs: 140-219.
   109. The method of any of embodiments 105-108, wherein the first targeting element includes an sgRNA having a sequence as set forth in SEQ ID NO: 181, SEQ ID NO: 142, or SEQ ID NO: 145 or a sequence having at least 90% sequence identity to SEQ ID NO: 181, SEQ ID NO: 142, or SEQ ID NO: 145.
   110. The method of any of embodiments 105-109, wherein the second targeting element targets a second particular area including a region upstream or downstream of an iEκ enhancer within the native antibody light chain gene.
   111. The method of any of embodiments 105-110, wherein the second particular area includes the sequence as set forth in SEQ ID NO: 18 or SEQ ID NO: 19.
   112. The method of any of embodiments 105-111, wherein the second particular area includes a gRNA target site including the sequence as set forth in one of SEQ ID NOs: 100-139.
   113. The method of any of embodiments 105-112, wherein the second targeting element includes an sgRNA having a sequence as set forth in SEQ ID NO: 225, SEQ ID NO: 224, SEQ ID NO: 221, SEQ ID NO: 244, SEQ ID NO: 242, or SEQ ID NO: 241 or a sequence having at least 90% sequence identity to SEQ ID NO: 225, SEQ ID NO: 224, SEQ ID NO: 221, SEQ ID NO: 244, SEQ ID NO: 242, or SEQ ID NO: 241.
   114. The method of any of embodiments 105-113, wherein the first genetic construct includes an upstream homology arm and a downstream homology arm.
   115. The method of embodiment 114, wherein the upstream homology arm of the first genetic construct includes a sequence as set forth in SEQ ID NO: 260 SEQ ID NO: 262, or SEQ ID NO: 264 or a sequence having at least 90% sequence identity to SEQ ID NO: 260 SEQ ID NO: 262, or SEQ ID NO: 264.
   116. The method of embodiments 114 or 115, wherein the downstream homology arm of the first genetic construct includes a sequence as set forth in SEQ ID NO: 261 SEQ ID NO: 263, or SEQ ID NO: 265 or a sequence having at least 90% sequence identity to SEQ ID NO: 261 SEQ ID NO: 263, or SEQ ID NO: 265.
   117. The method of any of embodiments 105-116, wherein the second genetic construct includes an upstream homology arm and a downstream homology arm.
   118. The method of embodiment 117, wherein the upstream homology arm of the second genetic construct includes a sequence as set forth in SEQ ID NO:266, SEQ ID NO: 268, SEQ ID NO: 270, SEQ ID NO: 272, SEQ ID NO: 274, or SEQ ID NO: 276 or a sequence having at least 90% sequence identity to SEQ ID NO:266, SEQ ID NO: 268, SEQ ID NO: 270, SEQ ID NO: 272, SEQ ID NO: 274, or SEQ ID NO: 276.
   119. The method of embodiments 117 or 118, wherein the downstream homology arm of the second genetic construct includes a sequence as set forth in SEQ ID NO: 267, SEQ ID NO: 269, SEQ ID NO: 271, SEQ ID NO: 273, SEQ ID NO: 275, or SEQ ID NO: 277 or a sequence having at least 90% sequence identity to SEQ ID NO: 267, SEQ ID NO: 269, SEQ ID NO: 271, SEQ ID NO: 273, SEQ ID NO: 275, or SEQ ID NO: 277.
   120. The method of any of embodiments 105-119, wherein the introducing into the B cell is ex vivo or in vivo.
   121. The method of any of embodiments 105-120, wherein the first targeting element, the first cutting element, and the first genetic construct are associated with a nanoparticle.
   122. The method of any of embodiments 105-121, wherein the second targeting element, the second cutting element, and the second genetic construct are associated with a nanoparticle.
   123. The method of embodiments 121 or 122, wherein the nanoparticle is a B-cell targeted nanoparticle.
   124. The method of any of embodiments 105-123, further including administering an antigen that binds the selected antibody to upregulate genetic of the gene product.
   125. The method of any of embodiments 105-124, wherein the first genetic construct includes a first promoter, a sequence encoding a signal peptide, a sequence encoding the variable light chain and constant light chain of the selected antibody, a sequence encoding a flexible linker or a skipping element, a sequence encoding the variable region of the heavy chain of the selected antibody, and/or a splice junction.
   126. The method of any of embodiments 105-125, wherein the first genetic construct further encodes a suicide switch.
   127. The method of embodiment 126, wherein the suicide switch includes an iCaspase 9 construct (iCasp9).
   128. The method of embodiments 126 or 127, wherein the suicide switch is activated by a suicide switch activation agent.
   129. The method of embodiment 128, wherein the suicide switch activation agent is rapamycin or an analog thereof.
   130. The method of any of embodiments 105-129, wherein the second genetic construct includes a second promoter and encodes a gene product.
   131. The method of any of embodiments 105-130, wherein the second genetic construct further encodes a suicide switch.
   132. The method of embodiment 131, wherein the suicide switch includes an iCaspase 9 construct (iCasp9).
   133. The method of embodiments 131 or 132, wherein the suicide switch is activated by a suicide switch activation agent.
   134. The method of embodiment 133, wherein the suicide switch activation agent is rapamycin or an analog thereof.
   135. A method of providing expression of a gene product in a subject including administering to the subject a therapeutically effective amount of the B cell of embodiment 1-57 and/or a therapeutically effective amount of nanoparticles associated with
a first targeting element, a first cutting element, and a first genetic construct encoding a selected antibody and
a second targeting element, a second cutting element, and a second genetic construct encoding a gene product
wherein
the first targeting element and the first cutting element result in insertion of the first genetic construct at a particular area within the native antibody heavy chain gene of the B cell's genome within the subject and
the second targeting element and the second cutting element result in insertion of the second genetic construct at a particular area within the native antibody light chain gene of the B cell's genome within the subject.
   136. The method of embodiment 135, including administering the therapeutically effective amount of the B cell of any of embodiments 1-57 by infusion, injection, perfusion, or lavage.
   137. The method of embodiment 135, including administering the therapeutically effective amount of the nanoparticles by infusion, injection, perfusion, or lavage.
   138. The method of any of embodiments 135-137, further including administering an antigen that binds the selected antibody.
   139. The method of embodiment 138, wherein the administering the antigen is on a dosing schedule.
   140. The method of embodiment 139, wherein the dosing schedule is daily, weekly, monthly, or annually.
   141. The method of any of embodiments 135-140, further including monitoring the subject for a condition.
   142. The method of embodiment 141, wherein the condition includes a side effect of a disorder.
   143. The method of embodiments 141 or 142, wherein the condition includes expression levels of the gene product falling below a lower threshold value.
   144. The method of any of embodiments 135-143, further including administering an antigen that binds the selected antibody upon detection of the condition when expression levels of the gene product fall below a lower threshold.
   145. The method of embodiment 144, wherein the administering of the antigen is through ingestion, injection, or inhalation.
   146. The method of embodiments 141 or 142, wherein the condition includes expression levels of the gene product exceed an upper threshold value.
   147. The method of any of embodiments 135-146, wherein the first genetic construct and/or second genetic construct encodes a suicide switch.
   148. The method of embodiment 147, wherein the suicide switch is activated by a suicide switch activation agent.
   149. The method of embodiment 148, wherein the suicide switch activation agent is administered when expression levels of the gene product exceeds an upper threshold.
   150. The method of any of embodiments 135-149, wherein the subject has a gene product deficiency.
   151. The method of embodiment 150, wherein the gene product deficiency is a lysosomal storage disease, a clotting disorder, diabetes, or an alpha-1 antitrypsin deficiency.
   152. A method of treating a lysosomal storage disease within a subject, the method including administering to the subject a therapeutically effective amount of the B cell of any of embodiments 1-57 thereby treating the lysosomal storage disease within the subject.
   153. A method of treating a clotting disorder within a subject, the method including administering to the subject a therapeutically effective amount of the B cell of any of embodiments 1-57 thereby treating the clotting disorder within the subject.
   154. A method of treating diabetes within a subject, the method including administering to the subject a therapeutically effective amount of the B cell of any of embodiments 1-57 thereby treating diabetes within the subject.
   155. A method of treating an alpha-1 antitrypsin deficiency within a subject, the method including administering to the subject a therapeutically effective amount of the B cell of any of embodiments 1-57 thereby treating the alpha-1 antitrypsin deficiency within the subject.

(ix) Closing Paragraphs. The nucleic acid and amino acid sequences provided herein are shown using letter abbreviations for nucleotide bases and amino acid residues, as defined in 37 C.F.R. § 1.831-1.835 and set forth in WIPO Standard ST.26 (implemented on Jul. 1, 2022). Only one strand of each nucleic acid sequence is shown, but the complementary strand is understood as included in embodiments where it would be appropriate. Any nucleic acid that encodes a selected antibody construct as described herein may be utilized. Variants of nucleic acid sequences disclosed herein include various sequence polymorphisms, mutations, and alterations wherein the differences in the sequence do not substantially affect the function of the encoded protein. The term nucleic acid or “gene” may include not only coding sequences but also regulatory regions such as promoters, enhancers, and termination regions. The term further can include all introns and other DNA sequences spliced from the mRNA transcript, along with variants resulting from alternative splice sites. Encoding nucleic acid can be DNA or RNA that directs the expression of the one or more selected antibody constructs. These nucleic acid sequences may be a DNA strand sequence that is transcribed into RNA or an RNA sequence that is translated into protein. The nucleic acid sequences include both the full-length nucleic acid sequences as well as non-full-length sequences derived from the full-length protein. The sequences can also include degenerate codons of the native sequence or sequences that may be introduced to provide codon preference in a specific cell type. Nucleic acid sequences encoding selected antibody constructs can be readily prepared from the relevant amino acid sequence of a selected antibody construct.

Variants of the sequences disclosed and referenced herein are also included. Variants of protein sequences include those having one or more amino acid additions, deletions, stop positions, or substitutions, as compared to a protein sequence disclosed elsewhere herein. Guidance in determining which amino acid residues can be substituted, inserted, or deleted without abolishing biological activity can be found using computer programs well known in the art, such as DNASTAR™ (Madison, Wisconsin) software. Preferably, amino acid changes in the protein variants disclosed herein are conservative amino acid changes, i.e., substitutions of similarly charged or uncharged amino acids. A conservative amino acid change involves substitution of one of a family of amino acids which are related in their side chains.

In a peptide or protein, suitable conservative substitutions of amino acids are known to those of skill in this art and generally can be made without altering a biological activity of a resulting molecule. Those of skill in this art recognize that, in general, single amino acid substitutions in non-essential regions of a polypeptide do not substantially alter biological activity (see, e.g., Watson et al. Molecular Biology of the Gene, 4th Edition, 1987, The Benjamin/Cummings Pub. Co., p. 224). Naturally occurring amino acids are generally divided into conservative substitution families as follows: Group 1: Alanine (Ala), Glycine (Gly), Serine (Ser), and Threonine (Thr); Group 2: (acidic): Aspartic acid (Asp), and Glutamic acid (Glu); Group 3: (acidic; also classified as polar, negatively charged residues and their amides): Asparagine (Asn), Glutamine (Gln), Asp, and Glu; Group 4: Gln and Asn; Group 5: (basic; also classified as polar, positively charged residues): Arginine (Arg), Lysine (Lys), and Histidine (His); Group 6 (large aliphatic, nonpolar residues): Isoleucine (lie), Leucine (Leu), Methionine (Met), Valine (Val) and Cysteine (Cys); Group 7 (uncharged polar): Tyrosine (Tyr), Gly, Asn, Gln, Cys, Ser, and Thr; Group 8 (large aromatic residues): Phenylalanine (Phe), Tryptophan (Trp), and Tyr; Group 9 (non-polar): Proline (Pro), Ala, Val, Leu, Ile, Phe, Met, and Trp; Group 11 (aliphatic): Gly, Ala, Val, Leu, and Ile; Group 10 (small aliphatic, nonpolar or slightly polar residues): Ala, Ser, Thr, Pro, and Gly; and Group 12 (sulfur-containing): Met and Cys. Additional information can be found in Creighton (1984) Proteins, W.H. Freeman and Company.

In making such changes, the hydropathic index of amino acids may be considered. The importance of the hydropathic amino acid index in conferring interactive biologic function on a protein is generally understood in the art (Kyte and Doolittle, 1982, J. Mol. Biol. 157(1), 105-32). Each amino acid has been assigned a hydropathic index on the basis of its hydrophobicity and charge characteristics (Kyte and Doolittle, 1982). These values are: Ile (+4.5); Val (+4.2); Leu (+3.8); Phe (+2.8); Cys (+2.5); Met (+1.9); Ala (+1.8); Gly (−0.4); Thr (−0.7); Ser (−0.8); Trp (−0.9); Tyr (−1.3); Pro (−1.6); His (−3.2); Glutamate (−3.5); Gln (−3.5); aspartate (−3.5); Asn (−3.5); Lys (−3.9); and Arg (−4.5).

It is known in the art that certain amino acids may be substituted by other amino acids having a similar hydropathic index or score and still result in a protein with similar biological activity, i.e., still obtain a biological functionally equivalent protein. In making such changes, the substitution of amino acids whose hydropathic indices are within ±2 is preferred, those within ±1 are particularly preferred, and those within ±0.5 are even more particularly preferred. It is also understood in the art that the substitution of like amino acids can be made effectively on the basis of hydrophilicity.

As detailed in U.S. Pat. No. 4,554,101, the following hydrophilicity values have been assigned to amino acid residues: Arg (+3.0); Lys (+3.0); aspartate (+3.0±1); glutamate (+3.0±1); Ser (+0.3); Asn (+0.2); Gln (+0.2); Gly (0); Thr (−0.4); Pro (−0.5±1); Ala (−0.5); His (−0.5); Cys (−1.0); Met (−1.3); Val (−1.5); Leu (−1.8); Ile (−1.8); Tyr (−2.3); Phe (−2.5); Trp (−3.4). It is understood that an amino acid can be substituted for another having a similar hydrophilicity value and still obtain a biologically equivalent, and in particular, an immunologically equivalent protein. In such changes, the substitution of amino acids whose hydrophilicity values are within ±2 is preferred, those within ±1 are particularly preferred, and those within ±0.5 are even more particularly preferred.

As outlined above, amino acid substitutions may be based on the relative similarity of the amino acid side-chain substituents, for example, their hydrophobicity, hydrophilicity, charge, size, and the like.

As indicated elsewhere, variants of gene sequences can include codon optimized variants, sequence polymorphisms, splice variants, and/or mutations that do not affect the function of an encoded product to a statistically-significant degree.

Variants of the protein, nucleic acid, and gene sequences disclosed herein also include sequences with 70% sequence identity, 80% sequence identity, 85% sequence identity, 90% sequence identity, 95% sequence identity, 96% sequence identity, 97% sequence identity, 98% sequence identity, or 99% sequence identity to the protein, nucleic acid, or gene sequences disclosed herein.

“% sequence identity” refers to a relationship between two or more sequences, as determined by comparing the sequences. In the art, “identity” also means the degree of sequence relatedness between protein, nucleic acid, or gene sequences as determined by the match between strings of such sequences. “Identity” (often referred to as “similarity”) can be readily calculated by known methods, including (but not limited to) those described in: Computational Molecular Biology (Lesk, A. M., ed.) Oxford University Press, NY (1988); Biocomputing: Informatics and Genome Projects (Smith, D. W., ed.) Academic Press, NY (1994); Computer Analysis of Sequence Data, Part I (Griffin, A. M., and Griffin, H. G., eds.) Humana Press, NJ (1994); Sequence Analysis in Molecular Biology (Von Heijne, G., ed.) Academic Press (1987); and Sequence Analysis Primer (Gribskov, M. and Devereux, J., eds.) Oxford University Press, NY (1992). Preferred methods to determine identity are designed to give the best match between the sequences tested. Methods to determine identity and similarity are codified in publicly available computer programs. Sequence alignments and percent identity calculations may be performed using the Megalign program of the LASERGENE bioinformatics computing suite (DNASTAR, Inc., Madison, Wisconsin). Multiple alignment of the sequences can also be performed using the Clustal method of alignment (Higgins and Sharp CABIOS, 5, 151-153 (1989) with default parameters (GAP PENALTY=10, GAP LENGTH PENALTY=10). Relevant programs also include the GCG suite of programs (Wisconsin Package Version 9.0, Genetics Computer Group (GCG), Madison, Wisconsin); BLASTP, BLASTN, BLASTX (Altschul, et al., J. Mol. Biol. 215:403-410 (1990); DNASTAR (DNASTAR, Inc., Madison, Wisconsin); and the FASTA program incorporating the Smith-Waterman algorithm (Pearson, Comput. Methods Genome Res., [Proc. Int. Symp.](1994), Meeting Date 1992, 111-20. Editor(s): Suhai, Sandor. Publisher: Plenum, New York, N.Y. Within the context of this disclosure it will be understood that where sequence analysis software is used for analysis, the results of the analysis are based on the “default values” of the program referenced. As used herein “default values” will mean any set of values or parameters, which originally load with the software when first initialized.

Variants also include nucleic acid molecules that hybridizes under stringent hybridization conditions to a sequence disclosed herein and provide the same function as the reference sequence. Exemplary stringent hybridization conditions include an overnight incubation at 42° C. in a solution including 50% formamide, 5×SSC (750 mM NaCl, 75 mM trisodium citrate), 50 mM sodium phosphate (pH 7.6), 5×Denhardt's solution, 10% dextran sulfate, and 20 μg/ml denatured, sheared salmon sperm DNA, followed by washing the filters in 0.1×SSC at 50° C. Changes in the stringency of hybridization and signal detection are primarily accomplished through the manipulation of formamide concentration (lower percentages of formamide result in lowered stringency); salt conditions, or temperature. For example, moderately high stringency conditions include an overnight incubation at 37° C. in a solution including 6×SSPE (20×SSPE=3M NaCl; 0.2M NaH2PO4; 0.02M EDTA, pH 7.4), 0.5% SDS, 30% formamide, 100 μg/ml salmon sperm blocking DNA; followed by washes at 50° C. with 1×SSPE, 0.1% SDS. In addition, to achieve even lower stringency, washes performed following stringent hybridization can be done at higher salt concentrations (e.g. 5×SSC). Variations in the above conditions may be accomplished through the inclusion and/or substitution of alternate blocking reagents used to suppress background in hybridization experiments. Typical blocking reagents include Denhardt's reagent, BLOTTO, heparin, denatured salmon sperm DNA, and commercially available proprietary formulations. The inclusion of specific blocking reagents may require modification of the hybridization conditions described above, due to problems with compatibility.

“Specifically binds” refers to an association of an antibody binding domain to its cognate antigen with an affinity or Kₐ (i.e., an equilibrium association constant of a particular binding interaction with units of 1/M) equal to or greater than 10⁵ M⁻¹, while not significantly associating with any other molecules or components in a relevant environment sample. Binding domains may be classified as “high affinity” or “low affinity”. In particular embodiments, “high affinity” binding domains refer to those binding domains with a Kₐ of at least 10⁷ M⁻¹, at least 10⁸ M⁻¹, at least 10⁹ M⁻¹, at least 10¹⁰ M⁻¹, at least 10¹¹ M⁻¹, at least 10¹² M⁻¹, or at least 10¹³ M⁻¹. In particular embodiments, “low affinity” binding domains refer to those binding domains with a Kₐ of up to 107 M⁻¹, up to 10⁶ M⁻¹, up to 105 M⁻¹. Alternatively, affinity may be defined as an equilibrium dissociation constant (K_{d}) of a particular binding interaction with units of M (e.g., 10-5 M to 10⁻¹³ M). In certain embodiments, a binding domain may have “enhanced affinity,” which refers to a selected or engineered binding domains with stronger binding to a cognate binding molecule than a wild type (or parent) binding domain. For example, enhanced affinity may be due to a Kₐ (equilibrium association constant) for the cognate binding molecule that is higher than the reference binding domain or due to a K_{d} (dissociation constant) for the cognate binding molecule that is less than that of the reference binding domain, or due to an off-rate (K_{off}) for the cognate binding molecule that is less than that of the reference binding domain. A variety of assays are known for detecting binding domains that specifically bind a particular cognate binding molecule as well as determining binding affinities, such as Western blot, ELISA, and BIACORE® analysis (see also, e.g., Scatchard, et al., 1949, Ann. N.Y. Acad. Sci. 51:660; and U.S. Pat. Nos. 5,283,173, 5,468,614, or the equivalent).

In particular embodiments, Kd can be characterized using BIAcore. For example, in particular embodiments, Kd can be measured using surface plasmon resonance assays using a BIACORE®-2000 or a BIACORE®-3000 (BIAcore, Inc., Piscataway, N.J.) at 25° C. with immobilized antigen CM5 chips at 10 response units (RU). Briefly, carboxymethylated dextran biosensor chips (CM5, BIACORE, Inc.) can be activated with N-ethyl-N′-(3-dimethylaminopropyl)-carbodiimide hydrochloride (EDC) and N-hydroxysuccinimide (NHS) according to the supplier's instructions. Antigen can be diluted with 10 mM sodium acetate, pH 4.8, to 5 μg/ml (0.2 μM) before injection at a flow rate of 5 μl/minute to achieve 10 response units (RU) of coupled protein. Following the injection of antigen, 1 M ethanolamine can be injected to block unreacted groups. For kinetics measurements, two-fold serial dilutions of Fab (0.78 nM to 500 nM) are injected in PBS with 0.05% polysorbate 20 (TWEEN-20™) surfactant (PBST) at 25° C. at a flow rate of 25 μl/min. Association rates (kon) and dissociation rates (k_{off}) can be calculated using a simple one-to-one Langmuir binding model (BIACORE® Evaluation Software version 3.2) by simultaneously fitting the association and dissociation sensorgrams. The equilibrium dissociation constant (Kd) can be calculated as the ratio k_{off}/kₒₙ. See, e.g., Chen et al., J. Mol. Biol. 293:865-881, 1999. If the on-rate exceeds 10⁶ M⁻¹ s⁻¹ by the surface plasmon resonance assay above, then the on-rate can be determined by using a fluorescent quenching technique that measures the increase or decrease in fluorescence emission intensity (excitation=295 nm; emission=340 nm, 16 nm band-pass) at 25° C. of a 20 nM anti-antigen antibody (Fab form) in PBS, pH 7.2, in the presence of increasing concentrations of antigen as measured in a spectrometer, such as a stop-flow equipped spectrophometer (Aviv Instruments) or a 8000-series SLM-AMINCO™ spectrophotometer (ThermoSpectronic) with a stirred cuvette.

As will be understood by one of ordinary skill in the art, each embodiment disclosed herein can comprise, consist essentially of or consist of its particular stated element, step, ingredient or component. Thus, the terms “include” or “including” should be interpreted to recite: “comprise, consist of, or consist essentially of.” The transition term “comprise” or “comprises” means has, but is not limited to, and allows for the inclusion of unspecified elements, steps, ingredients, or components, even in major amounts. The transitional phrase “consisting of” excludes any element, step, ingredient or component not specified. The transition phrase “consisting essentially of” limits the scope of the embodiment to the specified elements, steps, ingredients or components and to those that do not materially affect the embodiment. A material effect would cause a statistically significant reduction the ability to co-express an antibody and a gene product from a genetically modified B cell.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. When further clarity is required, the term “about” has the meaning reasonably ascribed to it by a person skilled in the art when used in conjunction with a stated numerical value or range, i.e. denoting somewhat more or somewhat less than the stated value or range, to within a range of ±20% of the stated value; ±19% of the stated value; ±18% of the stated value; ±17% of the stated value; ±16% of the stated value; ±15% of the stated value; ±14% of the stated value; ±13% of the stated value; ±12% of the stated value; ±11% of the stated value; ±10% of the stated value; ±9% of the stated value; ±8% of the stated value; ±7% of the stated value; ±6% of the stated value; ±5% of the stated value; ±4% of the stated value; ±3% of the stated value; ±2% of the stated value; or ±1% of the stated value.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to patents, printed publications, journal articles and other written text throughout this specification (referenced materials herein). Each of the referenced materials are individually incorporated herein by reference in their entirety for their referenced teaching.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

The particulars shown herein are by way of example and for purposes of illustrative discussion of the preferred embodiments of the present invention only and are presented in the cause of providing what is believed to be the most useful and readily understood description of the principles and conceptual aspects of various embodiments of the invention. In this regard, no attempt is made to show structural details of the invention in more detail than is necessary for the fundamental understanding of the invention, the description taken with the drawings and/or examples making apparent to those skilled in the art how the several forms of the invention may be embodied in practice.

Definitions and explanations used in the present disclosure are meant and intended to be controlling in any future construction unless clearly and unambiguously modified in the examples or when application of the meaning renders any construction meaningless or essentially meaningless. In cases where the construction of the term would render it meaningless or essentially meaningless, the definition should be taken from Webster's Dictionary, 3rd Edition or a dictionary known to those of ordinary skill in the art, such as the Oxford Dictionary of Biochemistry and Molecular Biology (Eds. Attwood T et al., Oxford University Press, Oxford, 2006).

What is claimed is:

1. A B cell comprising a first genetic construct encoding an anti-poliovirus antibody at
a) an intronic region upstream or downstream of an Eμ enhancer within the native antibody heavy chain gene at a site targeted by a gRNA sequence as set forth in SEQ ID NO: 145 and a second genetic construct encoding alpha-galactosidase A upstream or downstream of an iE_{κ} enhancer within the native antibody light chain gene at a site targeted by a gRNA sequence as set forth in SEQ ID NO: 221; or
b) an intronic region upstream or downstream of an Eμ enhancer within the native antibody heavy chain gene at a site targeted by a gRNA sequence as set forth in SEQ ID NO: 181 and a second genetic construct encoding alpha-galactosidase A upstream or downstream of an iE_{κ} enhancer within the native antibody light chain gene at a site targeted by a gRNA sequence as set forth in SEQ ID NO: 241.

2. A B cell comprising a first genetic construct at a first particular area within the native antibody heavy chain gene of the B cell's genome and a second genetic construct at a second particular area within the native antibody light chain gene of the B cell's genome.

3. The B cell of claim 2, wherein the first genetic construct encodes a selected antibody.

4. The B cell of claim 2, wherein the first genetic construct comprises a first promoter, a sequence encoding a signal peptide, a sequence encoding the variable light chain and constant light chain of the selected antibody, a sequence encoding a flexible linker or a skipping element, a sequence encoding the variable region of the heavy chain of the selected antibody, and/or a splice junction.

5. The B cell of claim 3 or 4, wherein the selected antibody comprises an anti-smallpox antibody or an anti-poliovirus antibody.

6. The B cell of claim 4, wherein the variable light chain of the selected antibody comprises the sequence as set forth in SEQ ID NO: 316, SEQ ID NO: 324, SEQ ID NO: 332, or SEQ ID NO: 428 or a sequence having at least 90% sequence identity to SEQ ID NO: 316, SEQ ID NO: 324, SEQ ID NO: 332, or SEQ ID NO: 428.

7. The B cell of claim 4, wherein the sequence encoding the variable light chain of the selected antibody comprises the sequence as set forth in SEQ ID NO: 419 or a sequence having at least 90% sequence identity to SEQ ID NO: 419.

8. The B cell of claim 4, wherein the variable light chain of the selected antibody comprises a variable light chain sequence having complementarity determining regions (CDRs) comprising a CDRL1 having the sequence as set forth in SEQ ID NO: 320, a CDRL2 having the sequence as set forth in SEQ ID NO: 321, and a CDRL3 having the sequence as set forth in SEQ ID NO: 322; a CDRL1 having the sequence as set forth in SEQ ID NO: 328, a CDRL2 having the sequence as set forth in SEQ ID NO: 329, and a CDRL3 having the sequence as set forth in SEQ ID NO: 330; or a CDRL1 having the sequence as set forth in SEQ ID NO: 336, a CDRL2 having the sequence as set forth in SEQ ID NO: 337, and a CDRL3 having the sequence as set forth in SEQ ID NO: 338.

9. The B cell of claim 4, wherein the sequence encoding the variable light chain of the selected antibody comprises a variable light chain sequence having complementarity determining regions (CDRs) comprising a CDRL1 having the sequence as set forth in SEQ ID NO: 423, a CDRL2 having the sequence as set forth in SEQ ID NO: 424, and a CDRL3 having the sequence as set forth in SEQ ID NO: 425.

10. The B cell of claim 4, wherein the variable region of the heavy chain of the selected antibody comprises the sequence as set forth in SEQ ID NO: 315, SEQ ID NO: 323, SEQ ID NO: 331, or SEQ ID NO: 427 or a sequence having at least 90% sequence identity to SEQ ID NO: 315, SEQ ID NO: 323, SEQ ID NO: 331, or SEQ ID NO: 427.

11. The B cell of claim 4, wherein the sequence encoding the variable region of the heavy chain of the selected antibody comprises the sequence as set forth in SEQ ID NO: 418 or a sequence having at least 90% sequence identity to SEQ ID NO: 418.

12. The B cell of claim 4, wherein the variable region of the heavy chain of the selected antibody comprises a variable heavy chain sequence having complementarity determining regions (CDRs) comprising a CDRH1 having the sequence as set forth in SEQ ID NO: 317, a CDRH2 having the sequence as set forth in SEQ ID NO: 318, and a CDRH3 having the sequence as set forth in SEQ ID NO: 319; a CDRH1 having the sequence as set forth in SEQ ID NO: 325, a CDRH2 having the sequence as set forth in SEQ ID NO: 326, and a CDRH3 having the sequence as set forth in SEQ ID NO: 327; or a CDRH1 having the sequence as set forth in SEQ ID NO: 333, a CDRH2 (SEQ ID NO: 334, and a CDRH3 having the sequence as set forth in SEQ ID NO: 335.

13. The B cell of claim 4, wherein the sequence encoding the variable region of the heavy chain of the selected antibody comprises a variable heavy chain sequence having complementarity determining regions (CDRs) comprising a CDRH1 having the sequence as set forth in SEQ ID NO: 420, a CDRH2 having the sequence as set forth in SEQ ID NO: 421, and a CDRH3 having the sequence as set forth in SEQ ID NO: 422.

14. The B cell of claim 4, wherein the first promoter is a heavy chain (VH) promoter.

15. The B cell of claim 4, wherein the flexible linker is between the variable light chain of the selected antibody and the variable region of the heavy chain of the selected antibody.

16. The B cell of claim 4, wherein the flexible linker is a Gly-Ser linker.

17. The B cell of claim 16, wherein the Gly-Ser linker comprises (GGS)n (SEQ ID NO: 343), (GGGS)n (SEQ ID NO: 344), or (GGGGS)n (SEQ ID NO: 345).

18. The B cell of claim 4, wherein the flexible linker comprises the sequence as set forth in SEQ ID NO: 347 or a sequence having at least 90% sequence identity to SEQ ID NO: 347.

19. The B cell of claim 4, wherein the flexible linker is encoded by the sequence as set forth in SEQ ID NO: 346 or a sequence having at least 90% sequence identity to SEQ ID NO: 346.

20. The B cell of claim 2, wherein the first genetic construct further encodes a suicide switch.

21. The B cell of claim 20, wherein the suicide switch comprises an iCaspase 9 construct (iCasp9).

22. The B cell of claim 2, wherein the first genetic construct further comprises a sequence encoding a tag.

23. The B cell of claim 22, wherein the tag comprises the sequence as set forth in SEQ ID NO: 365, SEQ ID NO: 366, SEQ ID NO: 367, SEQ ID NO: 368, SEQ ID NO: 369, SEQ ID NO: 370, SEQ ID NO: 371, SEQ ID NO: 372, SEQ ID NO: 373, or SEQ ID NO: 374 or a sequence having at least 90% sequence identity to SEQ ID NO: 365, SEQ ID NO: 366, SEQ ID NO: 367, SEQ ID NO: 368, SEQ ID NO: 369, SEQ ID NO: 370, SEQ ID NO: 371, SEQ ID NO: 372, SEQ ID NO: 373, or SEQ ID NO: 374.

24. The B cell of claim 4, wherein the skipping element comprises a self-cleaving peptide.

25. The B cell of claim 24, wherein the self-cleaving peptide comprises a T2A self-cleaving peptide, a P2A self-cleaving peptide, an E2A self-cleaving peptide, or an F2A self-cleaving peptide.

26. The B cell of claim 2, wherein the first genetic construct comprises the sequence as set forth in SEQ ID NO: 310 or SEQ ID NO: 311 or a sequence having at least 90% sequence identity to SEQ ID NO: 310 or SEQ ID NO: 311.

27. The B cell of claim 2, wherein the first particular area is an intronic region upstream or downstream of an Eμ enhancer within the native antibody heavy chain gene.

28. The B cell of claim 27, wherein the Eμ enhancer comprises the sequence set forth in SEQ ID NO: 10 or SEQ ID NO: 11.

29. The B cell of claim 2, wherein the first particular area comprises the sequence as set forth in SEQ ID NO: 14, SEQ ID NO: 15, SEQ ID NO: 16, or SEQ ID NO: 17.

30. The B cell of claim 2, wherein the first particular area comprises a gRNA target site comprising the sequence as set forth in one of SEQ ID NOs: 20-99.

31. The B cell of claim 30, wherein a gRNA sequence targeting the gRNA target site comprises the sequence as set forth in one of SEQ ID NOs: 140-219.

32. The B cell of claim 2, wherein the first genetic construct further comprises an upstream homology arm and a downstream homology arm.

33. The B cell of claim 32, wherein the upstream homology arm of the first genetic construct comprises a sequence as set forth in SEQ ID NO: 260, SEQ ID NO: 262, or SEQ ID NO: 264 or a sequence having at least 90% sequence identity to SEQ ID NO: 260 SEQ ID NO: 262, or SEQ ID NO: 264.

34. The B cell of claim 32, wherein the downstream homology arm of the first genetic construct comprises a sequence as set forth in SEQ ID NO: 261, SEQ ID NO: 263, or SEQ ID NO: 265 or a sequence having at least 90% sequence identity to SEQ ID NO: 261, SEQ ID NO: 263, or SEQ ID NO: 265.

35. The B cell of claim 2, wherein the second genetic construct comprises a second promoter and encodes a gene product.

36. The B cell of claim 35, wherein the gene product comprises a secreted protein, inhibitory nucleic acid molecule, or non-secreted protein.

37. The B cell of claim 36, wherein the secreted protein comprises alpha-galactosidase A, glucocerebrosidase (GC), acid sphingomyelinase, mucopolysaccharides, acid alpha-glucosidase, aspartylglucosaminidase, palmitoyl protein thioesterase, tripeptidyl peptidase, lysosomal transmembrane protein, cysteine transporter, acid ceramidase, acid alpha-L-fucosidase, cathepsin A, acid beta-glucosidase, acid beta-galactosidase, iduronate-2-sulfatase, alpha-L-iduronidase, galactocerebrosidase, acid alpha-mannosidase, acid beta-mannosidase, arylsulfatase B, arylsulfatase A, N-acetylgalactosamine-6-sulfate, N-acetylglucosamine-1-phosphotransferase, acid sphingomyelinase, NPC-1, alpha-glucosidase, beta-hexosaminidase B, heparan N-sulfatase, alpha-N-acetylglucosaminidase, acetyl-CoA: alpha-glucosaminide, N-acetylglucosamine-6-sulfate, alpha-N-acetylgalactosaminidase, alpha-neuramidase, beta-glucuronidase, beta-hexosaminidase A, or acid lipase.

38. The B cell of claim 36, wherein the secreted protein comprises factor VIII, factor IX, factor XI.

39. The B cell of claim 36, wherein the secreted protein comprises insulin, alpha-1 antitrypsin, lactase, glucose-6-phosphate dehydrogenase (G6PD), pyruvate kinase, glucocerebrosidase, ATPase7B, galactose-1-phosphate uridyl transferase (GALT), branched-chain α-ketoacid dehydrogenase (BCKD) complex, phenylalanine hydroxylase (PAH), glucose-6-phosphatase (G6Pase), debranching enzyme, glycogen-branching enzyme, glutaryl-CoA dehydrogenase, frataxin, peroxisome biogenesis disorders (PBDs) or peroxisomes, 5α-reductase, glucose phosphate isomerase, hexosaminidase A, activin receptor type-1 (ACVR1), pramlinitide acetate, growth hormone (GH), insulin-like growth factor, protein C, α1-proteinase inhibitor, erythropoietin, granulocyte colony-stimulating factor (G-CSF), Interleukin 11, human follicle-stimulating hormone (FSH), human chorionic gonadotropin (HCG), Lutropin-α, Interleukin 2 (IL2), Denileukin diftitox (fusion of IL2 and Diphtheria toxin), Interferon-α2a, Interferon-α2b, Interferon-αn3, Interferon-β1a, Interferon-β1b, Interferon-γ1b, human parathyroid hormone, glucagon-like peptide 1, somatostatin, bone morphogenic protein 2, bone morphogenic protein 7, gonadotropin-releasing hormone (GnRH), keratinocyte growth factor (KGF), platelet-derived growth factor (PDGF), B-type natriuretic peptide, or hirudin.

40. The B cell of claim 36, wherein the secreted protein comprises interleukin 2.

41. The B cell of claim 36, wherein the secreted protein comprises a fluorescent protein.

42. The B cell of claim 41, wherein the fluorescent protein comprises mCherry or green fluorescent protein (GFP).

43. The B cell of claim 35, wherein the second promoter comprises a CMV promoter, an MND promoter, or a Vk21E promoter.

44. The B cell of claim 2, wherein the second genetic construct further encodes a suicide switch.

45. The B cell of claim 44, wherein the suicide switch comprises an iCaspase 9 construct (iCasp9).

46. The B cell of claim 2, wherein the second genetic construct further comprises a tag.

47. The B cell of claim 46, wherein the tag comprises the sequence as set forth in SEQ ID NO: 365, SEQ ID NO: 366, SEQ ID NO: 367, SEQ ID NO: 368, SEQ ID NO: 369, SEQ ID NO: 370, SEQ ID NO: 371, SEQ ID NO: 372, SEQ ID NO: 373, or SEQ ID NO: 374 or a sequence having at least 90% sequence identity to SEQ ID NO: 365, SEQ ID NO: 366, SEQ ID NO: 367, SEQ ID NO: 368, SEQ ID NO: 369, SEQ ID NO: 370, SEQ ID NO: 371, SEQ ID NO: 372, SEQ ID NO: 373, or SEQ ID NO: 374.

48. The B cell of claim 2, wherein the second genetic construct comprises the sequence as set forth in SEQ ID NO: 293, SEQ ID NO: 294, SEQ ID NO: 295, SEQ ID NO: 296, SEQ ID NO: 297, SEQ ID NO: 298, SEQ ID NO: 299, SEQ ID NO: 300, SEQ ID NO: 301, SEQ ID NO: 302, SEQ ID NO: 303, SEQ ID NO: 304, SEQ ID NO: 305, SEQ ID NO: 306, SEQ ID NO: 307, SEQ ID NO: 308, or SEQ ID NO: 309 or a sequence having at least 90% sequence identity to SEQ ID NO: 293, SEQ ID NO: 294, SEQ ID NO: 295, SEQ ID NO: 296, SEQ ID NO: 297, SEQ ID NO: 298, SEQ ID NO: 299, SEQ ID NO: 300, SEQ ID NO: 301, SEQ ID NO: 302, SEQ ID NO: 303, SEQ ID NO: 304, SEQ ID NO: 305, SEQ ID NO: 306, SEQ ID NO: 307, SEQ ID NO: 308, or SEQ ID NO: 309.

49. The B cell of claim 2, wherein the second particular area is upstream or downstream of an iEκ enhancer within the native antibody light chain gene.

50. The B cell of claim 49, wherein the iEκ enhancer comprises the sequence as set forth in SEQ ID NO: 12 or SEQ ID NO: 13.

51. The B cell of claim 2, wherein the second particular area comprises the sequence as set forth in SEQ ID NO: 18 or SEQ ID NO: 19.

52. The B cell of claim 2, wherein the second particular area comprises a gRNA target site comprising the sequence as set forth in one of SEQ ID NOs: 100-139.

53. The B cell of claim 52, wherein a gRNA sequence targeting the gRNA target site comprises the sequence as set forth in one of SEQ ID NOs: 220-259.

54. The B cell of claim 2, wherein the second genetic construct further comprises an upstream homology arm and a downstream homology arm.

55. The B cell of claim 54, wherein the upstream homology arm of the second genetic construct comprises a sequence as set forth in SEQ ID NO: 266, SEQ ID NO: 268, SEQ ID NO: 270, SEQ ID NO: 272, SEQ ID NO: 274, or SEQ ID NO: 276 or a sequence having at least 90% sequence identity to SEQ ID NO: 266, SEQ ID NO: 268, SEQ ID NO: 270, SEQ ID NO: 272, SEQ ID NO: 274, or SEQ ID NO: 276.

56. The B cell of claim 54, wherein the downstream homology arm of the second genetic construct comprises a sequence as set forth in SEQ ID NO: 267, SEQ ID NO: 269, SEQ ID NO: 271, SEQ ID NO: 273, SEQ ID NO: 275, or SEQ ID NO: 277 or a sequence having at least 90% sequence identity to SEQ ID NO: 267, SEQ ID NO: 269, SEQ ID NO: 271, SEQ ID NO: 273, SEQ ID NO: 275, or SEQ ID NO: 277.

57. The B cell of claim 2, wherein the B cell is used to treat a subject in need thereof.

58. A formulation comprising the B cell of claim 2 and a pharmaceutically acceptable carrier.

59. A kit for genetically modifying a B cell, the kit comprising a first genetic construct encoding a selected antibody, a second genetic construct encoding a gene product, a first gRNA targeting an intronic region upstream or downstream of an Eμ enhancer within the native antibody heavy chain gene of the B cell, and a second gRNA targeting a region upstream or downstream of an iEκ enhancer within the native antibody light chain gene.

60. The kit of claim 59, wherein the first gRNA comprises SEQ ID NO: 181, SEQ ID NO: 142, or SEQ ID NO: 145 or a sequence having at least 90% sequence identity to SEQ ID NO: 181, SEQ ID NO: 142, or SEQ ID NO: 145.

61. The kit of claim 59, wherein the first gRNA comprises one of SEQ ID NOs: 140-219 or a sequence having at least 90% sequence identity to SEQ ID NOs: 140-219.

62. The kit of claim 60, wherein the first gRNA is sgRNA.

63. The kit of claim 59, wherein the first gRNA targets one of SEQ ID NOs: 20-99.

64. The kit of claim 59, wherein the second gRNA comprises the sequence as set forth in SEQ ID NO: 225, SEQ ID NO: 224, SEQ ID NO: 221, SEQ ID NO: 244, SEQ ID NO: 242, or SEQ ID NO: 241 or a sequence having at least 90% sequence identity to SEQ ID NO: 225, SEQ ID NO: 224, SEQ ID NO: 221, SEQ ID NO: 244, SEQ ID NO: 242, or SEQ ID NO: 241.

65. The kit of claim 59, wherein the second gRNA comprises one of SEQ ID NOs: 220-259 or a sequence having at least 90% sequence identity to SEQ ID NOs: 220-259.

66. The kit of claim 64, wherein the second gRNA is sgRNA.

67. The kit of claim 59, wherein the second gRNA targets one of SEQ ID NOs: 100-139.

68. The kit of claim 59, wherein the selected antibody comprises a smallpox virus antibody or a poliovirus antibody.

69. The kit of claim 59, wherein the selected antibody binds a synthetic antigen.

70. The kit of claim 59, further comprising an antigen that binds the selected antibody.

71. The kit of claim 70, wherein the antigen is a rare antigen.

72. The kit of claim 70, wherein the antigen comprises a smallpox virus antigen or a poliovirus antigen.

73. The kit of claim 70, wherein the antigen is a synthetic antigen.

74. The kit of claim 73, wherein the synthetic antigen comprises R-phycoerythrin or allophycocyanin.

75. The kit of claim 59, wherein the first genetic construct further comprises a first promoter.

76. The kit of claim 75, wherein the first promoter comprises a VH promoter.

77. The kit of claim 59, wherein the first genetic construct further comprises a sequence encoding a tag.

78. The kit of claim 77, wherein the tag comprises the sequence as set forth in SEQ ID NO: 365, SEQ ID NO: 366, SEQ ID NO: 367, SEQ ID NO: 368, SEQ ID NO: 369, SEQ ID NO: 370, SEQ ID NO: 371, SEQ ID NO: 372, SEQ ID NO: 373, or SEQ ID NO: 374 or a sequence having at least 90% sequence identity to SEQ ID NO: 365, SEQ ID NO: 366, SEQ ID NO: 367, SEQ ID NO: 368, SEQ ID NO: 369, SEQ ID NO: 370, SEQ ID NO: 371, SEQ ID NO: 372, SEQ ID NO: 373, or SEQ ID NO: 374.

79. The kit of claim 59, wherein the first genetic construct further comprises a skipping element, homology regions, and/or a splice junction.

80. The kit of claim 59, wherein the first genetic construct further encodes a suicide switch.

81. The kit of claim 80, wherein the suicide switch comprises an iCaspase 9 construct (iCasp9).

82. The kit of claim 59, wherein the first genetic construct comprises the sequence as set forth in SEQ ID NO: 310 or SEQ ID NO: 311 or a sequence having at least 90% sequence identity to SEQ ID NO: 310 or SEQ ID NO: 311.

83. The kit of claim 59, wherein the gene product is a secreted protein, non-secreted protein, or inhibitory nucleic acid molecule.

84. The kit of claim 83, wherein the secreted protein comprises glucocerebrosidase (GC), acid sphingomyelinase, mucopolysaccharides, acid alpha-glucosidase, aspartylglucosaminidase, alpha-galactosidase A, palmitoyl protein thioesterase, tripeptidyl peptidase, lysosomal transmembrane protein, cysteine transporter, acid ceramidase, acid alpha-L-fucosidase, cathepsin A, acid beta-glucosidase, acid beta-galactosidase, iduronate-2-sulfatase, alpha-L-iduronidase, galactocerebrosidase, acid alpha-mannosidase, acid beta-mannosidase, arylsulfatase B, arylsulfatase A, N-acetylgalactosamine-6-sulfate, N-acetylglucosamine-1-phosphotransferase, acid sphingomyelinase, NPC-1, alpha-glucosidase, beta-hexosaminidase B, heparan N-sulfatase, alpha-N-acetylglucosaminidase, acetyl-CoA: alpha-glucosaminide, N-acetylglucosamine-6-sulfate, alpha-N-acetylgalactosaminidase, alpha-neuramidase, beta-glucuronidase, beta-hexosaminidase A, or acid lipase.

85. The kit of claim 83, wherein the secreted protein comprises factor VIII, factor IX, or factor XI.

86. The kit of claim 83, wherein the secreted protein comprises insulin, alpha-1 antitrypsin, lactase, glucose-6-phosphate dehydrogenase (G6PD), pyruvate kinase, glucocerebrosidase, ATPase7B, galactose-1-phosphate uridyl transferase (GALT), branched-chain α-ketoacid dehydrogenase (BCKD) complex, phenylalanine hydroxylase (PAH), glucose-6-phosphatase (G6Pase), debranching enzyme, glycogen-branching enzyme, glutaryl-CoA dehydrogenase, frataxin, peroxisome biogenesis disorders (PBDs) or peroxisomes, 5α-reductase, glucose phosphate isomerase, hexosaminidase A, activin receptor type-1 (ACVR1), pramlinitide acetate, growth hormone (GH), insulin-like growth factor, protein C, α1-proteinase inhibitor, erythropoietin, granulocyte colony-stimulating factor (G-CSF), Interleukin 11, human follicle-stimulating hormone (FSH), human chorionic gonadotropin (HCG), Lutropin-α, Interleukin 2 (IL2), Denileukin diftitox (fusion of IL2 and Diphtheria toxin), Interferon-α2a, Interferon-α2b, Interferon-αn3, Interferon-β1a, Interferon-β1b, Interferon-γ1b, human parathyroid hormone, glucagon-like peptide 1, somatostatin, bone morphogenic protein 2, bone morphogenic protein 7, gonadotropin-releasing hormone (GnRH), keratinocyte growth factor (KGF), platelet-derived growth factor (PDGF), B-type natriuretic peptide, or hirudin.

87. The kit of claim 83, wherein the secreted protein comprises interleukin 2.

88. The kit of claim 83, wherein the secreted protein comprises a fluorescent protein.

89. The kit of claim 88, wherein the fluorescent protein comprises mCherry or green fluorescent protein (GFP).

90. The kit of claim 59, wherein the second genetic construct further comprises a second promoter.

91. The kit of claim 90, wherein the second promoter comprises a CMV promoter, an MND promoter, or a Vk21E promoter.

92. The kit of claim 59, wherein the second genetic construct further comprises a sequence encoding a tag.

93. The kit of claim 92, wherein the tag comprises the sequence as set forth in SEQ ID NO: 365, SEQ ID NO: 366, SEQ ID NO: 367, SEQ ID NO: 368, SEQ ID NO: 369, SEQ ID NO: 370, SEQ ID NO: 371, SEQ ID NO: 372, SEQ ID NO: 373, or SEQ ID NO: 374 or a sequence having at least 90% sequence identity to SEQ ID NO: 365, SEQ ID NO: 366, SEQ ID NO: 367, SEQ ID NO: 368, SEQ ID NO: 369, SEQ ID NO: 370, SEQ ID NO: 371, SEQ ID NO: 372, SEQ ID NO: 373, or SEQ ID NO: 374.

94. The kit of claim 59, wherein the second genetic construct further encodes a suicide switch.

95. The kit of claim 94, wherein the suicide switch comprises an iCasp9 construct.

96. The kit of claim 59, further comprising a suicide switch activating agent.

97. The kit of claim 59, further comprising a nuclease.

98. The kit of claim 97, wherein the nuclease is Cas9 or Cpf1.

99. The kit of claim 59, wherein the second genetic construct comprises the sequence as set forth in SEQ ID NO: 293, SEQ ID NO: 294, SEQ ID NO: 295, SEQ ID NO: 296, SEQ ID NO: 297, SEQ ID NO: 298, SEQ ID NO: 299, SEQ ID NO: 300, SEQ ID NO: 301, SEQ ID NO: 302, SEQ ID NO: 303, SEQ ID NO: 304, SEQ ID NO: 305, SEQ ID NO: 306, SEQ ID NO: 307, SEQ ID NO: 308, or SEQ ID NO: 309 or a sequence having at least 90% sequence identity to SEQ ID NO: 293, SEQ ID NO: 294, SEQ ID NO: 295, SEQ ID NO: 296, SEQ ID NO: 297, SEQ ID NO: 298, SEQ ID NO: 299, SEQ ID NO: 300, SEQ ID NO: 301, SEQ ID NO: 302, SEQ ID NO: 303, SEQ ID NO: 304, SEQ ID NO: 305, SEQ ID NO: 306, SEQ ID NO: 307, SEQ ID NO: 308, or SEQ ID NO: 309.

100. The kit of claim 59, further comprising a nanoparticle or adeno-associated viral vector.

101. The kit of claim 100, wherein the gRNA and nuclease are associated with a nanoparticle.

102. The kit of claim 100, wherein the nanoparticle is a B-cell targeted nanoparticle.

103. The kit of claim 59, wherein the first genetic construct and second genetic construct are part of an adeno-associated viral vector.

104. The kit of claim 59, wherein the genetic modification is performed in vivo or ex vivo.

105. A method of genetically modifying a B cell comprising introducing into the B cell
a first targeting element, a first cutting element, and a first genetic construct encoding a selected antibody and
a second targeting element, a second cutting element, and a second genetic construct encoding a gene product
wherein
the first targeting element and the first cutting element result in insertion of the first genetic construct at a first particular area within the native antibody heavy chain gene of the B cell's genome and
the second targeting element and the second cutting element result in insertion of the second genetic construct at a second particular area within the native antibody light chain gene of the B cell's genome.

106. The method of claim 105, wherein the first targeting element targets a first particular area comprising an intronic region upstream or downstream of an Eμ enhancer within the native antibody heavy chain gene.

107. The method of claim 105, wherein the first particular area comprises the sequence as set forth in SEQ ID NO: 14, SEQ ID NO: 15, SEQ ID NO: 16, or SEQ ID NO: 17.

108. The method of claim 105, wherein the first particular area comprises a gRNA target site comprising the sequence as set forth in one of SEQ ID NOs: 20-99.

109. The method of claim 105, wherein the first targeting element comprises an sgRNA having a sequence as set forth in SEQ ID NO: 181, SEQ ID NO: 142, or SEQ ID NO: 145 or a sequence having at least 90% sequence identity to SEQ ID NO: 181, SEQ ID NO: 142, or SEQ ID NO: 145.

110. The method of claim 105, wherein the second targeting element targets a second particular area comprising a region upstream or downstream of an iEκ enhancer within the native antibody light chain gene.

111. The method of claim 105, wherein the second particular area comprises the sequence as set forth in SEQ ID NO: 18 or SEQ ID NO: 19.

112. The method of claim 105, wherein the second particular area comprises a gRNA target site comprising the sequence as set forth in one of SEQ ID NOs: 100-139.

113. The method of claim 105, wherein the second targeting element comprises an sgRNA having a sequence as set forth in SEQ ID NO: 225, SEQ ID NO: 224, SEQ ID NO: 221, SEQ ID NO: 244, SEQ ID NO: 242, or SEQ ID NO: 241 or a sequence having at least 90% sequence identity to SEQ ID NO: 225, SEQ ID NO: 224, SEQ ID NO: 221, SEQ ID NO: 244, SEQ ID NO: 242, or SEQ ID NO: 241.

114. The method of claim 105, wherein the first genetic construct comprises an upstream homology arm and a downstream homology arm.

115. The method of claim 114, wherein the upstream homology arm of the first genetic construct comprises a sequence as set forth in SEQ ID NO: 260 SEQ ID NO: 262, or SEQ ID NO: 264 or a sequence having at least 90% sequence identity to SEQ ID NO: 260 SEQ ID NO: 262, or SEQ ID NO: 264.

116. The method of claim 114, wherein the downstream homology arm of the first genetic construct comprises a sequence as set forth in SEQ ID NO: 261 SEQ ID NO: 263, or SEQ NO: 265 or a sequence having at least 90% sequence identity to SEQ ID NO: 261 SEQ ID NO: 263, or SEQ NO: 265.

117. The method of claim 105, wherein the second expression construct comprises an upstream homology arm and a downstream homology arm.

118. The method of claim 117, wherein the upstream homology arm of the second genetic construct comprises a sequence as set forth in SEQ ID NO:266, SEQ ID NO: 268, SEQ ID NO: 270, SEQ ID NO: 272, SEQ ID NO: 274, or SEQ ID NO: 276 or a sequence having at least 90% sequence identity to SEQ ID NO:266, SEQ ID NO: 268, SEQ ID NO: 270, SEQ ID NO: 272, SEQ ID NO: 274, or SEQ ID NO: 276.

119. The method of claim 117, wherein the downstream homology arm of the second genetic construct comprises a sequence as set forth in SEQ ID NO: 267, SEQ ID NO: 269, SEQ ID NO: 271, SEQ ID NO: 273, SEQ ID NO: 275, or SEQ ID NO: 277 or a sequence having at least 90% sequence identity to SEQ ID NO: 267, SEQ ID NO: 269, SEQ ID NO: 271, SEQ ID NO: 273, SEQ ID NO: 275, or SEQ ID NO: 277.

120. The method of claim 105, wherein the introducing into the B cell is ex vivo or in vivo.

121. The method of claim 105, wherein the first targeting element, the first cutting element, and the first genetic construct are associated with a nanoparticle.

122. The method of claim 105, wherein the second targeting element, the second cutting element, and the second genetic construct are associated with a nanoparticle.

123. The method of claim 121 or 122, wherein the nanoparticle is a B-cell targeted nanoparticle.

124. The method of claim 105, further comprising administering an antigen that binds the selected antibody to upregulate expression of the gene product.

125. The method of claim 105, wherein the first genetic construct comprises a first promoter, a sequence encoding a signal peptide, a sequence encoding the variable light chain and constant light chain of the selected antibody, a sequence encoding a flexible linker or a skipping element, a sequence encoding the variable region of the heavy chain of the selected antibody, and/or a splice junction.

126. The method of claim 105, wherein the first genetic construct further encodes a suicide switch.

127. The method of claim 126, wherein the suicide switch comprises an iCaspase 9 construct (iCasp9).

128. The method of claim 126, wherein the suicide switch is activated by a suicide switch activation agent.

129. The method of claim 128, wherein the suicide switch activation agent is rapamycin or an analog thereof.

130. The method of claim 105, wherein the second genetic construct comprises a second promoter and encodes a gene product.

131. The method of claim 105, wherein the second genetic construct further encodes a suicide switch.

132. The method of claim 131, wherein the suicide switch comprises an iCaspase 9 construct (iCasp9).

133. The method of claim 131, wherein the suicide switch is activated by a suicide switch activation agent.

134. The method of claim 133, wherein the suicide switch activation agent is rapamycin or an analog thereof.

135. A method of providing expression of a gene product in a subject comprising administering to the subject a therapeutically effective amount of the B cell of claim 1 and/or a therapeutically effective amount of nanoparticles associated with
a first targeting element, a first cutting element, and a first genetic construct encoding a selected antibody and
a second targeting element, a second cutting element, and a second genetic construct encoding a gene product
wherein
the first targeting element and the first cutting element result in insertion of the first genetic construct at a particular area within the native antibody heavy chain gene of the B cell's genome within the subject and
the second targeting element and the second cutting element result in insertion of the second genetic construct at a particular area within the native antibody light chain gene of the B cell's genome within the subject.

136. The method of claim 135, comprising administering the therapeutically effective amount of the B cell of claim 1 by infusion, injection, perfusion, or lavage.

137. The method of claim 135, comprising administering the therapeutically effective amount of the nanoparticles by infusion, injection, perfusion, or lavage.

138. The method of claim 135, further comprising administering an antigen that binds the selected antibody.

139. The method of claim 138, wherein the administering the antigen is on a dosing schedule.

140. The method of claim 139, wherein the dosing schedule is daily, weekly, monthly, or annually.

141. The method of claim 135, further comprising monitoring the subject for a condition.

142. The method of claim 141, wherein the condition comprises a side effect of a disorder.

143. The method of claim 141, wherein the condition comprises expression levels of the gene product falling below a lower threshold value.

144. The method of claim 135, further comprising administering an antigen that binds the selected antibody upon detection of the condition when expression levels of the gene product fall below a lower threshold.

145. The method of claim 144, wherein the administering of the antigen is through ingestion, injection, or inhalation.

146. The method of claim 141, wherein the condition comprises expression levels of the gene product exceed an upper threshold value.

147. The method of claim 135, wherein the first genetic construct and/or second genetic construct encodes a suicide switch.

148. The method of claim 147, wherein the suicide switch is activated by a suicide switch activation agent.

149. The method of claim 148, wherein the suicide switch activation agent is administered when expression levels of the gene product exceeds an upper threshold.

150. The method of claim 135, wherein the subject has a gene product deficiency.

151. The method of claim 150, wherein the gene product deficiency is a lysosomal storage disease, a clotting disorder, diabetes, or an alpha-1 antitrypsin deficiency.

152. A method of treating a lysosomal storage disease within a subject, the method comprising administering to the subject a therapeutically effective amount of the B cell of claim 1 or 2 thereby treating the lysosomal storage disease within the subject.

153. A method of treating a clotting disorder within a subject, the method comprising administering to the subject a therapeutically effective amount of the B cell of claim 1 or 2 thereby treating the clotting disorder within the subject.

154. A method of treating diabetes within a subject, the method comprising administering to the subject a therapeutically effective amount of the B cell of claim 1 or 2 thereby treating diabetes within the subject.

155. A method of treating an alpha-1 antitrypsin deficiency within a subject, the method comprising administering to the subject a therapeutically effective amount of the B cell of claim 1 or 2 thereby treating the alpha-1 antitrypsin deficiency within the subject.
